(19) United States
(12) Patent Application Publication
(10) Pub. No.: US 2025/0129064 A1
(43) Pub. Date: Apr. 24, 2025

(54) PRODRUGS OF 2-BROMO-LSD (2-BROMOLYSERGIC ACID DIETHYLAMIDE)

(71) Applicant: Terran Biosciences Inc., Miami Beach, FL (US)

(72) Inventors: Matthew DUNCTON, Las Vegas, NV (US); Samuel CLARK, Miami, FL (US)

(21) Appl. No.: 18/832,269

(22) PCT Filed: Jan. 23, 2023
(86) PCT No.: PCT/US2023/061109

§ 371 Date: Jul. 23, 2024

Related U.S. Application Data
(60) Provisional application No. 63/302,513, filed on Jan. 24, 2022.

(51) Int. Cl. C07D 457/04; A61K 31/48; A61K 45/06

(52) U.S. Cl. CPC C07D 457/04; A61K 31/48; A61K 45/06

(57) ABSTRACT

Disclosed herein are 2-bromo-LSD analogs of Formula (I) or a pharmaceutically acceptable salt thereof, as well as methods for making and using the compounds to, for example, treat neuropsychiatric disorders.

### CROSS-REFERENCE TO RELATED APPLICATIONS

This application claims the benefit of U.S. Provisional Application No. 63/302,513, filed Jan. 24, 2022, which is incorporated herein by reference in its entirety.

### FIELD OF THE INVENTION

The present disclosure relates to compounds of Formula (I):

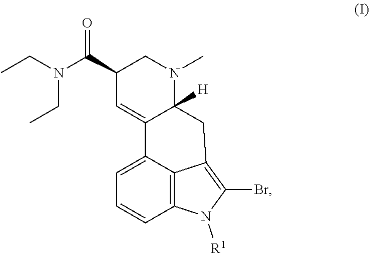

or a pharmaceutically acceptable salt thereof, and methods for their use to treat neuropsychiatric disorders.

### BACKGROUND OF THE INVENTION

Major depressive disorder and related neuropsychiatric diseases are among the leading causes of disability worldwide. Despite recent advances, there remains a need for new therapeutics to support treatment of debilitating neuropsychiatric diseases. Recently, psychedelic compounds have received renewed interest for the treatment of depression and other disorders. For example, the Food and Drug Administration (FDA) recently approved the dissociative anesthetic ketamine for treatment-resistant depression, making it the first mechanistically distinct medicine to be introduced to psychiatry in nearly thirty years. Ketamine is a member of a class of compounds known as psychoplastogens. Psychoplastogens promote neuronal growth through a mechanism involving the activation of AMPA receptors, the tropomyosin receptor kinase B (TrkB), and the mammalian target of rapamycin (mTOR). As pyramidal neurons in the PFC exhibit top-down control over areas of the brain controlling motivation, fear, and reward, these effects support clinical development of psychoplastogenic compounds for their antidepressant, anxiolytic, and anti-addictive effects properties.

Certain compounds from the psychedelic class of compounds have shown promise in therapeutic applications. Disclosed herein are analogs of the psychedelic 2-Br-LSD with improved properties.

### BRIEF SUMMARY OF THE INVENTION

Disclosed herein are analogs of 2-Br-LSD, such as a compound of Formula (I):

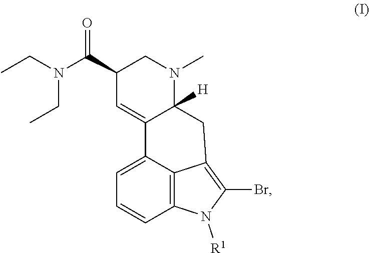

or a pharmaceutically acceptable salt thereof, wherein:
R¹ is —C(O)OR³, —C(O)R⁴, —CH(R⁵)OR⁶, —C(O)OCH(R⁵)OC(O)R⁴, —C(O)OCH(R⁵)OC(O)OR⁴, —C(O)OCH(R⁵)OC(O)NHR⁴, —CH(R⁵)NHC(O)R⁶, —CH(R⁵)C(O)R⁶, —S(O)₂R⁷, —S(O)₂OR⁷, —P(O)OR⁹[N(R⁹)R¹⁰], —C(O)N(R⁹)R¹⁰, —P(O)OR¹¹(OR¹²), —CH(R⁴)OP(O)OR₈[N(R⁹)R¹⁰], —CH(R⁴)OP(O)OR¹¹(OR¹²), —C(O)OCH(R⁵)OP(O)OR²⁰(OR²¹);
each of R³, R⁴, R⁵, R⁶, R⁷, and R⁸ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
or R³ is

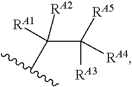

wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶,
or R⁴ is

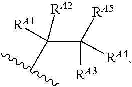

wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.
or R⁴ is

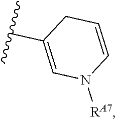

wherein R^{A7} is hydrogen or alkyl;
or R⁴ is —CH(R^{A1})NH₂, wherein R^{A1} is an amino acid side chain;
each of R⁹ and R¹⁰ is independently hydrogen, alkyl, heteroalkyl, haloalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
or R⁹ is

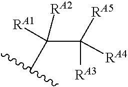

wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶;
each of R¹¹ and R¹² is independently hydrogen, alkyl, heteroalkyl, cycloalkyl, heterocycloalkyl, aryl, heteroaryl, —CH(R⁵)C(O)R¹⁴, —CH(R⁵)C(O)OR¹, —CH(R⁵)OC(O)R¹⁵, or —CH(R⁵)OC(O)OR¹⁶, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A}; each R^{A} is independently alkyl, heteroalkyl, cycloalkyl, heterocycloalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or —S(O)₂R²², wherein alkyl, heteroalkyl, cycloalkyl, heterocycloalkyl, aryl, or heteroaryl is unsubstituted or substituted with alkyl, aryl, halogen, oxo, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
or R¹⁴ or R¹⁶ is

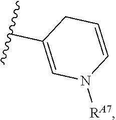

wherein R^{A7} is hydrogen or alkyl;
or R¹⁵ is —CH(R^{A1})NH₂, wherein R^{A1} is an amino acid side chain;
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰, R²¹ and R²² is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B};
each R^{B} is independently halogen, amino, cyano, hydroxyl, alkyl, heteroalkyl, cycloalkyl, heterocycloalkyl, aryl, heteroaryl, arylalkyl, acyl or heteroarylalkyl, wherein cycloalkyl, heterocycloalkyl, acyl, aryl, or heteroaryl is unsubstituted or substituted with one or more halogen, amino, cyano, hydroxyl, alkyl, acetyl, or benzoyl; and
each R^{C} is independently hydrogen or alkyl.

Also disclosed are methods for using the disclosed 2-Br-LSD analogs, such as in a method for treating a neuropsychiatric disorder.

The foregoing and other objects, features, and advantages of the invention will become more apparent from the following detailed description.

### BRIEF DESCRIPTION OF THE DRAWINGS

FIG. 1-A shows mean concentration-time profiles of 2-Br-LSD following IV dosing of 2-Br-LSD (1 mg/kg) to male Sprague Dawley (SD) rats.

FIG. 1-B shows mean concentration-time profiles of 2-Br-LSD following oral dosing of 2-Br-LSD (4 mg/Kg) to male Sprague Dawley (SD) rats.

FIG. 2 shows mean concentration-time profiles of 2-Br-LSD following oral dosing of 2-Br—N-Boc prodrug (4 mg/kg) to male SD rats.

FIG. 3 shows mean concentration-time profiles of 2-Br-LSD following oral dosing of 2-Br-LSD pivalamide prodrug (4 mg/kg) to male SD rats.

FIG. 4 shows mean concentration-time profiles of 2-Br-LSD following oral dosing of 2-Br-LSD oxymethyl pivalate prodrug (4 mg/kg) to male SD rats.

FIG. 5 shows mean concentration-time profiles of 2-Br-LSD following oral dosing of 2-Br-LSD acetamide prodrug (4 mg/kg) to male SD rats.

FIG. 6 shows mean concentration-time profiles of 2-Br-LSD following oral dosing of 2-Br-LSD ethyl carbamate prodrug (4 mg/kg) to male SD rats.

FIG. 7 shows mean concentration-time profiles of 2-Br-LSD following oral dosing of 2-Br-LSD methyl pivalate prodrug (4 mg/kg) to male SD rats.

FIG. 8 shows mean concentration-time profiles of 2-Br-LSD following oral dosing of 2-Br-LSD isopropyl carbamate prodrug (4 mg/kg) to male SD rats.

FIG. 9 shows mean concentration-time profiles of 2-Br-LSD following oral dosing of 2-Br-LSD-N-Boc-Ala prodrug (4 mg/kg) to male SD rats.

FIG. 10 shows mean concentration-time profiles of 2-Br-LSD following oral dosing of 2-Br-LSD N-isobutyramide prodrug (4 mg/kg) to male SD rats.

FIG. 11 shows mean concentration-time profiles of 2-Br-LSD following oral dosing of 2-Br-LSD tetrahydropyran carboxylic acid prodrug (4 mg/kg) to male SD rats.

FIG. 12 shows mean concentration-time profiles of 2-Br-LSD following oral dosing of 2-Br-LSD Boc-Val formate prodrug (4 mg/kg) to male SD rats.

FIG. 13 shows mean concentration-time profiles of 2-Br-LSD following oral dosing of 2-Br-LSD hydroxymethyl prodrug (4 mg/kg) to male SD rats.

FIG. 14 shows mean concentration-time profiles of 2-Br-LSD following oral dosing of 2-Br-LSD methyl ethyl carbonate prodrug (4 mg/kg) to male SD rats.

FIG. 15 shows mean concentration-time profiles of 2-Br-LSD following oral dosing of 2-Br-LSD methylene acetamide prodrug (4 mg/kg) to male SD rats.

FIG. 16 shows mean concentration-time profiles of 2-Br-LSD following oral dosing of 2-Br-LSD trimethyl lock prodrug (4 mg/kg) to male SD rats.

FIG. 17 shows mean concentration-time profiles of 2-Br-LSD following oral dosing of 2-Br-LSD-propylamide prodrug (4 mg/kg) to male SD rats.

FIG. 18 shows mean concentration-time profiles of 2-Br-LSD following oral dosing of 2-Br-LSD n-butyl amide prodrug (4 mg/kg) to male SD rats.

FIG. 19 shows mean concentration-time profiles of 2-Br-LSD following oral dosing of 2-Br-LSD oxymethyl tetrahydropyran prodrug (4 mg/kg) to male SD rats.

### DETAILED DESCRIPTION

### I. Terms

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference in their entirety, unless otherwise specified.

Unless otherwise indicated, all numbers expressing quantities of components, molecular weights, percentages, temperatures, times, and so forth, as used in the specification or claims, are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is expressly recited.

Unless explained otherwise, all technical and scientific terms used herein have the same meaning as commonly understood by one of ordinary skill in the art to which this disclosure pertains. Although methods and materials similar or equivalent to those described herein can be used in the practice or testing of the present disclosure, suitable methods and materials are described below. The materials, methods, and examples are illustrative only and not intended to be limiting.

“Administering” refers to any suitable mode of administration, including, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

“Subject” refers to an animal, such as a mammal, including, but not limited to, primates (e.g., humans), cows, sheep, goats, horses, dogs, cats, rabbits, rats, mice and the like. In certain embodiments, the subject is a human subject.

“Therapeutically effective amount” or “therapeutically sufficient amount” or “effective or sufficient amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins). In sensitized cells, the therapeutically effective dose can often be lower than the conventional therapeutically effective dose for non-sensitized cells.

“Neuronal plasticity” refers to the ability of the brain to change its structure and/or function continuously throughout a subject's life. Examples of the changes to the brain include, but are not limited to, the ability to adapt or respond to internal and/or external stimuli, such as due to an injury, and the ability to produce new neurites, dendritic spines, and synapses.

“Brain disorder” refers to a neurological disorder which affects the brain's structure and function. Brain disorders can include, but are not limited to, Alzheimer's, Parkinson's disease, psychological disorder, depression, treatment resistant depression, addiction, anxiety, post-traumatic stress disorder, suicidal ideation, major depressive disorder, bipolar disorder, schizophrenia, stroke, traumatic brain injury, and substance use disorder.

“Combination therapy” refers to a method of treating a disease or disorder, wherein two or more different pharmaceutical agents are administered in overlapping regimens so that the subject is simultaneously exposed to both agents. For example, the compounds of the invention can be used in combination with other pharmaceutically active compounds. The compounds of the invention can be administered simultaneously (as a single preparation or separate preparation) or sequentially to the other drug therapy. In general, a combination therapy envisions administration of two or more drugs during a single cycle or course of therapy.

“Neurotrophic factors” refers to a family of soluble peptides or proteins which support the survival, growth, and differentiation of developing and mature neurons.

“Modulate” or “modulating” or “modulation” refers to an increase or decrease in the amount, quality, or effect of a particular activity, function or molecule. By way of illustration and not limitation, agonists, partial agonists, antagonists, and allosteric modulators (e.g., a positive allosteric modulator) of a G protein-coupled receptor (e.g., 5HT_{2A}) are modulators of the receptor.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 mM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“Composition” refers to a product comprising the specified ingredients in the specified amounts, as well as any product, which results, directly or indirectly, from combination of the specified ingredients in the specified amounts. By “pharmaceutically acceptable” it is meant the carrier, diluent or excipient must be compatible with the other ingredients of the formulation.

“Pharmaceutically acceptable excipient” refers to a substance that aids the administration of an active agent to and absorption by a subject. Pharmaceutical excipients useful in the present invention include, but are not limited to, binders, fillers, disintegrants, lubricants, coatings, sweeteners, flavors and colors. One of skill in the art will recognize that other pharmaceutical excipients are useful in the present invention.

“2-Bromolysergic acid diethyl amide” refers to the compound (6aR,9R)-5-bromo-N,N-diethyl-7-methyl-6,6a,8,9-tetrahydro-4H-indolo[4,3-fg]quinoline-9-carboxamide. The compound may also be referred to as bromolysergide, bromolysergic acid diethylamide, D-2-bromolysergic acid diethylamide, 2-bromo-LSD, or 2-Br-LSD.

2-Bromolysergic acid diethyl amide has the formula

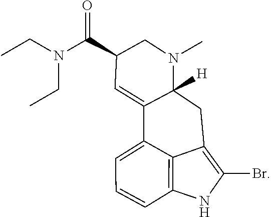 [SMILES: CCN(CC)C(=O)[C@@H]1C=C2c3cccc4[nH]c(Br)c(c34)C[C@H]2N(C)C1]

Compounds herein can include all stereoisomers, enantiomers, diastereomers, mixtures, racemates, atropisomers, and tautomers thereof.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocycloalkyl groups, heteroaryl groups, cycloalkyl groups, acyl groups, acyloxy groups, carbamate groups, amide groups, ureido groups, epoxy groups, and ester groups.

Non-limiting examples of alkyl groups include straight, branched, and cyclic alkyl and alkylene groups. An alkyl group can be, for example, a C₁, C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted.

Alkyl groups can include branched and unbranched alkyl groups. Non-limiting examples of straight alkyl groups include methyl, ethyl, propyl, butyl, pentyl, hexyl, heptyl, octyl, nonyl, and decyl.

Branched alkyl groups include any straight alkyl group substituted with any number of alkyl groups. Non-limiting examples of branched alkyl groups include isopropyl, isobutyl, sec-butyl, and t-butyl.

Non-limiting examples of substituted alkyl groups includes hydroxymethyl, chloromethyl, trifluoromethyl, aminomethyl, 1-chloroethyl, 2-hydroxyethyl, 1,2-difluoroethyl, and 3-carboxypropyl.

Non-limiting examples of cycloalkyl groups include cyclopropyl, cyclobutyl, cyclopentyl, cyclohexyl, cycloheptyl, and cyclooctyl groups. Cycloalkyl groups also include fused-, bridged-, and spiro-bicycles and higher fused-, bridged-, and spiro-systems. A cycloalkyl group can be substituted with any number of straight, branched, or cyclic alkyl groups. Non-limiting examples of cyclic alkyl groups include cyclopropyl, 2-methyl-cycloprop-1-yl, cycloprop-2-en-1-yl, cyclobutyl, 2,3-dihydroxycyclobut-1-yl, cyclobut-2-en-1-yl, cyclopentyl, cyclopent-2-en-1-yl, cyclopenta-2,4-dien-1-yl, cyclohexyl, cyclohex-2-en-1-yl, cycloheptyl, cyclooctanyl, 2,5-dimethylcyclopent-1-yl, 3,5-dichlorocyclohex-1-yl, 4-hydroxycyclohex-1-yl, 3,3,5-trimethylcyclohex-1-yl, octahydropentalenyl, octahydro-1H-indenyl, 3a,4,5,6,7,7a-hexahydro-3H-inden-4-yl, decahydroazulenyl, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

Non-limiting examples of alkenyl groups include straight, branched, and cyclic alkenyl groups. The olefin or olefins of an alkenyl group can be, for example, E, Z, cis, trans, terminal, or exo-methylene. An alkenyl group can be, for example, a C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkenyl and alkenylene groups include ethenyl, prop-1-en-1-yl, isopropenyl, but-1-en-4-yl; 2-chloroethenyl, 4-hydroxybuten-1-yl, 7-hydroxy-7-methyloct-4-en-2-yl, and 7-hydroxy-7-methyloct-3,5-dien-2-yl.

Non-limiting examples of alkynyl groups include straight, branched, and cyclic alkynyl groups. The triple bond of an alkynyl group can be internal or terminal. An alkynyl or alkynylene group can be, for example, a C₂, C₃, C₄, C₅, C₆, C₇, C₈, C₉, C₁₀, C₁₁, C₁₂, C₁₃, C₁₄, C₁₅, C₁₆, C₁₇, Cis, C₁₉, C₂₀, C₂₁, C₂₂, C₂₃, C₂₄, C₂₅, C₂₆, C₂₇, C₂₈, C₂₉, C₃₀, C₃₁, C₃₂, C₃₃, C₃₄, C₃₅, C₃₆, C₃₇, C₃₈, C₃₉, C₄₀, C₄₁, C₄₂, C₄₃, C₄₄, C₄₅, C₄₆, C₄₇, C₄₈, C₄₉, or C₅₀ group that is substituted or unsubstituted. Non-limiting examples of alkynyl groups include ethynyl, prop-2-yn-1-yl, prop-1-yn-1-yl, and 2-methyl-hex-4-yn-1-yl; 5-hydroxy-5-methylhex-3-yn-1-yl, 6-hydroxy-6-methylhept-3-yn-2-yl, and 5-hydroxy-5-ethylhept-3-yn-1-yl.

A halo-alkyl group can be any alkyl group substituted with any number of halogen atoms, for example, fluorine, chlorine, bromine, and iodine atoms. A halo-alkenyl group can be any alkenyl group substituted with any number of halogen atoms. A halo-alkynyl group can be any alkynyl group substituted with any number of halogen atoms.

An alkoxy group can be, for example, an oxygen atom substituted with any alkyl, alkenyl, or alkynyl group. An ether or an ether group comprises an alkoxy group. Non-limiting examples of alkoxy groups include methoxy, ethoxy, propoxy, isopropoxy, and isobutoxy.

A heterocycle can be any ring containing a ring atom that is not carbon, for example, N, O, S, P, Si, B, or any other heteroatom. A heterocycle can be substituted with any number of substituents, for example, alkyl groups and halogen atoms. A heterocycle can be aromatic (heteroaryl) or non-aromatic. Non-limiting examples of heterocycles include pyrrole, pyrrolidine, pyridine, piperidine, succinimide, maleimide, morpholine, imidazole, thiophene, furan, tetrahydrofuran, pyran, and tetrahydropyran.

Non-limiting examples of heterocycles include: heterocyclic units having a single ring containing one or more heteroatoms, non-limiting examples of which include, diazirinyl, aziridinyl, azetidinyl, pyrazolidinyl, imidazolidinyl, oxazolidinyl, isoxazolinyl, thiazolidinyl, isothiazolinyl, oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl, 2,3,4,5-tetrahydro-1H-azepinyl, 2,3-dihydro-1H-indole, and 1,2,3,4-tetrahydroquinoline; and ii) heterocyclic units having 2 or more rings one of which is a heterocyclic ring, non-limiting examples of which include hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, and decahydro-1H-cycloocta[b]pyrrolyl.

Non-limiting examples of heteroaryl include: i) heteroaryl rings containing a single ring, non-limiting examples of which include, 1,2,3,4-tetrazolyl, [1,2,3]triazolyl, [1,2,4]triazolyl, triazinyl, thiazolyl, 1H-imidazolyl, oxazolyl, isoxazolyl, isothiazolyl, furanyl, thiophenyl, pyrimidinyl, 2-phenylpyrimidinyl, pyridinyl, 3-methylpyridinyl, and 4-dimethylaminopyridinyl; and ii) heteroaryl rings containing 2 or more fused rings one of which is a heteroaryl ring, non-limiting examples of which include: 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 4,5,6,7-tetrahydro-1-H-indolyl, quinoxalinyl, quinazolinyl, quinolinyl, 8-hydroxy-quinolinyl, and isoquinolinyl.

The term “acyl” refers to the groups HC(O)—, alkyl-C(O)—, cycloalkyl-C(O)—, cycloalkenyl-C(O)—, aryl-C(O)—, heteroaryl-C(O)— and heterocyclyl-C(O)— where alkyl, cycloalkyl, cycloalkenyl, aryl, heteroaryl, and heterocyclyl are as described herein. By way of example acyl groups include acetyl and benzoyl groups.

“Alkyl” refers to an optionally substituted straight-chain, or optionally substituted branched-chain saturated hydrocarbon having from one to about ten carbon atoms, or from one to six carbon atoms, wherein an sp³-hybridized carbon of the alkyl residue is attached to the rest of the molecule by a single bond. Examples include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl, and hexyl, and longer alkyl groups, such as heptyl, octyl, and the like. Whenever it appears herein, a numerical range such as “C₁-C₆ alkyl” means that the alkyl group consists of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁-C₁₀ alkyl, a C₁-C₉ alkyl, a C₁-C₈ alkyl, a C₁-C₇ alkyl, a C₁-C₆ alkyl, a C₁-C₅ alkyl, a C₁-C₄ alkyl, a C₁-C₃ alkyl, a C₁-C₂ alkyl, or a C₁ alkyl. Unless stated otherwise specifically in the specification, an alkyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —CF₃, —OH, —OMe, —NH₂, or —NO₂. In some embodiments, the alkyl is optionally substituted with oxo, halogen, —CN, —CF₃, —OH, or —OMe. In some embodiments, the alkyl is optionally substituted with halogen.

“Alkenyl” refers to an optionally substituted straight-chain, or optionally substituted branched-chain hydrocarbon having one or more carbon-carbon double-bonds and having from two to about ten carbon atoms, more preferably two to about six carbon atoms, wherein an sp²-hybridized carbon of the alkenyl residue is attached to the rest of the molecule by a single bond. The group may be in either the cis or trans conformation about the double bond(s), and should be understood to include both isomers. Examples include, but are not limited to, ethenyl (—CH═CH₂), 1-propenyl (—CH₂CH═CH₂), isopropenyl[—C(CH₃)═CH₂], butenyl, 1,3-butadienyl, and the like. Whenever it appears herein, a numerical range such as “C₂-C₆ alkenyl” means that the alkenyl group may consist of 2 carbon atoms, 3 carbon atoms, 4 carbon atoms, 5 carbon atoms, or 6 carbon atoms, although the present definition also covers the occurrence of the term “alkenyl” where no numerical range is designated. In some embodiments, the alkenyl is a C₂-C₁₀ alkenyl, a C₂-C₉ alkenyl, a C₂-C₅ alkenyl, a C₂-C₇ alkenyl, a C₂-C₆ alkenyl, a C₂-C₅ alkenyl, a C₂-C₄ alkenyl, a C₂-C₃ alkenyl, or a C₂ alkenyl. Unless stated otherwise specifically in the specification, an alkenyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkenyl is optionally substituted with oxo, halogen, —CN, —CF₃, —OH, —OMe, —NH₂, or —NO₂. In some embodiments, an alkenyl is optionally substituted with oxo, halogen, —CN, —CF₃, —OH, or —OMe. In some embodiments, the alkenyl is optionally substituted with halogen.

“Alkynyl” refers to an optionally substituted straight-chain or optionally substituted branched-chain hydrocarbon having one or more carbon-carbon triple-bonds and having from two to about ten carbon atoms, more preferably from two to about six carbon atoms. Examples include, but are not limited to, ethynyl, 2-propynyl, 2-butynyl, 1,3-butadiynyl, and the like. Whenever it appears herein, a numerical range such as “C₂-C₆ alkynyl” means that the alkynyl group may consist of 2 carbon atoms, 3 carbon atoms, 4 carbon atoms, 5 carbon atoms, or 6 carbon atoms, although the present definition also covers the occurrence of the term “alkynyl” where no numerical range is designated. In some embodiments, the alkynyl is a C₂-C₁₀ alkynyl, a C₂-C₉ alkynyl, a C₂-C₈ alkynyl, a C₂-C₇ alkynyl, a C₂-C₆ alkynyl, a C₂-C₅ alkynyl, a C₂-C₄ alkynyl, a C₂-C₃ alkynyl, or a C₂ alkynyl. Unless stated otherwise specifically in the specification, an alkynyl group is optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkynyl is optionally substituted with oxo, halogen, —CN, —CF₃, —OH, —OMe, —NH₂, or —NO₂. In some embodiments, an alkynyl is optionally substituted with oxo, halogen, —CN, —CF₃, —OH, or —OMe. In some embodiments, the alkynyl is optionally substituted with halogen.

“Alkoxy” refers to a radical of the formula —OR^{a} where R^{a} is an alkyl radical as defined. Non-limiting examples of alkoxy groups include methoxy, ethoxy, propoxy, isopropoxy, and isobutoxy. In some embodiments, alkoxy is C₁-C₆ alkoxy. Unless stated otherwise specifically in the specification, an alkoxy group may be optionally substituted, for example, with oxo, halogen, amino, nitrile, nitro, hydroxyl, haloalkyl, alkoxy, aryl, cycloalkyl, heterocycloalkyl, heteroaryl, and the like. In some embodiments, an alkoxy is optionally substituted with oxo, halogen, —CN, —CF₃, —OH, —OMe, —NH₂, or —NO₂. In some embodiments, an alkoxy is optionally substituted with oxo, halogen, —CN, —CF₃, —OH, or —OMe. In some embodiments, the alkoxy is optionally substituted with halogen.

“Aminoalkyl” refers to an alkyl radical, as defined above, that is substituted by one or more amines. In some embodiments, the alkyl is substituted with one amine. In some embodiments, the alkyl is substituted with one, two, or three amines. Hydroxyalkyl include, for example, aminomethyl, aminoethyl, aminopropyl, aminobutyl, or aminopentyl. In some embodiments, the hydroxyalkyl is aminomethyl.

“Aryl” refers to a radical derived from a hydrocarbon ring system comprising hydrogen, 6 to 30 carbon atoms, and at least one aromatic ring. The aryl radical may be a monocyclic, bicyclic, tricyclic, or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl. Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In some embodiments, the aryl is phenyl. Unless stated otherwise specifically in the specification, an aryl may be optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, an aryl is optionally substituted with halogen, methyl, ethyl, —CN, —CF₃, —OH, —OMe, —NH₂, or —NO₂. In some embodiments, an aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom), bridged, or spiro ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₅ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls or carbocycles include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example, cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Deuteroalkyl” refers to an alkyl radical, as defined above, that is substituted by one or more deuteriums. In some embodiments, the alkyl is substituted with one deuterium. In some embodiments, the alkyl is substituted with one, two, or three deuteriums. In some embodiments, the alkyl is substituted with one, two, three, four, five, or six deuteriums. Deuteroalkyl include, for example, CD₃, CH₂D, CHD₂, CH₂CD₃, CD₂CD₃, CHDCD₃, CH₂CH₂D, or CH₂CHD₂. In some embodiments, the deuteroalkyl is CD₃.

“Haloalkyl” refers to an alkyl radical, as defined above, that is substituted by one or more halogens. In some embodiments, the alkyl is substituted with one, two, or three halogens. In some embodiments, the alkyl is substituted with one, two, three, four, five, or six halogens. Haloalkyl include, for example, trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl is trifluoromethyl. In some embodiments, haloalkyl is C₁-C₆ haloalkyl.

“Halo” or “halogen” refers to bromo, chloro, fluoro, or iodo. In some embodiments, halogen is fluoro or chloro. In some embodiments, halogen is fluoro.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, or —CH(CH₃)OCH₃. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Heterocycloalkyl” refers to a stable 3- to 24-membered partially or fully saturated ring radical comprising 2 to 23 carbon atoms and from one to 8 heteroatoms selected from the group consisting of nitrogen, oxygen, phosphorous, and sulfur. Unless stated otherwise specifically in the specification, the heterocycloalkyl radical may be a monocyclic, bicyclic, tricyclic, or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized.

Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₅ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). In some embodiments, the heterocycloalkyl is a 3- to 6-membered heterocycloalkyl. In some embodiments, the cycloalkyl is a 5- to 6-membered heterocycloalkyl. Examples of such heterocycloalkyl radicals include, but are not limited to, aziridinyl, azetidinyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. In some embodiments, heterocycloalkyl is aziridinyl, azetidinyl, morpholinyl, piperidinyl, piperazinyl, oxetanyl, tetrahydrofuryl, tetrahydropyranyl, or thiomorpholinyl. The term heterocycloalkyl also includes all ring forms of the carbohydrates, including but not limited to, the monosaccharides, the disaccharides, and the oligosaccharides. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). Unless stated otherwise specifically in the specification, a heterocycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heterocycloalkyl is optionally substituted with oxo, halogen, methyl, ethyl, —CN, —CF₃, —OH, —OMe, —NH₂, or —NO₂. In some embodiments, a heterocycloalkyl is optionally substituted with oxo, halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous, and sulfur, and at least one aromatic ring. he heteroaryl radical may be a monocyclic, bicyclic, tricyclic, or tetracyclic ring system, which may include fused (when fused with a cycloalkyl or heterocycloalkyl ring, the heteroaryl is bonded through an aromatic ring atom) or bridged ring systems; and the nitrogen, carbon, or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). In some embodiments, heteroaryl is imidazolyl, indazolyl, indolyl, pyrrolyl, pyrazolyl, pyridinyl, pyrazinyl, pyrimidinyl, pyridazinyl, triazolyl, or tetrazolyl. Unless stated otherwise specifically in the specification, a heteroaryl is optionally substituted, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroaryl is optionally substituted with halogen, methyl, ethyl, —CN, —CF₃, —OH, —OMe, —NH₂, or —NO₂. In some embodiments, a heteroaryl is optionally substituted with halogen, methyl, ethyl, —CN, —CF₃, —OH, or —OMe. In some embodiments, the heteroaryl is optionally substituted with halogen.

Certain compounds according to Formula (I) disclosed herein are isotopically enriched, meaning that they have an isotope present in greater than its natural abundance at one or more position. The term “isotopic enrichment factor” as used herein means the ratio between the isotopic abundance and the natural abundance of a specified isotope. In a compound of this disclosure, when a particular position is designated as having a particular isotope, such as deuterium, it is understood that the abundance of deuterium at that position is substantially greater than the natural abundance of deuterium, which is about 0.015% (on a mol/mol basis). A position designated as a particular isotope will have a minimum isotopic enrichment factor of at least 3000 (45% incorporation of the indicated isotope). Thus, isotopically enriched compounds disclosed herein having deuterium will have a minimum isotopic enrichment factor of at least 3000 (45% deuterium incorporation) at each atom designated as deuterium in the compound. Such compounds may be referred to herein as “deuterated” compounds. In one embodiment, deuterated compounds disclosed herein have an isotopic enrichment factor for each designated atom of at least 3500 (52.5%), at least 4000 (60% deuterium incorporation), at least 4500 (670.5% deuterium incorporation), at least 5000 (75% deuterium), at least 5500 (820.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (990.5% deuterium incorporation).

In some embodiments, the present disclosure provides a deuterated analogue of any compound disclosed herein. A deuterated analogue can include a compound herein where one or more ¹H atoms is replaced with a deuterium atom.

### II. Compounds

Disclosed herein are analogs of 2Br-LSD. In one embodiment the disclosed analogs function as prodrugs of 2Br-LSD, releasing 2-Br-LSD as an active metabolite under suitable conditions, such as physiological conditions following administration of the analog to a subject. In one embodiment, the disclosed 2-Br-LSD analogs have Formula (I):

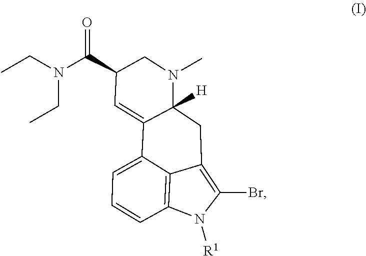

or a pharmaceutically acceptable salt thereof, wherein R¹ is a promoiety, or group that is cleaved under physiological conditions to yield an active metabolite.

In certain embodiments disclosed herein, the compound is a compound of Formula (I), or a pharmaceutically acceptable salt thereof, wherein:
R¹ is —C(O)OR³, —C(O)R⁴, —CH(R⁵)OR⁶, —C(O)OCH(R⁵)OC(O)R⁴, —C(O)OCH(R⁵)OC(O)OR⁴, —C(O)OCH(R⁵)OC(O)NHR⁴, —CH(R⁵)NHC(O)R⁶, —CH(R⁵)C(O)R⁶, —S(O)₂R⁷, —S(O)₂OR⁷, —P(O)OR⁸[N(R⁹)R¹⁰], —C(O)N(R⁹)R¹⁰, —P(O)OR¹¹(OR¹²), —CH(R⁴)OP(O)OR⁸[N(R⁹)R¹⁰], —CH(R⁴)OP(O)OR¹¹(OR¹²), —C(O)OCH(R⁵)OP(O)OR²⁰(OR²¹);
each of R³, R⁴, R⁵, R⁶, R⁷, and R⁸ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
or R³ is

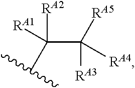

wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶,
or R⁴ is

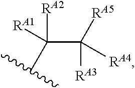

wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.
or R⁴ is

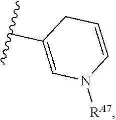

wherein R^{A7} is hydrogen or alkyl;
or R⁴ is —CH(R^{A1})NH₂, wherein R^{A1} is an amino acid side chain;
each of R⁹ and R¹⁰ is independently hydrogen, alkyl, heteroalkyl, haloalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
or R⁹ is

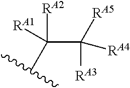

wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁵)R¹⁹, —N(R¹³)C(O)OR¹⁴, —N(R¹³)C(O)R¹⁴, —C(O)R¹⁴, —OC(O)R¹⁵, or —OC(O)OR¹⁶;
each of R¹¹ and R¹² is independently hydrogen, alkyl, heteroalkyl, cycloalkyl, heterocycloalkyl, aryl, heteroaryl, —CH(R⁵)C(O)R¹⁴, —CH(R⁵)C(O)OR¹, —CH(R⁵)OC(O)R¹⁵, or —CH(R⁵)OC(O)OR¹⁶, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or —S(O)₂R²², wherein alkyl, heteroalkyl, cycloalkyl, heterocycloalkyl, aryl, or heteroaryl is unsubstituted or substituted with alkyl, aryl, halogen, oxo, —OR¹³, —NR(R¹⁸)R¹⁹, —C(O)R¹⁴, —OC(O)R¹⁵, —OC(O)OR¹⁶, or —OC(O)N(R¹⁵)R¹⁹;
each of R³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
or R¹⁴ or R¹⁶ is

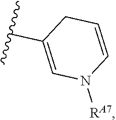

wherein R^{A7} is hydrogen or alkyl;
or R¹⁵ is —CH(R^{A1})NH₂, wherein R^{A1} is an amino acid side chain;
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰, R²¹ and R²² is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B};
each R^{B} is independently halogen, amino, cyano, hydroxyl, alkyl, heteroalkyl, cycloalkyl, heterocycloalkyl, aryl, heteroaryl, arylalkyl, acyl or heteroarylalkyl, wherein cycloalkyl, heterocycloalkyl, acyl, aryl, or heteroaryl is unsubstituted or substituted with one or more halogen, amino, cyano, hydroxyl, alkyl, acetyl, or benzoyl; and
each R^{C} is independently hydrogen or alkyl. In some embodiments, each of R³, R⁴, R⁵, R⁶, R⁷, and R⁸ is independently hydrogen, C₁-C₁₀ alkyl, C₂-C₁₀ alkenyl, C₁-C₆ haloalkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}. In some embodiments, each of R³, R⁴, R⁵, R⁶, R⁷, and R⁸ is independently C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}.

In some embodiments, R³ is

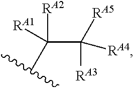

wherein each of R^{A1}, R^{A2}, R^{A3}, and R^{A4} is independently hydrogen or C₁-C₁₀ alkyl; and R^{A5} is C₃-C₆ heteroalkyl, 3- to 6-membered heterocycloalkyl, monocyclic heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶. In some embodiments, R³ is

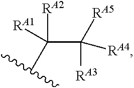

wherein each of R^{A1}, R^{A2}, R^{A3}, and R^{A4} is independently hydrogen or C₁-C₆ alkyl; and R^{A5} is C₃-C₆ heteroalkyl, 3- to 6-membered heterocycloalkyl, 5-membered monocyclic heteroaryl, 6-membered monocyclic heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶.

In some embodiments, R⁴ is

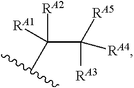

wherein each of R^{A1} and R^{A2} is independently hydrogen, C₁-C₁₀ alkyl, C₃-C₆ cycloalkyl, phenyl, or monocyclic heteroaryl, or R^{A1} and R^{A2} together with the atom to which they are attached form a C₃-C₆ cycloalkyl ring; each of R^{A3} and R^{A4} is independently hydrogen, C₁-C₁₀ alkyl, C₃-C₆ cycloalkyl, phenyl, or monocyclic heteroaryl, or R^{A3} and R^{A4} together with the atom to which they are attached form a C₃-C₆ cycloalkyl ring; and R^{A5} is C₃-C₆ heteroalkyl, 3- to 6-membered heterocycloalkyl, monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶. In some embodiments, R⁴ is

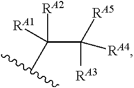

wherein each of R^{A1} and R^{A2} is independently hydrogen, C₁-C₆ alkyl, C₃-C₆ cycloalkyl, phenyl, 5-membered monocyclic heteroaryl, or 6-membered monocyclic heteroaryl, or R^{A1} and R^{A2} together with the atom to which they are attached form a C₃-C₆ cycloalkyl ring; each of R^{A3} and R^{A4} is independently hydrogen, C₁-C₆ alkyl, C₃-C₆ cycloalkyl, phenyl, 5-membered monocyclic heteroaryl, or 6-membered monocyclic heteroaryl, or R^{A3} and R^{A4} together with the atom to which they are attached form a C₃-C₆ cycloalkyl ring; and R^{A5} is C₃-C₆ heteroalkyl, 3- to 6-membered heterocycloalkyl, 5-membered monocyclic heteroaryl, 6-membered monocyclic heteroaryl, —C(O)OR 1, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.

In some embodiments, R⁴ is

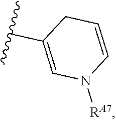

wherein R^{A7} is hydrogen or C₁-C₁₀ alkyl. In some embodiments, R⁴ is

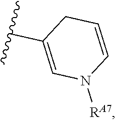

wherein R^{A7} is hydrogen or C₁-C₆ alkyl.

In some embodiments, R⁴ is —CH(R^{A1})NH₂, wherein R^{A1} is an amino acid side chain.

In some embodiments, each of R⁹ and R¹⁰ is independently hydrogen, C₁-C₁₀ alkyl, C₃-C₆ heteroalkyl, C₁-C₆ haloalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R⁹ and R¹⁰ together with the atom to which they are attached form a 3- to 6-membered heterocycloalkyl ring or a heteroaryl ring that is unsubstituted or substituted with one to five R^{A}. In some embodiments, each of R⁹ and R¹⁰ is independently hydrogen, C₁-C₆ alkyl, C₃-C₆ heteroalkyl, C₁-C₆ haloalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R⁹ and R¹⁰ together with the atom to which they are attached form a 3- to 6-membered heterocycloalkyl ring or a heteroaryl ring that is unsubstituted or substituted with one to three R^{A}.

In some embodiments, R⁹ is

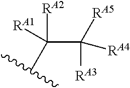

wherein each of R^{A1}, R^{A2}, R^{A3}, and R^{A4} is independently hydrogen or C₁-C₁₀ alkyl, and R^{A5} is C₃-C₆ heteroalkyl, 3- to 6-membered heterocycloalkyl, monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶. In some embodiments, R⁹ is

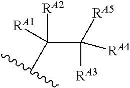

wherein each of R^{A1}, R^{A2}, R^{A3}, and R^{A4} is independently hydrogen or C₁-C₆ alkyl, and R^{A5} is C₃-C₆ heteroalkyl, 3- to 6-membered heterocycloalkyl, 5-membered, 6-membered monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶;

In some embodiments, each of each of R¹¹ and R¹² is independently hydrogen, C₁-C₁₀ alkyl, C₃-C₆ heteroalkyl, C₃-C₅ cycloalkyl, 3- to 6-membered heterocycloalkyl, phenyl, monocyclic heteroaryl, —CH(R⁵)C(O)R¹⁴, —CH(R⁵)C(O)OR¹³, —CH(R⁵)OC(O)R¹⁵, or —CH(R⁵)OC(O)OR¹⁶, wherein alkyl, heteroalkyl, cycloalkyl, heterocycloalkyl, phenyl, or heteroaryl is unsubstituted or substituted with one to five R^{A}, or R¹¹ and R¹² together with the atoms to which they are attached form a 3- to 6-membered heterocycloalkyl ring that is unsubstituted or substituted with one to five R^{A}. In some embodiments, each of R¹¹ and R¹² is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, —CH(R⁵)C(O)R¹⁴, —CH(R⁵)C(O)OR¹³, —CH(R⁵)OC(O)R¹⁵, or —CH(R⁵)OC(O)OR¹⁶, wherein alkyl, heteroalkyl, cycloalkyl, heterocycloalkyl, phenyl, or heteroaryl is unsubstituted or substituted with one to three R^{A}, or R¹¹ and R¹² together with the atoms to which they are attached form a 3- to 6-membered heterocycloalkyl ring that is unsubstituted or substituted with one to three R^{A}.

In some embodiments, each R^{A} is independently C₁-C₁₀ alkyl, C₃-C₆ heteroalkyl, C₃-C₈ cycloalkyl, 3- to 6-membered heterocycloalkyl, phenyl, monocyclic heteroaryl, an amino acid side chain, —R⁵, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or S(O)₂R²², wherein alkyl, heteroalkyl, cycloalkyl, heterocycloalkyl, phenyl, or heteroaryl is unsubstituted or substituted with C₁-C₆ alkyl, phenyl, halogen, oxo, —OR¹³, —NR(R¹⁸)R¹⁹, —C(O)R¹⁴, —OC(O)R¹⁵, —OC(O)OR¹⁶, or —OC(O)N(R¹⁸)R¹⁹. In some embodiments, each R^{A} is independently C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or S(O)₂R²² wherein alkyl, heteroalkyl, cycloalkyl, heterocycloalkyl, phenyl, or heteroaryl is unsubstituted or substituted with C₁-C₆ alkyl, phenyl, halogen, oxo, —OR¹³, —NR(R¹⁸)R¹⁹, —C(O)R¹⁴, —OC(O)R¹⁵, —OC(O)OR¹⁶, or —OC(O)N(R¹⁸)R¹⁹.

In some embodiments, each of R¹³, R¹⁴, R¹⁵, R¹⁶, or R¹⁷ is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, and heteroaryl is unsubstituted or substituted with one to five R^{B}. In some embodiments, each of R¹³, R¹⁴, R¹⁵, R¹⁶, or R¹⁷ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, and heteroaryl is unsubstituted or substituted with one to three R^{B}.

In some embodiments, R¹⁴ or R¹⁶ is

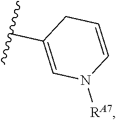

wherein R^{A7} is hydrogen or C₁-C₁₀ alkyl. In some embodiments, R¹⁴ or R¹⁶ is

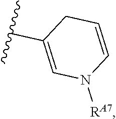

wherein R^{A7} is hydrogen or C₁-C₆ alkyl.

In some embodiments, R¹⁵ is —CH(R^{A1})NH₂, wherein R^{A1} is an amino acid side chain

In some embodiments, each of R¹⁸ and R¹⁹ is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}; or R¹⁸ and R¹⁹ together with the atom to which they are attached form a 3- to 6-membered heterocycloalkyl ring or heteroaryl ring, each of which is unsubstituted or substituted with one to five R^{B}. In some embodiments, each of R¹⁸ and R¹⁹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}; or R¹⁸ and R¹⁹ together with the atom to which they are attached form a 3- to 6-membered heterocycloalkyl ring or heteroaryl ring, each of which is unsubstituted or substituted with one to three R^{B}.

In some embodiments, each of R²⁰, R²¹, and R²² is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}, or R²⁰ and R²¹ together with the atoms to which they are attached form a 3- to 6-membered heterocycloalkyl ring that is unsubstituted or substituted with one to five R^{B}. In some embodiments, each of R²⁰, R²¹, and R²² is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}, or R²⁰ and R²¹ together with the atoms to which they are attached form a 3- to 6-membered heterocycloalkyl ring that is unsubstituted or substituted with one to three R^{B}.

In some embodiments, each R^{B} is independently halogen, amino, cyano, hydroxyl, C₁-C₁₀ alkyl, C₃-C₆ heteroalkyl, C₃-C₅ cycloalkyl, 3- to 6-membered heterocycloalkyl, phenyl, monocyclic heteroaryl, benzyl, —C(O)CH₃, —C(O)Ph, or (monocyclic heteroaryl)-C₁-C₄ alkyl wherein cycloalkyl, heterocycloalkyl, phenyl, —C(O)CH₃, or heteroaryl is unsubstituted or substituted with one to five halogen, amino, cyano, hydroxyl, C₁-C₆ alkyl, C₁-C₆ acetyl, or benzoyl. In some embodiments, each R^{B} is independently halogen, amino, cyano, hydroxyl, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, benzyl, —C(O)CH₃, —C(O)Ph, or (5- or 6-membered monocyclic heteroaryl)-CH₂—, wherein cycloalkyl, heterocycloalkyl, phenyl, —C(O)CH₃, or heteroaryl is unsubstituted or substituted with one to three halogen, amino, cyano, hydroxyl, C₁-C₆ alkyl, C₁-C₆ acetyl, or benzoyl.

In some embodiments, each R^{C} is independently hydrogen or C₁-C₁₀ alkyl. In some embodiments, each R^{C} is independently hydrogen or C₁-C₆ alkyl.

In certain embodiments of the compounds disclosed herein, including compounds according to Formula (I), the compounds are enriched in deuterium.

In some embodiments of Formula (I), R¹ is —C(O)OR³, wherein R³ is alkyl, alkenyl, haloalkyl, heteroalkyl, cycloalkyl, heterocycloalkyl, aryl, or heteroaryl, each of which is substituted or unsubstituted.

In one such embodiment, compounds of Formula (I) have a structure according to Formula (Ia):

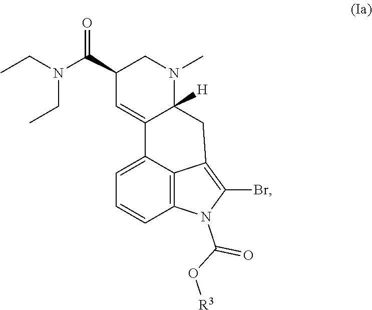

or pharmaceutically acceptable salt thereof, wherein R³ is alkyl, alkenyl, haloalkyl, heteroalkyl, cycloalkyl, heterocycloalkyl, aryl, or heteroaryl, each of which is substituted or unsubstituted.

In some embodiments of Formulas (I) and (Ia), R³ is alkyl that is substituted. In some embodiments is a compound of Formula (I), R³ is alkyl substituted with heteroalkyl, heterocycloalkyl, or heteroaryl, wherein each of heteroalkyl, heterocycloalkyl, and heteroaryl is unsubstituted or substituted.

In some embodiments of Formula (Ia) R³ is alkyl that is unsubstituted. In some embodiments of Formula (Ia), R³ is heteroalkyl. In some embodiments of Formula (Ia), R³ is heteroalkyl that is unsubstituted. In some embodiments of Formula (I), R³ is ethyl.

In some embodiments of Formulas (I) and (Ia), R¹ is —C(O)OR³, wherein R³ is alkyl substituted with heterocycloalkyl. In some embodiments of Formula (I), R¹ is —C(O)OR³, wherein R³ is alkyl substituted with —N(R¹³)C(O)OR¹⁴. In some embodiments of Formula (I), R¹³ is hydrogen or alkyl. In some embodiments of Formula (I), R¹⁴ is alkyl, aryl, or heteroaryl.

In some embodiments of compounds of Formula (Ia,) R³ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl. In some embodiments is a compound of Formula (Ia), wherein R³ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In one embodiment of Formulas (I) and (Ia), disclosed compounds have the structure of Formula (Ia1):

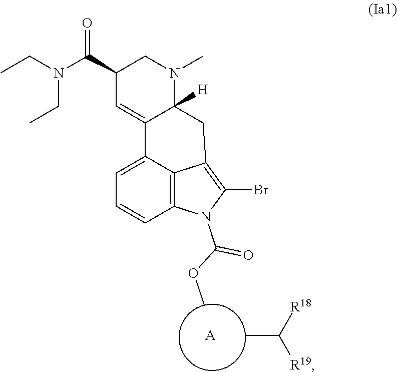

or a pharmaceutically acceptable salt thereof, wherein

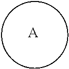

is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In one embodiment, compounds of Formulas (I), (Ia) and (Ia1) have the structure of Formula (Ia2):

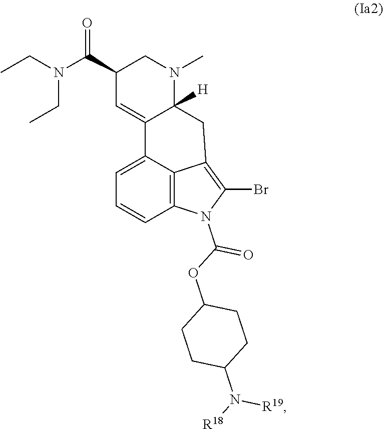

or a pharmaceutically acceptable salt thereof, wherein each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In one embodiment, compounds of Formulas (I) and (Ia) have the structure of Formula (Ia3):

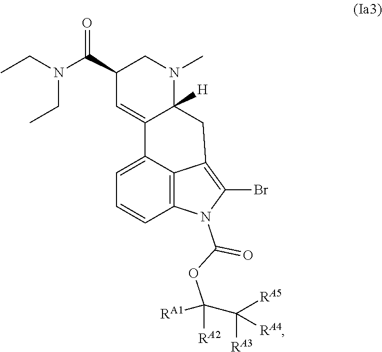

or a pharmaceutically acceptable salt thereof, wherein each of R^{A1}, R^{A2}, R^{A3}, and R^{A4} is independently hydrogen or alkyl, or R^{A1} and R^{A2} or R^{A3} and R^{A4} together with the atom to which they are attached form a cycloalkyl ring; and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.

In some embodiments of Formula (Ia3) one of R^{A1}, R^{A2}, R^{A3}, and R^{A4} is alkyl, and each of R^{A1}, R^{A2}, R^{A3}, and R^{A4} that is not alkyl is hydrogen. In some of Formula (Ia3), two of R^{A1}, R^{A2}, R^{A3}, and R^{A4} are alkyl, and each of R^{A1}, R^{A2}, R^{A3}, and R^{A4} that is not alkyl is hydrogen. In some embodiments of Formula (Ia3), each of R^{A1}, R^{A2}, R^{A3}, and R^{A4} is hydrogen. In some embodiments of Formula (Ia3), R^{A3}, and R^{A4} together with the atom to which they are attached form a cycloalkyl ring, and R^{A1} and R^{A2} are each hydrogen.

In some embodiments of a compound of Formula (Ia3), R^{A5} is C(O)OR¹³, and R¹³ is selected from methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, and 6-pyrimidyl. In some embodiments is a compound of Formula (Ia3), wherein each of R^{A1}, R^{A2}, R^{A3} and R^{A4} is hydrogen.

In one embodiment, compounds of Formulas (I) and (Ia) have the structure:

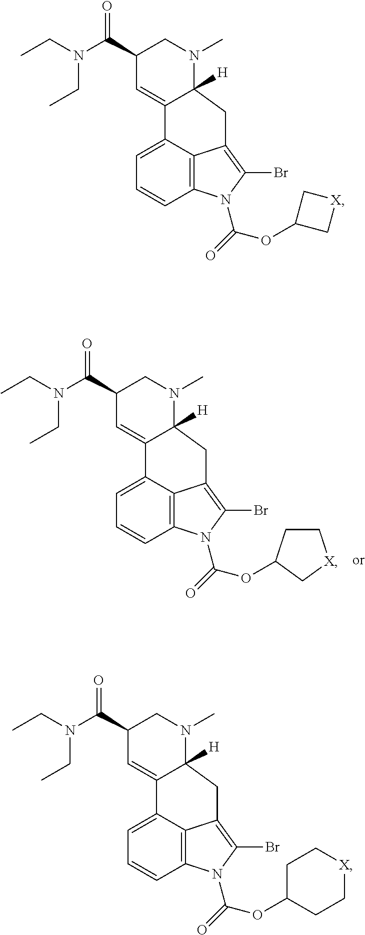

or a pharmaceutically acceptable salt thereof, wherein each X is independently —O—, —S—, —S(O)—, —S(O)₂—, —NH—, or —NR^{A}.

In one embodiment, compounds have Formulas (I) and (Ia) wherein R³ is alkyl, or cycloalkyl optionally substituted with one or two R^{A}.

In one embodiment of compounds according to Formula (I) and (Ia), R³ is cycloalkyl, such as in compounds having the structure:

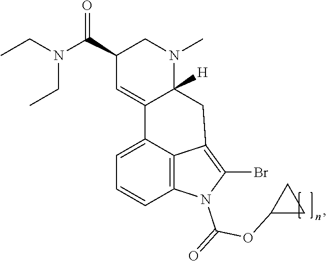

or a pharmaceutically acceptable salt thereof, wherein n is an integer from 1 to 6.

In one embodiment of compounds according to Formula (I) and (Ia), R³ is alkyl, such as in compounds having the structure

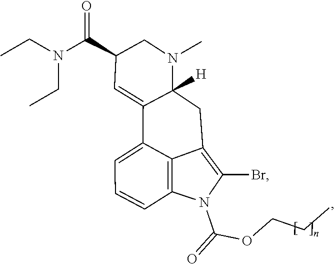

or a pharmaceutically acceptable salt thereof, wherein n is an integer from 1 to 20.

In particular embodiments of compounds of Formulas (I) and (Ia) have a structure:

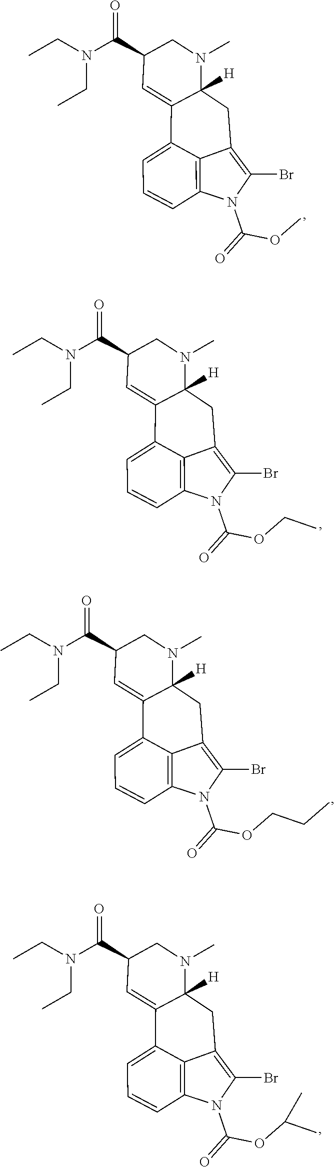

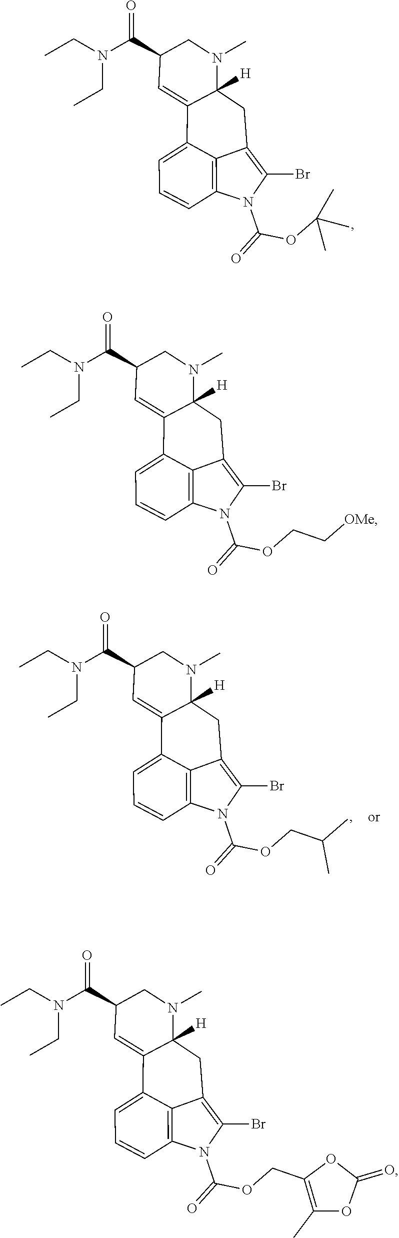

or a pharmaceutically acceptable salt thereof.

In particular embodiments of Formula (I), R¹ forms an amide with the nitrogen to which it is attached. Such compounds of Formula (I) may be represented by Formula (Ib):

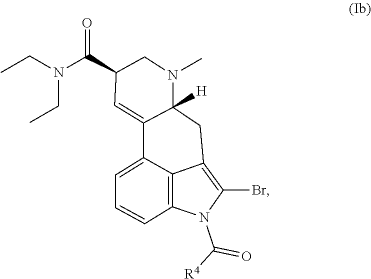

or a pharmaceutically acceptable salt thereof, wherein R⁴ is alkyl, alkenyl, haloalkyl, heteroalkyl, cycloalkyl, heterocycloalkyl, aryl, or heteroaryl, each of which is substituted or unsubstituted.

In some embodiments of Formulas (I) and (Ib), R⁴ is alkyl. In some embodiments of a compound of Formula (Ib), R⁴ is CH₂CF₃. In some embodiments of a compound of Formula (Ib), R⁴ is unsubstituted alkyl. In some embodiments of Formula (Ib), R⁴ is methyl, ethyl, n-propyl, isopropyl, n-butyl, tert-butyl, 3-methyl-1-butyl, n-pentyl, n-hexyl, n-heptyl, n-octyl, or n-nonyl. In some embodiments a compound of Formula (Ib) is one wherein R⁴ is cycloalkyl. In some embodiments of compound of Formula (Ib), R⁴ is unsubstituted cycloalkyl. In some embodiments of compounds of Formula (Ib), R⁴ is cyclopropyl, cyclobutyl, cyclopentyl, cyclohexyl, or cycloheptyl. In some embodiments of a compound of Formula (Ib), the compound is one wherein R⁴ is aryl. In some embodiments of Formula (Ib), wherein R⁴ is substituted or unsubstituted phenyl. In other embodiments of Formula (Ib), R⁴ is heteroaryl and in certain such some embodiments of Formula (Ib), R⁴ is 2-pyridyl, 3-pyridyl, 4-pyridyl, 2-pyrimidyl, 3-pyrimidyl, or 6-pyrimidyl.

In some embodiments of Formulas (I) and (Ib), R⁴ is R^{A} substituted with —OR¹³, —N(R¹⁸)R¹⁹, or —C(O)OR¹³, such as wherein R⁴ is alkyl, substituted with —OR¹³, —N(R¹⁸)R¹⁹, or —C(O)OR¹³. In some embodiments of Formula (Ib), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (Ib) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (Ib) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (Ib), R⁴ is heteroalkyl. In some embodiments of Formulas (I) and (Ib), R⁴ is CH₂CH₂OMe or CH₂CH₂SO₂Me. In some embodiments of Formulas (I) and (Ib), R⁴ is —(CH₂)ₙCO₂H, wherein n is 1, 2, 3, 4, 5, or 6. In some embodiments of Formulas (I) and (Ib), R⁴ is —(CH₂)ₙCO₂R¹³, wherein n is 1, 2, 3, 4, 5, or 6. In some embodiments of Formulas (I) and (Ib), R⁴ is —(CH₂)ₙCO₂R¹³, wherein R¹³ is alkyl. In some embodiments of Formulas (I) and (Ib), R⁴ is —(CH₂)ₙCO₂R¹³, wherein R¹³ is unsubstituted alkyl. In some embodiments of Formulas (I) and (Ib), R⁴ is —(CH₂)ₙCO₂R¹³, wherein R¹³ is methyl, ethyl, n-propyl, isopropyl, n-butyl, tert-butyl, or —CH(Et)₂.

In some embodiments of Formulas (I) and (Ib), R⁴ is —(CH₂)ₙOR¹³, wherein n is 1, 2, 3, 4, 5, 6 or 7. In some embodiments of Formulas (I) and (Ib), R⁴ is —(CH₂)ₙOR¹³, wherein R¹³ is alkyl. In some embodiments of Formulas (I) and (Ib), R⁴ is —(CH₂)ₙOR¹³, wherein R¹³ is unsubstituted alkyl. In some embodiments of Formulas (I) and (Ib), R⁴ is —(CH₂)ₛOR¹³, wherein R¹³ is methyl, ethyl, n-propyl, isopropyl, n-butyl, tert-butyl, or —CH(Et)₂.

In one embodiment of Formula (Ib), R⁴ and the carbonyl atom to which R⁴ is attached form an amino acid residue. In one embodiment of such compounds, the amino acid residue is an α-amino acid residue, such as a naturally occurring α-amino acid residue. In one embodiment of Formula (Ib), R⁴ is —CH(R^{A1})NH₂, wherein R^{A1} is an amino acid side chain. In some embodiments, the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

In one embodiment compounds of Formulas (I) and (Ib) have the structure of Formula (Ib1):

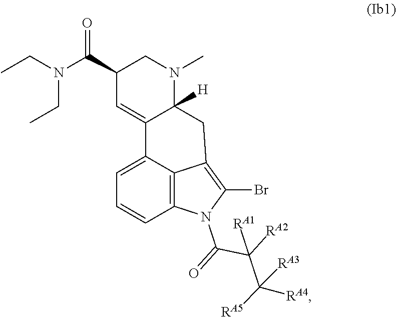

or a pharmaceutically acceptable salt thereof, wherein: each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.

In certain embodiments of compounds of Formulas (I) and (Ib), R⁴ is

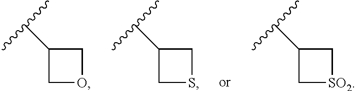 [SMILES: *C1COC1 ; *C1CSC1 ; *C1CS(=O)(=O)C1]

In some embodiments of Formula (Ib), R⁴ is

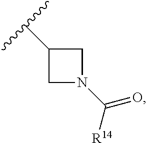

and in certain such embodiments of a compound of Formula (Ib), wherein R⁴ is

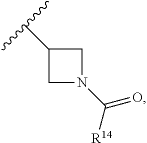

R¹⁴ is alkyl, cycloalkyl, or aryl, such as compounds wherein R¹⁴ is methyl, ethyl, n-propyl, isopropyl, or CH₂CH₂OMe. In some embodiments of Formula (Ib), wherein R⁴ is

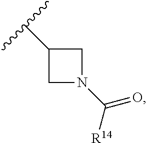

R¹⁴ is phenyl.

In some embodiments of Formulas (I) and (Ib), the compound is:

In some embodiments of compounds of Formula (Ib), R⁴ is

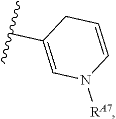

wherein R^{A7} is hydrogen or alkyl. In some embodiments of such compounds of Formula (Ib), R⁴ is

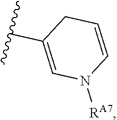

wherein R^{A7} is hydrogen. In some embodiments of Formula (Ib), R⁴ is

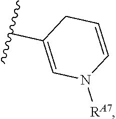

wherein R^{A7} is alkyl. In some embodiments of Formula (Ib), R⁴ is

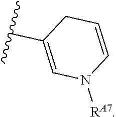

wherein R^{A7} is unsubstituted alkyl. In some embodiments of Formula (Ib), R⁴ is

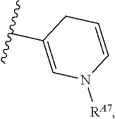

and R^{A7} is methyl, ethyl, n-propyl, isopropyl, or n-butyl.

In some embodiments of Formulas (I) and (Ib), R⁴ is —(CH₂)ₙ N(R⁸)R¹⁹, wherein n is 1, 2, 3, 4, 5, 6 or 7. In some embodiments of Formulas (I) and (Ib), R⁴ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring, and in certain such embodiments the heterocycloalkyl ring is substituted with one or more R^{B}, such as wherein R^{B} is selected from alkyl, heteroalkyl, —C(O)CH₃ and —C(O)Ph.

In one embodiment, compounds according to Formulas (I), (Ib) and (Ib1) the structure:

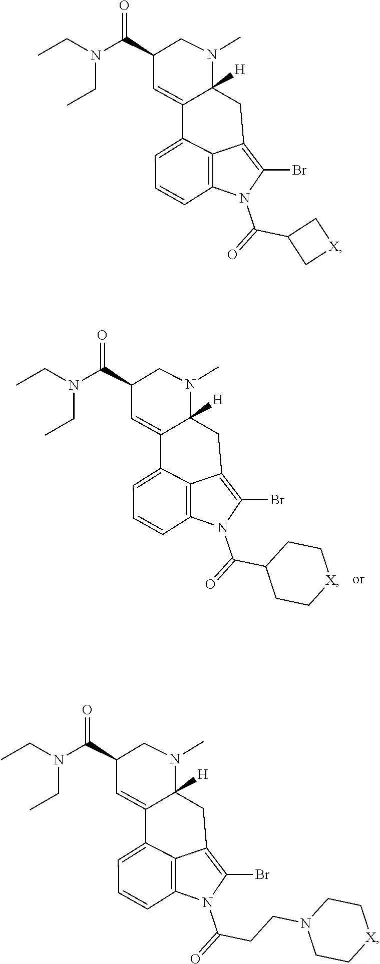

or a pharmaceutically acceptable salt thereof, wherein X is independently —O—, —S—, —S(O)—, —S(O)₂—, —NH—, or —NR^{A}.

In one embodiment, compounds according to Formula (I) and (Ib), R⁴ is alkyl, such as in compounds having the structure:

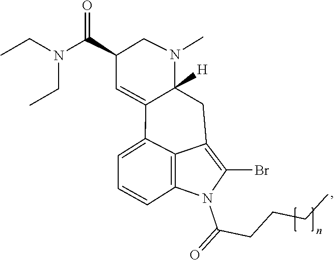

or a pharmaceutically acceptable salt thereof, wherein n is an integer from 1 to 20.

In particular embodiments, compounds according to Formulas (I), (Ib) and/or (Ib1) have a structure:

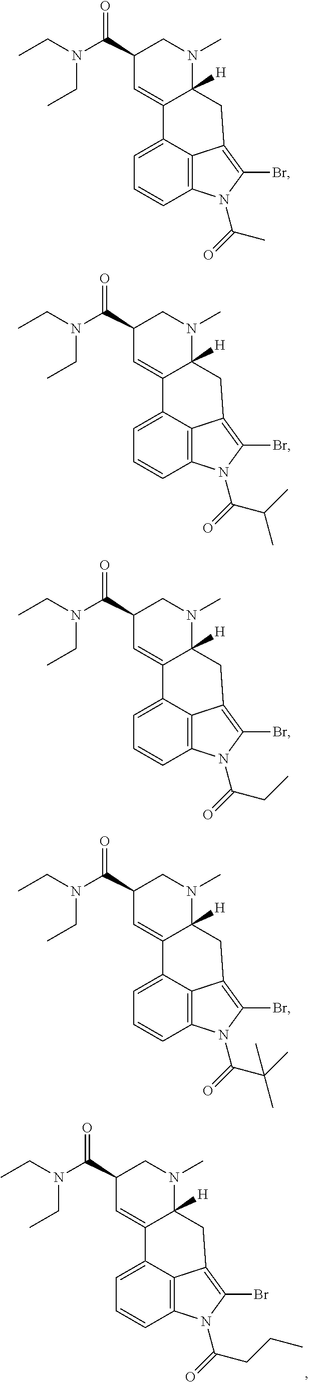

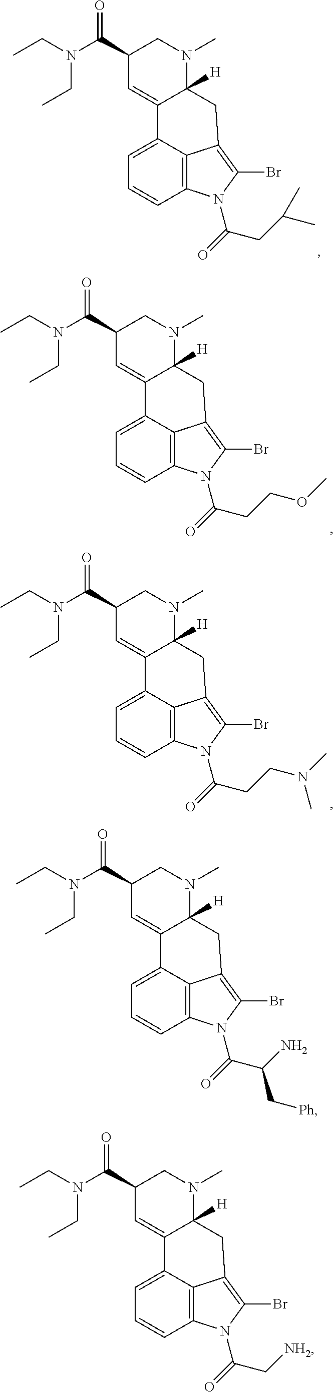

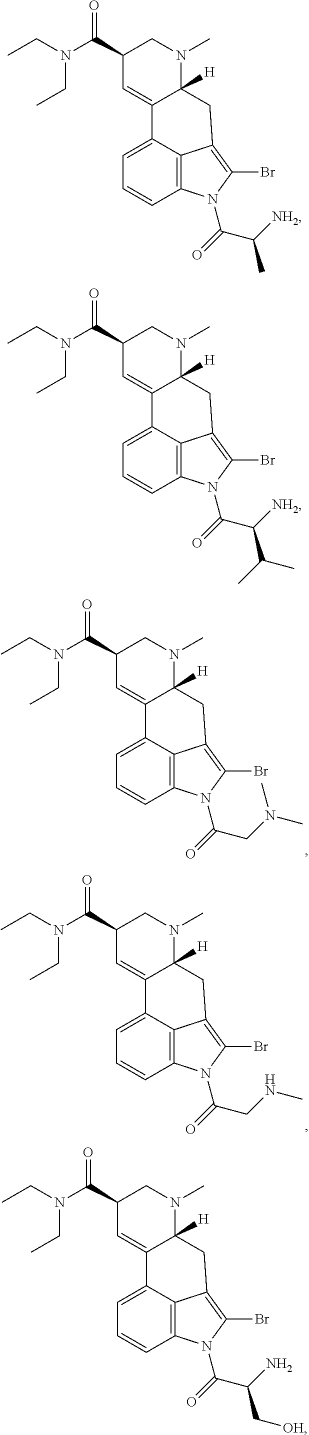

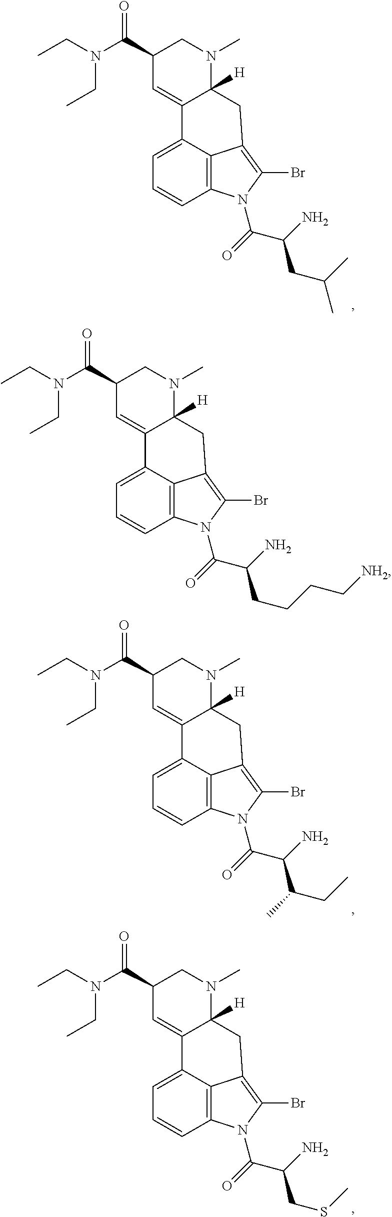

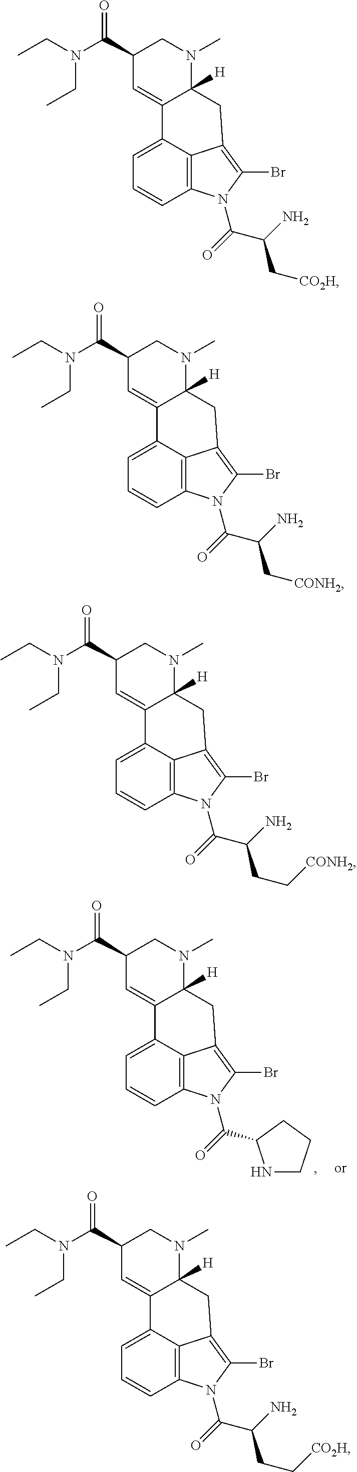 [SMILES: CCN(CC)C(=O)[C@@H]1C=C2c3cccc4c3c(c(Br)n4C(=O)[C@@H](N)CC(=O)O)C[C@H]2N(C)C1 ; CCN(CC)C(=O)[C@@H]1C=C2c3cccc4c3c(c(Br)n4C(=O)[C@@H](N)CC(N)=O)C[C@H]2N(C)C1 ; CCN(CC)C(=O)[C@@H]1C=C2c3cccc4c3c(c(Br)n4C(=O)[C@@H](N)CCC(N)=O)C[C@H]2N(C)C1 ; CCN(CC)C(=O)[C@@H]1C=C2c3cccc4c3c(c(Br)n4C(=O)[C@@H]3CCCN3)C[C@H]2N(C)C1 ; CCN(CC)C(=O)[C@@H]1C=C2c3cccc4c3c(c(Br)n4C(=O)[C@@H](N)CCC(=O)O)C[C@H]2N(C)C1]

or a pharmaceutically acceptable salt thereof.

In certain embodiments of compounds of Formula (I) are provided compounds of Formula (Ic):

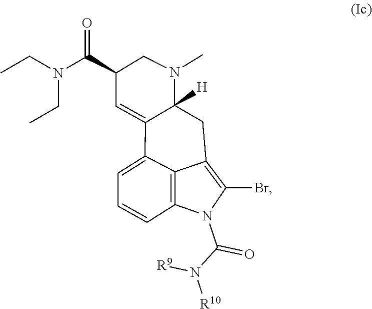

or a pharmaceutically acceptable salt thereof, wherein each of R⁹ and R¹⁰ is independently hydrogen, alkyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A}.

In some embodiments of Formulas (I) and (Ic), R⁹ is alkyl that is substituted. In some embodiments of a compound of Formula (Ic), R⁹ is alkyl substituted with heteroalkyl, heterocycloalkyl, or heteroaryl, wherein each of heteroalkyl, heterocycloalkyl, and heteroaryl is unsubstituted or substituted.

In some embodiments of Formula (Ic) R⁹ is alkyl that is unsubstituted and R¹⁰ is hydrogen. In some embodiments of Formula (Ic), R⁹ is heteroalkyl. In some embodiments of Formula (Ic), R⁹ is heteroalkyl that is unsubstituted. In some embodiments of Formula (I), R⁹ is ethyl.

In some embodiments of Formulas (I) and (Ic), R¹ is —C(O)NR⁹R¹⁰, wherein one of R⁹ and R¹⁰ is unsubstituted alkyl or alkyl substituted with heterocycloalkyl and the other of R⁹ and R¹⁰ is hydrogen. In some embodiments of Formula (I), R¹ is —C(O)NR⁹R¹⁰, wherein R⁹ is alkyl substituted with —N(R¹³)C(O)OR¹⁴ and R¹³ is hydrogen or alkyl. In some embodiments of Formula (Ic), R¹³ is hydrogen or alkyl. In some embodiments of Formula (Ic), R¹⁴ is alkyl, aryl, or heteroaryl. In one embodiment of a compound according to Formulas (I) and (Ic), R⁹ is haloalkyl.

In some embodiments of compounds of Formula (Ia,) R⁹ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl and R¹⁰ is hydrogen or alkyl. In some embodiments is a compound of Formula (Ic), wherein R⁹ is methyl, ethyl, n-propyl, isopropyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, vinyl, phenyl, 2-pyridyl, 3-pyridyl, 4-pyridyl, 2-pyrimidyl, 4-pyrimidyl, 5-pyrimidyl, or 6-pyrimidyl.

In one embodiment, of compounds of Formula (I), compounds of Formula (Ic) have the structure of Formula (Ic1):

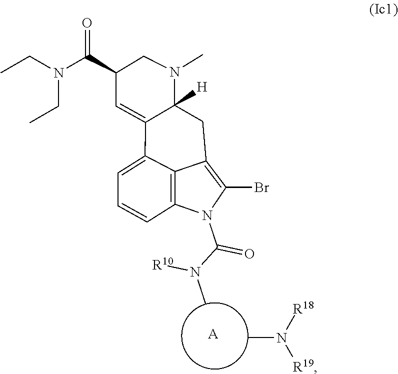

or a pharmaceutically acceptable salt thereof, wherein

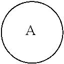

is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

In one embodiment R¹⁸ and R¹⁹ together with the atom to which they are attached form a heterocycloalkyl ring that is substituted, such as with one or more R^{A} or R^{B}. In one embodiment of Formula (Ic1), R¹⁰ is alkyl, cycloalkyl, heteroalkyl or hydrogen. In one embodiment of Formula (Ic1), R¹⁰ is alkyl that is substituted or unsubstituted, or hydrogen. In some embodiments of a compound of Formula (Ic1), each of R¹⁸ and R¹⁹ is independently hydrogen methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, or —CH₂cPr.

In some embodiments of a compound of Formula (Ic1), R¹⁸ and R¹⁹ together with the atom to which they are attached form an azetidine ring, piperidine ring, piperazine ring, a morpholine ring, or a pyrrolidine ring, each of which is substituted or unsubstituted.

In one embodiment of Formulas (I), (Ic) and (Ic1), compounds have the structure:

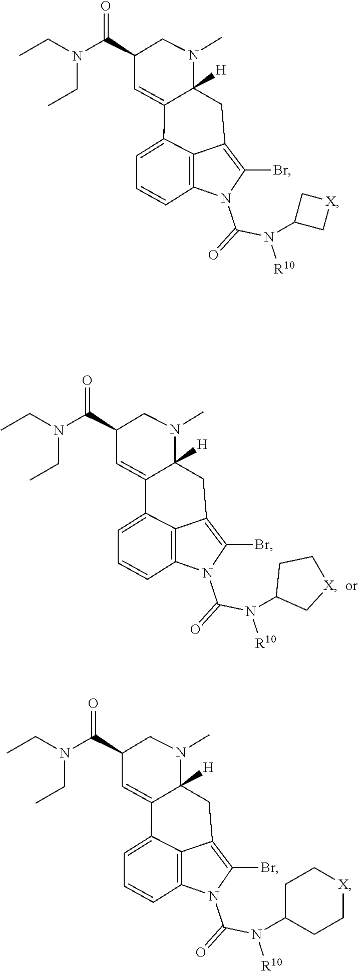

or a pharmaceutically acceptable salt thereof, and each X is independently —O—, —S—, —S(O)—, —S(O)₂—, —NH—, or —NR^{A}.

In one embodiment of compounds of Formula (Ic1), compounds have the structure of Formula (Ic2):

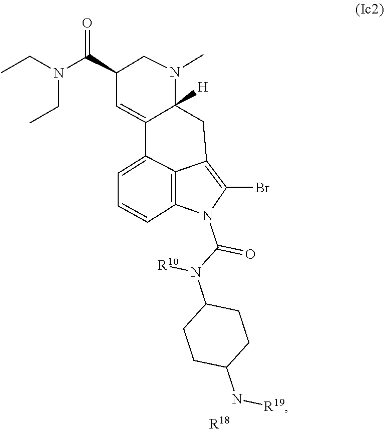

or a pharmaceutically acceptable salt thereof, wherein each of R¹⁸ and R¹⁹ is independently hydrogen, alkyl, cycloalkyl, or heteroalkyl; or R¹⁸ and R¹⁹ together with the atom to which they are attached form a heterocycloalkyl ring optionally substituted with one or more R^{B}.

In one embodiment of compounds according to Formula (Ic) compounds having the structure of Formula (Ic3) are provided:

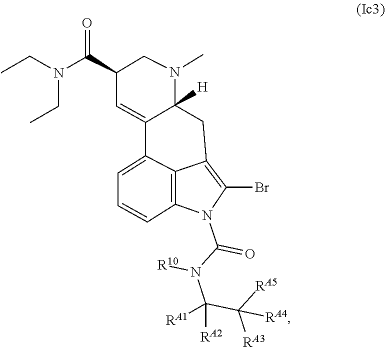

or a pharmaceutically acceptable salt thereof, wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.

In some embodiments of Formula (Ic3) one of R^{A1}, R^{A2}, R^{A3}, and R^{A4} is alkyl, and each of R^{A1}, R^{A2}, R^{A3}, and R^{A4} that is not alkyl is hydrogen. In some of Formula (Ic3), two of R^{A1}, R^{A2}, R^{A3}, and R^{A4} are alkyl, and each of R^{A1}, R^{A2}, R^{A3}, and R^{A4} that is not alkyl is hydrogen. In some embodiments of Formula (Ia3), each of R^{A1}, R^{A2}, R^{A3}, and R^{A4} is hydrogen. In some embodiments of Formula (Ic3), R^{A3}, and R^{A4} together with the atom to which they are attached form a cycloalkyl ring, and R^{A1} and R^{A2} are each hydrogen.

In some embodiments of a compound of Formula (Ic3), R^{A1} is C(O)OR¹³, and R¹³ is selected from methyl, ethyl, n-propyl, isopropyl, n-butyl, tert-butyl, n-pentyl, iso-amyl, phenyl, 2-pyridyl, 3-pyridyl, 4-pyridyl, 2-pyrimidyl, 4-pyrimidyl, 5-pyrimidyl, and 6-pyrimidyl. In some embodiments of a compound of Formula (Ic3), each of R^{A1}, R^{A2}, R^{A5}, and R^{A4} is hydrogen.

In one embodiment of Formula (Ic), disclosed compounds have a structure:

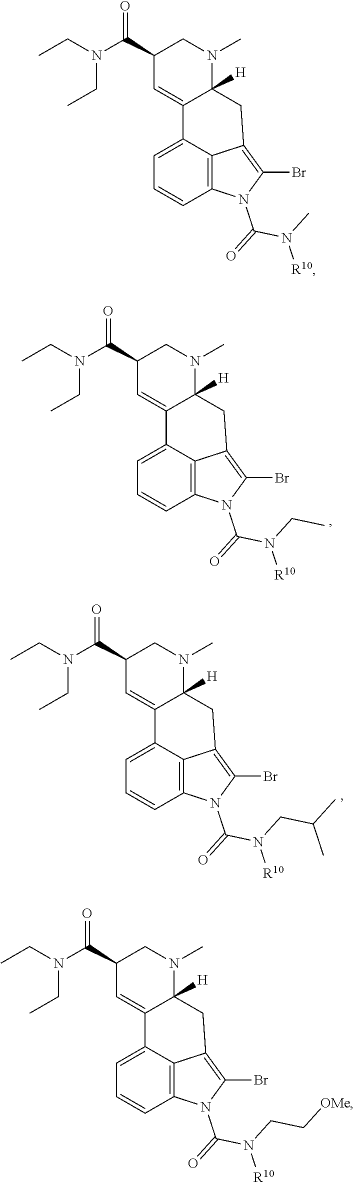

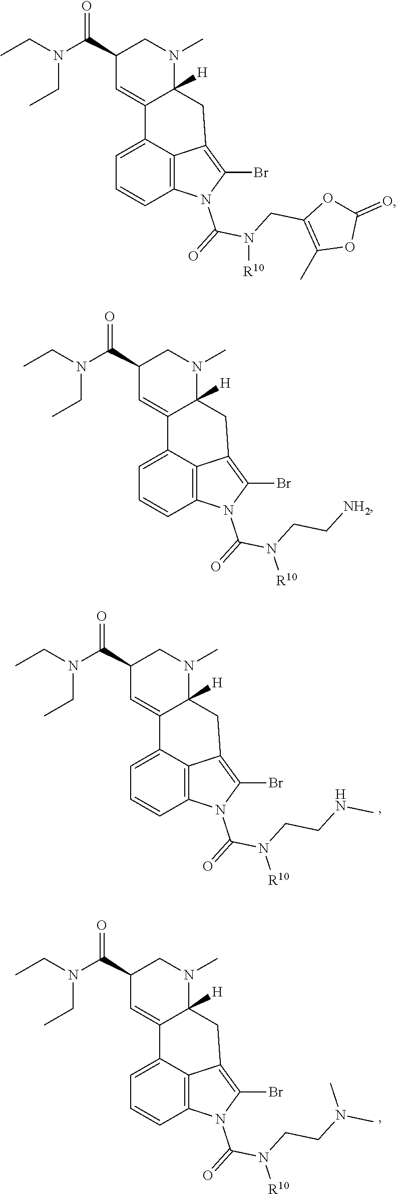

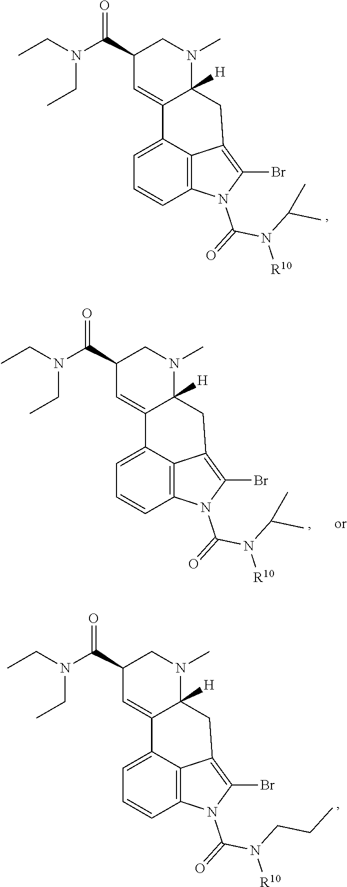

or a pharmaceutically acceptable salt thereof.

In one embodiment of Formula (Ic) compounds wherein R⁹ is alkyl, R⁹ is a straight chain alkyl, such as of the structure

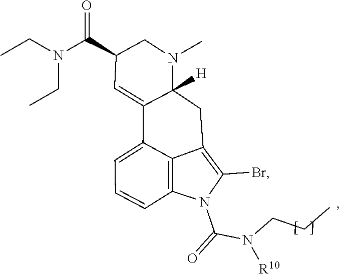

or a pharmaceutically acceptable salt thereof, wherein n is an integer from 1 to 20.

In one embodiment of Formula (I), R¹ is —CH(R⁴)OP(O)OR¹¹(OR¹²), such as illustrated in the structure of Formula (Id)

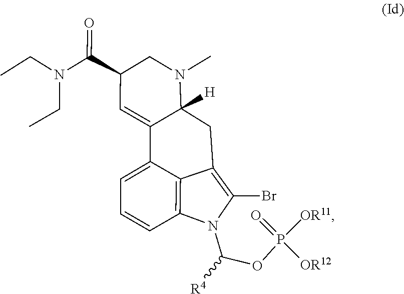

or a pharmaceutically acceptable salt thereof, wherein R⁴ is hydrogen, alkyl, cycloalkyl, or heteroalkyl.

In some embodiments of Formula (I), R¹ is —CH(R⁴)OP(O)OR¹¹(OR¹²), wherein R⁴ is hydrogen, unsubstituted alkyl, unsubstituted cycloalkyl, unsubstituted heteroalkyl, or alkyl substituted with heteroaryl. In some embodiments of a compound of Formula (I), R¹ is —CH(R⁴)OP(O)OR¹¹(OR¹²), wherein each of R¹¹ and R¹² is independently selected from hydrogen, alkyl, cycloalkyl, aryl, heteroaryl, or alkyl. In some embodiments of Formula (I), R¹ is —CH(R⁴)OP(O)OR¹¹(OR¹²), wherein each of R¹¹ and R¹² is independently selected from hydrogen, unsubstituted alkyl, unsubstituted cycloalkyl, unsubstituted aryl, unsubstituted heteroaryl, unsubstituted alkyl, or alkyl substituted with aryl or heteroaryl. In some embodiments of a compound of Formula (I), R¹ is —CH(R⁴)OP(O)OR¹¹(OR¹²), wherein each of R¹¹ and R¹² is hydrogen. In some embodiments of a compound of Formula (I), R¹ is —CH(R⁴)OP(O)OR¹¹(OR¹²), wherein at least one of R¹¹ and R¹² is alkyl. In some embodiments of a compound of Formula (I), R² is —CH(R⁴)OP(O)OR¹¹(OR¹²), and at least one of R¹¹ and R¹² is unsubstituted alkyl. In some embodiments of a compound of Formula (I), R¹ is —CH(R⁴)OP(O)OR¹¹(OR¹²), wherein at least one of R¹¹ and R¹² is alkyl substituted with —OC(O)R¹⁴. In some embodiments of a compound of Formula (I), R¹ is —CH(R⁴)OP(O)OR¹¹(OR¹²), wherein at least one of R¹¹ and R¹² is alkyl substituted with —OC(O)R¹⁴, wherein each R¹⁴ is alkyl, cycloalkyl, heterocycloalkyl, aryl, or heteroaryl. In some embodiments of a compound of Formula (I), R¹ is —CH(R⁴)OP(O)OR¹¹(OR¹²), wherein each of R¹¹ and R¹² is alkyl substituted with —OC(O)R¹⁵, wherein each R¹⁴ is unsubstituted alkyl, unsubstituted cycloalkyl, unsubstituted heterocycloalkyl, unsubstituted aryl, or unsubstituted heteroaryl. In some embodiments of a compound of Formula (I), R¹ is —CH(R⁴)OP(O)OR¹¹(OR¹²), wherein each of R¹¹ and R¹² is alkyl substituted with —OC(O)R¹⁵, and each R¹⁵ is heterocycloalkyl substituted with alkyl or arylalkyl.

In one embodiment of a compound of Formula (I), R¹ is —CH(R⁴)OP(O)OR¹¹(OR¹²), wherein at least one of R¹¹ and R¹² is —CH(R⁵)OC(O)R¹⁴, wherein each R¹⁴ is alkyl, cycloalkyl, heterocycloalkyl, aryl, or heteroaryl. In another embodiment, at least one of R¹¹ and R¹² is —CH(R⁵)C(O)R¹⁴, wherein each R¹⁴ is alkyl, cycloalkyl, heterocycloalkyl, aryl, or heteroaryl.

In one embodiment of Formulas (I) and (Id) described above, compounds have the structure of Formula (Id1):

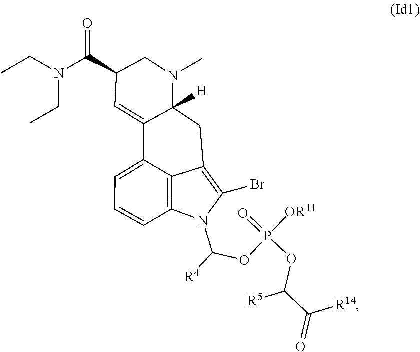

or a pharmaceutically acceptable salt thereof, wherein R⁵ is hydrogen, alkyl, cycloalkyl, or heteroalkyl.

In one embodiment of Formulas (I), (Id) and (Id1), compounds having the structure of Formula (Id2), or a pharmaceutically acceptable salt thereof,

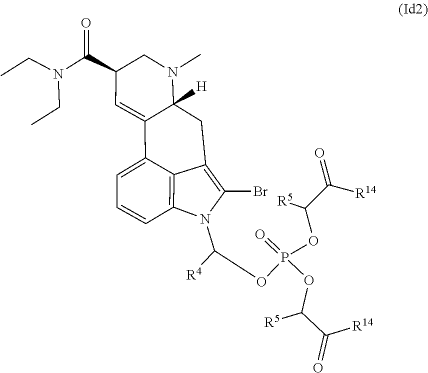

are provided.

In one embodiment of Formulas (Id), (Id1) and (Id2), R¹⁴ is

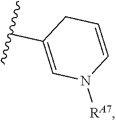

and R^{A7} is hydrogen or alkyl.

In one embodiment of a compound of Formula (I), R¹ is —CH(R⁴)OP(O)OR¹¹(OR¹²), wherein at least one of R¹¹ and R¹² is —CH(R⁵)OC(O)OR¹³ or —CH(R⁵)C(O)OR¹³, wherein each R¹³ is alkyl, cycloalkyl, heterocycloalkyl, aryl, or heteroaryl. In another embodiment, at least one of R¹¹ and R¹² is —CH(R⁵)C(O)OR¹³, wherein each R¹³ is alkyl, cycloalkyl, heterocycloalkyl, aryl, or heteroaryl.

In one embodiment of Formula (Id), compounds have the structure of Formula (Id3)

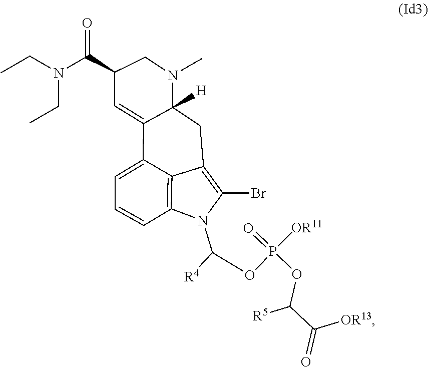

or a pharmaceutically acceptable salt thereof.

In one embodiment of Formulas (Id) and (Id3), compounds have the structure of Formula (Id4)

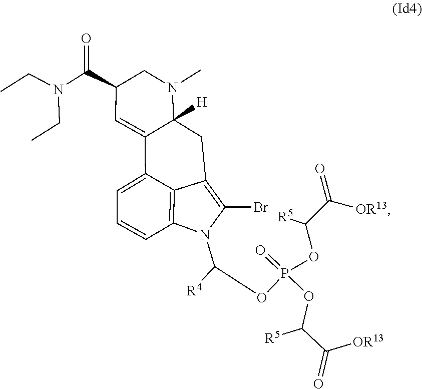

or a pharmaceutically acceptable salt thereof.

In some embodiments of a compound of Formula (I), R¹ is —P(O)OR¹¹(OR¹²). In some embodiments of Formula (I), R¹ is —P(O)OR¹¹(OR¹²), wherein each of R¹¹ and R¹² is independently hydrogen or alkyl. In some embodiments of Formula (I), R¹ is —P(O)OR¹¹(OR¹²), wherein one or both of R¹¹ and R¹² are hydrogen. In some embodiments of Formula (I), R¹ is —P(O)OR¹¹(OR¹²), wherein each of R¹¹ and R¹² is unsubstituted alkyl. In some embodiments of Formula (I), R¹ is —P(O)OR¹¹(OR¹²), wherein each of R¹¹ and R¹² is alkyl substituted with one or more R^{A}. In some embodiments of Formula (I), R¹ is —P(O)OR¹“(OR¹²), wherein each of R” and R¹² is alkyl substituted with —C(O)OR¹³. In some embodiments of Formula (I), R¹ is —P(O)OR¹¹(OR¹²), wherein each of R¹¹ and R¹² is alkyl substituted with —C(O)OR¹³ and R¹³ is alkyl, cycloalkyl, aryl, or heteroaryl. In some embodiments of Formula (I), R¹ is —P(O)OR¹¹(OR¹²), wherein each of R¹¹ and R¹² is alkyl substituted with —C(O)OR¹³ and R¹³ is unsubstituted alkyl, unsubstituted cycloalkyl, unsubstituted aryl, or unsubstituted heteroaryl. In some embodiments of Formula (I), R¹ is —P(O)OR¹¹(OR¹²), wherein each of R¹¹ and R¹² is alkyl substituted with —OC(O)R¹⁵. In some embodiments of Formula (I), R¹ is —P(O)OR¹¹(OR¹²), wherein each of R¹¹ and R¹² is alkyl substituted with —OC(O)R¹⁵, wherein R¹⁵ is alkyl, cycloalkyl, heteroaryl, or heterocycloalkyl. In some embodiments of Formula (I), R¹ is —P(O)OR¹¹(OR¹²), wherein each of R¹¹ and R¹² is alkyl substituted with —OC(O)R¹¹, wherein R¹¹ is unsubstituted alkyl, unsubstituted cycloalkyl, unsubstituted heteroaryl, or unsubstituted heterocycloalkyl. In some embodiments of Formula (I), R¹ is —P(O)OR¹¹(OR¹²), wherein each of R¹¹ and R¹² is alkyl substituted with —OC(O)R¹⁶, wherein R¹⁶ is heterocycloalkyl substituted with alkyl or arylalkyl. In some embodiments of Formula (I), R¹ is —P(O)OR¹¹(OR¹²), wherein each of R¹¹ and R¹² is alkyl substituted with —OC(O)OR¹⁶, wherein R¹⁶ is alkyl, cycloalkyl, heteroaryl, or heterocycloalkyl. In some embodiments of a compound of Formula (I), R¹ is —P(O)OR¹¹(OR¹²), wherein each of R¹¹ and R¹² is alkyl substituted with —OC(O)OR¹⁶, wherein R¹⁶ is unsubstituted alkyl, unsubstituted cycloalkyl, unsubstituted heteroaryl, or unsubstituted heterocycloalkyl. In some embodiments of a compound of Formula (I), R¹ is —P(O)OR¹¹(OR¹²), wherein each of R¹¹ and R¹² is alkyl substituted with —OC(O)OR¹⁶, wherein R¹⁶ is unsubstituted alkyl.

In some embodiments of Formula (I), R¹ is —P(O)OR¹¹(OR¹²), wherein R¹¹ and R¹² together with the atoms to which they are attached form a heterocycloalkyl ring. In some embodiments Formula (I), R¹ is —P(O)OR¹¹(OR¹²), wherein R¹¹ and R¹² together with the atoms to which they are attached form a heterocycloalkyl ring that is unsubstituted. In some embodiments of Formula (I), R¹ is —P(O)OR¹¹(OR¹²), wherein R¹¹ and R¹² together with the atom to which they are attached form a heterocycloalkyl ring that is substituted with aryl. In some embodiments of Formula (I), R¹ is —P(O)OR¹¹(OR¹²), wherein R¹¹ and R¹² together with the atom to which they are attached form a heterocycloalkyl ring that is substituted with unsubstituted aryl. In some embodiments of Formula (I), R¹ is —P(O)OR¹¹(OR¹²), wherein R¹¹ and R¹² together with the atom to which they are attached form a heterocycloalkyl ring that is substituted with aryl, wherein the aryl is substituted with halogen.

In one embodiment of Formula (I) described above, compounds according to the structure of Formula (Ie), or a pharmaceutically acceptable salt thereof, are provided:

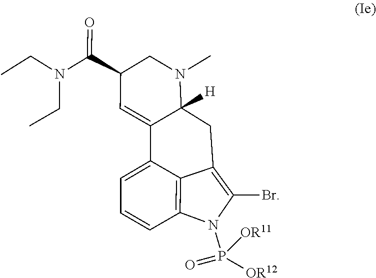

In one embodiment of compounds of Formulas (I) and (Ie), compounds having the structure of Formula (Ie1), or a pharmaceutically acceptable salt thereof, are provided:

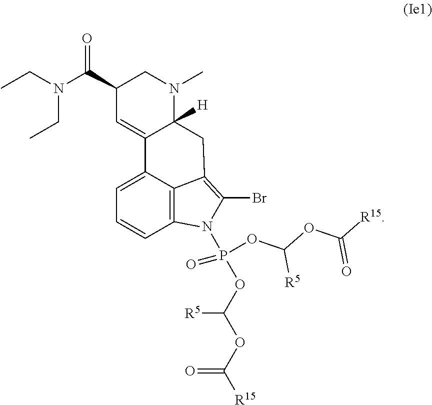

In one embodiment of Formula (Ie1), wherein R⁵ is hydrogen, alkyl, cycloalkyl, or heteroalkyl. In one embodiment of Formula (Ie1), R¹⁴ is

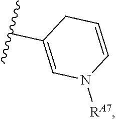

and R^{A7} is hydrogen or alkyl.

In one embodiment of compounds of Formulas (I) and (Ie), compounds having the structure of Formula (Ie2), or a pharmaceutically acceptable salt thereof, are provided

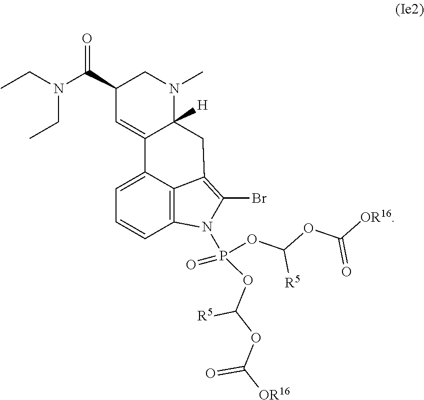

In one embodiment of Formulas (Ie1) and (Ie2), R⁵ is hydrogen or alkyl. In some embodiments of Formulas (Ie1) and (Ie2), R⁵ is hydrogen.

In one embodiment of Formula (Ie) compounds have R¹¹ and R¹² together with the atoms to which they are attached form a heterocycloalkyl ring that is unsubstituted or substituted with one or more R^{A}. In one embodiment, R^{A} is aryl, including substituted aryl, such as aryl substituted with halogen.

In one embodiment of Formula (I), compounds having the structure of Formula (If), or a pharmaceutically acceptable salt thereof, are provided:

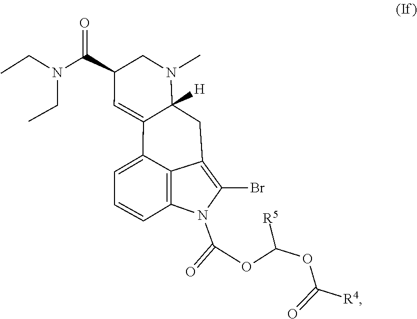

wherein R⁴ is alkyl, alkenyl, haloalkyl, cycloalkyl, heterocycloalkyl, aryl, heteroaryl, or heteroalkyl; and R⁵ is hydrogen, alkyl, or cycloalkyl.

In some embodiments of compounds of Formula (If), R⁴ is heteroalkyl. In some embodiments of Formula (If), R⁴ is heterocycloalkyl. In some embodiments of Formula (If), R⁵ is hydrogen and R⁴ is heteroalkyl. In some embodiments of a compound of Formula (If), R⁵ is hydrogen and R⁴ is heterocycloalkyl. In some embodiments of Formula (If), R⁵ is hydrogen or C₁₋₆ alkyl and R⁴ is heteroalkyl. In some embodiments of Formula (If), R⁴ is heterocycloalkyl.

In some embodiments of Formulas (I) and (If), R⁴ is alkyl. In some embodiments of a compound of Formula (If), R⁴ is CH₂CF₃. In some embodiments of a compound of Formula (If), R⁴ is unsubstituted alkyl. In some embodiments of Formula (If), R⁴ is methyl, ethyl, n-propyl, isopropyl, n-butyl, tert-butyl, 3-methyl-1-butyl, n-pentyl, n-hexyl, n-heptyl, n-octyl, or n-nonyl.

In some embodiments a compound of Formula (If) is one wherein R⁴ is cycloalkyl. In some embodiments of compound of Formula (If), R⁴ is unsubstituted cycloalkyl. In some embodiments of compounds of Formula (If), R⁴ is cyclopropyl, cyclobutyl, cyclopentyl, cyclohexyl, or cycloheptyl. In some embodiments of a compound of Formula (If), the compound is one wherein R⁴ is aryl. In some embodiments of Formula (If), wherein R⁴ is substituted or unsubstituted phenyl. In other embodiments of Formula (If), R⁴ is heteroaryl and in certain such some embodiments of Formula (If), R⁴ is 2-pyridyl, 3-pyridyl, 4-pyridyl, 2-pyrimidyl, 3-pyrimidyl, or 6-pyrimidyl.

In some embodiments of Formulas (I) and (If), R⁴ is R^{A} substituted with —OR¹³, —N(R¹⁸)R¹⁹, or —C(O)OR¹³, such as wherein R⁴ is alkyl, substituted with —OR¹³, —N(R¹⁸)R¹⁹, or —C(O)OR¹³. In some embodiments of Formula (If), R⁴ is alkyl substituted with —N(R¹⁸)R¹⁹, R⁴ is alkyl substituted with —N(R¹⁸)R¹⁹, each of R¹⁸ and R¹⁹ is independently alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B}. In one embodiment of Formula (If) R⁴ is alkyl substituted with —N(R¹⁸)R¹⁹, wherein R¹⁸ and R¹⁹ together with the atom to which they are attached form a heterocycloalkyl ring, for example a heterocycloalkyl ring substituted with substituted with one or more R^{B}, such as wherein R^{B} is selected from alkyl, arylalkyl and —C(O)CH₃. In one embodiment of Formula (If) R⁴ is alkyl substituted with —N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments of Formulas (I) and (If), R⁴ is heteroalkyl. In some embodiments of Formulas (I) and (If), R⁴ is CH₂CH₂OMe or CH₂CH₂SO₂Me. In some embodiments of Formulas (I) and (If), R⁴ is —(CH₂)ₙCO₂H, wherein n is 1, 2, 3, 4, 5, or 6. In some embodiments of Formulas (I) and (If), R⁴ is —(CH₂)ₙCO₂R¹³, wherein n is 1, 2, 3, 4, 5, or 6. In some embodiments of Formulas (I) and (If), R⁴ is —(CH₂)ₙCO₂R¹³, wherein R¹³ is alkyl. In some embodiments of Formulas (I) and (If), R⁴ is —(CH₂)ₙCO₂R¹³, wherein R¹³ is unsubstituted alkyl. In some embodiments of Formulas (I) and (If), R⁴ is —(CH₂)ₙCO₂R¹³, wherein R¹³ is methyl, ethyl, n-propyl, isopropyl, n-butyl, tert-butyl, or —CH(Et)₂.

In some embodiments of Formulas (I) and (If), R⁴ is —(CH₂)ₙOR¹³, wherein n is 1, 2, 3, 4, 5, 6 or 7. In some embodiments of Formulas (I) and (If), R⁴ is —(CH₂)ₙOR¹³, wherein R¹³ is alkyl. In some embodiments of Formulas (I) and (If), R⁴ is —(CH₂)ₙOR¹³, wherein R¹³ is unsubstituted alkyl. In some embodiments of Formulas (I) and (If), R⁴ is —(CH₂)ₛOR¹³, wherein R¹³ is methyl, ethyl, n-propyl, isopropyl, n-butyl, tert-butyl, or —CH(Et)₂.

In some embodiments of Formulas (I) and (If), R⁴ is —(CH₂)ₙ N(R¹⁸)R¹⁹, wherein n is 1, 2, 3, 4, 5, 6 or 7. In some embodiments of Formulas (I) and (If), R⁴ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring.

In some embodiments is a compound of Formula (If), R⁵ is unsubstituted alkyl. In some embodiments of Formula (If), R⁵ is methyl, ethyl, n-propyl, isopropyl, tert-butyl, or hydrogen. In some embodiments of Formula (If), R⁵ is methyl or hydrogen. In some embodiments of a compound of Formula (If), R⁵ is methyl. In some embodiments of a compound of Formula (If) R⁵ is hydrogen.

In some embodiments of Formula (If), wherein R⁴ is methyl, ethyl, n-propyl, isopropyl, tert-butyl, n-pentyl, iso-amyl, n-hexyl, n-heptyl, n-octyl, n-nonyl, cyclopropyl, cyclobutyl, cyclopentyl, cyclohexyl, cycloheptyl, cyclooctyl, —CH₂CF₃, —CH₂cPr, —CH₂CH₂OMe, vinyl, phenyl, 2-pyridyl, 3-pyridyl, 4-pyridyl, 2-pyrimidyl, 4-pyrimidyl, 5-pyrimidyl, or 6-pyrimidyl, R⁵ is hydrogen. In some embodiments of a compound of Formula (If), R⁵ is methyl, ethyl, n-propyl, isopropyl, or —CH(Et)₂.

In one embodiment of a compound according to Formula (If), R⁴ is

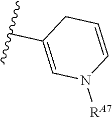

and R^{A7} is hydrogen or alkyl.

In one embodiment, compounds according to Formula (If) have a structure:

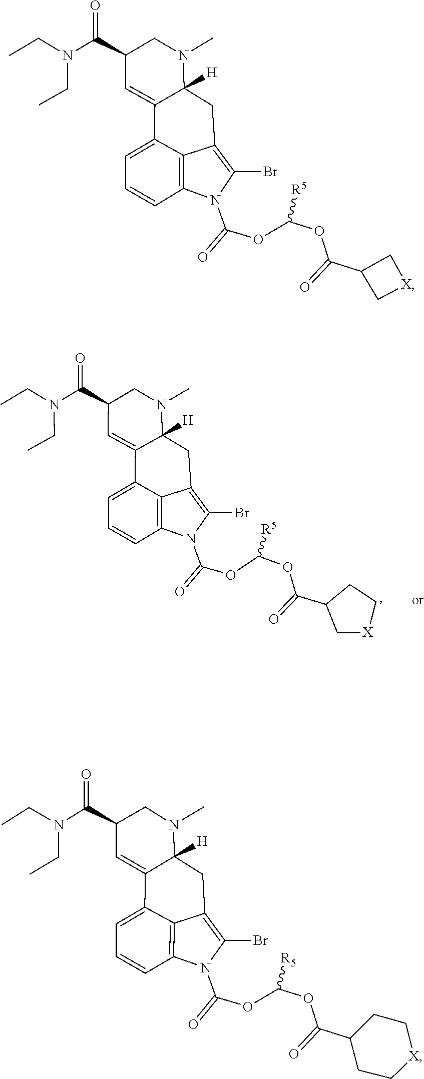

or a pharmaceutically acceptable salt thereof, wherein each X is independently —O—, —S—, —S(O)—, —S(O)₂—, —NH—, or —NR^{B}.

With continued reference to Formula (If), in one embodiment, R⁴, together with the carbonyl to which it is attached is an amino acid residue. For example, in one embodiment of Formula (If), R⁴ is —CH(R^{A1})NH₂; and R^{A1} is an amino acid side chain. In some embodiments, the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

In one embodiment of Formula (I), compounds having the structure of Formula (Ig) are provided:

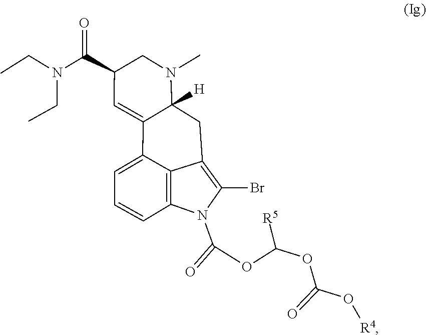

or a pharmaceutically acceptable salt thereof, wherein R⁴ is selected from alkyl, alkenyl, heteroalkyl, cycloalkyl, haloalkyl, heterocycloalkyl, aryl, and heteroaryl, each of which is substituted or unsubstituted; and R⁵ is alkyl that is substituted or unsubstituted, or hydrogen.

In some embodiments of Formulas (I) and (Ig), R⁴ is —(CH₂)ₙ N(R¹⁸)R¹⁹, wherein n is 1, 2, 3, 4, 5, 6 or 7. In some embodiments of Formulas (I) and (Ig), R⁴ is —(CH₂)ₙ—N(R¹⁸)R¹⁹, wherein R¹⁸ and R¹⁹ together with the atom to which they are attached form a heterocycloalkyl ring, such as an azetidinyl, pyrrolidinyl, morpholinyl, piperazinyl or piperidinyl ring optionally substituted with one or more R^{B}. In some embodiments of Formula (Ig), R⁵ is methyl, and in some embodiments R⁵ is hydrogen.

In some embodiments of Formulas (I) and (Ig), R⁴ is selected from

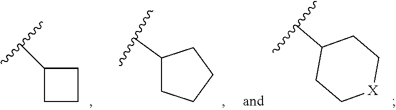

and each X is independently —O—, —S—, —S(O)—, —S(O)₂—, —NH—, or —NR^{A}, and in some embodiments of Formula (Ig), R⁴ is selected from

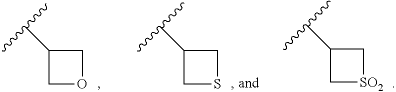 [SMILES: *C1COC1 ; *C1CSC1 ; *C1CS(=O)(=O)C1]

In a particular embodiment of Formula (Ig), R⁴ is

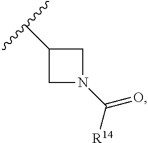

and R¹⁴ is alkyl, cycloalkyl, or aryl, such as wherein R¹⁴ is selected from methyl, ethyl, n-propyl, isopropyl, phenyl and CH₂CH₂OMe.

In a particular embodiment of Formula (Ig), R⁴ is

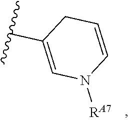

wherein R^{A7} is hydrogen or alkyl.

In one embodiment of Formula (I), compounds having the structure of Formula (Ih) are provided:

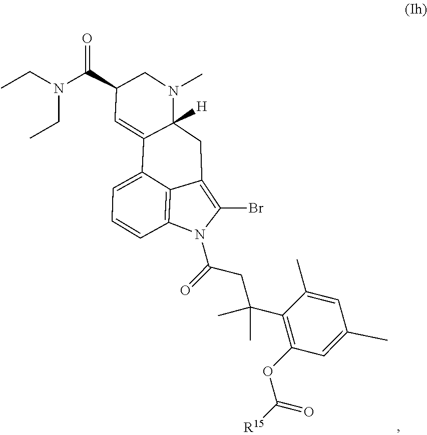

or a pharmaceutically acceptable salt thereof, wherein R⁵ is selected from alkyl, alkenyl, haloalkyl, heteroalkyl, cycloalkyl, heterocycloalkyl, aryl, and heteroaryl. In a particular embodiment of Formula (Ih), wherein R¹⁵ and the carbonyl to which R¹⁵ is attached form an amino acid residue. In some embodiments, R¹⁵ is —CH(R^{A1})NH₂, wherein R^{A1} is an amino acid side chain. In some embodiments, the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

In some embodiments of a compound of Formula (I), R¹ is —S(O)₂OR⁷. In some embodiments of Formula (I), R¹ is —S(O)₂OR⁷, wherein R⁷ is alkyl. In some embodiments of Formula (I), R¹ is —S(O)₂OR⁷, wherein R⁷ is alkyl substituted with —C(O)R¹⁴. In some embodiments of Formula (I), R¹ is —S(O)₂OR⁷, wherein R⁷ is alkyl substituted with —C(O)R¹⁴, wherein R¹⁴ is alkyl, heteroalkyl, cycloalkyl, heterocycloalkyl, aryl, or heteroaryl. In some embodiments of Formula (I), R¹ is —S(O)₂OR⁷, wherein R⁷ is alkyl substituted with —C(O)R¹⁴. In some embodiments of Formula (I), R¹ is —S(O)₂OR⁷, wherein R⁷ is alkyl substituted with —C(O)R¹⁴, wherein R¹⁴ is heterocycloalkyl. In some embodiments of Formula (I), R¹ is —S(O)₂OR⁷, wherein R⁷ is alkyl substituted with —C(O)R¹⁴. In some embodiments of Formula (I), R⁷ is alkyl substituted with —C(O)R¹⁴, wherein R¹⁴ is heterocycloalkyl substituted with alkyl, —C(O)CH₃, or C(O)Ph.

In one embodiment, such compounds of Formula (I), described above wherein R¹ is —S(O)₂OR⁷, such compounds have Formula (Ii):

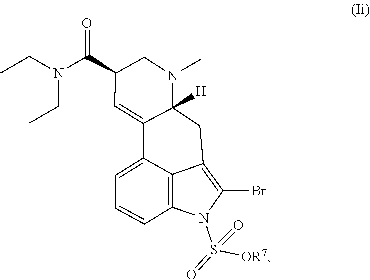

or a pharmaceutically acceptable salt thereof, wherein R⁷ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}.

In one embodiment of Formulas (I) and (Ii), compounds according to Formula (Ii1) are provided:

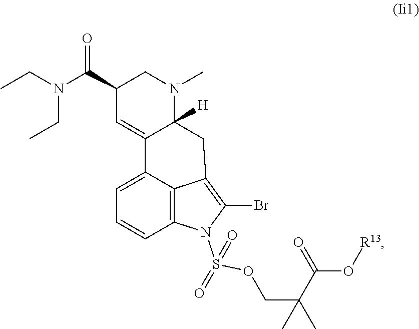

or a pharmaceutically acceptable salt thereof, wherein R¹³ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B}.

In one embodiment of Formulas (I), (Ii) and/or (Ii1), compounds are:

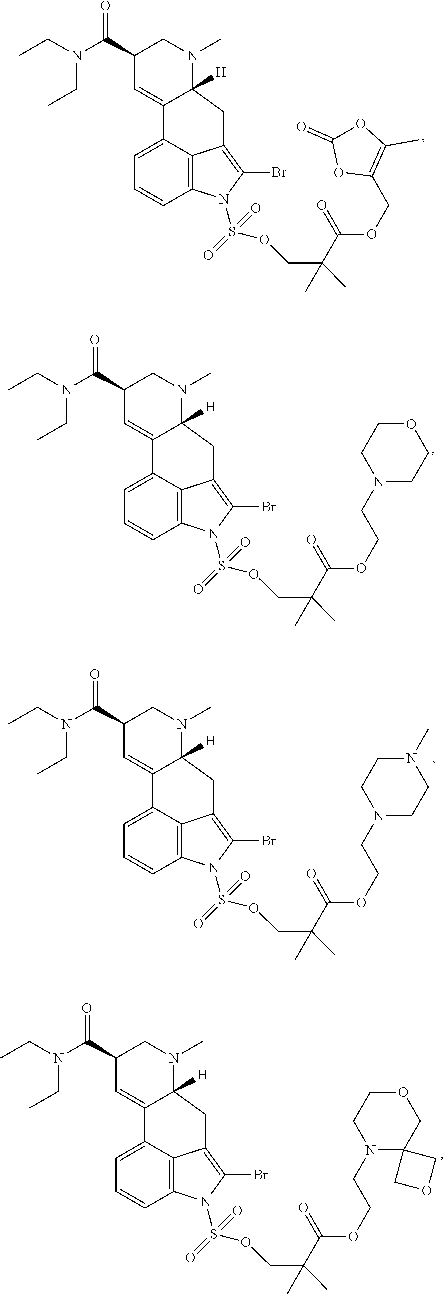

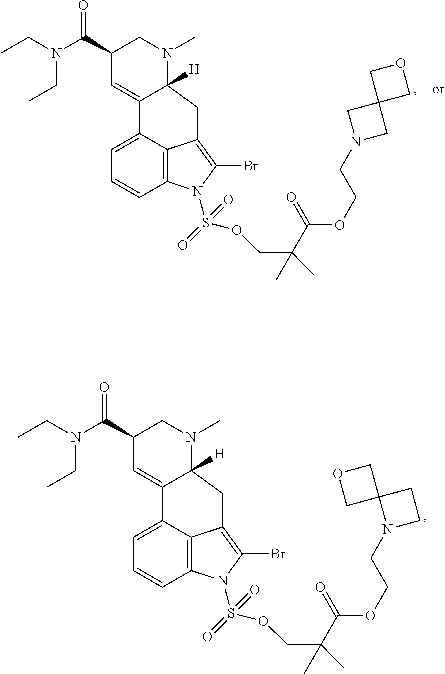

or a pharmaceutically acceptable salt thereof.

In certain embodiments of compounds according to Formula (Ii1), R¹³ is selected from

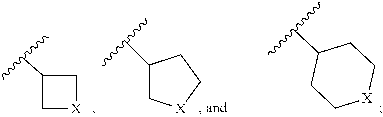

and each X is independently —O—, —S—, —S(O)—, —S(O)₂—, —NH—, or —NR^{A}.

In particular embodiments of Formulas (I), (Ii) and (Ii1), compounds having Formula (Ii2) are provided:

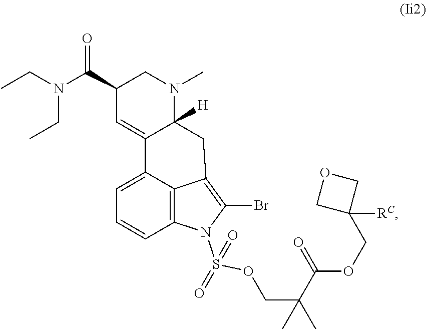

or a pharmaceutically acceptable salt thereof, wherein R^{C} is selected from hydrogen and alkyl.

In a particular embodiment of Formula (I), compounds according to Formula (Ij) are provided:

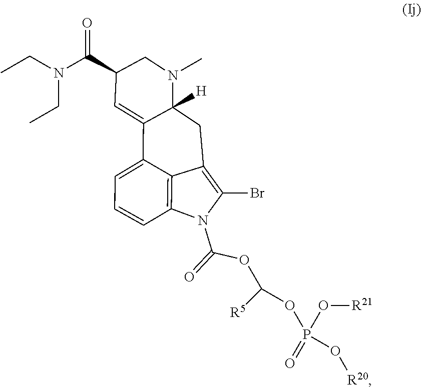

or a pharmaceutically acceptable salt thereof, wherein each of R²⁰ and R²¹ is independently alkyl, alkyl, heteroalkyl, cycloalkyl, heterocycloalkyl, aryl, heteroaryl, or hydrogen, wherein alkyl, heteroalkyl, cycloalkyl, heterocycloalkyl, aryl, or heteroaryl is unsubstituted or substituted with one or more R^{B}, or R²⁰ and R²¹ together with the atoms to which they are attached form a heterocycloalkyl ring that is unsubstituted or substituted with one or more R^{B}; and each R^{B} is independently halogen, amino, cyano, hydroxyl, alkyl, heteroalkyl, cycloalkyl, heterocycloalkyl, acyl, aryl, heteroaryl, arylalkyl, or heteroarylalkyl.

Certain compounds according to Formula (I) disclosed herein are isotopically enriched, examples of such compounds include:

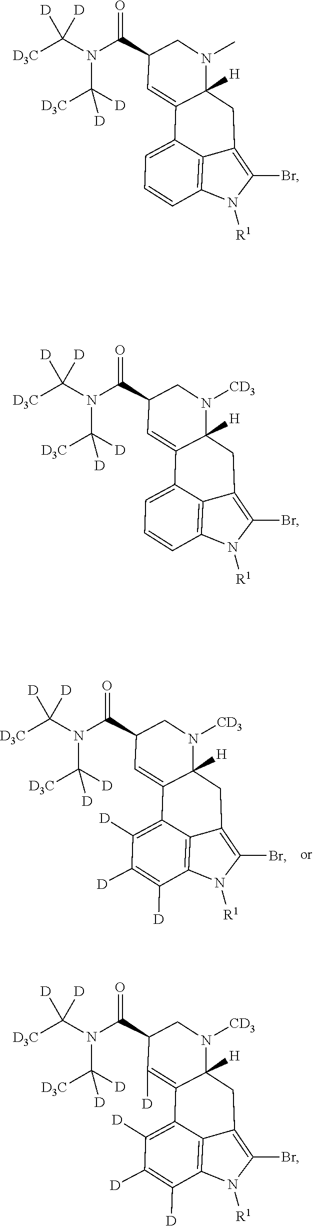

or a pharmaceutically acceptable salt thereof, wherein R¹ is as described herein.

Selected compounds of the disclosure are provided in TABLE 1.

TABLE 1

| Cpd | Structure |
|---|---|
| 1 | 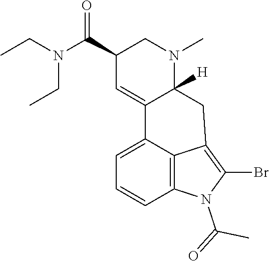 [SMILES: CCN(CC)C(=O)[C@@H]1C=C2c3cccc4c3c(c(Br)n4C(C)=O)C[C@H]2N(C)C1] |
| 2 | 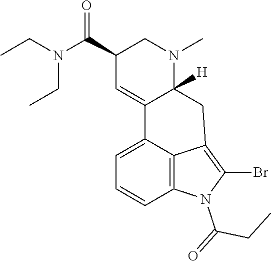 [SMILES: CCC(=O)n1c(Br)c2c3c(cccc31)C1=C[C@@H](C(=O)N(CC)CC)CN(C)[C@@H]1C2] |
| 3 | 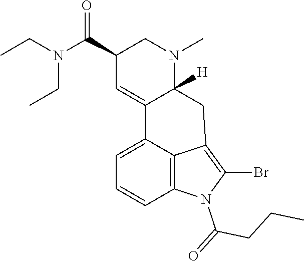 [SMILES: CCCC(=O)n1c(Br)c2c3c(cccc31)C1=C[C@@H](C(=O)N(CC)CC)CN(C)[C@@H]1C2] |
| 4 | 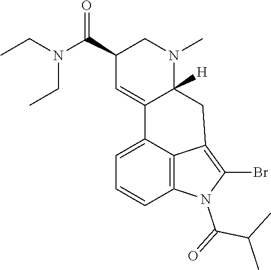 [SMILES: CCN(CC)C(=O)[C@@H]1C=C2c3cccc4c3c(c(Br)n4C(=O)C(C)C)C[C@H]2N(C)C1] |
| 5 | 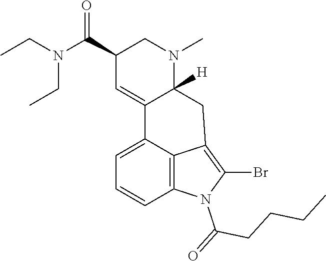 [SMILES: CCCCC(=O)n1c(Br)c2c3c(cccc31)C1=C[C@@H](C(=O)N(CC)CC)CN(C)[C@@H]1C2] |
| 6 | 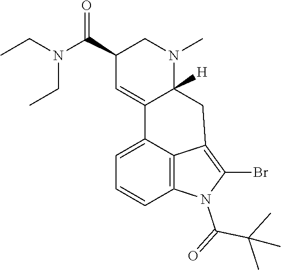 [SMILES: CCN(CC)C(=O)[C@@H]1C=C2c3cccc4c3c(c(Br)n4C(=O)C(C)(C)C)C[C@H]2N(C)C1] |
| 7 | 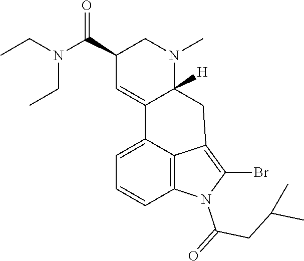 [SMILES: CCN(CC)C(=O)[C@@H]1C=C2c3cccc4c3c(c(Br)n4C(=O)CC(C)C)C[C@H]2N(C)C1] |
| 8 | 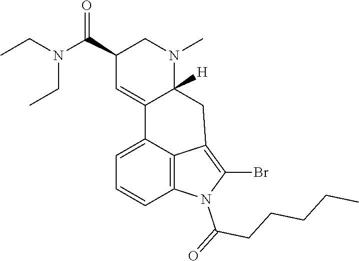 [SMILES: CCCCCC(=O)n1c(Br)c2c3c(cccc31)C1=C[C@@H](C(=O)N(CC)CC)CN(C)[C@@H]1C2] |
| 9 | 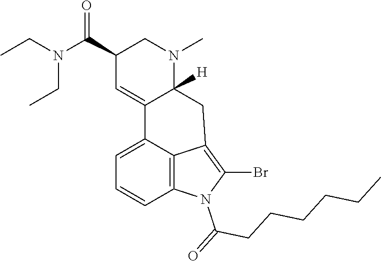 [SMILES: CCCCCCC(=O)n1c(Br)c2c3c(cccc31)C1=C[C@@H](C(=O)N(CC)CC)CN(C)[C@@H]1C2] |
| 10 | 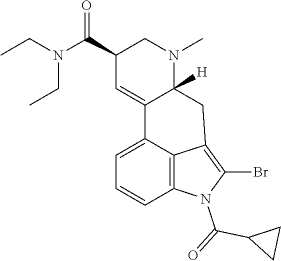 [SMILES: CCN(CC)C(=O)[C@@H]1C=C2c3cccc4c3c(c(Br)n4C(=O)C3CC3)C[C@H]2N(C)C1] |
| 11 | 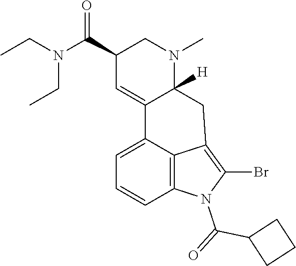 [SMILES: CCN(CC)C(=O)[C@@H]1C=C2c3cccc4c3c(c(Br)n4C(=O)C3CCC3)C[C@H]2N(C)C1] |
| 12 | 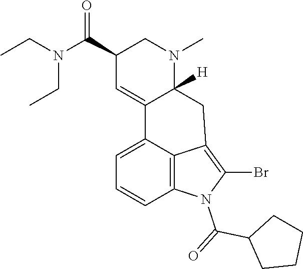 [SMILES: CCN(CC)C(=O)[C@@H]1C=C2c3cccc4c3c(c(Br)n4C(=O)C3CCCC3)C[C@H]2N(C)C1] |
| 13 | 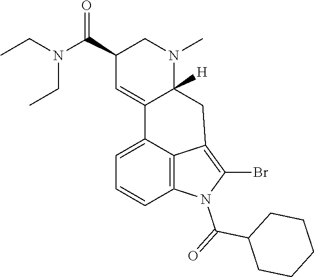 [SMILES: CCN(CC)C(=O)[C@@H]1C=C2c3cccc4c3c(c(Br)n4C(=O)C3CCCCC3)C[C@H]2N(C)C1] |
| 14 | 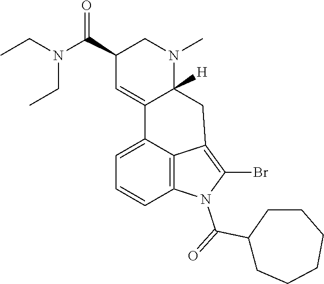 [SMILES: CCN(CC)C(=O)[C@@H]1C=C2c3cccc4c3c(c(Br)n4C(=O)C3CCCCCC3)C[C@H]2N(C)C1] |
| 15 | 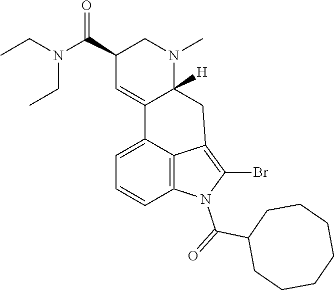 [SMILES: CCN(CC)C(=O)[C@@H]1C=C2c3cccc4c3c(c(Br)n4C(=O)C3CCCCCCC3)C[C@H]2N(C)C1] |
| 16 | 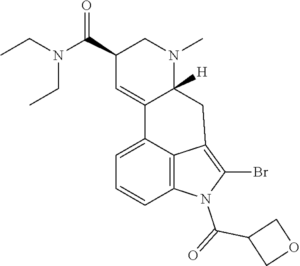 [SMILES: CCN(CC)C(=O)[C@@H]1C=C2c3cccc4c3c(c(Br)n4C(=O)C3COC3)C[C@H]2N(C)C1] |
| 17 | 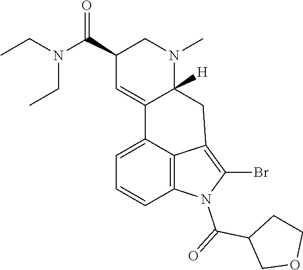 [SMILES: CCN(CC)C(=O)[C@@H]1C=C2c3cccc4c3c(c(Br)n4C(=O)C3CCOC3)C[C@H]2N(C)C1] |
| 18 | 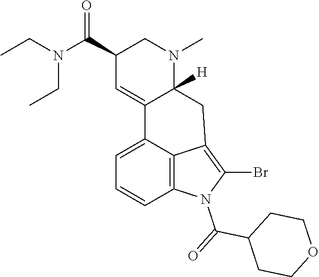 [SMILES: CCN(CC)C(=O)[C@@H]1C=C2c3cccc4c3c(c(Br)n4C(=O)C3CCOCC3)C[C@H]2N(C)C1] |
| 19 | 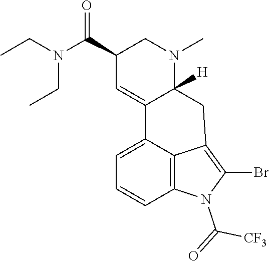 [SMILES: CCN(CC)C(=O)[C@@H]1C=C2c3cccc4c3c(c(Br)n4C(=O)C(F)(F)F)C[C@H]2N(C)C1] |
| 20 | 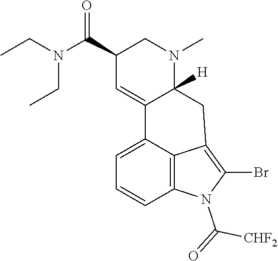 [SMILES: CCN(CC)C(=O)[C@@H]1C=C2c3cccc4c3c(c(Br)n4C(=O)C(F)F)C[C@H]2N(C)C1] |
| 21 | 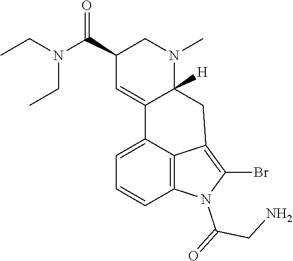 [SMILES: CCN(CC)C(=O)[C@@H]1C=C2c3cccc4c3c(c(Br)n4C(=O)CN)C[C@H]2N(C)C1] |
| 22 | 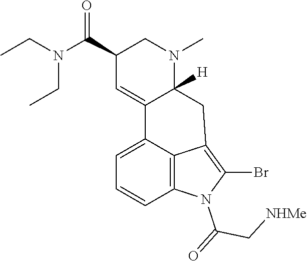 [SMILES: CCN(CC)C(=O)[C@@H]1C=C2c3cccc4c3c(c(Br)n4C(=O)CNC)C[C@H]2N(C)C1] |
| 23 | 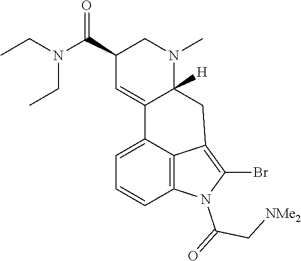 [SMILES: CCN(CC)C(=O)[C@@H]1C=C2c3cccc4c3c(c(Br)n4C(=O)CN(C)C)C[C@H]2N(C)C1] |
| 24 | 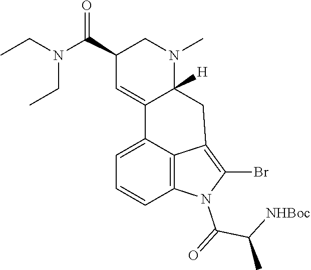 [SMILES: CCN(CC)C(=O)[C@@H]1C=C2c3cccc4c3c(c(Br)n4C(=O)[C@H](C)NC(=O)OC(C)(C)C)C[C@H]2N(C)C1] |
| 25 | 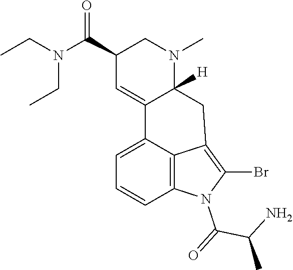 [SMILES: CCN(CC)C(=O)[C@@H]1C=C2c3cccc4c3c(c(Br)n4C(=O)[C@H](C)N)C[C@H]2N(C)C1] |
| 26 | 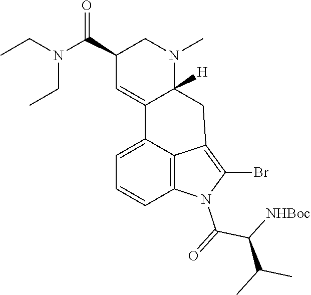 [SMILES: CCN(CC)C(=O)[C@@H]1C=C2c3cccc4c3c(c(Br)n4C(=O)[C@@H](NC(=O)OC(C)(C)C)C(C)C)C[C@H]2N(C)C1] |
| 27 | 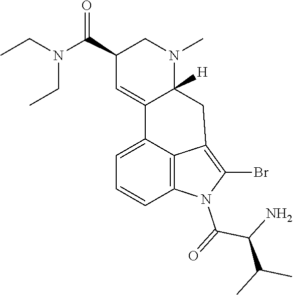 [SMILES: CCN(CC)C(=O)[C@@H]1C=C2c3cccc4c3c(c(Br)n4C(=O)[C@@H](N)C(C)C)C[C@H]2N(C)C1] |
| 28 | 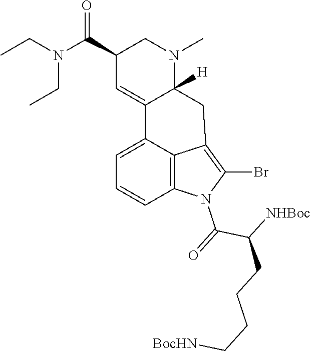 [SMILES: CCN(CC)C(=O)[C@@H]1C=C2c3cccc4c3c(c(Br)n4C(=O)[C@H](CCCCNC(=O)OC(C)(C)C)NC(=O)OC(C)(C)C)C[C@H]2N(C)C1] |
| 29 | 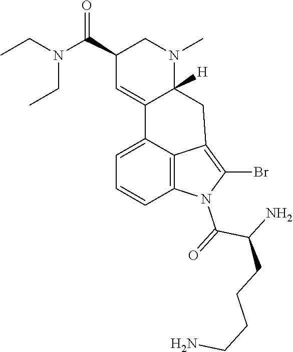 [SMILES: CCN(CC)C(=O)[C@@H]1C=C2c3cccc4c3c(c(Br)n4C(=O)[C@@H](N)CCCCN)C[C@H]2N(C)C1] |
| 30 | 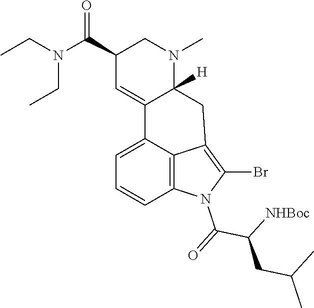 [SMILES: CCN(CC)C(=O)[C@@H]1C=C2c3cccc4c3c(c(Br)n4C(=O)[C@H](CC(C)C)NC(=O)OC(C)(C)C)C[C@H]2N(C)C1] |
| 31 | 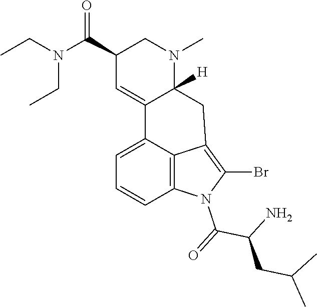 [SMILES: CCN(CC)C(=O)[C@@H]1C=C2c3cccc4c3c(c(Br)n4C(=O)[C@@H](N)CC(C)C)C[C@H]2N(C)C1] |
| 32 | 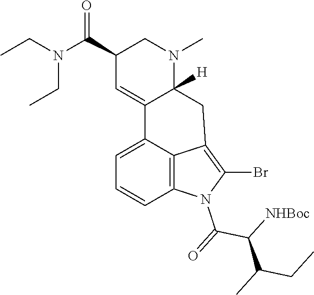 [SMILES: CCC(C)[C@H](NC(=O)OC(C)(C)C)C(=O)n1c(Br)c2c3c(cccc31)C1=C[C@@H](C(=O)N(CC)CC)CN(C)[C@@H]1C2] |
| 33 | 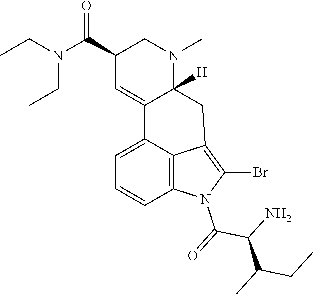 [SMILES: CCC(C)[C@H](N)C(=O)n1c(Br)c2c3c(cccc31)C1=C[C@@H](C(=O)N(CC)CC)CN(C)[C@@H]1C2] |
| 34 | 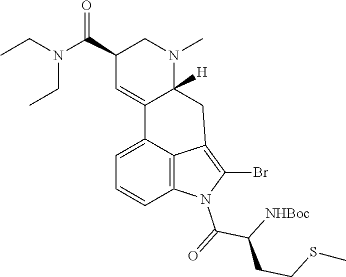 [SMILES: CCN(CC)C(=O)[C@@H]1C=C2c3cccc4c3c(c(Br)n4C(=O)[C@H](CCSC)NC(=O)OC(C)(C)C)C[C@H]2N(C)C1] |
| 35 | 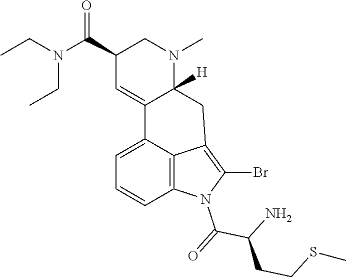 [SMILES: CCN(CC)C(=O)[C@@H]1C=C2c3cccc4c3c(c(Br)n4C(=O)[C@@H](N)CCSC)C[C@H]2N(C)C1] |
| 36 | 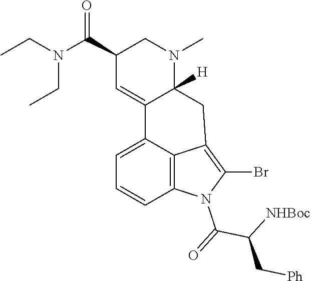 [SMILES: CCN(CC)C(=O)[C@@H]1C=C2c3cccc4c3c(c(Br)n4C(=O)[C@H](Cc3ccccc3)NC(=O)OC(C)(C)C)C[C@H]2N(C)C1] |
| 37 | 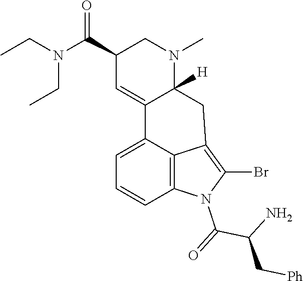 [SMILES: CCN(CC)C(=O)[C@@H]1C=C2c3cccc4c3c(c(Br)n4C(=O)[C@@H](N)Cc3ccccc3)C[C@H]2N(C)C1] |
| 38 | 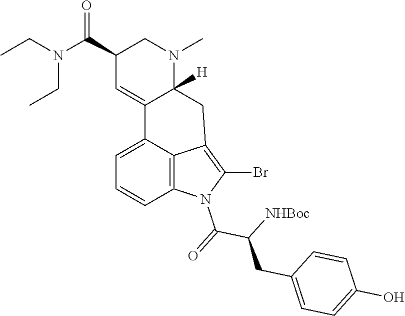 [SMILES: CCN(CC)C(=O)[C@@H]1C=C2c3cccc4c3c(c(Br)n4C(=O)[C@H](Cc3ccc(O)cc3)NC(=O)OC(C)(C)C)C[C@H]2N(C)C1] |
| 39 | 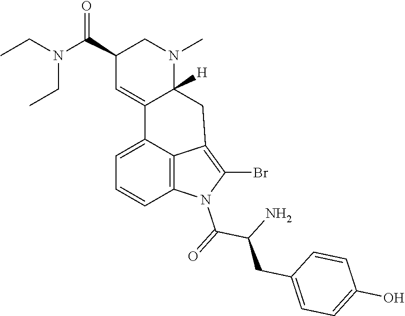 [SMILES: CCN(CC)C(=O)[C@@H]1C=C2c3cccc4c3c(c(Br)n4C(=O)[C@@H](N)Cc3ccc(O)cc3)C[C@H]2N(C)C1] |
| 40 | 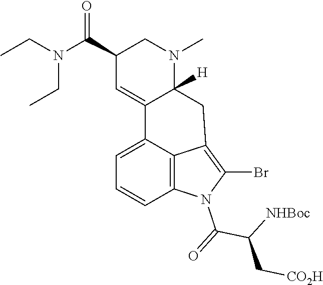 [SMILES: CCN(CC)C(=O)[C@@H]1C=C2c3cccc4c3c(c(Br)n4C(=O)[C@H](CC(=O)O)NC(=O)OC(C)(C)C)C[C@H]2N(C)C1] |
| 41 | 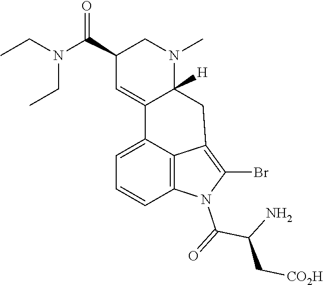 [SMILES: CCN(CC)C(=O)[C@@H]1C=C2c3cccc4c3c(c(Br)n4C(=O)[C@@H](N)CC(=O)O)C[C@H]2N(C)C1] |
| 42 | 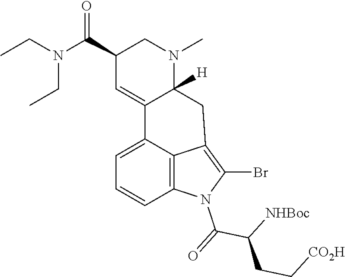 [SMILES: CCN(CC)C(=O)[C@@H]1C=C2c3cccc4c3c(c(Br)n4C(=O)[C@H](CCC(=O)O)NC(=O)OC(C)(C)C)C[C@H]2N(C)C1] |
| 43 | 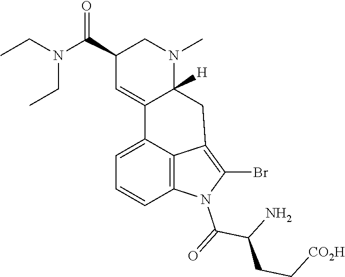 [SMILES: CCN(CC)C(=O)[C@@H]1C=C2c3cccc4c3c(c(Br)n4C(=O)[C@@H](N)CCC(=O)O)C[C@H]2N(C)C1] |
| 44 | 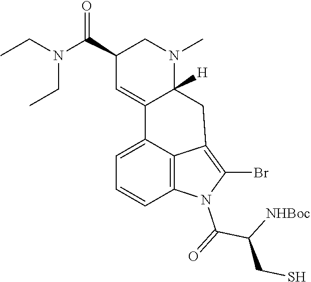 [SMILES: CCN(CC)C(=O)[C@@H]1C=C2c3cccc4c3c(c(Br)n4C(=O)[C@H](CS)NC(=O)OC(C)(C)C)C[C@H]2N(C)C1] |
| 45 | 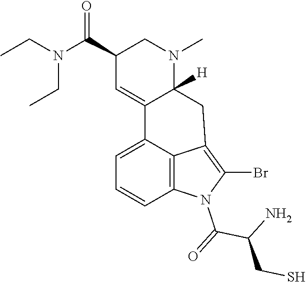 [SMILES: CCN(CC)C(=O)[C@@H]1C=C2c3cccc4c3c(c(Br)n4C(=O)[C@@H](N)CS)C[C@H]2N(C)C1] |
| 46 | 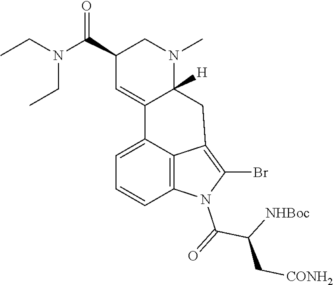 [SMILES: CCN(CC)C(=O)[C@@H]1C=C2c3cccc4c3c(c(Br)n4C(=O)[C@H](CC(N)=O)NC(=O)OC(C)(C)C)C[C@H]2N(C)C1] |
| 47 | 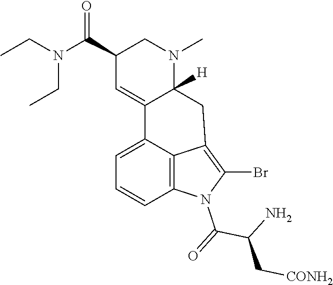 [SMILES: CCN(CC)C(=O)[C@@H]1C=C2c3cccc4c3c(c(Br)n4C(=O)[C@@H](N)CC(N)=O)C[C@H]2N(C)C1] |
| 48 | 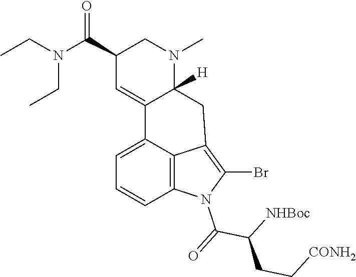 [SMILES: CCN(CC)C(=O)[C@@H]1C=C2c3cccc4c3c(c(Br)n4C(=O)[C@H](CCC(N)=O)NC(=O)OC(C)(C)C)C[C@H]2N(C)C1] |
| 49 | 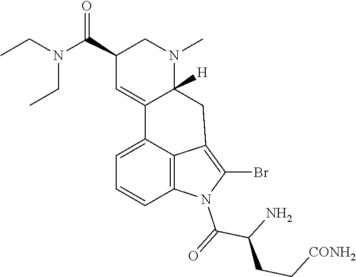 [SMILES: CCN(CC)C(=O)[C@@H]1C=C2c3cccc4c3c(c(Br)n4C(=O)[C@@H](N)CCC(N)=O)C[C@H]2N(C)C1] |
| 50 | 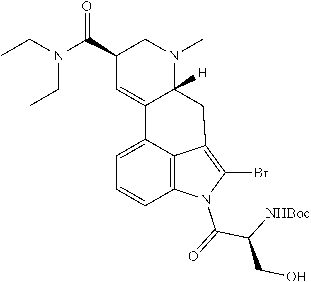 [SMILES: CCN(CC)C(=O)[C@@H]1C=C2c3cccc4c3c(c(Br)n4C(=O)[C@H](CO)NC(=O)OC(C)(C)C)C[C@H]2N(C)C1] |
| 51 | 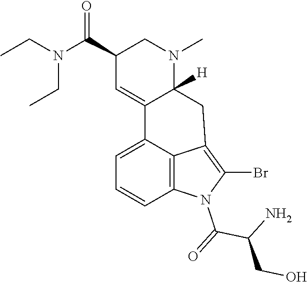 [SMILES: CCN(CC)C(=O)[C@@H]1C=C2c3cccc4c3c(c(Br)n4C(=O)[C@@H](N)CO)C[C@H]2N(C)C1] |
| 52 | 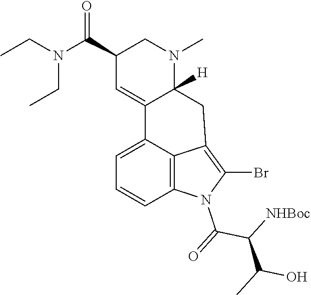 [SMILES: CCN(CC)C(=O)[C@@H]1C=C2c3cccc4c3c(c(Br)n4C(=O)[C@@H](NC(=O)OC(C)(C)C)C(C)O)C[C@H]2N(C)C1] |
| 53 | 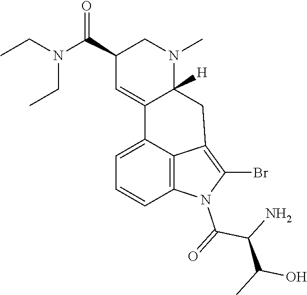 [SMILES: CCN(CC)C(=O)[C@@H]1C=C2c3cccc4c3c(c(Br)n4C(=O)[C@@H](N)C(C)O)C[C@H]2N(C)C1] |
| 54 | 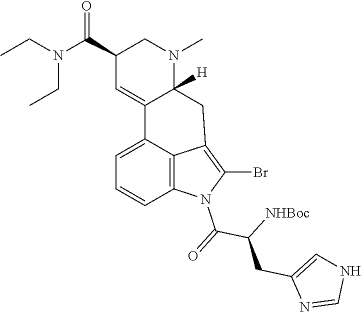 [SMILES: CCN(CC)C(=O)[C@@H]1C=C2c3cccc4c3c(c(Br)n4C(=O)[C@H](Cc3c[nH]cn3)NC(=O)OC(C)(C)C)C[C@H]2N(C)C1] |
| 55 | 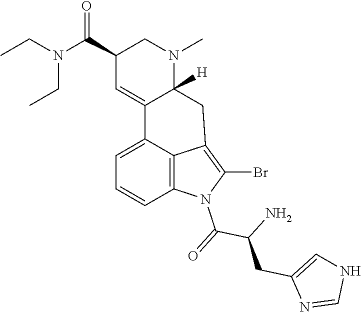 [SMILES: CCN(CC)C(=O)[C@@H]1C=C2c3cccc4c3c(c(Br)n4C(=O)[C@@H](N)Cc3c[nH]cn3)C[C@H]2N(C)C1] |
| 56 | 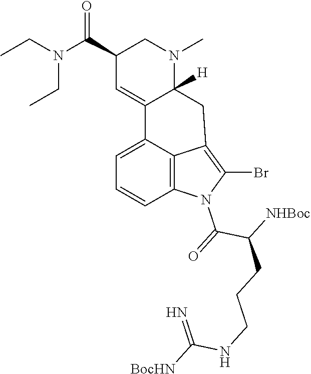 [SMILES: CCN(CC)C(=O)[C@@H]1C=C2c3cccc4c3c(c(Br)n4C(=O)[C@H](CCCNC(=N)NC(=O)OC(C)(C)C)NC(=O)OC(C)(C)C)C[C@H]2N(C)C1] |
| 57 | 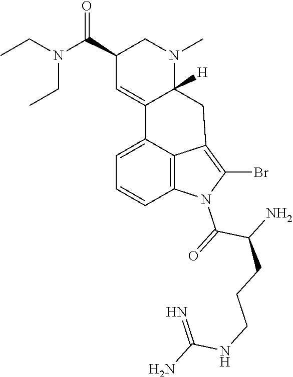 [SMILES: CCN(CC)C(=O)[C@@H]1C=C2c3cccc4c3c(c(Br)n4C(=O)[C@@H](N)CCCNC(=N)N)C[C@H]2N(C)C1] |
| 58 | 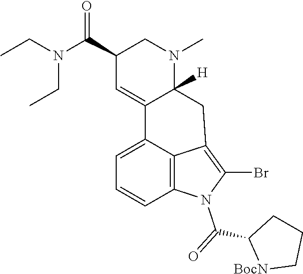 [SMILES: CCN(CC)C(=O)[C@@H]1C=C2c3cccc4c3c(c(Br)n4C(=O)[C@@H]3CCCN3C(=O)OC(C)(C)C)C[C@H]2N(C)C1] |
| 59 | 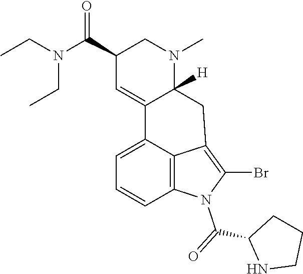 [SMILES: CCN(CC)C(=O)[C@@H]1C=C2c3cccc4c3c(c(Br)n4C(=O)[C@@H]3CCCN3)C[C@H]2N(C)C1] |
| 60 | 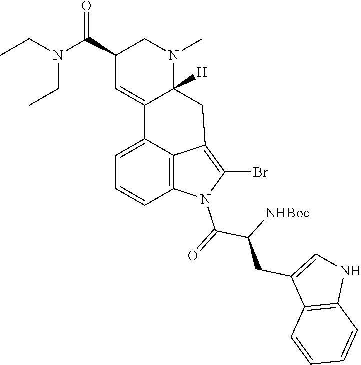 [SMILES: CCN(CC)C(=O)[C@@H]1C=C2c3cccc4c3c(c(Br)n4C(=O)[C@H](Cc3c[nH]c4ccccc34)NC(=O)OC(C)(C)C)C[C@H]2N(C)C1] |
| 61 | 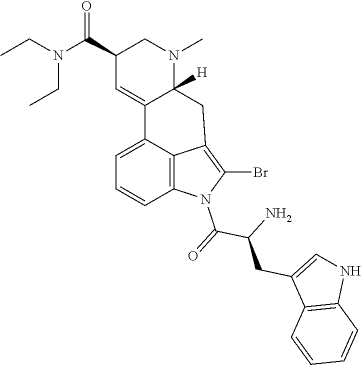 [SMILES: CCN(CC)C(=O)[C@@H]1C=C2c3cccc4c3c(c(Br)n4C(=O)[C@@H](N)Cc3c[nH]c4ccccc34)C[C@H]2N(C)C1] |
| 62 | 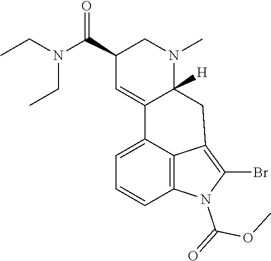 [SMILES: CCN(CC)C(=O)[C@@H]1C=C2c3cccc4c3c(c(Br)n4C(=O)OC)C[C@H]2N(C)C1] |
| 63 | 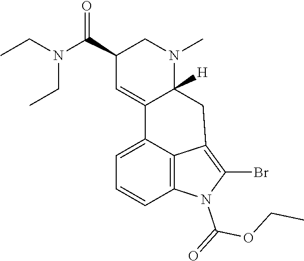 [SMILES: CCOC(=O)n1c(Br)c2c3c(cccc31)C1=C[C@@H](C(=O)N(CC)CC)CN(C)[C@@H]1C2] |
| 64 | 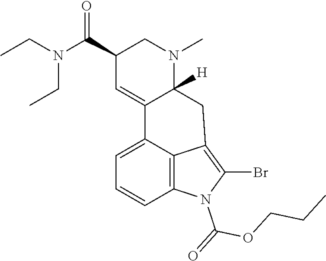 [SMILES: CCCOC(=O)n1c(Br)c2c3c(cccc31)C1=C[C@@H](C(=O)N(CC)CC)CN(C)[C@@H]1C2] |
| 65 | 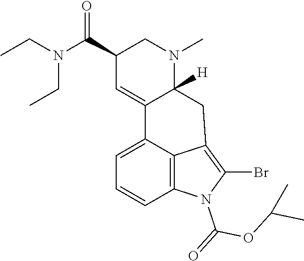 [SMILES: CCN(CC)C(=O)[C@@H]1C=C2c3cccc4c3c(c(Br)n4C(=O)OC(C)C)C[C@H]2N(C)C1] |
| 66 | 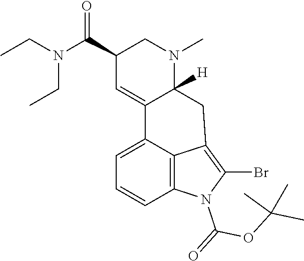 [SMILES: CCN(CC)C(=O)[C@@H]1C=C2c3cccc4c3c(c(Br)n4C(=O)OC(C)(C)C)C[C@H]2N(C)C1] |
| 67 | 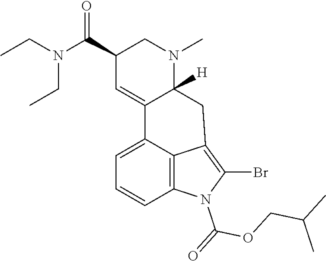 [SMILES: CCN(CC)C(=O)[C@@H]1C=C2c3cccc4c3c(c(Br)n4C(=O)OCC(C)C)C[C@H]2N(C)C1] |
| 68 | 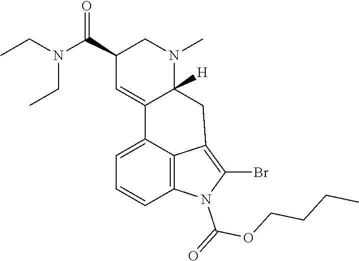 [SMILES: CCCCOC(=O)n1c(Br)c2c3c(cccc31)C1=C[C@@H](C(=O)N(CC)CC)CN(C)[C@@H]1C2] |
| 69 | 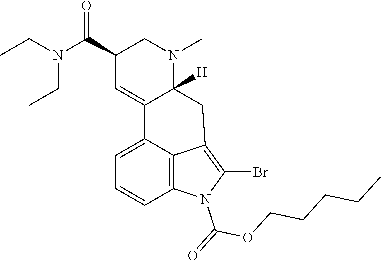 [SMILES: CCCCCOC(=O)n1c(Br)c2c3c(cccc31)C1=C[C@@H](C(=O)N(CC)CC)CN(C)[C@@H]1C2] |
| 70 | 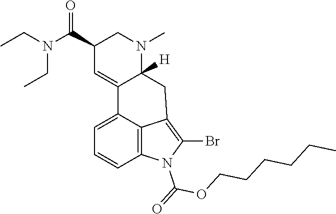 [SMILES: CCCCCCOC(=O)n1c(Br)c2c3c(cccc31)C1=C[C@@H](C(=O)N(CC)CC)CN(C)[C@@H]1C2] |
| 71 | 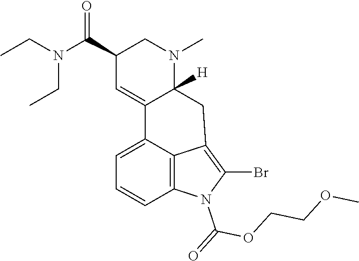 [SMILES: CCN(CC)C(=O)[C@@H]1C=C2c3cccc4c3c(c(Br)n4C(=O)OCCOC)C[C@H]2N(C)C1] |
| 72 | 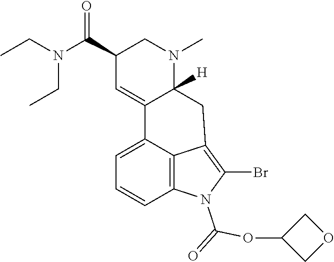 [SMILES: CCN(CC)C(=O)[C@@H]1C=C2c3cccc4c3c(c(Br)n4C(=O)OC3COC3)C[C@H]2N(C)C1] |
| 73 | 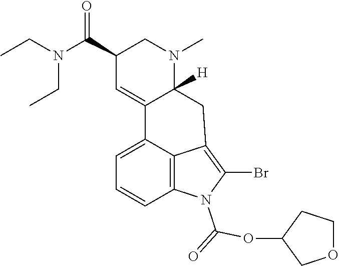 [SMILES: CCN(CC)C(=O)[C@@H]1C=C2c3cccc4c3c(c(Br)n4C(=O)OC3CCOC3)C[C@H]2N(C)C1] |
| 74 | 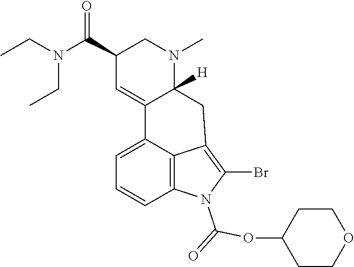 [SMILES: CCN(CC)C(=O)[C@@H]1C=C2c3cccc4c3c(c(Br)n4C(=O)OC3CCOCC3)C[C@H]2N(C)C1] |
| 75 | 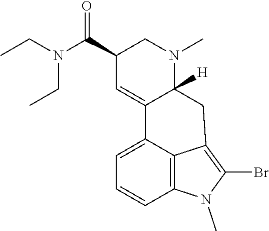 [SMILES: CCN(CC)C(=O)[C@@H]1C=C2c3cccc4c3c(c(Br)n4C)C[C@H]2N(C)C1] |
| 76 | 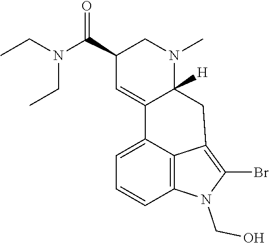 [SMILES: CCN(CC)C(=O)[C@@H]1C=C2c3cccc4c3c(c(Br)n4CO)C[C@H]2N(C)C1] |
| 77 | 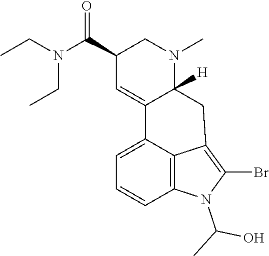 [SMILES: CCN(CC)C(=O)[C@@H]1C=C2c3cccc4c3c(c(Br)n4C(C)O)C[C@H]2N(C)C1] |
| 78 | 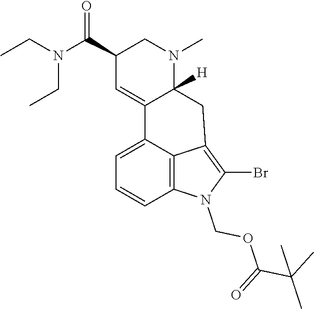 [SMILES: CCN(CC)C(=O)[C@@H]1C=C2c3cccc4c3c(c(Br)n4COC(=O)C(C)(C)C)C[C@H]2N(C)C1] |
| 79 | 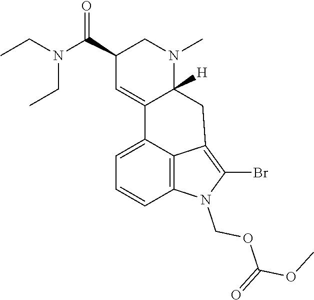 [SMILES: CCN(CC)C(=O)[C@@H]1C=C2c3cccc4c3c(c(Br)n4COC(=O)OC)C[C@H]2N(C)C1] |
| 80 | 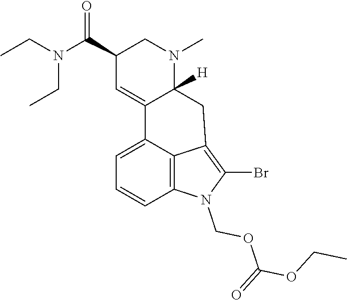 [SMILES: CCOC(=O)OCn1c(Br)c2c3c(cccc31)C1=C[C@@H](C(=O)N(CC)CC)CN(C)[C@@H]1C2] |
| 81 | 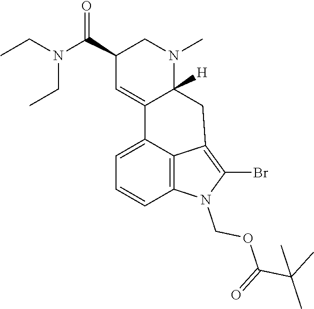 [SMILES: CCN(CC)C(=O)[C@@H]1C=C2c3cccc4c3c(c(Br)n4COC(=O)C(C)(C)C)C[C@H]2N(C)C1] |
| 82 | 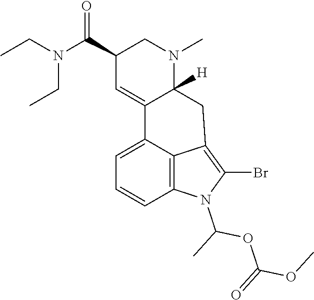 [SMILES: CCN(CC)C(=O)[C@@H]1C=C2c3cccc4c3c(c(Br)n4C(C)OC(=O)OC)C[C@H]2N(C)C1] |
| 83 | 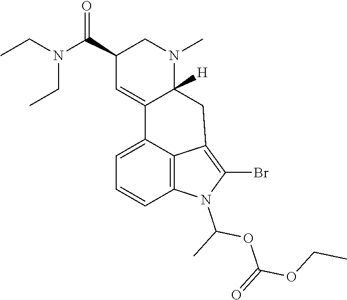 [SMILES: CCOC(=O)OC(C)n1c(Br)c2c3c(cccc31)C1=C[C@@H](C(=O)N(CC)CC)CN(C)[C@@H]1C2] |
| 84 | 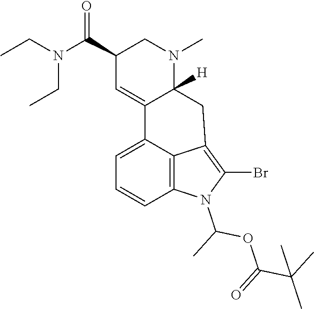 [SMILES: CCN(CC)C(=O)[C@@H]1C=C2c3cccc4c3c(c(Br)n4C(C)OC(=O)C(C)(C)C)C[C@H]2N(C)C1] |
| 85 | 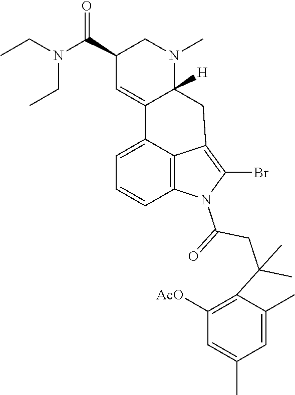 [SMILES: CCN(CC)C(=O)[C@@H]1C=C2c3cccc4c3c(c(Br)n4C(=O)CC(C)(C)c3c(C)cc(C)cc3OC(C)=O)C[C@H]2N(C)C1] |
| 86 | 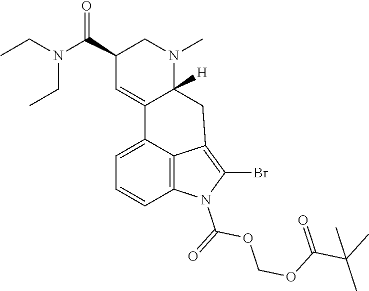 [SMILES: CCN(CC)C(=O)[C@@H]1C=C2c3cccc4c3c(c(Br)n4C(=O)OCOC(=O)C(C)(C)C)C[C@H]2N(C)C1] |
| 87 | 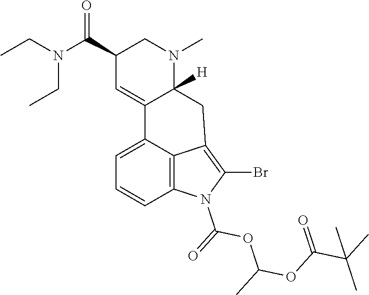 [SMILES: CCN(CC)C(=O)[C@@H]1C=C2c3cccc4c3c(c(Br)n4C(=O)OC(C)OC(=O)C(C)(C)C)C[C@H]2N(C)C1] |
| 88 | 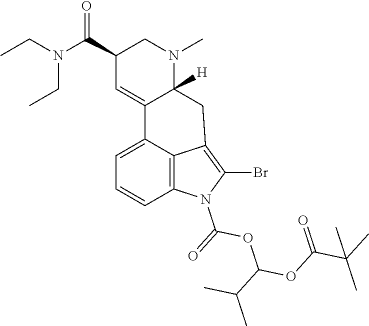 [SMILES: CCN(CC)C(=O)[C@@H]1C=C2c3cccc4c3c(c(Br)n4C(=O)OC(OC(=O)C(C)(C)C)C(C)C)C[C@H]2N(C)C1] |
| 89 | 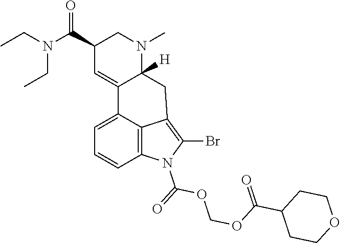 [SMILES: CCN(CC)C(=O)[C@@H]1C=C2c3cccc4c3c(c(Br)n4C(=O)OCOC(=O)C3CCOCC3)C[C@H]2N(C)C1] |
| 90 | 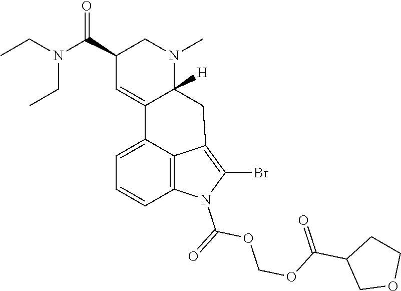 [SMILES: CCN(CC)C(=O)[C@@H]1C=C2c3cccc4c3c(c(Br)n4C(=O)OCOC(=O)C3CCOC3)C[C@H]2N(C)C1] |
| 91 | 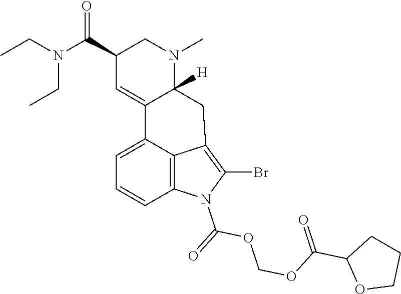 [SMILES: CCN(CC)C(=O)[C@@H]1C=C2c3cccc4c3c(c(Br)n4C(=O)OCOC(=O)C3CCCO3)C[C@H]2N(C)C1] |
| 92 | 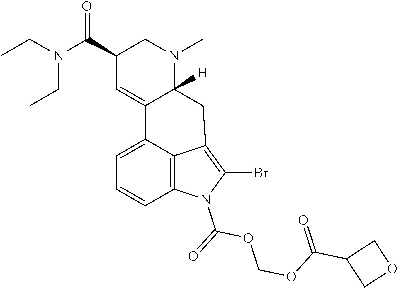 [SMILES: CCN(CC)C(=O)[C@@H]1C=C2c3cccc4c3c(c(Br)n4C(=O)OCOC(=O)C3COC3)C[C@H]2N(C)C1] |
| 93 | 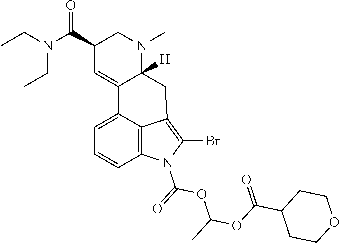 [SMILES: CCN(CC)C(=O)[C@@H]1C=C2c3cccc4c3c(c(Br)n4C(=O)OC(C)OC(=O)C3CCOCC3)C[C@H]2N(C)C1] |
| 94 | 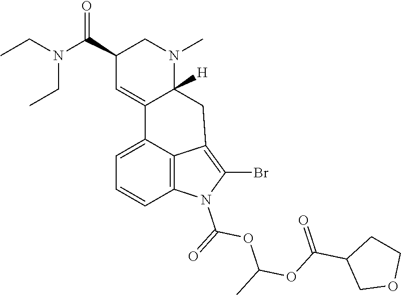 [SMILES: CCN(CC)C(=O)[C@@H]1C=C2c3cccc4c3c(c(Br)n4C(=O)OC(C)OC(=O)C3CCOC3)C[C@H]2N(C)C1] |
| 95 | 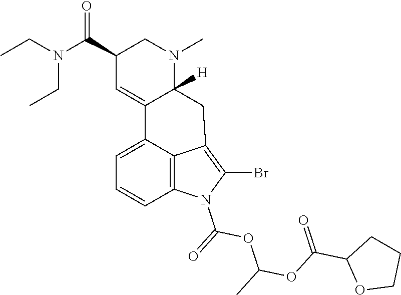 [SMILES: CCN(CC)C(=O)[C@@H]1C=C2c3cccc4c3c(c(Br)n4C(=O)OC(C)OC(=O)C3CCCO3)C[C@H]2N(C)C1] |
| 96 | 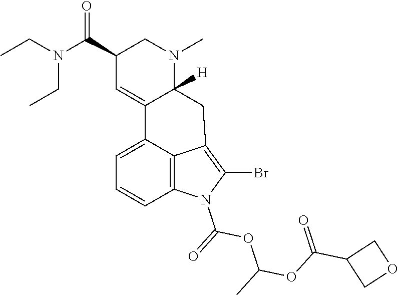 [SMILES: CCN(CC)C(=O)[C@@H]1C=C2c3cccc4c3c(c(Br)n4C(=O)OC(C)OC(=O)C3COC3)C[C@H]2N(C)C1] |
| 97 | 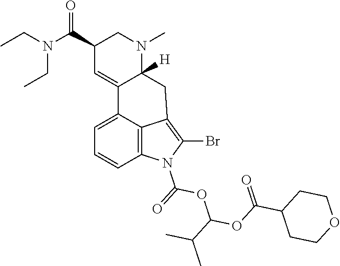 [SMILES: CCN(CC)C(=O)[C@@H]1C=C2c3cccc4c3c(c(Br)n4C(=O)OC(OC(=O)C3CCOCC3)C(C)C)C[C@H]2N(C)C1] |
| 98 | 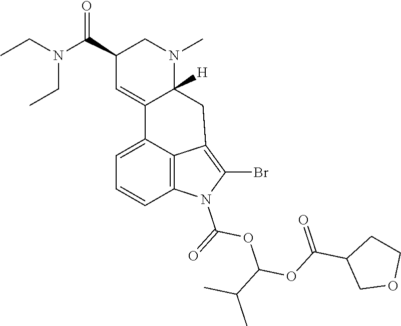 [SMILES: CCN(CC)C(=O)[C@@H]1C=C2c3cccc4c3c(c(Br)n4C(=O)OC(OC(=O)C3CCOC3)C(C)C)C[C@H]2N(C)C1] |
| 99 | 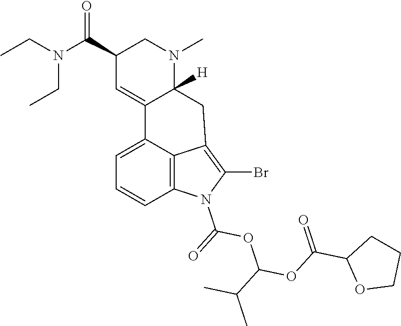 [SMILES: CCN(CC)C(=O)[C@@H]1C=C2c3cccc4c3c(c(Br)n4C(=O)OC(OC(=O)C3CCCO3)C(C)C)C[C@H]2N(C)C1] |
| 100 | 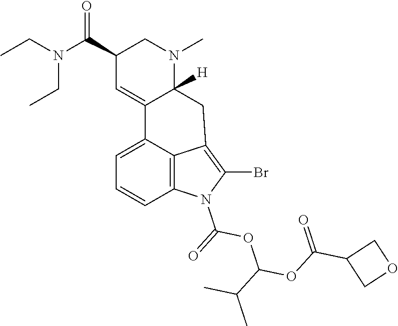 [SMILES: CCN(CC)C(=O)[C@@H]1C=C2c3cccc4c3c(c(Br)n4C(=O)OC(OC(=O)C3COC3)C(C)C)C[C@H]2N(C)C1] |
| 101 | 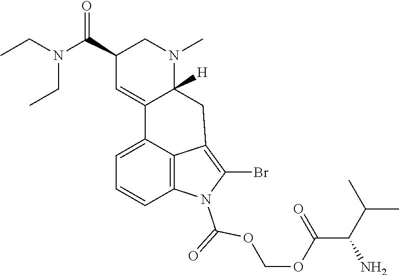 [SMILES: CCN(CC)C(=O)[C@@H]1C=C2c3cccc4c3c(c(Br)n4C(=O)OCOC(=O)[C@@H](N)C(C)C)C[C@H]2N(C)C1] |
| 102 | 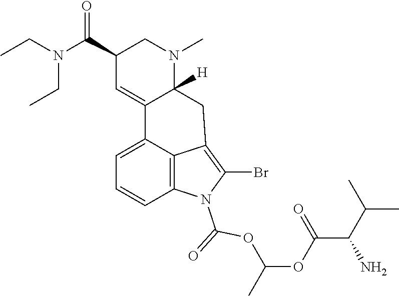 [SMILES: CCN(CC)C(=O)[C@@H]1C=C2c3cccc4c3c(c(Br)n4C(=O)OC(C)OC(=O)[C@@H](N)C(C)C)C[C@H]2N(C)C1] |
| 103 | 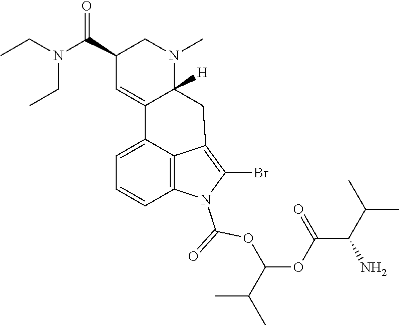 [SMILES: CCN(CC)C(=O)[C@@H]1C=C2c3cccc4c3c(c(Br)n4C(=O)OC(OC(=O)[C@@H](N)C(C)C)C(C)C)C[C@H]2N(C)C1] |
| 104 | 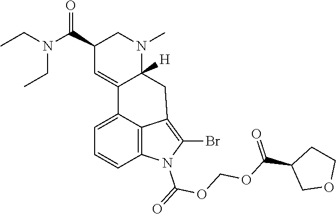 [SMILES: CCN(CC)C(=O)[C@@H]1C=C2c3cccc4c3c(c(Br)n4C(=O)OCOC(=O)[C@H]3CCOC3)C[C@H]2N(C)C1] |
| 105 | 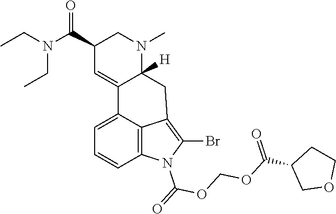 [SMILES: CCN(CC)C(=O)[C@@H]1C=C2c3cccc4c3c(c(Br)n4C(=O)OCOC(=O)[C@@H]3CCOC3)C[C@H]2N(C)C1] |
| 106 | 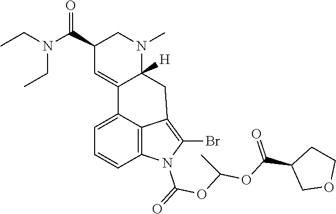 [SMILES: CCN(CC)C(=O)[C@@H]1C=C2c3cccc4c3c(c(Br)n4C(=O)OC(C)OC(=O)[C@H]3CCOC3)C[C@H]2N(C)C1] |
| 107 | 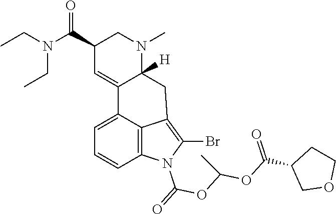 [SMILES: CCN(CC)C(=O)[C@@H]1C=C2c3cccc4c3c(c(Br)n4C(=O)OC(C)OC(=O)[C@@H]3CCOC3)C[C@H]2N(C)C1] |
| 108 | 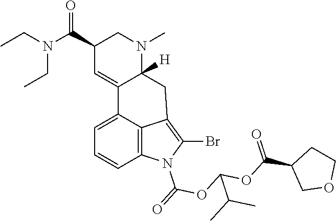 [SMILES: CCN(CC)C(=O)[C@@H]1C=C2c3cccc4c3c(c(Br)n4C(=O)OC(OC(=O)[C@H]3CCOC3)C(C)C)C[C@H]2N(C)C1] |
| 109 | 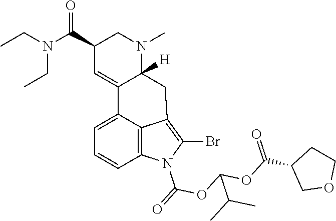 [SMILES: CCN(CC)C(=O)[C@@H]1C=C2c3cccc4c3c(c(Br)n4C(=O)OC(OC(=O)[C@@H]3CCOC3)C(C)C)C[C@H]2N(C)C1] |
| 110 | 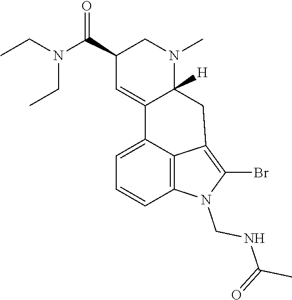 [SMILES: CCN(CC)C(=O)[C@@H]1C=C2c3cccc4c3c(c(Br)n4CNC(C)=O)C[C@H]2N(C)C1] |
| 111 | 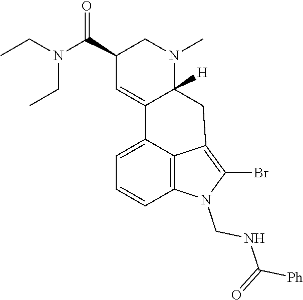 [SMILES: CCN(CC)C(=O)[C@@H]1C=C2c3cccc4c3c(c(Br)n4CNC(=O)c3ccccc3)C[C@H]2N(C)C1] |
| 112 | 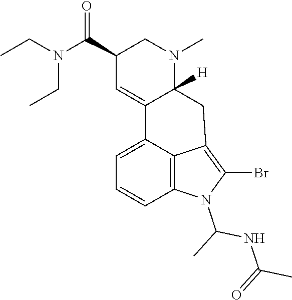 [SMILES: CCN(CC)C(=O)[C@@H]1C=C2c3cccc4c3c(c(Br)n4C(C)NC(C)=O)C[C@H]2N(C)C1] |
| 113 | 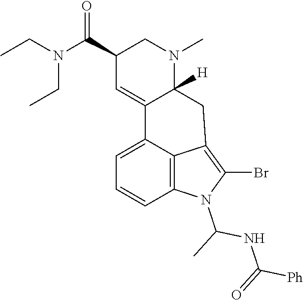 [SMILES: CCN(CC)C(=O)[C@@H]1C=C2c3cccc4c3c(c(Br)n4C(C)NC(=O)c3ccccc3)C[C@H]2N(C)C1] |

The present disclosure provides for pharmaceutically-acceptable salts of any compound described herein as well as the use of such salts. As is understood by those of skill in the art, any compound with an ionizable group, such as an acidic hydrogen, or a basic nitrogen, can be provided in the form of a salt, and pharmaceutically acceptable salt forms of such compounds are specifically contemplated herein. Pharmaceutically-acceptable salts include, for example, acid-addition salts and base-addition salts. The acid that is added to the compound to form an acid-addition salt can be an organic acid or an inorganic acid. A base that is added to the compound to form a base-addition salt can be an organic base or an inorganic base. In some embodiments, a pharmaceutically-acceptable salt is a metal salt. In some embodiments, a pharmaceutically-acceptable salt is an ammonium salt.

Metal salts can arise from the addition of an inorganic base to a compound of the present disclosure. The inorganic base consists of a metal cation paired with a basic counterion, such as, for example, hydroxide, carbonate, bicarbonate, or phosphate. The metal can be an alkali metal, alkaline earth metal, transition metal, or main group metal. In some embodiments, the metal is lithium, sodium, potassium, cesium, cerium, magnesium, manganese, iron, calcium, strontium, cobalt, titanium, aluminum, copper, cadmium, or zinc.

In some embodiments, a metal salt is a lithium salt, a sodium salt, a potassium salt, a cesium salt, a cerium salt, a magnesium salt, a manganese salt, an iron salt, a calcium salt, a strontium salt, a cobalt salt, a titanium salt, an aluminum salt, a copper salt, a cadmium salt, or a zinc salt.

Ammonium salts can arise from the addition of ammonia or an organic amine to a compound of the present disclosure. In some embodiments, the organic amine is trimethyl amine, triethyl amine, diisopropyl amine, ethanol amine, diethanol amine, triethanol amine, morpholine, N-methylmorpholine, piperidine, N-methylpiperidine, N-ethylpiperidine, dibenzylamine, piperazine, pyridine, pyrazole, pyrazolidine, pyrazoline, pyridazine, pyrimidine, imidazole, or pyrazine.

In some embodiments, an ammonium salt is a triethyl amine salt, trimethyl amine salt, a diisopropyl amine salt, an ethanol amine salt, a diethanol amine salt, a triethanol amine salt, a morpholine salt, an N-methylmorpholine salt, a piperidine salt, an N-methylpiperidine salt, an N-ethylpiperidine salt, a dibenzylamine salt, a piperazine salt, a pyridine salt, a pyrazole salt, a pyridazine salt, a pyrimidine salt, an imidazole salt, or a pyrazine salt.

Acid addition salts can arise from the addition of an acid to a compound of the present disclosure. In some embodiments, the acid is organic. In some embodiments, the acid is inorganic. In some embodiments, the acid is hydrochloric acid, hydrobromic acid, hydroiodic acid, nitric acid, nitrous acid, sulfuric acid, sulfurous acid, a phosphoric acid, isonicotinic acid, lactic acid, salicylic acid, tartaric acid, ascorbic acid, gentisic acid, gluconic acid, glucuronic acid, saccharic acid, formic acid, benzoic acid, glutamic acid, pantothenic acid, acetic acid, propionic acid, butyric acid, fumaric acid, succinic acid, methanesulfonic acid, ethanesulfonic acid, benzenesulfonic acid, p-toluenesulfonic acid, citric acid, oxalic acid, xinafoic acid, or maleic acid.

In some embodiments, the salt is a hydrochloride salt, a hydrobromide salt, a hydroiodide salt, a nitrate salt, a nitrite salt, a sulfate salt, a sulfite salt, a phosphate salt, isonicotinate salt, a lactate salt, a salicylate salt, a tartrate salt, an ascorbate salt, a gentisate salt, a gluconate salt, a glucuronate salt, a saccharate salt, a formate salt, a benzoate salt, a glutamate salt, a pantothenate salt, an acetate salt, a propionate salt, a butyrate salt, a fumarate salt, a succinate salt, a methanesulfonate salt, an ethanesulfonate salt, a benzenesulfonate salt, a p-toluenesulfonate salt, a citrate salt, an oxalate salt, a xinafoate salt, or a maleate salt.

### III. Pharmaceutical Compositions and Formulations

In some embodiments, the present invention provides a pharmaceutical composition comprising a compound of the present invention, such as a composition comprising a compound of any of Table 1, the Formulas illustrated above, including any of Formulas (I), (Ia), (Ia1), (Ia2), (1a3), (Ib), (Ib1), (Ic), (Ic1), (Ic2), (1c3), (Id), (Ic1), (Ic2), (1c3), (Id4), (Ie), (Ie1), (Ie2), (Ig), (Ih), (Ii), (Ii1), (Ii2) and (Ij), and a pharmaceutically acceptable excipient. Such compositions are suitable for administration to a subject, such as a human subject.

The presently disclosed pharmaceutical compositions can be prepared in a wide variety of oral, parenteral and topical dosage forms. Oral preparations include tablets, pills, powder, capsules, liquids, lozenges, cachets, gels, syrups, slurries, suspensions, etc., suitable for ingestion by the patient. The compositions of the present invention can also be administered by injection, that is, intravenously, intramuscularly, intracutaneously, subcutaneously, intraduodenally, or intraperitoneally. Also, the compositions described herein can be administered by inhalation, for example, intranasally. Additionally, the compositions of the present invention can be administered transdermally. The compositions of this invention can also be administered by intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Accordingly, the present invention also provides pharmaceutical compositions including a pharmaceutically acceptable carrier or excipient and the compounds of the present invention.

For preparing pharmaceutical compositions from the compounds disclosed herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Mack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired. The powders and tablets preferably contain from 5% to 70% or 10% to 70% of the compounds of the present invention.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethylcellulose, or sodium carboxymethylcellulose; and gums including arabic and tragacanth; as well as proteins including, but not limited to, gelatin and collagen.

If desired, disintegrating or solubilizing agents may be added, such as the cross-linked polyvinyl pyrrolidone, agar, alginic acid, or a salt thereof, such as sodium alginate.

For preparing suppositories, a low melting wax, such as a mixture of fatty acid glycerides or cocoa butter, is first melted and the compounds of the present invention are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compounds of the present invention in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of the present invention in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compositions of the present invention can also be delivered as microspheres for slow release in the body. For example, microspheres can be formulated for administration via intradermal injection of drug-containing microspheres, which slowly release subcutaneously (see Rao, J. Biomater Sci. Polym. Ed 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

In some embodiments, the pharmaceutical compositions of the present invention can be formulated for parenteral administration, such as intravenous (IV) administration or administration into a body cavity or lumen of an organ. The formulations for administration will commonly comprise a solution of the compositions of the present invention dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compositions of the present invention in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In some embodiments, the formulations of the compositions of the present invention can be delivered by the use of liposomes which fuse with the cellular membrane or are endocytosed, for example, by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compositions of the present invention into the target cells in vivo. (See, e.g., A1-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

### IV. Administration:

The compositions of the present invention can be administered by any suitable means, including oral, parenteral and topical methods. Transdermal administration methods, by a topical route, can be formulated as applicator sticks, solutions, suspensions, emulsions, gels, creams, ointments, pastes, jellies, paints, powders, and aerosols.

The pharmaceutical preparation is preferably in unit dosage form. In such form the preparation is subdivided into unit doses containing appropriate quantities of the compounds of the present invention. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The compound of the present invention can be present in any suitable amount, and can depend on various factors including, but not limited to, weight and age of the subject, state of the disease, and the like as is known to those of ordinary skill in the art. Suitable dosage ranges for the compounds disclosed herein include from about 0.1 mg to about 10,000 mg, or about 1 mg to about 1000 mg, or about 10 mg to about 750 mg, or about 25 mg to about 500 mg, or about 50 mg to about 250 mg. Suitable dosages for the compound of the present invention include about 1 mg, 5, 10, 20, 30, 40, 50, 60, 70, 80, 90, 100, 200, 300, 400, 500, 600, 700, 800, 900 or 1000 mg.

The compounds disclosed herein can be administered at any suitable frequency, interval and duration. For example, the compounds can be administered once an hour, or two, three or more times an hour, once a day, or two, three, or more times per day, or once every 2, 3, 4, 5, 6, or 7 days, so as to provide the preferred dosage level. When the compound of the present invention is administered more than once a day, representative intervals include 5, 10, 15, 20, 30, 45 and 60 minutes, as well as 1, 2, 4, 6, 8, 10, 12, 16, 20, and 24 hours. The compound of the present invention can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The composition can also contain other compatible therapeutic agents. The compounds described herein can be used in combination with one another, with other active agents known to be useful in modulating a glucocorticoid receptor, or with adjunctive agents that may not be effective alone, but may contribute to the efficacy of the active agent.

The compounds of the present invention can be co-administered with a second active agent. Co-administration includes administering the compound of the present invention and active agent within 0.5, 1, 2, 4, 6, 8, 10, 12, 16, 20, or 24 hours of each other. Co-administration also includes administering the compound of the present invention and active agent simultaneously, approximately simultaneously (e.g., within about 1, 5, 10, 15, 20, or 30 minutes of each other), or sequentially in any order. Moreover, the compound of the present invention and the active agent can each be administered once a day, or two, three, or more times per day so as to provide the preferred dosage level per day.

In some embodiments, co-administration can be accomplished by co-formulation, such as by preparing a single pharmaceutical composition including both the compound of the present invention and a second active agent. In other embodiments, the compound of the present invention and the second active agent can be formulated separately.

The disclosed compounds and the second active agent can be present in the compositions of the present invention in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present invention and the second active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present invention and the active agent are suitable in the compositions and methods disclosed herein.

### V. Methods of Treatment

The compounds of the present invention, such as a compound of any of Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) and (Ij), can be used for increasing neuronal plasticity. The compounds of the present invention can also be used to treat any brain disease. The compounds of the present invention can also be used for increasing at least one of translation, transcription or secretion of neurotrophic factors.

In some embodiments, a compound of the present invention, such as a compound of Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij), is used to treat neurological diseases. In some embodiments, the compounds have, for example, anti-addictive properties, antidepressant properties, anxiolytic properties, or a combination thereof. In some embodiments, the neurological disease is a neuropsychiatric disease. In some embodiments, the neuropsychiatric disease is a mood or anxiety disorder. In some embodiments, the neurological disease is a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder). In some embodiments, the neurological disease is a migraine or cluster headache. In some embodiments, the neurological disease is a neurodegenerative disorder, Alzheimer's disease, or Parkinson's disease. In some embodiments, the neurological disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease is a psychological disorder, treatment resistant depression, suicidal ideation, major depressive disorder, bipolar disorder, schizophrenia, post-traumatic stress disorder (PTSD), addiction (e.g., substance use disorder), depression, or anxiety. In some embodiments, the neuropsychiatric disease or neurological disease is post-traumatic stress disorder (PTSD), addiction (e.g., substance use disorder), schizophrenia, depression, or anxiety. In some embodiments, the neuropsychiatric disease or neurological disease is addiction (e.g., substance use disorder). In some embodiments, the neuropsychiatric disease or neurological disease is depression. In some embodiments, the neuropsychiatric disease or neurological disease is anxiety. In some embodiments, the neuropsychiatric disease or neurological disease is post-traumatic stress disorder (PTSD). In some embodiments, the neurological disease is stroke or traumatic brain injury. In some embodiments, the neuropsychiatric disease or neurological disease is schizophrenia.

In some embodiments, a compound of the present invention is used for increasing neuronal plasticity. In some embodiments, the compounds described herein are used for treating a brain disorder. In some embodiments, the compounds described herein are used for increasing at least one of translation, transcription, or secretion of neurotrophic factors.

In some embodiments, the present invention provides a method of treating a disease, including administering to a subject in need thereof, a therapeutically effective amount of a compound of the present invention, such as a compound of Table 1, Formulas (I), (Ia), (Ia1), (Ia2), (1a3), (Ib), (Ib1), (Ic), (Ic1), (Ic2), (1c3), (Id), (Ic1), (Ic2), (1c3), (Id4), (Ie), (Ie1), (Ie2), (Ig), (Ih), (Ii), (Ii1), (Ii2) or (Ij). In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause.

In some embodiments, the compounds of the present invention, such as a compound of Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij), have activity as 5-HT_{2A} modulators. In some embodiments, the compounds of the present invention elicit a biological response by activating the 5-HT_{2A} receptor (e.g., allosteric modulation or modulation of a biological target that activates the 5-HT_{2A} receptor). 5-HT_{2A} agonism has been correlated with the promotion of neural plasticity (Ly et al., 2018). 5-HT_{2A} antagonists abrogate the neuritogenesis and spinogenesis effects of hallucinogenic compounds with 5-HT_{2A} agonist activity, for example, DMT, LSD, and DOI. In some embodiments, the compounds of the present invention release 2-Br-LSD in vivo and thus function as 5-HT_{2A} modulators and promote neural plasticity (e.g., cortical structural plasticity). In some embodiments, the compounds of the present invention are selective 5-HT_{2A} modulators and promote neural plasticity (e.g., cortical structural plasticity). In some embodiments, promotion of neural plasticity includes, for example, increased dendritic spine growth, increased synthesis of synaptic proteins, strengthened synaptic responses, increased dendritic arbor complexity, increased dendritic branch content, increased spinogenesis, increased neuritogenesis, or any combination thereof. In some embodiments, increased neural plasticity includes, for example, increased cortical structural plasticity in the anterior parts of the brain.

In some embodiments, the 5-HT_{2A} modulators (e.g., 5-HT_{2A} agonists) are non-hallucinogenic. In some embodiments, non-hallucinogenic 5-HT_{2A} modulators (e.g., 5-HT_{2A} agonists) are used to treat neurological diseases, which modulators do not elicit dissociative side-effects. In some embodiments, the hallucinogenic potential of the compounds described herein is assessed in vitro. In some embodiments, the hallucinogenic potential assessed in vitro of the compounds described herein is compared to the hallucinogenic potential assessed in vitro of hallucinogenic homologs. In some embodiments, the compounds described herein elicit less hallucinogenic potential in vitro than the hallucinogenic homologs.

In some embodiments, serotonin receptor modulators, such as modulators of serotonin receptor 2A (5-HT_{2A} modulators, e.g., 5-HT_{2A} agonists), are used to treat a brain disorder. The presently disclosed compounds of Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), ((Ig), (Ih), (Ii), (Ii1), (Ii2) or (Ij) can function as 5-HT_{2A} agonists alone, or in combination with a second therapeutic agent that also is a 5-HT_{2A} modulator. In such cases the second therapeutic agent can be an agonist or an antagonist. In some instances, it may be helpful administer a 5-HT_{2A} antagonist in combination with a compound of the present invention to mitigate undesirable effects of 5-HT_{2A} agonism, such as potential hallucinogenic effects. Serotonin receptor modulators useful as second therapeutic agents for combination therapy as described herein are known to those of skill in the art and include, without limitation, ketanserin, volinanserin (MDL-100907), eplivanserin (SR-46349), pimavanserin (ACP-103), glemanserin (MDL-11939), ritanserin, flibanserin, nelotanserin, blonanserin, mianserin, mirtazapine, roluperiodone (CYR-101, MIN-101), quetiapine, olanzapine, altanserin, acepromazine, nefazodone, risperidone, pruvanserin, AC-90179, AC-279, adatanserin, fananserin, HY10275, benanserin, butanserin, manserin, iferanserin, lidanserin, pelanserin, seganserin, tropanserin, lorcaserin, ICI-169369, methiothepin, methysergide, trazodone, cinitapride, cyproheptadine, brexpiprazole, cariprazine, agomelatine, setoperone, 1-(1-Naphthyl)piperazine, LY-367265, pirenperone, metergoline, deramciclane, amperozide, cinanserin, LY-86057, GSK-215083, cyamemazine, mesulergine, BF-1, LY-215840, sergolexole, spiramide, LY-53857, amesergide, LY-108742, pipamperone, LY-314228, 5-I-R91150, 5-MeO-NBpBrT, 9-Aminomethyl-9,10-dihydroanthracene, niaprazine, SB-215505, SB-204741, SB-206553, SB-242084, LY-272015, SB-243213, SB-200646, RS-102221, zotepine, clozapine, chlorpromazine, sertindole, iloperidone, paliperidone, asenapine, amisulpride, aripiprazole, lurasidone, ziprasidone, lumateperone, perospirone, mosapramine, AMDA (9-Aminomethyl-9,10-dihydroanthracene), methiothepin, xanomeline, buspirone, an extended-release form of olanzapine (e.g., ZYPREXA RELPREVV), an extended-release form of quetiapine, an extended-release form of risperidone (e.g., Risperdal Consta), an extended-release form of paliperidone (e.g., Invega Sustenna and Invega Trinza), an extended-release form of fluphenazine decanoate including Prolixin Decanoate, an extended-release form of aripiprazole lauroxil including Aristada, an extended-release form of aripiprazole including Abilify Maintena, 3-(2-(4-(4-Fluorobenzoyl)piperazin-1-yl)ethyl)-5-methyl-5-phenylimidazolidine-2,4-dione, 3-(2-(4-Benzhydrylpiperazin-1-yl)ethyl)-5-methyl-5-phenylimidazolidine-2,4-dione, 3-(3-(4-(2-Fluorophenyl)piperazin-1-yl)propyl)-5-methyl-5-phenylimidazolidine-2,4-dione, 3-(3-(4-(3-Fluorophenyl)piperazin-1-yl)propyl)-5-methyl-5-phenylimidazolidine-2,4-dione, 3-(3-(4-(4-Fluorophenyl)piperazin-1-yl)propyl)-5-methyl-5-phenylimidazolidine-2,4-dione, 3-(3-(4-(4-Fluorobenzoyl)piperazin-1-yl)propyl)-5-methyl-5-phenylimidazolidine-2,4-dione, 3-(2-(4-(4-Fluorobenzoyl)piperazin-1-yl)ethyl)-8-phenyl-1,3-diazaspiro[4.5]decane-2,4-dione, 3-(2-(4-Benzhydrylpiperazin-1-yl)ethyl)-8-phenyl-1,3-diazaspiro[4.5]decane-2,4-dione, 3-(3-(4-(2-Fluorophenyl)piperazin-1-yl)propyl)-8-phenyl-1,3-diazaspiro[4.5]decane-2,4-dione, 3-(3-(4-(3-Fluorophenyl)piperazin-1-yl)propyl)-8-phenyl-1,3-diazaspiro[4.5]decane-2,4-dione, 3-(3-(4-(4-Fluorophenyl)piperazin-1-yl)propyl)-8-phenyl-1,3-diazaspiro[4.5]decane-2,4-dione, and 3-(3-(4-(4-Fluorobenzoyl)piperazin-1-yl)propyl)-8-phenyl-1,3-diazaspiro[4.5]decane-2,4-dione, or a pharmaceutically acceptable salt, solvate, metabolite, deuterated analog, derivative, prodrug, or combinations thereof. In some embodiments, the serotonin receptor modulator used as a second therapeutic is pimavanserin or a pharmaceutically acceptable salt, solvate, metabolite, derivative, or prodrug thereof. In some embodiments, the serotonin receptor modulator is administered simultaneous with a compound disclosed herein. In some embodiments, the serotonin receptor modulator is co-administered in the same formulation as a compound disclosed herein. In some embodiments, the serotonin receptor modulator is administered prior to a compound disclosed herein, such as about 8 hours to about 30 minutes prior, or about three hours, or about two hours, or about one hour prior to administration of a compound according to Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij). In some embodiments, the serotonin receptor modulator is administered at most about one hour prior to the presently disclosed compound. In some embodiments, the serotonin receptor modulator is administered at most about two hours prior to the presently disclosed compound. In some embodiments, the serotonin receptor modulator is administered after a compound disclosed herein, such as about one to about 30 minutes, or about 1 hour, or about 2 hours, or about 3 hours, or about 4 hours, or about 5 hours, or about 6 hours, or about 7 hours, or about 8 hours, or about 9 hours, or about 10 hours, or about 11 hours, or about 12 hours, after the administration of a compound according to Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij). Thus, in some embodiments of combination therapy with the presently disclosed compounds, the second therapeutic agent is a serotonin receptor modulator. In some embodiments the second therapeutic agent serotonin receptor modulator is provided at a dose of from about 10 mg to about 350 mg. In some embodiments, the serotonin receptor modulator is provided at a dose of from about 20 mg to about 200 mg. In some embodiments, the serotonin receptor modulator is provided at a dose of from about 10 mg to about 100 mg. In certain such embodiments, the compound of the present invention is provided at a dose of from about 10 mg to about 100 mg, or from about 20 to about 200 mg, or from about 15 to about 300 mg, and the serotonin receptor modulator is provided at a dose of about 10 mg to about 100 mg. In some embodiments, the compound of the present invention is provided at a dose of from about 10 micrograms to about 1.0 mg, or about 100 micrograms or about 200 micrograms, or about 300 micrograms, or about 400 micrograms, or about 500 micrograms, or about 600 micrograms, or about 700 micrograms, or about 800 micrograms, or about 900 micrograms, or about 1.8 mg, or about 2 mg, or about 3 mg, or about 4 mg, or about 5 mg, or about 6 mg, or about 7 mg, or about 8 mg, or about 9 mg, or about 10 mg, or about 30 micrograms per kg, or about 0.1 mg to about 50 mg, or about 1 mg to about 20 mg, or about 20 mg to about 30 mg, and the serotonin receptor modulator is provided at a dose of about 10 mg to about 100 mg.

In some embodiments, non-hallucinogenic 5-HT_{2A} modulators (e.g., 5-HT_{2A} agonists) are used to treat neurological diseases. In some embodiments, the neurological diseases comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, non-hallucinogenic 5-HT_{2A} modulators (e.g., 5-HT_{2A} agonists) are used for increasing neuronal plasticity. In some embodiments, non-hallucinogenic 5-HT_{2A} modulators (e.g., 5-HT_{2A} agonists) are used for treating a brain disorder. In some embodiments, non-hallucinogenic 5-HT_{2A} modulators (e.g., 5-FIT_{2A} agonists) are used for increasing at least one of translation, transcription, or secretion of neurotrophic factors.

In some embodiments the presently disclosed compounds of Table 1, Formulas (I), (Ia), (Ia1), (Ia2), (1a3), (Ib), (Ib1), (Ic), (Ic1), (Ic2), (1c3), (Id), (Ic1), (Ic2), (1c3), (Id4), (Ie), (Ie1), (Ie2), (Ig), (Ih), (Ii), (Ii1), (Ii2) or (Ij) are given to patients in a low dose that is lower than would produce noticeable psychedelic effects but high enough to provide a therapeutic benefit. This dose range is predicted to be between 200 ug (micrograms) and 2 mg.

### Methods for Increasing Neuronal Plasticity

Neuronal plasticity refers to the ability of the brain to change structure and/or function throughout a subject's life. New neurons can be produced and integrated into the central nervous system throughout the subject's life. Increasing neuronal plasticity includes, but is not limited to, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing neuronal plasticity comprises promoting neuronal growth, promoting neuritogenesis, promoting synaptogenesis, promoting dendritogenesis, increasing dendritic arbor complexity, and increasing dendritic spine density.

In some embodiments, increasing neuronal plasticity by treating a subject with a compound of Table 1, Formulas (I), (Ia), (Ia1), (Ia2), (Ia3), (Ib), (Ib1), (Ic), (Ic1), (Ic2), (Ic3), (Id), (Ic1), (Ic2), (1c3), (Id4), (Ie), (Ie1), (Ie2), (Ig), (Ih), (Ii), (Ii1), (Ii2) or (Ij) can treat neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the present invention provides methods for increasing neuronal plasticity, comprising contacting a neuronal cell with a compound of the present invention, such as a compound of Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij). In some embodiments, increasing neuronal plasticity improves a brain disorder described herein.

In some embodiments, a compound of the present invention is used to increase neuronal plasticity. In some embodiments, the compounds used to increase neuronal plasticity have, for example, anti-addictive properties, antidepressant properties, anxiolytic properties, or a combination thereof. In some embodiments, decreased neuronal plasticity is associated with a neuropsychiatric disease. In some embodiments, the neuropsychiatric disease is a mood or anxiety disorder. In some embodiments, the neuropsychiatric disease includes, for example, migraine, cluster headache, post-traumatic stress disorder (PTSD), schizophrenia, anxiety, depression, and addiction (e.g., substance abuse disorder). In some embodiments, brain disorders include, for example, migraines, addiction (e.g., substance use disorder), depression, and anxiety.

In some embodiments, the experiment or assay to determine increased neuronal plasticity of any compound of the present invention is a phenotypic assay, a dendritogenesis assay, a spinogenesis assay, a synaptogenesis assay, a Sholl analysis, a concentration-response experiment, a 5-HT_{2A} agonist assay, a 5-HT_{2A} antagonist assay, a 5-HT_{2A} binding assay, or a 5-HT_{2A} blocking experiment (e.g., ketanserin blocking experiments). In some embodiments, the experiment or assay to determine the hallucinogenic potential of any compound of the present invention is a mouse head-twitch response (HTR) assay. In some embodiments, the present invention provides a method for increasing neuronal plasticity, comprising contacting a neuronal cell with a compound of Table 1, Formulas (I), (Ia), (Ia1), (Ia2), (Ia3), (Ib), (Ib1), (Ic), (Ic1), (Ic2), (Ic), (Id), (Ic1), (Ic2), (Ic), (Id4), (Ie), (Ie1), (Ie2), (Ig), (Ih), (Ii), (Ii1), (Ii2) or (Ij).

### Methods of Treating a Brain Disorder

In some embodiments, the present invention provides a method of treating a disease, including administering to a subject in need thereof, a therapeutically effective amount of a compound of the present invention, such as a compound of Table 1, Formulas (I), (Ia), (Ia1), (Ia2), (1a3), (Ib), (Ib1), (Ic), (Ic1), (Ic2), (1c3), (Id), (Ic1), (Ic2), (1c3), (Id4), (Ie), (Ie1), (Ie2), (Ig), (Ih), (Ii), (Ii1), (Ii2) or (Ij). In some embodiments, the disease is a musculoskeletal pain disorder including fibromyalgia, muscle pain, joint stiffness, osteoarthritis, rheumatoid arthritis, muscle cramps. In some embodiments, the present invention provides a method of treating a disease of women's reproductive health including premenstrual dysphoric disorder (PMDD), premenstrual syndrome (PMS), post-partum depression, and menopause. In some embodiments, the present invention provides a method of treating a brain disorder, including administering to a subject in need thereof, a therapeutically effective amount of a compound of the present invention. In some embodiments, the present invention provides a method of treating a brain disorder with combination therapy, including administering to a subject in need thereof, a therapeutically effective amount of a compound of the present invention and at least one additional therapeutic agent.

In some embodiments, 5-HT_{2A} modulators (e.g., 5-HT_{2A} agonists) are used to treat a brain disorder. In some embodiments, the brain disorders comprise decreased neural plasticity, decreased cortical structural plasticity, decreased 5-HT_{2A} receptor content, decreased dendritic arbor complexity, loss of dendritic spines, decreased dendritic branch content, decreased spinogenesis, decreased neuritogenesis, retraction of neurites, or any combination thereof.

In some embodiments, a compound of the present invention, such as a compound of Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij), is used to treat brain disorders. In some embodiments, the compounds have, for example, anti-addictive properties, antidepressant properties, anxiolytic properties, or a combination thereof. In some embodiments, the brain disorder is a neuropsychiatric disease. In some embodiments, the neuropsychiatric disease is a mood or anxiety disorder. In some embodiments, brain disorders include, for example, migraine, cluster headache, post-traumatic stress disorder (PTSD), anxiety, depression, panic disorder, suicidality, schizophrenia, and addiction (e.g., substance abuse disorder). In some embodiments, brain disorders include, for example, migraines, addiction (e.g., substance use disorder), depression, and anxiety.

In some embodiments, the present invention provides a method of treating a brain disorder, comprising administering to a subject in need thereof a therapeutically effective amount of a compound disclosed herein, such as a compound of Table 1, Formulas (I), (Ia), (Jal), (Ia2), (1a3), (Ib), (Ib1), (Ic), (Ic1), (Ic2), (1c3), (Id), (Ic1), (Ic2), (1c3), (Id4), (Ie), (Ie1), (Ie2), (Ig), (Ih), (Ii), (Ii1), (Ii2) or (Ij).

In some embodiments, the brain disorder is a neurodegenerative disorder, Alzheimer's, Parkinson's disease, psychological disorder, depression, addiction, anxiety, post-traumatic stress disorder, treatment resistant depression, suicidal ideation, major depressive disorder, bipolar disorder, schizophrenia, stroke, traumatic brain injury, or substance use disorder.

In some embodiments, the brain disorder is a neurodegenerative disorder, Alzheimer's, or Parkinson's disease. In some embodiments, the brain disorder is a psychological disorder, depression, addiction, anxiety, or a post-traumatic stress disorder. In some embodiments, the brain disorder is depression. In some embodiments, the brain disorder is addiction. In some embodiments, the brain disorder is treatment resistant depression, suicidal ideation, major depressive disorder, bipolar disorder, schizophrenia, stroke, traumatic brain injury or substance use disorder. In some embodiments, the brain disorder is treatment resistant depression, suicidal ideation, major depressive disorder, bipolar disorder, schizophrenia, or substance use disorder. In some embodiments, the brain disorder is stroke or traumatic brain injury. In some embodiments, the brain disorder is treatment resistant depression, suicidal ideation, major depressive disorder, bipolar disorder, or substance use disorder. In some embodiments, the brain disorder is schizophrenia. In some embodiments, the brain disorder is alcohol use disorder.

In some embodiments, the method further comprises administering one or more additional therapeutic agent that is lithium, olanzapine (Zyprexa), quetiapine (Seroquel), risperidone (Risperdal), ariprazole (Abilify), ziprasidone (Geodon), clozapine (Clozaril), divalproex sodium (Depakote), lamotrigine (Lamictal), valproic acid (Depakene), carbamazepine (Equetro), topiramate (Topamax), levomilnacipran (Fetzima), duloxetine (Cymbalta, Yentreve), venlafaxine (Effexor), citalopram (Celexa), fluvoxamine (Luvox), escitalopram (Lexapro), fluoxetine (Prozac), paroxetine (Paxil), sertraline (Zoloft), clomipramine (Anafranil), amitriptyline (Elavil), desipramine (Norpramin), imipramine (Tofranil), nortriptyline (Pamelor), phenelzine (Nardil), tranylcypromine (Parnate), diazepam (Valium), alprazolam (Xanax), or clonazepam (Klonopin).

In certain embodiments of the method for treating a brain disorder disclosed herein with a compound according to Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij), a second therapeutic agent that is an empathogenic agent is administered. Examples of suitable empathogenic agents for use in combination with a compound according to Table 1, Formulas (I), (Ia), (Ia1), (Ia2), (Ia3), (Ib), (Ib1), (Ic), (Ic1), (Ic2), (Ic3), (Id), (Ic1), (Ic2), (Ic3), (Id4), (Ie), (Ie1), (Ie2), (Ig), (Ih), (Ii), (Ii1), (Ii2) or (Ij) are selected from the phenethylamines, such as 3,4-methylenedioxymethamphetamine (MDMA) and analogs thereof. Other suitable empathogenic agents for use in combination with the presently disclosed compounds include, without limitation,
N-Allyl-3,4-methylenedioxy-amphetamine (MDAL)
N-Butyl-3,4-methylenedioxyamphetamine (MDBU)
N-Benzyl-3,4-methylenedioxyamphetamine (MDBZ)
N-Cyclopropylmethyl-3,4-methylenedioxyamphetamine (MDCPM)
N,N-Dimethyl-3,4-methylenedioxyamphetamine (MDDM)
N-Ethyl-3,4-methylenedioxyamphetamine (MDE; MDEA)
N-(2-Hydroxyethyl)-3,4-methylenedioxy amphetamine (MDHOET)
N-Isopropyl-3,4-methylenedioxyamphetamine (MDIP)
N-Methyl-3,4-ethylenedioxyamphetamine (MDMC)
N-Methoxy-3,4-methylenedioxyamphetamine (MDMEO)
N-(2-Methoxyethyl)-3,4-methylenedioxyamphetamine (MDMEOET)
alpha,alpha,N-Trimethyl-3,4-methylenedioxyphenethylamine (MDMP;
3,4-Methylenedioxy-N-methylphentermine)
N-Hydroxy-3,4-methylenedioxyamphetamine (MDOH)
3,4-Methylenedioxyphenethylamine (MDPEA)
alpha,alpha-Dimethyl-3,4-methylenedioxyphenethylamine (MDPH; 3,4-methylenedioxyphentermine)
N-Propargyl-3,4-methylenedioxyamphetamine (MDPL)
Methylenedioxy-2-aminoindane (MDAI)
N-methyl-1,3-benzodioxolylbutanamine (MBDB)
3,4-methylenedioxy-N-methyl-α-ethylphenylethylamine
3,4-Methylenedioxyamphetamine MDA
Methylone (also known as “3,4-methylenedioxy-N-methylcathinone)
Ethylone, also known as 3,4-methylenedioxy-N-ethylcathinone
GHB or Gamma Hydroxybutyrate or sodium oxybate
N-Propyl-3,4-methylenedioxyamphetamine (MDPR), and the like.

In some embodiments, the compounds of the present invention are used in combination with the standard of care therapy for a neurological disease described herein. Non-limiting examples of the standard of care therapies, may include, for example, lithium, olanzapine, quetiapine, risperidone, ariprazole, ziprasidone, clozapine, divalproex sodium, lamotrigine, valproic acid, carbamazepine, topiramate, levomilnacipran, duloxetine, venlafaxine, citalopram, fluvoxamine, escitalopram, fluoxetine, paroxetine, sertraline, clomipramine, amitriptyline, desipramine, imipramine, nortriptyline, phenelzine, tranylcypromine, diazepam, alprazolam, clonazepam, or any combination thereof. Nonlimiting examples of standard of care therapy for depression are sertraline, fluoxetine, escitalopram, venlafaxine, or aripiprazole. Non-limiting examples of standard of care therapy for depression are citralopram, escitalopram, fluoxetine, paroxetine, diazepam, or sertraline. Additional examples of standard of care therapeutics are known to those of ordinary skill in the art.

### Methods of Increasing at Least One of Translation, Transcription, or Secretion of Neurotrophic Factors

Neurotrophic factors refers to a family of soluble peptides or proteins which support the survival, growth, and differentiation of developing and mature neurons. Increasing at least one of translation, transcription, or secretion of neurotrophic factors can be useful for, but not limited to, increasing neuronal plasticity, promoting neuronal growth, promoting neuritogenesis, promoting synaptogenesis, promoting dendritogenesis, increasing dendritic arbor complexity, increasing dendritic spine density, and increasing excitatory synapsis in the brain. In some embodiments, increasing at least one of translation, transcription, or secretion of neurotrophic factors can increasing neuronal plasticity. In some embodiments, increasing at least one of translation, transcription, or secretion of neurotrophic factors can promoting neuronal growth, promoting neuritogenesis, promoting synaptogenesis, promoting dendritogenesis, increasing dendritic arbor complexity, and/or increasing dendritic spine density.

In some embodiments, 5-HT_{2A} modulators (e.g., 5-HT_{2A} agonists) are used to increase at least one of translation, transcription, or secretion of neurotrophic factors. In some embodiments, a compound of the present invention, such as a compound of Formula I, is used to increase at least one of translation, transcription, or secretion of neurotrophic factors. In some embodiments, increasing at least one of translation, transcription or secretion of neurotrophic factors treats a migraine, headaches (e.g., cluster headache), post-traumatic stress disorder (PTSD), anxiety, depression, neurodegenerative disorder, Alzheimer's disease, Parkinson's disease, psychological disorder, treatment resistant depression, suicidal ideation, major depressive disorder, bipolar disorder, schizophrenia, stroke, traumatic brain injury, and addiction (e.g., substance use disorder).

In some embodiments, the experiment or assay used to determine increase translation of neurotrophic factors includes ELISA, western blot, immunofluorescence assays, proteomic experiments, and mass spectrometry. In some embodiments, the experiment or assay used to determine increase transcription of neurotrophic factors includes gene expression assays, PCR, and microarrays. In some embodiments, the experiment or assay used to determine increase secretion of neurotrophic factors includes ELISA, western blot, immunofluorescence assays, proteomic experiments, and mass spectrometry.

In some embodiments, the present invention provides a method for increasing at least one of translation, transcription or secretion of neurotrophic factors, comprising contacting a neuronal cell with a compound disclosed herein, such as a compound of Table 1, Formulas (I), (Ia), (Ia1), (Ia2), (1a3), (Ib), (Ib1), (Ic), (Ic1), (Ic2), (1c3), (Id), (Ic1), (Ic2), (1c3), (Id4), (Ie), (Ie1), (Ie2), (Ig), (Ih), (Ii), (Ii1), (Ii2) or (Ij).

### EXAMPLES

Exemplary compounds disclosed herein are prepared from the isotopically enriched building blocks analogous to those used to synthesize the unenriched compounds.

### I. Chemical Synthesis

Chemicals were purchased and used without further purification.

Solvents were purchased as anhydrous. Petrol was the fraction boiling between 40-60° C.

TLC was carried out using aluminium plates pre-coated with silica gel. Visualisation was by UV light and potassium permanganate staining.

¹H NMR were recorded on a Bruker Avance BVT3200 spectrometer using the residual proton(s) in the deuterated solvents as internal standards.

HPLC analyses were performed on a Shimadzu Prominence instrument with diode array detection.

LC-MS analyses were performed on a Shimadzu 2010EV instrument operating in positive or negative electrospray (ESI) mode.

Automated chromatography was performed on a Biotage Selekt purification system.

### Example 1: Synthesis of (6aR,9R)-5-bromo-4-butyryl-N,N-diethyl-7-methyl-4,6,6a,7,8,9-hexahydroindolo[4,3-fg]quinoline-9-carboxamide

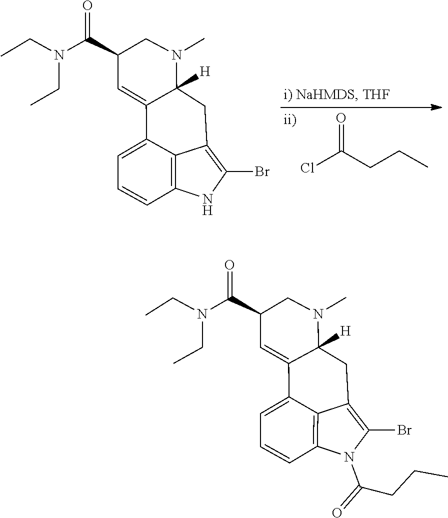

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂. The mixture was stirred at −78° C. for 30 min. Butyryl chloride (29 mg, 29 μL, 0.27 mmol, 1.1 equiv.) in THE (1 mL) was added dropwise and the reaction mixture was warmed to rt and stirred for 16 h. The mixture was concentrated to give a solid, which was purified by column chromatography on silica gel, eluting with a gradient of MeOH in DCM to give (6aR,9R)-5-bromo-4-butyryl-N,N-diethyl-7-methyl-4,6,6a,7,8,9-hexahydroindolo[4,3-fg]quinoline-9-carboxamide (22.6 mg, 19%) as a semi-solid. LC-MS (+ve mode): m/z 472.15 and 474.15 [M+H]⁺; ¹HNMR (CDCl₃, 300 MHz) δ 7.96 (d, 1H, J=8.0 Hz, ArH), 7.37 (d, 1H, J=8.0 Hz, ArH), 7.27 (m, 1H, ArH), 6.36 (s, 1H, C═CH), 3.86 (m, 1H, CHCO), 3.41 (m, 5H, CHN and 2×CH₂Me), 3.15 (m, 3H, CHH and CH₂CO), 3.05 (dd, 1H, J=11.3, 4.4 Hz, CHH), 2.85 (t, 1H, J=10.9 Hz, CHH), 2.60 (s, 3H, NCH₃), 2.46 (dd, 1H, J=15.5, 11.5 Hz, CHH), 1.87 (m, 2H, CH₂CH₂Me), 1.26 (m, 3H, CH₃), 1.17 (t, 3H, J=7.1 Hz, CH₃), 1.06 (t, 3H, J=7.1 Hz, CH₃).

### Example 2: Synthesis of (pivaloyloxy)methyl (6aR,9R)-5-bromo-9-(diethylcarbamoyl)-7-methyl-6a,7,8,9-tetrahydroindolo[4,3-fg]quinoline-4(61)-carboxylate

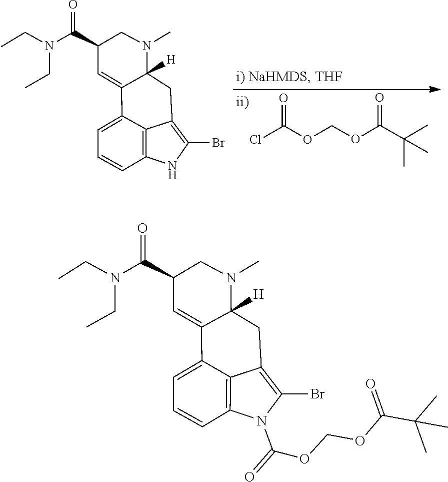

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 275 μL, 0.28 mmol, 1.1 equiv.) was added under an atmosphere of N₂. The mixture was stirred at −78° C. for 30 min. A solution of [(chlorocarbonyl)oxy]methyl 2,2-dimethylpropanoate (49 mg, 0.25 mmol, 1 equiv.) in anhydrous THE (1 mL) was added dropwise. The mixture was stirred at −78° C. for 30 min, then allowed to reach rt over 1 h. The mixture was quenched with water (0.5 mL) and concentrated. The residue was purified by column chromatography on silica gel, eluting with a gradient of MeOH in DCM, to afford (pivaloyloxy)methyl (6aR,9R)-5-bromo-9-(diethylcarbamoyl)-7-methyl-6a,7,8,9-tetrahydroindolo[4,3-fg]quinoline-4(6H)-carboxylate (100 mg, 71%) as a semi-solid. LC-MS (+ve mode): m/z=560.20 and 562.20 [M+H]⁺; ¹H NMR (CDCl₃, 300 MHz) δ 7.69 (d, 1H, J=8.1 Hz, ArH), 7.30 (d, 1H, J=7.4 Hz, ArH), 7.20 (m, 1H, ArH), 6.30 (s, 1H, C═CH), 6.02 (s, 2H, OCH₂O), 3.78 (m, 1H, CHCO), 3.36 (m, 5H, CHN and 2×CH₂Me) 3.10 (m, 1H, CHH), 2.98 (dd, 1H, J=11.6, 5.0 Hz, CHH), 2.77 (t, 1H, J=10.9 Hz, CHH), 2.53 (s, 3H, NCH₃), 2.37 (dd, 1H, J=15.3, 11.5 Hz, CHH), 1.19 (m, 12H, 4×CH₃), 1.10 (t, 3H, J=7.1 Hz, CH₃); ¹³C NMR (75 MHz, CDCl₃) δ 177.1, 171.3, 149.5, 134.5, 134.3, 127.5, 126.7, 126.1, 121.7, 120.5, 117.6, 114.8, 104.4, 80.7, 61.7, 55.7, 43.7, 42.1, 40.4, 39.7, 38.8, 27.7, 26.9, 15.0, 13.2.

### Example 3: Synthesis of ((6aR,9R)-5-bromo-N,N-diethyl-7-methyl-4-pivaloyl-4,6,6a,7,8,9-hexahydroindolo[4,3-fg]quinoline-9-carboxamide

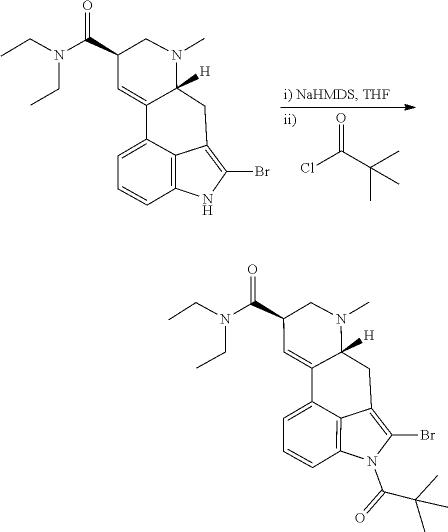

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂. The mixture was stirred at −78° C. for 30 min. Pivaloyl chloride (33 mg, 33 μL, 0.28 mmol, 1.1 equiv.) was added dropwise and the reaction mixture was stirred at −78° C. for 30 min, then allowed to reach rt over 2 h. The mixture was quenched with water (0.5 mL) and concentrated. The residue was purified by column chromatography on silica gel, eluting with a gradient of MeOH in DCM, to afford ((6aR,9R)-5-bromo-N,N-diethyl-7-methyl-4-pivaloyl-4,6,6a,7,8,9-hexahydroindolo[4,3-fg]quinoline-9-carboxamide (56 mg, 46%) as a semi-solid. LC-MS (+ve mode): m/z=486.15 and 488.15 [M+H]⁺; ¹H NMR (CDCl₃, 300 MHz) δ 7.30 (d, 1H, J=7.4 Hz, ArH), 7.08 (m, 2H, 2×ArH), 6.30 (s, 1H, C═CH), 3.82 (m, 1H, CHCO), 3.36 (m, 5H, CHN and 2×CH₂Me), 3.15 (m, 1H, CHH), 2.99 (dd, 1H, J=11.7, 4.6 Hz, CHH), 2.80 (t, 1H, J=10.9 Hz, CHH), 2.54 (s, 3H, NCH₃), 2.45 (dd, 1H, J=15.0, 11.4 Hz, CHH), 1.38 (s, 9H, 3×CH₃), 1.19 (m, 3H, CH₃), 1.10 (t, 3H, J=7.1 Hz, CH₃); ¹³C NMR (75 MHz, CDCl₃) δ 184.4, 171.4, 134.8, 129.7, 127.7, 126.1, 124.5, 121.3, 115.7, 114.9, 110.6, 62.2, 55.9, 43.8, 42.1, 40.4, 39.8, 28.1, 27.6, 27.1, 15.0, 13.2.

### Example 4: Synthesis of isopropyl (6aR,9R)-5-bromo-9-(diethylcarbamoyl)-7-methyl-6a,7,8,9-tetrahydroindolo[4,3-fg]quinoline-4(6H)-carboxylate

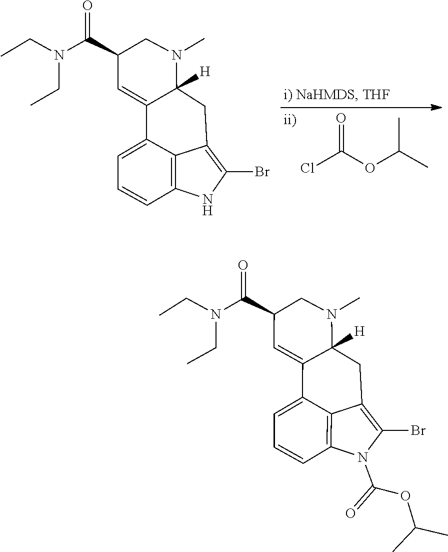

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂. The mixture was stirred at −78° C. for 30 min. Isopropyl chloroformate (1.0 μM in toluene, 275 μL, 0.275 mmol, 1.1 equiv.) was added dropwise. The mixture was stirred at −78° C. for 30 min, followed by rt for 16 h. The mixture was quenched with water (0.5 mL) and concentrated to give a semi-solid. The residue was purified by column chromatography on silica gel, eluting with a gradient of MeOH in DCM, to afford isopropyl (6aR,9R)-5-bromo-9-(diethylcarbamoyl)-7-methyl-6a,7,8,9-tetrahydroindolo[4,3-fg]quinoline-4(6H)-carboxylate (36 mg, 29%) as a semi-solid. LC-MS (+ve mode): m/z=488.15 and 490.15 [M+H]⁺; ¹H NMR (CDCl₃, 300 MHz) δ 7.71 (d, 1H, J=8.2 Hz, ArH), 7.28 (d, 1H, J=7.3 Hz, ArH), 7.19 (m, 1H, ArH), 6.29 (s, 1H, C═CH), 5.23 (m, 1H, CH(CH₃)₂) 3.82 (m, 1H, CHCO), 3.36 (m, 5H, CHN and 2×CH₂Me), 3.13 (m, 1H, CHH), 2.99 (m, 1H, CHH), 2.80 (m, 1H, CHH), 2.54 (s, 3H, NCH₃), 2.38 (m, 1H, CHH), 1.42 (d, 6H, J=6.3 Hz, 2×CH₃), 1.19 (t, 3H, J=7.1 Hz, CH₃), 1.10 (t, 3H, J=7.1 Hz, CH₃).

### Example 5: Synthesis of (6aR,9R)-5-bromo-N,N-diethyl-4-(hydroxymethyl)-7-methyl-4,6,6a,7,8,9-hexahydroindolo[4,3-fg]quinoline-9-carboxamide

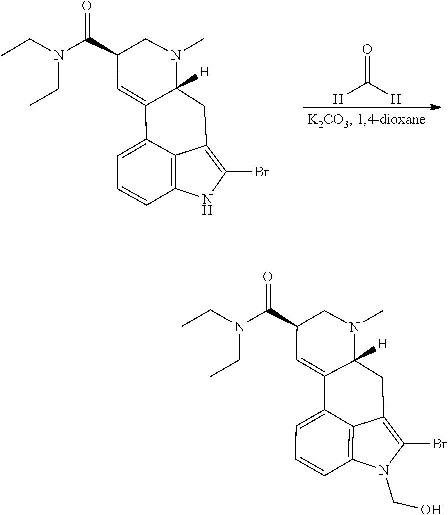 [SMILES: CCN(CC)C(=O)[C@@H]1C=C2c3cccc4[nH]c(Br)c(c34)C[C@H]2N(C)C1 ; C=O ; CCN(CC)C(=O)[C@@H]1C=C2c3cccc4c3c(c(Br)n4CO)C[C@H]2N(C)C1]

2-Br-LSD (100 mg, 0.25 mmol, 1 equiv.) was dissolved in anhydrous 1,4-dioxane (3 mL). K₂CO₃ (34 mg, 0.25 mmol, 1 equiv.) and paraformaldehyde (7 mg, 0.25 mmol, 1 equiv.) were added and the mixture was heated at 60° C. for 16 h. The mixture was concentrated and the residue was purified by column chromatography on silica gel, eluting with a gradient of MeOH in DCM, to afford (6aR,9R)-5-bromo-N,N-diethyl-4-(hydroxymethyl)-7-methyl-4,6,6a,7,8,9-hexahydroindolo[4,3-fg]quinoline-9-carboxamide (70 mg, 64%) as a semi-solid. LC-MS (+ve mode): m/z=432.15 and 434.15 [M+H]⁺; ¹H NMR (CDCl₃, 300 MHz) δ 7.12 (m, 3H, 2×ArH), 6.23 (s, 3H, C═CH), 5.58 (s, 2H, CH₂OH) 3.80 (m, 1H, CHCO), 3.40 (m, 4H, 2×CH₂Me), 3.23 (dd, 1H, J=14.8, 5.7 Hz, CHN), 2.96 (m, 2H, CH₂), 2.76 (t, 1H, J=10.9 Hz, CHH), 2.50 (s, 3H, NCH₃), 2.33 (dd, 1H, J=14.8, 11.4 Hz, CHH), 1.19 (t, 3H, J=7.1 Hz, CH₃), 1.11 (t, 3H, J=7.1 Hz, CH₃); ¹³C NMR (75 MHz, CDCl₃) δ 171.9, 135.0, 134.4, 126.9, 125.8, 123.6, 119.9, 113.8, 111.8, 108.2, 106.6, 67.7, 61.9, 55.8, 43.6, 42.2, 40.5, 39.7, 26.7, 15.0, 13.2.

### Example 6: Synthesis of ((6aR,9R)-5-bromo-9-(diethylcarbamoyl)-7-methyl-6a,7,8,9-tetrahydroindolo[4,3-fg]quinolin-4(6H)-yl) methyl pivalate

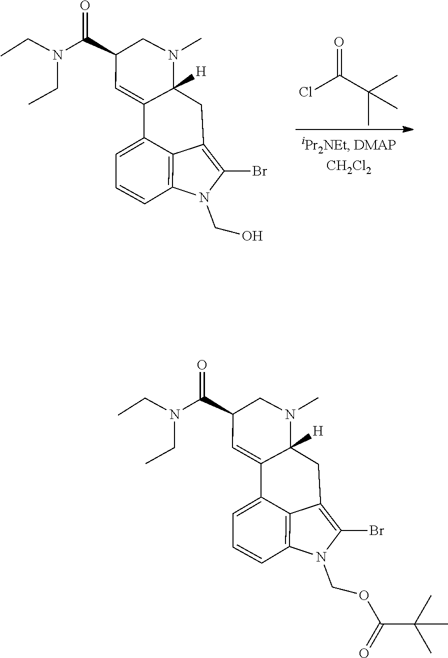

To a solution of (6aR,9R)-5-bromo-N,N-diethyl-4-(hydroxymethyl)-7-methyl-4,6,6a,7,8,9-hexahydroindolo[4,3-fg]quinoline-9-carboxamide (35 mg, 0.08 mmol, 1 equiv.) in DCM (3 mL) was added N,N-diisopropylethylamine (12 mg, 15 μL, 0.09 mmol, 1.1 equiv.), 4-dimethylaminopyridine (2 mg, 0.002 mmol, 0.25 equiv.) and pivaloyl chloride (10 mg, 10 μL, 0.09 mmol, 1.1 equiv). The mixture was stirred at rt for 1 h. The mixture was concentrated and the residue was purified by column chromatography on silica gel, eluting with a gradient of MeOH in DCM, to afford ((6aR,9R)-5-bromo-9-(diethylcarbamoyl)-7-methyl-6a,7,8,9-tetrahydroindolo[4,3-fg]quinolin-4(6H)-yl) methyl pivalate (32 mg, 77%) as a semi-solid. LC-MS (+ve mode): m/z=516.25 and 518.25 [M+H]⁺; ¹H NMR (CDCl₃, 300 MHz) δ 7.32 (m, 1H, ArH), 7.22 (m, 2H, ArH), 6.38 (s, 1H, C═CH), 6.14 (s, 2H, NCH₂O), 3.91 (m, 1H, CHCO), 3.44 (m, 5H, 2×CH₂Me and CHN), 3.25 (m, 1H, CHH), 3.10 (dd, 1H, J=11.4, 4.3 Hz, CHH), 2.90 (t, 1H, J=11.0 Hz, CHH), 2.64 (s, 3H, NCH₃), 2.58 (dd, 1H, J=15.0, 11.3 Hz, CHH), 1.27 (t, 3H, J=7.10 Hz, CH₃), 1.18 (m, 12H, 4×CH₃); ¹³C NMR (75 MHz, CDCl₃) δ 178.0, 173.4, 171.4, 135.0, 127.3, 126.1, 124.0, 120.9, 114.36, 113.1, 108.6, 107.3, 67.4, 62.4, 55.9, 43.8, 42.1, 40.3, 39.7, 39.0, 27.0, 15.0, 13.2.

### Example 7: Synthesis of ((6aR,9R)-5-bromo-9-(diethylcarbamoyl)-7-methyl-6a,7,8,9-tetrahydroindolo[4,3-fg]quinolin-4(6H)-yl)methyl ethyl carbonate

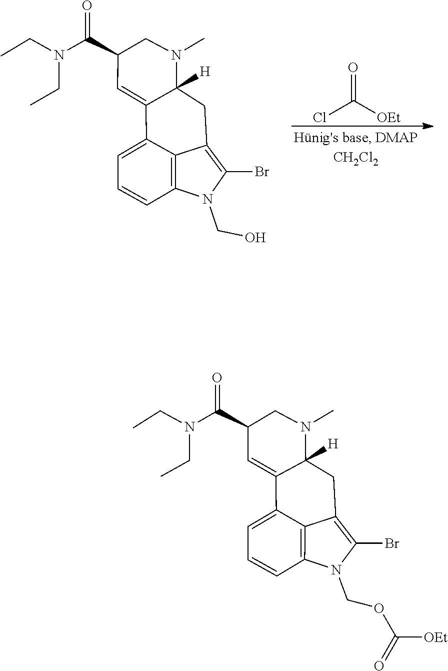 [SMILES: CCN(CC)C(=O)[C@@H]1C=C2c3cccc4c3c(c(Br)n4CO)C[C@H]2N(C)C1 ; CCOC(=O)Cl ; CCOC(=O)OCn1c(Br)c2c3c(cccc31)C1=C[C@@H](C(=O)N(CC)CC)CN(C)[C@@H]1C2]

To a solution of (6aR,9R)-5-bromo-N,N-diethyl-4-(hydroxymethyl)-7-methyl-4,6,6a,7,8,9-hexahydroindolo[4,3-fg]quinoline-9-carboxamide (35 mg, 0.08 mmol, 1 equiv.) in DCM (3 mL) was added N,N-diisopropylethylamine (12 mg, 15 μL, 0.09 mmol, 1.1 equiv.), 4-dimethylaminopyridine (2 mg, 0.002 mmol, 0.25 equiv.) and ethyl chloroformate (9.8 mg, 8.5 μL, 0.09 mmol, 1.1 equiv.). The mixture was stirred at rt for 1 h. The mixture was concentrated and the residue was purified by column chromatography on silica gel, eluting with a gradient of MeOH in DCM, to afford ((6aR,9R)-5-bromo-9-(diethylcarbamoyl)-7-methyl-6a,7,8,9-tetrahydroindolo[4,3-fg]quinolin-4(6H)-yl)methyl ethyl carbonate (23 mg, 56%) as a semi-solid. LC-MS (+ve mode): m/z=504.20 and 506.20 [M+H]⁺; ¹H NMR (CDCl₃, 300 MHz) δ 7.31 (d, 1H, J=8.5 Hz, ArH), 7.16 (m, 2H, 2×ArH), 6.29 (s, 1H, C═CH), 6.11 (s, 2H, NCH₂O), 4.13 (m, 2H, CH₂), 3.77 (m, 1H, CHCO), 3.36 (m, 5H, 2×CH₂Me and CHN), 3.13 (m, 1H, CHH), 2.98 (dd, 1H, J=10.4, 4.3 Hz, CHH), 2.80 (m, 1H, CHH), 2.54 (s, 3H, NCH₃), 1.32 (m, 1H, CHH), 1.21 (m, 6H, 2×CH₃), 1.10 (t, 3H, J=7.1 Hz, CH₃); ¹³C NMR (75 MHz, CDCl₃) δ 171.4, 154.4, 135.0, 129.7, 127.4, 126.2, 124.2, 120.9, 114.7, 113.7, 108.9, 107.2, 69.9, 64.6, 62.3, 55.9, 43.9, 42.1, 40.3, 39.8, 27.1, 15.0, 14.1, 13.2.

### Example 8-A: Synthesis of benzamidomethyl acetate

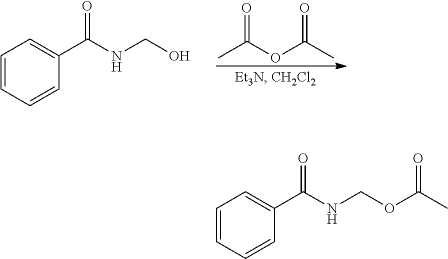

To a suspension of N-hydroxymethylbenzamide (0.50 g, 3.30 mmol, 1 equiv.) in DCM (5 mL) was added Et₃N (1.01 g, 1.38 mL, 9.90 mmol, 3 equiv.). Ac₂₀ (0.68 g, 0.63 mL, 6.60 mmol, 2 equiv.) was added dropwise and the mixture was stirred at rt for 72 h. The resulting solution was diluted with DCM (20 mL) and washed with water (2×20 mL). The organic phase was dried (Na₂SO₄) and concentrated. The residue was purified by column chromatography on silica gel, eluting with a gradient of EtOAc in petrol, to give benzamidomethyl acetate (220 mg, 71%) as an oil. LC-MS (+ve mode): m/z=216.05 [M+Na]⁺; ¹H NMR (300 MHz, CDCl₃) δ 7.72 (m, 2H, 2×ArH), 7.48 (m, 1H, ArH), 7.39 (m, 2H, 2×ArH), 5.39 (d, 2H, CH₂, J=7.3 Hz, CH₂), 2.02 (s, 3H, CH₃).

### Example 8: Synthesis of (6aR,9R)-4-(benzamidomethyl)-5-bromo-N,N-diethyl-7-methyl-4,6,6a,7,8,9-hexahydroindolo[4,3-fg]quinoline-9-carboxamide

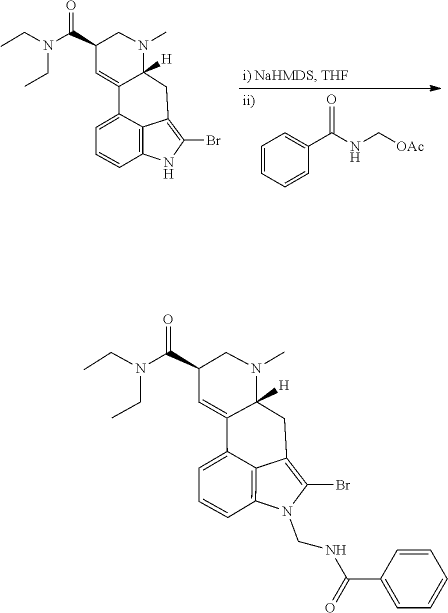 [SMILES: CCN(CC)C(=O)[C@@H]1C=C2c3cccc4[nH]c(Br)c(c34)C[C@H]2N(C)C1 ; CC(=O)OCNC(=O)c1ccccc1 ; CCN(CC)C(=O)[C@@H]1C=C2c3cccc4c3c(c(Br)n4CNC(=O)c3ccccc3)C[C@H]2N(C)C1]

2-Br-LSD (100 mg, 0.25 mmol, 1 equiv.) in anhydrous THE (5 mL) was cooled to −78° C. and sodium bis(trimethylsilyl)amide (1.0 μM in THF, 240 μL, 0.24 mmol, 0.95 equiv.) was added dropwise under an atmosphere of N₂. The mixture was stirred at −78° C. for 30 min. Benzamidomethyl acetate (44 mg, 0.24 mmol, 0.95 equiv.) was added and the resulting mixture was stirred at rt for 2 h. The mixture was concentrated and the residue was purified by column chromatography on silica gel, eluting with a gradient of MeOH in DCM, to give (6aR,9R)-4-(benzamidomethyl)-5-bromo-N,N-diethyl-7-methyl-4,6,6a,7,8,9-hexahydroindolo[4,3-fg]quinoline-9-carboxamide (62 mg, 46%) as a semi-solid. LC-MS (+ve mode): m/z=516.25 and 518.25 [M+H]⁺; ¹H NMR (300 MHz, CDCl₃) δ 7.67 (m, 2H, 2×ArH), 7.43 (m, 4H, 4×ArH), 7.14 (m, 1H, ArH), 6.66 (t, 1H, J 6.19 Hz, ArH), 6.27 (s, 1H, C═CH), 5.79 (dd, 2H, J=6.2, 1.7 Hz, NCH₂N), 3.80 (m, 1H, CHCO), 3.35 (m, 5H, 2×CH₂Me and CHN), 3.13 (m, 1H, CHH), 2.98 (dd, 1H, J=11.1, 4.7 Hz, CHH), 2.81 (t, 1H, J=10.9 Hz, CHH), 2.54 (s, 3H, NCH₃), 2.47 (dd, 1H, J=14.7, 11.5 Hz, CHH), 1.18 (t, 3H, J=7.11 Hz, CH₃), 1.10 (t, 3H, J=7.11 Hz, CH₃); ¹³C NMR (75 MHz, CDCl₃) δ 171.5, 167.1, 135.3, 134.7, 133.5, 132.0, 128.6, 127.1, 125.73, 123.9, 120.6, 113.8, 112.2, 109.2, 106.3, 62.5, 56.0, 48.9, 43.9, 42.1, 40.3, 39.9, 27.2, 15.0, 13.2.

### Example 9: Synthesis of 2-(4-((6aR,9R)-5-bromo-9-(diethylcarbamoyl)-7-methyl-6a,7,8,9-tetrahydroindolo[4,3-fg]quinolin-4(6H)-yl)-2-methyl-4-oxobutan-2-yl)-3,5-dimethylphenyl acetate (2-Br-LSD-trimethyl lock)

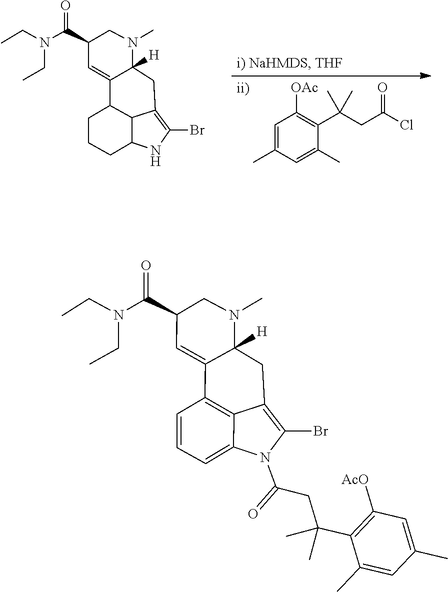 [SMILES: CCN(CC)C(=O)[C@@H]1C=C2C3CCCC4NC(Br)=C(C[C@H]2N(C)C1)C43 ; CC(=O)Oc1cc(C)cc(C)c1C(C)(C)CC(=O)Cl ; CCN(CC)C(=O)[C@@H]1C=C2c3cccc4c3c(c(Br)n4C(=O)CC(C)(C)c3c(C)cc(C)cc3OC(C)=O)C[C@H]2N(C)C1]

### Step 1: 2-(4-Chloro-2-methyl-4-oxobutan-2-yl)-3,5-dimethylphenyl acetate

To a solution of 3-(2-acetoxy-4,6-dimethylphenyl)-3-methylbutyric acid (144 mg, 0.54 mmol) in anhydrous DCM (0.6 mL) at 0° C. under an atmosphere of N₂ was added oxalyl chloride (70 mg, 48 μL, 0.54 mmol) and the mixture was stirred at rt for 3 h. The mixture was concentrated to dryness, azeotroped with DCM (5×4 mL), and dried under high vacuum for 20 min to afford 2-(4-chloro-2-methyl-4-oxobutan-yl)-3,5-dimethylphenyl acetate as a yellow oil, which was used directly in the next step.

### Step 2: 2-(4-((6aR,9R)-5-Bromo-9-(diethylcarbamoyl)-7-methyl-6a,7,8,9-tetrahydroindolo[4,3-fg]quinolin-4(6H)-yl)-2-methyl-4-oxobutan-2-yl)-3,5-dimethylphenyl acetate

To a solution of 2-Br-LSD (200 mg, 0.49 mmol, 1 equiv.) in anhydrous THE (6.0 mL) at −78° C. under an atmosphere of N₂ was added NaHMDS (1 μM in THF, 0.47 mL, 0.47 mmol, 0.95 equiv.) and the mixture was stirred at −78° C. for 30 min. 2-(4-Chloro-2-methyl-4-oxobutan-2-yl)-3,5-dimethylphenyl acetate solution (1M in THF, 0.54 mL, 0.54 mmol, 1.1 equiv.) was added and the reaction mixture was stirred at −78° C. for 30 min, followed by rt overnight. The mixture was quenched with water (1.0 mL) and concentrated to give a semi-solid. The residue was purified by column chromatography on silica gel, eluting with a gradient of MeOH in DCM, to afford 2-(4-((6aR,9R)-5-bromo-9-(diethylcarbamoyl)-7-methyl-6a,7,8,9-tetrahydroindolo[4,3-fg]quinolin-4(6H)-yl)-2-methyl-4-oxobutan-2-yl)-3,5-dimethylphenyl acetate (13 mg, 4%) as a semi-solid. LC-MS (+ve mode): m/z=648.25 and 650.25 [M+H]⁺; ¹H NMR (CDCl₃, 300 MHz) δ 7.72 (d, 1H, J=8.2 Hz, ArH), 7.26 (d, 1H, J=7.6 Hz, ArH), 7.13 (app t, 1H, ArH), 6.74 (d, 1H, J=2.0 Hz, ArH), 6.41 (d, 1H, J=2.0 Hz, ArH), 6.28 (s, 1H, C═CH), 3.79 (m, 1H, CHCO), 3.65 and 3.64 (s, 2H, CH₂), 3.38 (m, 5H, CHN and 2×CH₂Me), 3.07 (m, 1H, CHH), 2.98 (m, 1H, CHH), 2.99 (m, 1H, CHH), 2.78 (m, 1H, CHH), 2.11 and 2.02 (2×s, 6H, 2×CH₃), 2.09 (s, 3H, COCH₃), 1.65 (s, 6H, 2×CCH₃), 1.26 (t, J=7.2, 1.1 Hz, 6H, 2×CH₃).

### Example 10: Synthesis of (6aR,9R)-5-bromo-4-(dimethylglycyl)-N,N-diethyl-7-methyl-4,6,6a,7,8,9-hexahydroindolo[4,3-fg]quinoline-9-carboxamide

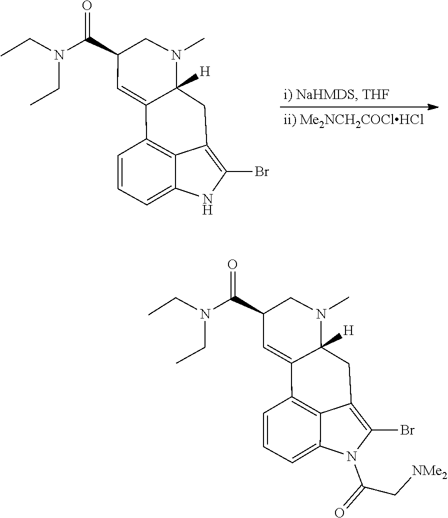 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4c3c(c(Br)n4C(=O)CN(C)C)C[C@H]2N(C)C1]

2-Br-LSD (100 mg, 0.25 mmol, 1 equiv.) was dissolved in anhydrous THE (2 mL) and cooled to −78° C. Sodium bis(trimethylsilyl)amide (1.0 μM in THF, 240 μL, 0.24 mmol, 0.95 equiv.) was added under nan atmosphere of N₂. The mixture was stirred at −78° C. for 30 min. A suspension of 2-(dimethylamino)acetyl chloride hydrochloride (43 mg, 0.27 mmol, 1.1 equiv.) was added. and the reaction mixture was stirred at −78° C. for 30 min, followed by rt overnight. The mixture was quenched with water (1.0 mL) and concentrated to give (6aR,9R)-5-bromo-4-(dimethylglycyl)-N,N-diethyl-7-methyl-4,6,6a,7,8,9-hexahydroindolo[4,3-fg]quinoline-9-carboxamide (2-Br-LSD N,N-dimethylglycinate) (160 mg) as a semi-solid. LC-MS (+ve mode): m/z=489.2 and 490.2 [M+H]⁺.

### Example 11: Synthesis of (6aR,9R)-5-bromo-N,N-diethyl-7-methyl-4-propionyl-4,6,6a,7,8,9-hexahydroindolo[4,3-fg]quinoline-9-carboxamide

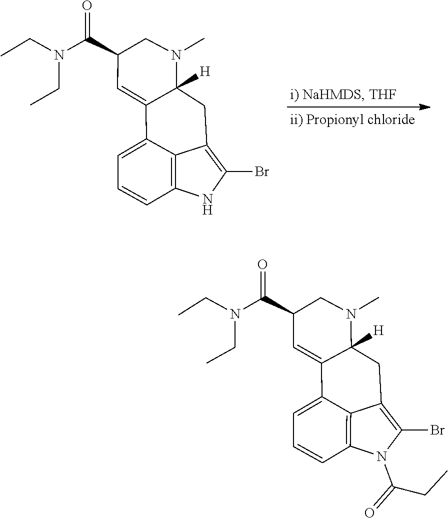 [SMILES: CCN(CC)C(=O)[C@@H]1C=C2c3cccc4[nH]c(Br)c(c34)C[C@H]2N(C)C1 ; CCC(=O)n1c(Br)c2c3c(cccc31)C1=C[C@@H](C(=O)N(CC)CC)CN(C)[C@@H]1C2]

2-Br-LSD (50 mg, 0.12 mmol, 1 equiv.) was dissolved in anhydrous THE (5 mL) and cooled to −78° C. Sodium bis(trimethylsilyl)amide (1.0 μM in THF, 120 μL, 0.12 mmol, 0.95 equiv.) was added under an atmosphere of N₂. The mixture was stirred at −78° C. for 30 min. Propionyl chloride (11 mg, 11 μL, 0.12 mmol, 1 equiv.) was added dropwise. The mixture was stirred at −78° C. for 30 min, followed by rt overnight. The mixture was concentrated and the residue was purified twice by column chromatography on silica gel, eluting with a gradient of MeOH in DCM, to afford (6aR,9R)-5-bromo-N,N-diethyl-7-methyl-4-propionyl-4,6,6a,7,8,9-hexahydroindolo[4,3-fg]quinoline-9-carboxamide (44 mg, 77%) as an orange-brown semi-solid. LC-MS (+ve mode): m/z=456.10 and 458.10 [M+H]⁺; ¹H NMR (CDCl₃, 300 MHz) δ 7.98 (d, 1H, J=8.2 Hz, ArH), 7.37 (d, 1H, J=7.3 Hz, ArH), 7.29 (d, 1H, J=8.0 Hz, ArH), 6.36 (s, 1H, C═CH), 3.86 (m, 1H, CHCO), 3.45 (m, 5H, CHN and 2×CH₂), 3.19 (m, 3H, CHH and N—CH₂), 3.03 (dd, 1H, J=4.8 Hz, CHH), 2.87 (t, 1H, J=10.7 Hz, CHH), 2.61 (s, 3H, N—CH₃), 2.45 (m, 1H, CHH), 1.34 (t, 3H, J=7.3 Hz, CH₃), 1.26 (t, 3H, J=7.1 Hz, CH₃), 1.17 (t, 3H, J=7.6 Hz, CH₃).

### Example 12: Synthesis of propyl (6aR,9R)-5-bromo-9-(diethylcarbamoyl)-7-methyl-6a,7,8,9-tetrahydroindolo[4,3-fg]quinoline-4(6H)-carboxylate

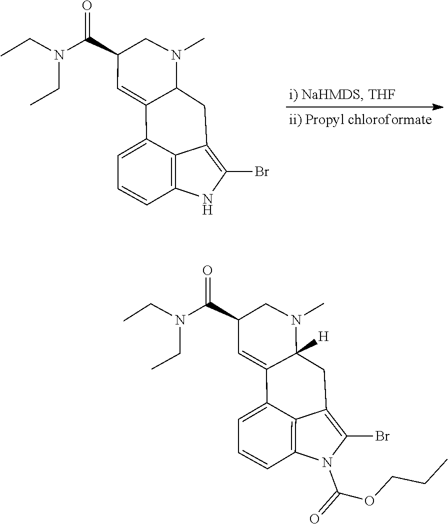 [SMILES: CCN(CC)C(=O)[C@@H]1C=C2c3cccc4[nH]c(Br)c(c34)CC2N(C)C1 ; CCCOC(=O)n1c(Br)c2c3c(cccc31)C1=C[C@@H](C(=O)N(CC)CC)CN(C)[C@@H]1C2]

2-Br-LSD (50 mg, 0.12 mmol, 1 equiv.) was dissolved in anhydrous THE (6 mL) and cooled to −78° C. Sodium bis(trimethylsilyl)amide (1.0 μM in THF, 120 μL, 0.12 mmol, 0.95 equiv.) was added under an atmosphere of N₂ and the mixture was stirred at −78° C. for 30 min. A solution of propyl chloroformate (15 mg, 14 μL, 0.12 mmol, 1 equiv.) in anhydrous THE (2 mL) was added dropwise. The mixture was stirred at −78° C. for 30 min, followed by rt overnight. The mixture was quenched with water (0.5 mL) and concentrated. The residue was purified twice by column chromatography on silica gel, eluting with a gradient of MeOH in DCM, to afford propyl 6aR,9R)-5-bromo-9-(diethylcarbamoyl)-7-methyl-6a,7,8,9-tetrahydroindolo[4,3-fg]quinoline-4(6H)-carboxylate (16 mg, 26%) as a semi-solid. LC-MS (+ve mode): m/z=488.15 and 490.15 [M+H]⁺; ¹HNMR (CDCl₃, 300 MHz) δ 7.78 (d, 1H, J=8.0 Hz, ArH), 7.35 (d, 1H, J=7.3 Hz, ArH), 7.24 (obscured, 1H, ArH), 6.36 (s, 1H, C═CH), 5.28 (obscured, 2 H, CH₂), 3.86 (m, 1H, CHCO), 3.45 (m, 5H, CHN and 2×CH₂), 3.15 (m, 1H, CHH), 3.04 (dd, 1H, J=10.5, 4.9 Hz, CHH), 2.85 (t, 1H, J=10.8 Hz, CHH), 2.60 (s, 3H, NCH₃), 2.44 (dd, 2H, J=15.3, 11.7 Hz), 1.50 (br. s, 3H, CH₃), 1.48 (br. s, 2H, CH₂), 1.26 (t, 3H, J=7.1 Hz, CH₃), 1.17 (t, 3H, J=7.1 Hz, CH₃).

### Example 13: Synthesis of tert-butyl (6aR,9R)-5-bromo-9-(diethylcarbamoyl)-7-methyl-6a,7,8,9-tetrahydroindolo[4,3-fg]quinoline-4(6H)-carboxylate

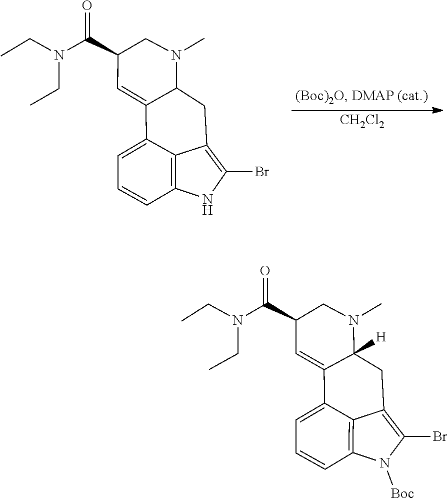 [SMILES: CCN(CC)C(=O)[C@@H]1C=C2c3cccc4[nH]c(Br)c(c34)CC2N(C)C1 ; "CH2Cl2" ; CCN(CC)C(=O)[C@@H]1C=C2c3cccc4c3c(c(Br)n4C(=O)OC(C)(C)C)C[C@H]2N(C)C1]

2-Br-LSD (100 mg, 0.25 mmol, 1 equiv.), di-tert-butyl carbonate (59 mg, 0.28 mmol, 1.1 equiv.) and DMAP (3 mg, 0.025 mmol, 0.1 equiv.) in DCM (2 mL) were stirred at rt for 4 h under an atmosphere of N₂. The reaction mixture was diluted with DCM (20 mL), washed with water (25 mL) and the aqueous phase was extracted with DCM (2×25 mL). The combined organic phases were washed with saturated brine (25 mL), dried (MgSO₄) and concentrated. The residue was purified by column chromatography on silica gel, eluting with a gradient of MeOH in DCM to give tert-butyl (6aR,9R)-5-bromo-9-(diethylcarbamoyl)-7-methyl-6a,7,8,9-tetrahydroindolo[4,3-fg]quinoline-4(6H)-carboxylate (118 mg, 96%) as a semi-solid. LC-MS (+ve mode): m/z=502.20 and 504.20 [M+H]⁺; ¹H NMR (CDCl₃, 300 MHz) δ 7.77 (dd, 1H, J=8.1, 0.7 Hz, ArH), 7.34 (dd, 1H, J=7.6, 0.8 Hz, ArH), 7.23 (m, 1H, obscured, ArH), 6.35 (s, 1H, C═CH), 4.02 (m, 1H, CHCO), 3.87 (m, 1H, CHN), 3.42 (m, 4H, 2×CH₂Me), 3.15 (dddd, 1H, J=11.2, 5.4, 3.5, 2.0 Hz, CHH), 3.04 (ddd, 1H, J=11.1, 5.0, 1.3 Hz, CHH), 2.85 (t, 1H, J=10.9 Hz, CHH) 2.60 (s, 3H, NCH₃), 2.44 (dd, 1H, J=15.3, 11.5 Hz, CHH), 1.69 (s, 9H, 3×CH₃), 1.25 (t, 3H, J=7.1 Hz, CH₃), 1.17 (t, 3H, J=7.0 Hz, CH₃).

### Example 14: Synthesis of (6aR,9R)-5-bromo-N,N-diethyl-4,7-dimethyl-4,6,6a,7,8,9-hexahydroindolo[4,3-fg]quinoline-9-carboxamide

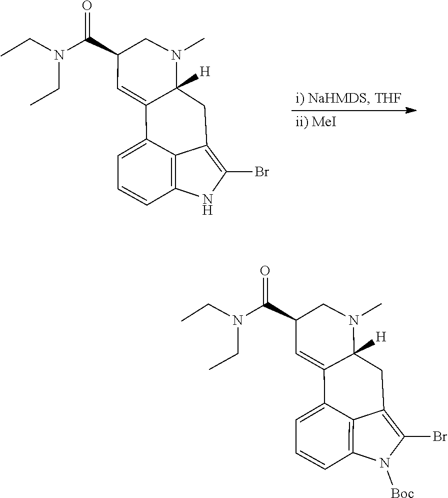 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4c3c(c(Br)n4C(=O)OC(C)(C)C)C[C@H]2N(C)C1]

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂ and the mixture was stirred at −78° C. for 30 min. Methyl iodide (39 mg, 17 μL, 0.27 mmol, 1.1 equiv.) was added dropwise. The mixture was allowed to reach rt over 3 h. The mixture was quenched with water (5 mL) and the aqueous phase was extracted with DCM (3×5 mL). The combined organic layers were washed with saturated aqueous NaHCO₃ (20 mL), saturated brine (20 mL), dried (Na₂SO₄) and concentrated. The residue was purified by column chromatography on silica gel, eluting with a gradient of MeOH in DCM, to give (6aR,9R)-5-bromo-N,N-diethyl-4,7-dimethyl-4,6,6a,7,8,9-hexahydroindolo[4,3-fg]quinoline-9-carboxamide (61 mg) as a semi-solid. LC-MS (+ve mode): m/z=416.10 and 418.10 [M+H]⁺.

### Example 15: Synthesis of (6aR,9R)-5-bromo-N,N-diethyl-4-isobutyryl-7-methyl-4,6,6a,7,8,9-hexahydroindolo[4,3-fg]quinoline-9-carboxamide

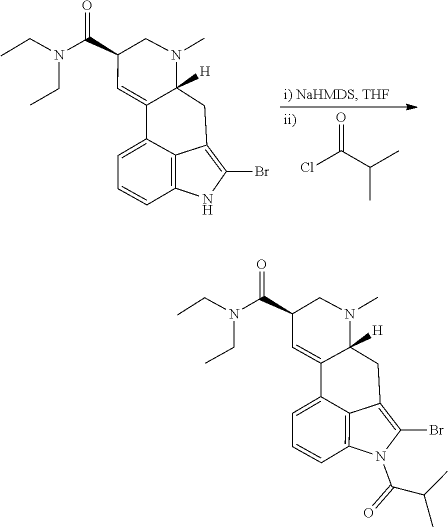

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂ and the mixture was stirred at −78° C. for 30 min. Isobutyryl chloride (29 mg, 29 μL, 0.27 mmol, 1.1 equiv.) was added dropwise. The mixture was allowed to reach rt over 3 h, then quenched with water (5 mL) and the aqueous phase was extracted with DCM (3×3 mL). The combined organic layers were washed with water (10 mL), saturated brine (10 mL), dried (Na₂SO₄) and concentrated to give a residue (89 mg). The residue was purified by column chromatography on silica gel, eluting with a gradient of MeOH in DCM, to afford (6aR,9R)-5-bromo-N,N-diethyl-4-isobutyryl-7-methyl-4,6,6a,7,8,9-hexahydroindolo[4,3-fg]quinoline-9-carboxamide (70 mg, 60%) as a semi-solid. LC-MS (+ve mode): m/z=472.15 and 474.15 [M+H]⁺; ¹H NMR (CDCl₃, 300 MHz) δ 7.88 (dd, 1H, J=8.1, 0.7 Hz, ArH), 7.36 (dd, 1H, J=7.6, 0.8 Hz, ArH), 7.28 (m, 1H, obscured, ArH), 6.36 (s, 1H, C═CH), 3.90 (m, 2H, 2×CHCO), 3.42 (m, 5H, CHN and 2×CH₂Me), 3.19 (m, 1H, CHH), 3.05 (ddd, 1H, J=11.2, 5.1, 1.3 Hz, CHH), 2.86 (t, 1H, J=10.9 Hz, CHH), 2.61 (s, 3H, NCH₃), 2.47 (dd, 1H, J=15.4, 11.5 Hz, CHH), 1.33 (dd, 6H, J=6.7, 0.9 Hz, 2×CH₃), 1.26 (t, 3H, J=7.1 Hz, CH₃), 1.19 (t, 3H, J=7.4 Hz, CH₃).

### Example 16: Synthesis of (6aR,9R)-5-bromo-N,N-diethyl-7-methyl-4-(tetrahydro-2H-pyran-4-carbonyl)-4,6,6a,7,8,9-hexahydroindolo[4,3-fg]quinoline-9-carboxamide

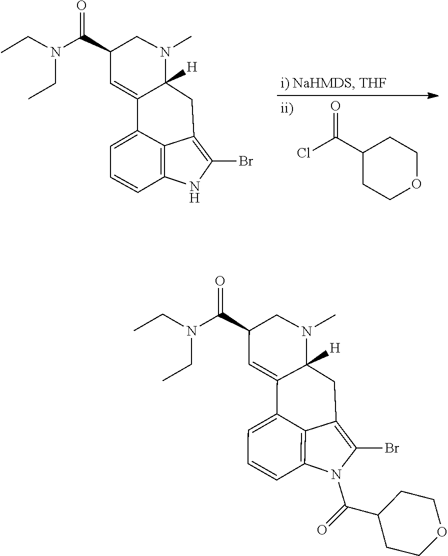 [SMILES: CCN(CC)C(=O)[C@@H]1C=C2c3cccc4[nH]c(Br)c(c34)C[C@H]2N(C)C1 ; O=C(Cl)C1CCOCC1 ; CCN(CC)C(=O)[C@@H]1C=C2c3cccc4c3c(c(Br)n4C(=O)C3CCOCC3)C[C@H]2N(C)C1]

2-Br-LSD (76 mg, 0.19 mmol, 1 equiv.) was dissolved in anhydrous THE (3.8 mL) and cooled to −78° C. Sodium bis(trimethylsilyl)amide (1.0 μM in THF, 180 μL, 0.18 mmol, 0.95 equiv.) was added under an atmosphere of N₂ and the mixture was stirred at −78° C. for 30 min. Oxane-4-carbonyl chloride (31 mg, 26 μL, 0.21 mmol, 1.1 equiv.) was added dropwise. The mixture was allowed to reach rt over 3.5 h. The mixture was quenched with water (5 mL) and the aqueous phase was extracted with DCM (3×3 mL). The combined organic layers were washed with water (10 mL), saturated brine (10 mL), dried (Na₂SO₄) and concentrated to give a residue (˜140 mg). The residue was purified by column chromatography on silica gel, eluting with a gradient of MeOH in DCM to afford (6aR,9R)-5-bromo-N,N-diethyl-7-methyl-4-(tetrahydro-2H-pyran-4-carbonyl)-4,6,6a,7,8,9-hexahydroindolo[4,3-fg]quinoline-9-carboxamide (85 mg, 87%) as a semi-solid. LC-MS (+ve mode): m/z=514.20 and 516.20 [M+H]⁺; ¹H NMR (CDCl₃, 300 MHz) δ 7.91 (dd, 1H, J=8.1, 0.7 Hz, ArH), 7.37 (dd, 1H, J=7.6, 0.7 Hz, ArH), 7.28 (m, 1H, obscured, ArH), 6.37 (s, 1H, C═CH), 4.06 (m, 2H, 2×0.5 CH₂), 3.91 (m, 2H, 2×CHCO), 3.47 (m, 7H, 2×0.5 CH₂, 2×CH₂Me and CHN), 3.17 (m, 1H, CHH), 3.05 (ddd, 1H, J=11.3, 5.1, 1.3 Hz, CHH), 2.85 (t, 1H, J=10.9 Hz, CHH), 2.61 (s, 3H, NCH₃), 2.47 (dd, 1H, J=15.4, 11.5 Hz, CHH), 1.98 (m, 4H, 2×CH₂), 1.21 (m, 6H, 2×CH₃).

### Example 17: Synthesis of (6aR,9R)-4-acetyl-5-bromo-N,N-diethyl-7-methyl-4,6,6a,7,8,9-hexahydroindolo[4,3-fg]quinoline-9-carboxamide

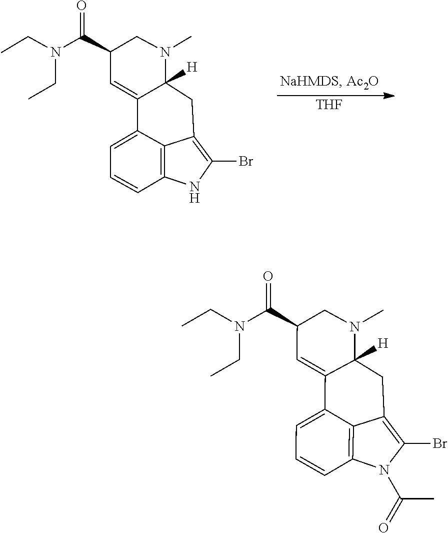 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4c3c(c(Br)n4C(C)=O)C[C@H]2N(C)C1 ; "THF"]

A solution of 2-Br-LSD (100 mg, 0.25 mmol, 1 equiv.) in anhydrous THE (2 mL) was cooled to 0° C. before sodium bis(trimethylsilyl)amide (1M in THF, 236 μL, 0.24 mmol, 0.95 equiv.) was added and the mixture was stirred at 0° C. for 30 min under an atmosphere of N₂. Ac₂O (28 mg, 26 μL, 0.27 mmol, 1.1 equiv.) was added and the reaction mixture was stirred for a further 30 min at 0° C. followed by overnight at rt. The reaction mixture was concentrated and the residue was reconstituted in a mixture of saturated aqueous sodium bicarbonate (2 mL) and DCM (2 mL). The phases were separated and the aqueous phase extracted with dcm (2 mL). The combined organic layers were washed with water (2×2 mL), saturated brine (2 mL), dried (MgSO₄) and concentrated. The residue was purified by column chromatography on silica gel, eluting with a gradient of MeOH in DCM, to give (6aR,9R)-4-acetyl-5-bromo-N,N-diethyl-7-methyl-4,6,6a,7,8,9-hexahydroindolo[4,3-fg]quinoline-9-carboxamide (80.9 mg, 73%) as a solid. LC-MS (+ve mode): m/z=444.10 and 446.10 [M+H]⁺; ¹H NMR (CDCl₃, 300 MHz) δ 7.92 (d, 1H, J=7.8 Hz, ArH), 7.31 (d, 1H, J=7.3 Hz, ArH), 7.21 (obscured m, 1H, ArH), 6.30 (s, 1H, C═CH), 3.80 (m, 1H, CHCO), 3.38 (m, 5H, 2×CH₂ and CHN), 3.10 (m, 1H, CHH), 2.98 (ddd, 1H, J=11.3, 5.0, 1.3 Hz, CHH), 2.77 (m, 4H, NCH₃ and CHH), 2.54 (s, 3H, CH₃), 2.40 (dd, 1H, J=15.4, 11.5 Hz, CHH), 1.15 (m, 6H, 2×CH₃).

### Example 18: Synthesis of ethyl (6aR,9R)-5-bromo-9-(diethylcarbamoyl)-7-methyl-6a,7,8,9-tetrahydroindolo[4,3-fg]quinoline-4(6H)-carboxylate

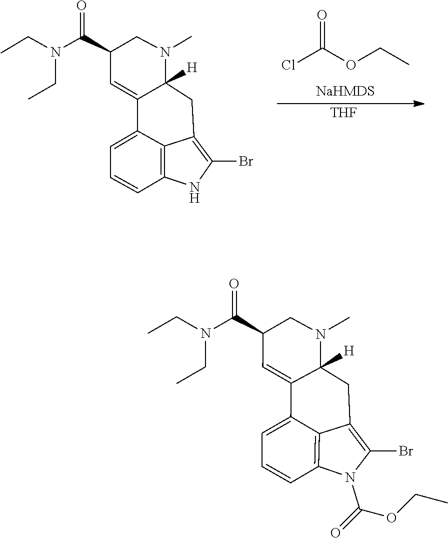

A solution of 2-Br-LSD (100 mg, 0.25 mmol, 1 equiv.) in anhydrous THE (2 mL) was cooled to 0° C. before sodium bis(trimethylsilyl)amide (1M in THF, 236 μL, 0.24 mmol, 0.95 equiv.) was added and the mixture was stirred at 0° C. for 30 min under an atmosphere of N₂. Ethyl chloroformate (30 mg, 26 μL, 0.27 mmol, 1.1 equiv.) was added and the reaction mixture was stirred for 30 min at 0° C. followed by overnight at rt. The reaction mixture was concentrated and the residue was reconstituted in a mixture of saturated aqueous sodium bicarbonate (2 mL) and DCM (2 mL). The phases were separated and the aqueous phase extracted with DCM (2 mL). The combined organic layers were washed with water (2×2 mL), saturated brine (2 mL), dried (MgSO₄) and concentrated. The residue was purified by column chromatography on silica gel, eluting with a gradient of MeOH in DCM, to give ethyl (6aR,9R)-5-bromo-9-(diethylcarbamoyl)-7-methyl-6a,7,8,9-tetrahydroindolo[4,3-fg]quinoline-4(6H)-carboxylate (84.0 mg, 71%) as a viscous oil. LC-MS (+ve mode): m/z=474.15 and 476.15 [M+H]⁺; ¹H NMR (CDCl₃, 300 MHz) δ 7.71 (d, 1H, J=8.1 Hz, ArH), 7.29 (d, 1H, J=6.9 Hz, ArH), 7.19 (obscured m, 1H, ArH), 6.29 (s, 1H, C═CH), 4.44 (q, 2H, J=7.1 Hz, CH₂), 3.80 (m, 1H, CHCO), 3.35 (m, 5H, 2×CH₂ and CHN), 3.09 (m, 1H, CHH), 2.98 (dd, 1H, J=11.3 Hz, CHH), 2.78 (t, 1H, J=10.9 Hz, CHH), 2.53 (s, 3H, NCH₃), 2.40 (dd, 1H, J=15.4, 11.6 Hz, CHH), 1.44 (t, 3H, J=7.1 Hz, CH₃), 1.19 (t, 3H, J=7.1, CH₃), 1.10 (t, 3H, J=7.1 Hz, CH₃).

### Example 19-A: Synthesis of acetamidomethyl acetate

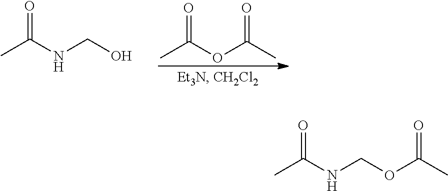

To a suspension of N-(hydroxymethyl)acetamide (294 mg, 3.30 mmol, 1 equiv.) in DCM (5 mL) was added Et₃N (0.94 g, 1.3 mL, 9.90 mmol, 3 equiv.) and Ac₂O (0.64 g, 0.63 mL, 6.23 mmol, 2 equiv.) and the reaction mixture was stirred at rt overnight. The solvent was removed under vacuum, azeotroping with toluene to afford acetamidomethyl acetate (838 mg) as a solid that was used without further purification.

### Example 19: Synthesis of (6aR,9R)-4-(acetamidomethyl)-5-bromo-N,N-diethyl-7-methyl-4,6,6a,7,8,9-hexahydroindolo[4,3-fg]quinoline-9-carboxamide

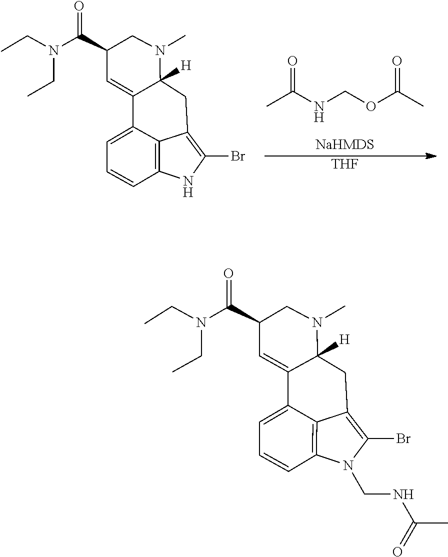

A solution of 2-Br-LSD (50 mg, 0.12 mmol, 1 equiv.) in anhydrous THE (1 mL) was cooled to −78° C. before sodium bis(trimethylsilyl)amide (1M in THF, 118 μL, 0.12 mmol, 0.95 equiv.) was added and the mixture was stirred at −78° C. for 30 min under nitrogen. Acetamidomethyl acetate (17.8 mg 0.14 mmol, 1.1 equiv.) was added and the reaction mixture was stirred for a further 30 min at 0° C. followed by overnight at rt. The reaction mixture was concentrated and the residue was purified by column chromatography on silica gel, eluting with a gradient of MeOH in DCM, to give (6aR,9R)-4-(acetamidomethyl)-5-bromo-N,N-diethyl-7-methyl-4,6,6a,7,8,9-hexahydroindolo[4,3-fg]quinoline-9-carboxamide (30.5 mg, 53%) as a semi-solid. LC-MS (+ve mode): m/z=473.15 and 475.15 [M+H]⁺; ¹H NMR (CDCl₃, 300 MHz) δ 7.92 (dd, 1H, J=6.9, 1.8 Hz, ArH), 7.21 (m, 2H, 2×ArH), 6.34 (br, 1H, C═CH), 6.05 (br, 1H NH), 5.64 (d, 2H, J=6.3 Hz, NCH₂N), 3.86 (m, 1H, CHCO), 3.42 (m, 5H, 2×CH₂ and CHN), 3.19 (m, 1H, CHH), 3.06 (m, 1H, CHH), 2.88 (t, 1H, J=10.8 Hz, CHH), 2.60 (s, 3H, NCH₃), 2.53 (m, 1H, CHH), 1.97 (s, 3H, CH₃CO), 1.25 (t, 3H, J=7.1 Hz, CH₃), 1.17 (t, 3H, J=7.1 Hz, CH₃).

### Example 20: Synthesis of chloromethyl (6aR,9R)-5-bromo-9-(diethylcarbamoyl)-7-methyl-6a,7,8,9-tetrahydroindolo[4,3-fg]quinoline-4(6H)-carboxylate

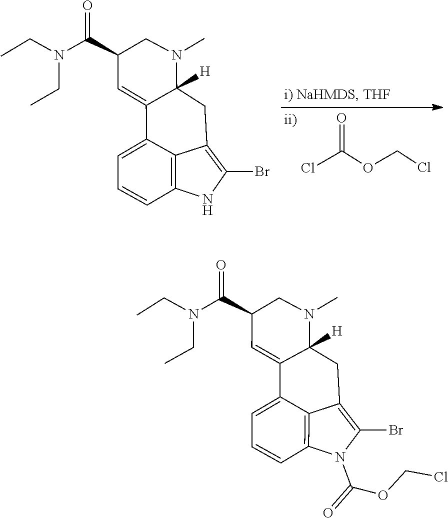 [SMILES: CCN(CC)C(=O)[C@@H]1C=C2c3cccc4[nH]c(Br)c(c34)C[C@H]2N(C)C1 ; O=C(Cl)OCCl ; CCN(CC)C(=O)[C@@H]1C=C2c3cccc4c3c(c(Br)n4C(=O)OCCl)C[C@H]2N(C)C1]

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂ and the mixture was stirred at −78° C. for 30 min. Chloromethyl chloroformate (1.0 μM in THF, 25 μL, 0.27 mmol, 1.1 equiv.) was added dropwise. The mixture was stirred at −78° C. for 30 min, then allowed to reach rt over 3 h. The mixture was quenched with water (5 mL) and the aqueous phase was extracted with DCM (3×5 mL). The combined organic layers were washed with saturated brine (20 mL), dried (Na₂SO₄) and concentrated to give a residue (115 mg). The residue was purified by column chromatography on silica gel, eluting with a gradient of MeOH in DCM, to afford chloromethyl (6aR,9R)-5-bromo-9-(diethylcarbamoyl)-7-methyl-6a,7,8,9-tetrahydroindolo[4,3-fg]quinoline-4(6H)-carboxylate (120 mg, 97%) as a semi-solid. LC-MS (+ve mode): m/z=494.10 and 496.10 [M+H]⁺; ¹H NMR (CDCl₃, 300 MHz) δ 7.79 (d, 1H, J=8.1 Hz, ArH), 7.39 (d, 1H, J=7.6 Hz, ArH), 7.30 (d, 1H, J=8.0 Hz, ArH), 6.38 (s, 1H, C═CH), 6.05 (s, 2H, CH₂), 3.86 (m, 1H, CHCO), 3.43 (m, 5H, 2×CH₂Me and 1×CHN), 3.16 (m, 1H, CHH), 3.04 (m, 1H, CHH), 2.84 (t, 1H, J=10.9 Hz, CHH), 2.60 (s, 3H, NCH₃), 2.44 (dd, 1H, J=15.5, 11.6 Hz, CHH), 1.21 (m, 6H, 2×CH₃).

### Example 21: Synthesis of ((oxetane-3-carbonyl)oxy)methyl (6aR,9R)-5-bromo-9-(diethylcarbamoyl)-7-methyl-6a,7,8,9-tetrahydroindolo[4,3-fg]quinoline-4(6H)-carboxylate

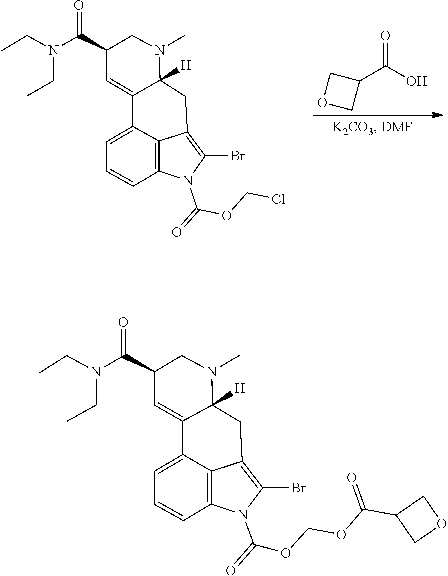 [SMILES: CCN(CC)C(=O)[C@@H]1C=C2c3cccc4c3c(c(Br)n4C(=O)OCCl)C[C@H]2N(C)C1 ; O=C(O)C1COC1 ; CCN(CC)C(=O)[C@@H]1C=C2c3cccc4c3c(c(Br)n4C(=O)OCOC(=O)C3COC3)C[C@H]2N(C)C1]

To a solution of 3-oxetanecarboxylic acid (27 mg, 0.27 mmol, 1.1 equiv.) in DMF (3.5 mL) was added K₂CO₃ (51 mg, 0.37 mmol, 1.5 equiv.). The resulting suspension was stirred at rt for 10 min under an atmosphere of N₂. A solution of chloromethyl (6aR,9R)-5-bromo-9-(diethylcarbamoyl)-7-methyl-6a,7,8,9-tetrahydroindolo[4,3-fg]quinoline-4(6H)-carboxylate (120 mg, 0.24 mmol, 1 equiv.) in DMF (3.0 mL) was added to the suspension and the mixture was stirred at rt for 16 h. The mixture was concentrated to give a residue. LC-MS (+ve mode): m/z=560.15 and 562.15 [M+H]⁺.

### Example 22: Synthesis of (tetrahydro-2H-pyran-4-carbonyl)oxy)methyl (6aR,9R)-5-bromo-9-(diethylcarbamoyl)-7-methyl-6a,7,8,9-tetrahydroindolo[4,3-fg]quinoline-4(6H)-carboxylate

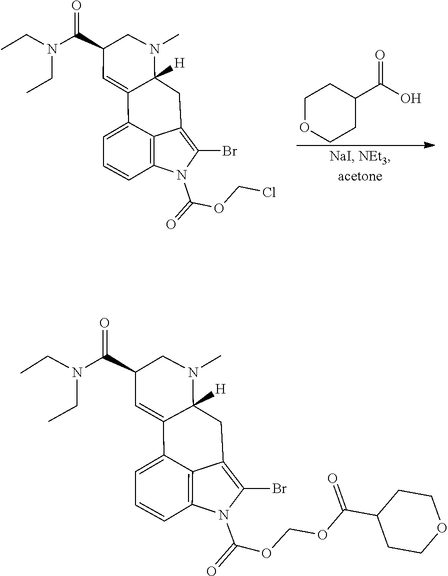 [SMILES: CCN(CC)C(=O)[C@@H]1C=C2c3cccc4c3c(c(Br)n4C(=O)OCCl)C[C@H]2N(C)C1 ; O=C(O)C1CCOCC1 ; CCN(CC)C(=O)[C@@H]1C=C2c3cccc4c3c(c(Br)n4C(=O)OCOC(=O)C3CCOCC3)C[C@H]2N(C)C1]

To a solution of chloromethyl (6aR,9R)-5-bromo-9-(diethylcarbamoyl)-7-methyl-6a,7,8,9-tetrahydroindolo[4,3-fg]quinoline-4(6H)-carboxylate (115 mg, 0.23 mmol, 1 equiv.) in acetone (4.5 mL) was added NaI (42 mg, 0.28 mmol, 1.2 equiv.) and tetrahydro-2H-pyran-4-carboxylic acid (36 mg, 0.28 mmol, 1.2 equiv.). Et₃N (83 mg, 114 μL, 0.82 mmol, 3.5 equiv.) was added and the mixture was heated at 70° C. for 16 h in the dark under an atmosphere of N₂. After cooling to rt, the mixture was concentrated and reconstituted in DCM (10 mL). Addition of water (5 mL) was followed by extraction of the aqueous phase with DCM (3×5 mL). The combined organic layers were washed with saturated aqueous NaHCO₃ (20 mL), saturated brine (20 mL), dried (Na₂SO₄) and concentrated to give a residue. The material was purified by column chromatography on silica gel, eluting with a gradient of MeOH in DCM to give (tetrahydro-2H-pyran-4-carbonyl)oxy)methyl (6aR,9R)-5-bromo-9-(diethylcarbamoyl)-7-methyl-6a,7,8,9-tetrahydroindolo[4,3-fg]quinoline-4(6H)-carboxylate (10 mg, 7%). LC-MS (+ve mode): m/z=588.20 and 590.20 [M+H]⁺; ¹H NMR (CDCl₃, 300 MHz) δ 7.75 (d, 1H, J=8.1 Hz, ArH), 7.38 (d, 1H, J=7.5 Hz, ArH), 7.27 (m, 1H, obscured, ArH), 6.37 (s, 1H, C═CH), 6.10 (s, 2H, CH₂), 3.95 (m, 2H, 2×0.5 CH₂), 3.87 (m, 1H, obscured, CHCO), 3.42 (m, 7H, 2×0.5 CH₂, 2×CH₂Me and CHN), 3.14 (m, 1H, CHH), 3.04 (m, 1H, CHH), 2.84 (t, 1H, J=10.9 Hz, CHH), 2.65 (m, 1H, obscured, CHCO), 2.60 (s, 3H, NCH₃), 2.44 (dd, 1H, J=15.5, 11.6 Hz, CHH), 1.82 (m, 4H, 2×CH₂), 1.25 (t, 3H, J=7.1 Hz, CH₃), 1.17 (t, 3H, J=7.1 Hz, CH₃).

### Example 23: Synthesis of (((6aR,9R)-5-bromo-9-(diethylcarbamoyl)-7-methyl-4,6,6a,7,8,9-hexahydroindolo[4,3-fg]quinoline-4-carbonyl)oxy)methyl tert-butyl succinate

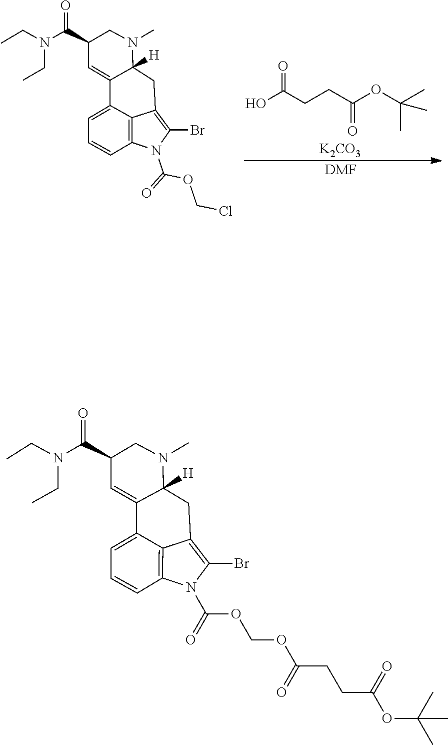

A mixture of chloromethyl (6aR,9R)-5-bromo-9-(diethylcarbamoyl)-7-methyl-6a,7,8,9-tetrahydroindolo[4,3-fg]quinoline-4(6H)-carboxylate (60 mg, 0.12 mmol, 1 equiv.), potassium carbonate (25 mg, 0.18 mmol, 1.5 equiv.) and 4-(tert-butoxy)-4-oxobutanoic acid (27 mg, 0.13 mmol, 1.1 equiv) in anhydrous DMF (1 mL) was stirred at rt for 16 h under an atmosphere of N₂. The reaction was quenched with water (1 mL) and diluted with EtOAc (1 mL). The phases were separated and the aqueous phase extracted with EtOAc (1 mL). The combined organic layers were washed with water (2×1 mL), saturated brine (2 mL), dried (MgSO₄) and concentrated. The residue was purified by column chromatography on silica gel, eluting with a gradient of MeOH in DCM, to give (((6aR,9R)-5-bromo-9-(diethylcarbamoyl)-7-methyl-4,6,6a,7,8,9-hexahydroindolo[4,3-fg]quinoline-4-carbonyl)oxy)methyl tert-butyl succinate (40.2 mg) as a viscous oil. LC-MS (+ve mode): m/z=632.20 and 634.20 [M+H]⁺.

### Example 24: Synthesis of 4-((((6aR,9R)-5-bromo-9-(diethylcarbamoyl)-7-methyl-4,6,6a,7,8,9-hexahydroindolo[4,3-fg]quinoline-4-carbonyl)oxy)methoxy)-4-oxobutanoic acid formate

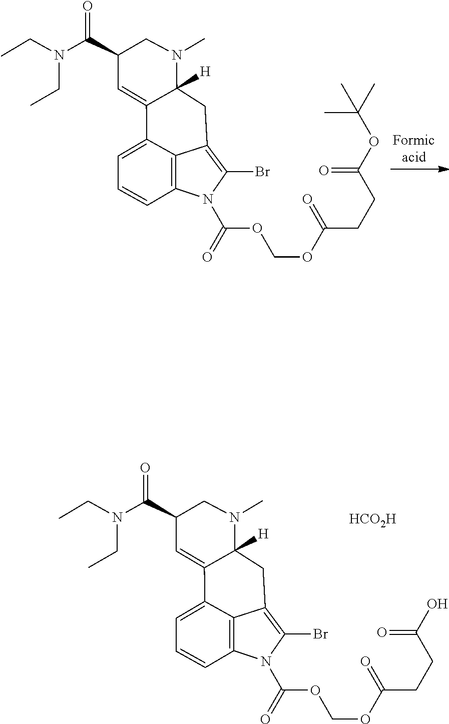

(((6aR,9R)-5-Bromo-9-(diethylcarbamoyl)-7-methyl-4,6,6a,7,8,9-hexahydroindolo[4,3-fg]quinoline-4-carbonyl)oxy)methyl tert-butyl succinate (40.2 mg, 0.06 mmol, 1 equiv.) was dissolved in formic acid (0.5 mL) at rt and stirred for 16 h. The mixture was concentrated to give 4-((((6aR,9R)-5-bromo-9-(diethylcarbamoyl)-7-methyl-4,6,6a,7,8,9-hexahydroindolo[4,3-fg]quinoline-4-carbonyl)oxy)methoxy)-4-oxobutanoic acid formate as a viscous oil. LC-MS (+ve mode): m/z=576.15 and 578.15 [M+H]⁺.

### Example 25: Synthesis of 1-chloroethyl (6aR,9R)-5-bromo-9-(diethylcarbamoyl)-7-methyl-6a,7,8,9-tetrahydroindolo[4,3-fg]quinoline-4(6H)-carboxylate

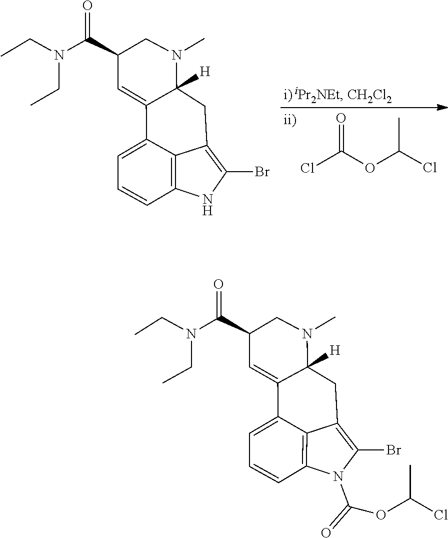

2-Br-LSD (55 mg, 0.14 mmol, 1 equiv.) was dissolved in DCM (1 mL). N,N-diisopropylethylamine (71 mg, 95 μL, 0.55 mmol, 4 equiv.) was added under an atmosphere of N₂. The mixture was stirred at rt for 10 min, and then cooled to −10° C. 1-Chloroethyl chloroformate (39 mg, 30 μL, 0.27 mmol, 2 equiv.) was added dropwise. The mixture was allowed to reach rt over 1 h. The mixture was concentrated to give intermediate 1-chloroethyl (6aR,9R)-5-bromo-9-(diethylcarbamoyl)-7-methyl-6a,7,8,9-tetrahydroindolo[4,3-fg]quinoline-4(6H)-carboxylate (70 mg) as a semi-solid. This material was used in the next step without further purification. LC-MS (+ve mode): m/z=508.15 and 510.15 [M+H]⁺.

### Example 26: Synthesis of 1-((tetrahydro-2H-pyran-4-carbonyl)oxy)ethyl (6aR,9R)-5-bromo-9-(diethylcarbamoyl)-7-methyl-6a,7,8,9-tetrahydroindolo[4,3-fg]quinoline-4(6H)-carboxylate

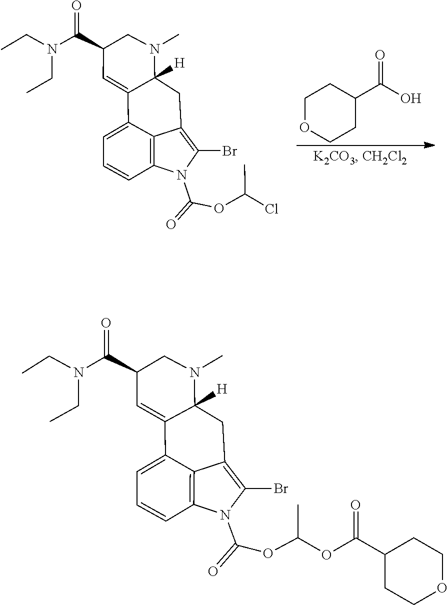 [SMILES: CCN(CC)C(=O)[C@@H]1C=C2c3cccc4c3c(c(Br)n4C(=O)OC(C)Cl)C[C@H]2N(C)C1 ; O=C(O)C1CCOCC1 ; CCN(CC)C(=O)[C@@H]1C=C2c3cccc4c3c(c(Br)n4C(=O)OC(C)OC(=O)C3CCOCC3)C[C@H]2N(C)C1]

A solution of 1-chloroethyl (6aR,9R)-5-bromo-9-(diethylcarbamoyl)-7-methyl-6a,7,8,9-tetrahydroindolo[4,3-fg]quinoline-4(6H)-carboxylate (70 mg), tetrahydro-2H-pyran-4-carboxylic acid (20 mg, 0.15 mmol, 1.1 equiv.) and K₂CO₃ (29 mg, 0.21 mmol, 1.5 equiv.) in anhydrous DCM (3 mL) was stirred at rt for 18 h under an atmosphere of N₂. The mixture was concentrated to give a residue. LC-MS (+ve mode): m/z=602.25 and 604.25 [M+H]⁺.

### Example 27: Synthesis of tert-butyl ((S)-1-((6aR,9R)-5-bromo-9-(diethylcarbamoyl)-7-methyl-6a,7,8,9-tetrahydroindolo[4,3-fg]quinolin-4(6H)-yl)-1-oxopropan-2-yl)carbamate

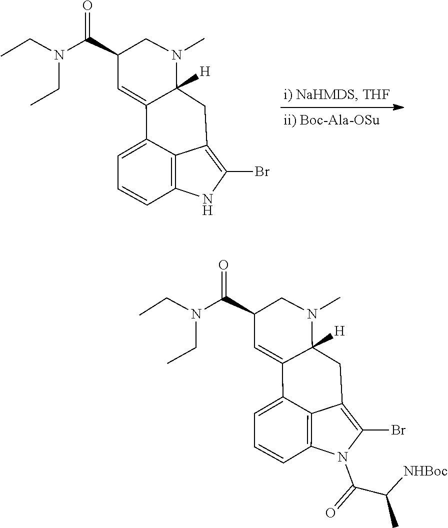 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4c3c(c(Br)n4C(=O)[C@H](C)NC(=O)OC(C)(C)C)C[C@H]2N(C)C1]

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 275 μL, 0.28 mmol, 1.1 equiv.) was added under an atmosphere of N₂. The mixture was stirred at −78° C. for 30 min. A solution of Boc-Ala-OSu (72 mg, 0.25 mmol, 1 equiv) in anhydrous THE (1 mL) was added dropwise and the mixture was stirred at −78° C. for 30 min, followed by rt for 48 h. The reaction quenched with water (0.5 mL) and concentrated to give a residue. The residue was purified by column chromatography on silica gel, eluting with a gradient of MeOH in DCM, to afford tert-butyl ((S)-1-((6aR,9R)-5-bromo-9-(diethylcarbamoyl)-7-methyl-6a,7,8,9-tetrahydroindolo[4,3-fg]quinolin-4(6H)-yl)-1-oxopropan-2-yl)carbamate (40 mg, 28%) as a semi-solid. LC-MS (+ve mode): m/z=573.20 and 575.20 [M+H]⁺; ¹H NMR (CDCl₃, 300 MHz) δ 7.90 (d, 1H, J=8.1 Hz, ArH), 7.32 (d, 1H, J=7.5 Hz, ArH), 7.23 (d, 1H, J=8.0 Hz, ArH), 6.30 (s, 1H, C═CH), 5.77 (m, 1H, NH) 5.44 (m, 1H, CHCO), 3.78 (m, 1H, CHN), 3.38 (m, 4H, 2×CH₂Me) 3.29 (m, 1H, CHCO), 3.11 (m, 1H, CHH), 2.98 (dd, 1H, J=11.0, 4.7 Hz, CHH), 2.78 (t, 1H, J=10.9 Hz, CHH), 2.54 (s, 3H, NCH₃), 2.39 (m, 1H, CHH), 1.42 (Obscured, 3H, CH₃), 1.40 (s, 9H, J=6.3 Hz, 3×CH₃), 1.19 (t, 3H, J=7.0 Hz, CH₃), 1.10 (t, 3H, J=7.4 Hz, CH₃).

### Example 28: Synthesis of (6aR,9R)-4-(D-alanyl)-5-bromo-N,N-diethyl-7-methyl-4,6,6a,7,8,9-hexahydroindolo[4,3-fg]quinoline-9-carboxamide di-trifluoroacetate

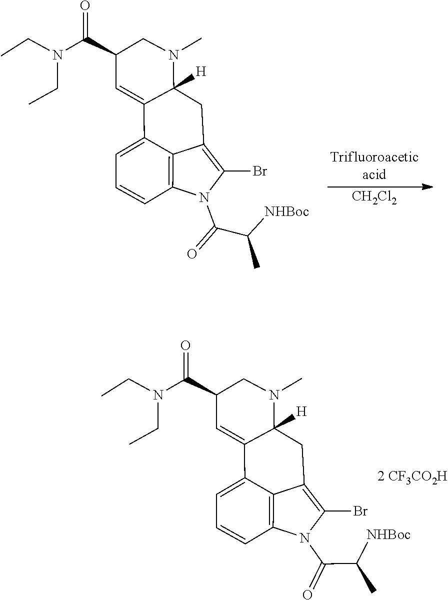 [SMILES: CCN(CC)C(=O)[C@@H]1C=C2c3cccc4c3c(c(Br)n4C(=O)[C@H](C)NC(=O)OC(C)(C)C)C[C@H]2N(C)C1 ; "CH2Cl2" ; CCN(CC)C(=O)[C@@H]1C=C2c3cccc4c3c(c(Br)n4C(=O)[C@H](C)NC(=O)OC(C)(C)C)C[C@H]2N(C)C1]

To a solution of tert-butyl ((R)-1-((6aR,9R)-5-bromo-9-(diethylcarbamoyl)-7-methyl-6a,7,8,9-tetrahydroindolo[4,3-fg]quinolin-4(6H)-yl)-1-oxopropan-2-yl)carbamate (29 mg, 0.05 mmol, 1 equiv.) in DCM (2 mL) was added TFA (285 mg, 191 μL, 2.50 mmol, 50 equiv.) at rt and the mixture was stirred for 2 h. The mixture was concentrated and azeotroped with chloroform (4×10 mL) to give (6aR,9R)-4-(D-alanyl)-5-bromo-N,N-diethyl-7-methyl-4,6,6a,7,8,9-hexahydroindolo[4,3-fg]quinoline-9-carboxamide di-trifluoroacetate as a viscous oil. LC-MS (+ve mode): m/z=473.15 and 475.15 [M+H]⁺.

### Example 29: Synthesis of tert-butyl ((S)-1-((6aR,9R)-5-bromo-9-(diethylcarbamoyl)-7-methyl-6a,7,8,9-tetrahydroindolo[4,3-fg]quinolin-4(6H)-yl)-3-methyl-1-oxobutan-2-yl)carbamate formate

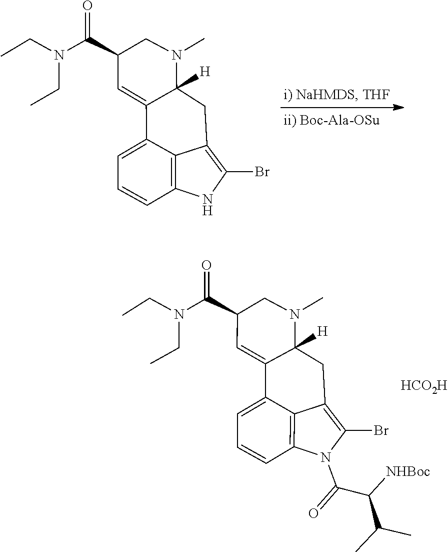 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4c3c(c(Br)n4C(=O)[C@@H](NC(=O)OC(C)(C)C)C(C)C)C[C@H]2N(C)C1 ; "HCO2H"]

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 275 μL, 0.28 mmol, 1.1 equiv.) was added under an atmosphere of N₂ and the mixture was stirred at −78° C. for 30 min. A solution of Boc-Val-OSu (78 mg, 0.25 mmol, 1 equiv.) in anhydrous THE (3 mL) was added dropwise. The mixture was stirred at −78° C. for 90 min, followed by rt overnight. The mixture was quenched with water (1 mL) and concentrated. The residue was purified by reversed phase chromatography on silica, eluting with a gradient of acetonitrile in water (0.1% formic acid) to afford tert-butyl ((S)-1-((6aR,9R)-5-bromo-9-(diethylcarbamoyl)-7-methyl-6a,7,8,9-tetrahydroindolo[4,3-fg]quinolin-4(6H)-yl)-3-methyl-1-oxobutan-2-yl)carbamate formate (39 mg, 26%) as a semi-solid. LC-MS (+ve mode): m/z=601.20 and 603.20 [M+H]⁺; ¹H NMR (CDCl₃, 300 MHz) δ 8.11 (s, 1H, HCO₂H), 7.97 (d, 1H, J=8.0 Hz, ArH), 7.38 (d, 1H, J=7.5 Hz, ArH), 7.32 (d, 1H, J=7.9 Hz, ArH), 6.41 (s, 1H, C═CH), 5.92 (d, 1H, J=9.7 Hz, NH), 5.41 (d, 1H, J=9.3 Hz, CHCO), 4.08 (m, 1H, CHCO), 3.44 (m, 6H, CHH, CHN, 2×CH₂), 3.29 (dd, 1H, J=11.5, 5.1 Hz, CHH), 3.02 (t, 1H, J=11.1 Hz, CHH), 2.74 (m, 4H, NCH₃ and CHH), 1.48 (s, 9H, 3×CH₃), 1.26 (t, 3H, J=7.1 Hz, CH₃), 1.17 (t, 3H, J=7.1 Hz, CH₃), 1.06 (dd, 3H, J=6.8, 2.2 Hz, CH₃), 0.79 (d, 3H, J=6.9 Hz, CH₃).

### Example 30: Synthesis of (6aR,9R)-4-((S)-2-amino-3-methylbutanoyl)-5-bromo-N,N-diethyl-7-methyl-4,6,6a,7,8,9-hexahydroindolo[4,3-fg]quinoline-9-carboxamide di-trifluoroacetate

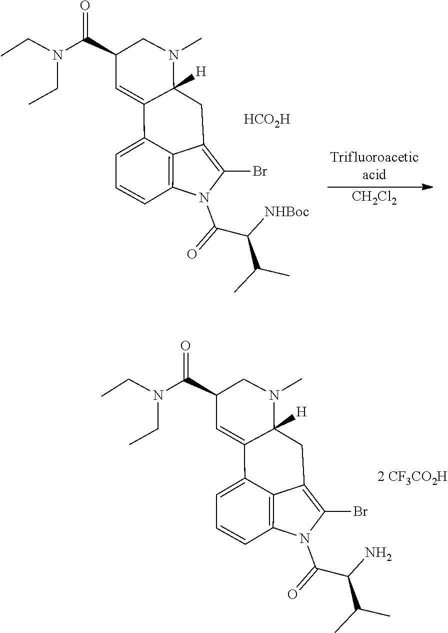

To a solution of tert-butyl ((S)-1-((6aR,9R)-5-bromo-9-(diethylcarbamoyl)-7-methyl-6a,7,8,9-tetrahydroindolo[4,3-fg]quinolin-4(6H)-yl)-3-methyl-1-oxobutan-2-yl)carbamate formate (19 mg, 0.03 mmol, 1 equiv.) in DCM (1 mL) was added TFA (18 mg, 13 μL, 0.16 mmol, 5 equiv.) at rt and the mixture was stirred for 1 h. Added extra TFA (18 mg, 13 μL, 0.16 mmol, 5 equiv.) at rt and the mixture was stirred for 1.5 h. The mixture was concentrated and azeotroped with chloroform (4×5 mL) to give (6aR,9R)-4-((S)-2-amino-3-methylbutanoyl)-5-bromo-N,N-diethyl-7-methyl-4,6,6a,7,8,9-hexahydroindolo[4,3-fg]quinoline-9-carboxamide di-trifluoroacetate as a semi-solid. LC-MS (+ve mode): m/z=501.20 and 503.20 [M+H]⁺.

### Example 31: Synthesis of di-tert-butyl ((5S)-6-((9S)-5-bromo-9-(diethylcarbamoyl)-7-methyl-6a,7,8,9-tetrahydroindolo[4,3-fg]quinolin-4(6H)-yl)-6-oxohexane-1,5-diyl)dicarbamate

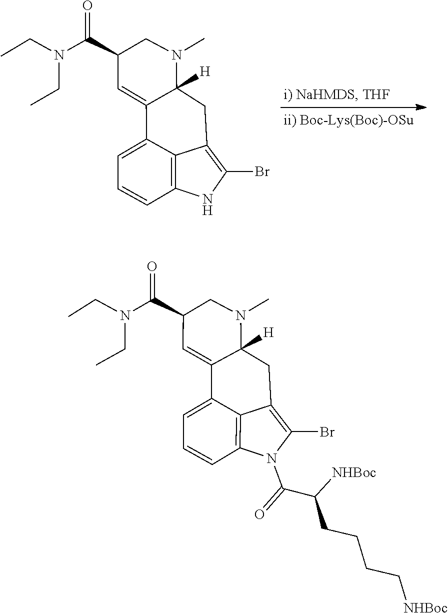 [SMILES: CCN(CC)C(=O)[C@@H]1C=C2c3cccc4[nH]c(Br)c(c34)C[C@H]2N(C)C1 ; CCN(CC)C(=O)[C@@H]1C=C2c3cccc4c3c(c(Br)n4C(=O)[C@H](CCCCNC(=O)OC(C)(C)C)NC(=O)OC(C)(C)C)C[C@H]2N(C)C1]

2-Br-LSD (100 mg, 0.25 mmol, 1 equiv.) was dissolved in anhydrous THE (5 mL) and cooled to −78° C. Sodium bis(trimethylsilyl)amide (1.0 μM in THF, 240 μL, 0.24 mmol, 0.95 equiv.) was added under an atmosphere of N₂ and the mixture was stirred at −78° C. for 1 h. Boc-Lys(Boc)-OSu (110 mg, 0.25 mmol, 1.0 equiv.) in anhydrous THE (3 mL) was added dropwise. The mixture was stirred at −78° C. for 10 min, followed by rt for 5 min and concentrated. The residue was purified by column chromatography on silica gel, eluting with a gradient of MeOH in DCM to give di-tert-butyl ((5S)-6-((9S)-5-bromo-9-(diethylcarbamoyl)-7-methyl-6a,7,8,9-tetrahydroindolo[4,3-fg]quinolin-4(6H)-yl)-6-oxohexane-1,5-diyl)dicarbamate (124 mg) as a semi-solid. This material was used in the next step without further purification. LC-MS (+ve mode): m/z=730.30 and 732.30 [M+H]⁺.

### Example 32: Synthesis of (6aR,9R)-4-(L-lysyl)-5-bromo-N,N-diethyl-7-methyl-4,6,6a,7,8,9-hexahydroindolo[4,3-fg]quinoline-9-carboxamide tri-trifluoroacetate

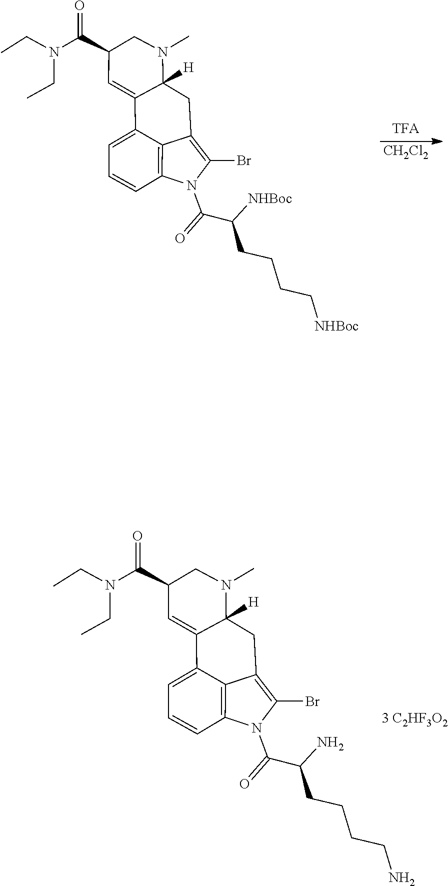 [SMILES: CCN(CC)C(=O)[C@@H]1C=C2c3cccc4c3c(c(Br)n4C(=O)[C@H](CCCCNC(=O)OC(C)(C)C)NC(=O)OC(C)(C)C)C[C@H]2N(C)C1 ; "CH2Cl2" ; CCN(CC)C(=O)[C@@H]1C=C2c3cccc4c3c(c(Br)n4C(=O)[C@@H](N)CCCCN)C[C@H]2N(C)C1]

Di-tert-butyl ((S)-6-((6aR,9R)-5-bromo-9-(diethylcarbamoyl)-7-methyl-6a,7,8,9-tetrahydroindolo[4,3-fg]quinolin-4(6H)-yl)-6-oxohexane-1,5-diyl)dicarbamate (124 mg, 0.17 mmol, 1 equiv.) was dissolved in anhydrous DCM (1 mL) and TFA (242 mg, 163 μL, 2.13 mmol, 12.5 equiv.) was added. The mixture was stirred at rt for 2 h. The mixture was concentrated and azeotroped with chloroform (4×10 mL) to give (6aR,9R)-4-(L-lysyl)-5-bromo-N,N-diethyl-7-methyl-4,6,6a,7,8,9-hexahydroindolo[4,3-fg]quinoline-9-carboxamide tri-trifluoroacetate as a residue. LC-MS (+ve mode): m/z=530.25 and 532.25 [M+H]⁺.

### II. Biological Evaluation

### Example A: Pharmacokinetics of Selected Compounds Following a Single Intravenous or Oral Administration in Rats

A pharmacokinetic (PK) study was performed in three male Sprague-Dawley (SD) rats following intravenous (IV) and oral (PO) administration of 2-Br-LSD at 1 mg/kg (IV) and 4 mg/kg (PO) respectively, or test compounds (prodrugs of 2-Br-LSD) at 4 mg/kg (PO). Parent compound (2-Br-LSD) was measured in plasma.

### In Vivo Methods

### Regulatory

All animal experiments were performed under UK Home Office Licenses and with local ethical committee clearance. All experiments were performed by technicians that have completed parts A and B of the Home Office Personal License course and hold a current personal license. All experiments were performed in dedicated Biohazard 2 facilities with full AAALAC accreditation.

TABLE 2
Protocol: Serial tail vein bleed PK study of 2-Br-LSD Prodrugs in SD rats

| Protocol | | Serial PK study at 1 dose level | |
|---|---|---|---|
| Test Compound(s) | | 2-Br-LSD, 2-Br-LSD Prodrugs | |
| Dosing Route | | PO (all compounds) & IV (2-Br-LSD only) | |
| Overnight food withdrawal | | Yes | |
| Animals | Type | rat | |
| | Strain | Sprague Dawley rats | |
| | Sex | male | |
| | Weight (g) | 160-300 | g |

| | |
|---|---|
| N per cpd | 3 |
| Preparation | None |
| Cage | PK cages |

| Dose | 4 mg/kg PO & 1 mg/kg IV | |
|---|---|---|
| Dosing Soln. Conc. | 0.8 mg/mL (PO) or 0.5 mg/mL (IV) | |
| Dosing Volume | 5 mL/kg (PO) or 2 mL/kg (IV) | |
| Formulation checks required? | No | |
| Vehicle | 10% DMSO/20% PEG400/ 70% water | |
| | 2-Br-LSD (IV): 10% DMSO/90% HPCD (20% in water) | |
| Sampling time points (h) | PO: 0.5, 1, 2, 4, 7 & 24 h | |
| | IV: 0.25, 0.5, 0.75, 1, 2, 4, 7 & 24 h | |
| Blood sampling method | Serial via tail vein | |
| Alternative method if required | n/a | |
| Sample format required | >230 μL blood + 5 μL EDTA (93 mg/mL) to give 2 × 50 | |
| | μL plasma | |
| Sample processing | Centrifugation for plasma ASAP at 4° C. Place 110 μL | |
| | plasma into Eppendorf tube on ice containing 11 μL 10% | |
| | phosphoric acid. Gently mix before taking 2 × 50 μL | |
| | aliquots into duplicate 96 well plates on dry ice. | |
| Anticoagulant | EDTA (93 mg/mL): 5 μL per tube | |
| Centrifugation | 10,000 rpm × 3 min at 4° C. | |
| Additional samples | n/a | |
| Perfusion/rinsing required | n/a | |
| Euthanasia method | n/a | |
| Plasma sample tubes | 96 | well plates |
| Pre-freezer storage | Blood: ice (<30 min), | |
| | Acidified Plasma: dry ice | |
| Freezer storage | −80° | C. |

| Dose formulation samples | 100 μL from vortex dose solution in Eppendorf |
|---|---|
| Number of samples per cpd at | PO: 18 × acidified plasma (50 μL in duplicate), 1 dose |
| 1 dose level | soln |
| | IV: 24 × acidified plasma (50 μL in duplicate), 1 dose |
| | soln |

### Analysis

Samples were sent for method optimization and measurement of parent compound (2-Br-LSD) via unique calibration lines and following acceptance QC's. Dose formulation concentrations were also measured, and PK parameters were determined (Cmax (ng/mL), Tmax (hr), Cl (ml/min/kg), Vdss (L/kg), t1/2 (hr), AUCO-t (ng/mL*hr), AUCO-inf (ng/mL*hr), MRT (hr), Bioavailability (% F) where warranted) using WinNon Lin software. Data (including bioanalytical results and assay performance) were reported in a tabulated format.

### Additional Formulation Details for PK Study

Phosphoric acid. Diluted 85% phosphoric acid 8.5-fold to give a 10% solution.

Formulation for PO Administration: For PO dosing, the prodrug was formulated in 10% DMSO/20% PEG400/70% water to a concentration of 0.8 mg free metabolite material/mL. This provided a dose of 4 mg free metabolite/kg when the prodrug was administered PO in 5 mL/kg dosing volumes.

Formulation for IV administration: For IV dosing, 2-Br-LSD was formulated as solution in 10% DMSO/90% HPCD (20% in water) to a concentration of 0.5 mg free metabolite material/mL. This provided a dose of 1 mg free metabolite/kg when administered IV in 2 mL/kg dosing volumes.

### Example A-1: Measurement of Concentration of 2-Br-LSD after IV or Oral Administration of 2-Br-LSD Prodrugs In Vivo

The pharmacokinetic properties of the synthesized 2-Br-LSD prodrugs after oral administration in a rat model were assessed. The concentration of 2-Br-LSD was measured in each rat at various sampling timepoints after IV or oral administration of 2-Br-LSD or the synthesized 2-Br-LSD prodrugs to rats.

Dose formulations were made at equivalent concentrations of active compound (2-Br-LSD) adjusted for molecular weight of the compounds. The synthesized 2-Br-LSD prodrugs were dosed at 4 mg/kg oral (PO) nominal dose. Nominal doses were used in PK parameter determinations. The parent compound (2-Br-LSD) was dosed at 1 mg/kg intravenous (IV) and 4 mg/kg (PO).

### Example A-1-1: 2-Br-LSD Parent Compound (IV & PO)

| | |
|---|---|
| Species | Rat |
| Dose Route: | IV & PO |
| Nominal Dose Concentration: | 1 mg/kg (IV), 4 mg/kg (PO) |

Chemical name: 2-Br-LSD
Structural class: parent
Mechanistic class: n/a - parent compound

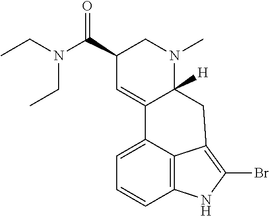 [SMILES: CCN(CC)C(=O)[C@@H]1C=C2c3cccc4[nH]c(Br)c(c34)C[C@H]2N(C)C1]

TABLE 3
2-Br-LSD (IV & PO) Pharmacokinetic Parameters

| | | Animal | T½ | Cmax | Tmax | Tlast | AUC0-last | AUC0-inf |
|---|---|---|---|---|---|---|---|---|
| Analyte | Dose | ID | (h) | (ng/mL) | (h) | (h) | (h*ng/mL) | (h*ng/mL) |
| 2-Br-LSD | IV | R49 | 0.738 | 577 | 0.250 | 4.00 | 278 | 280 |
| | | R50 | 0.629 | 658 | 0.250 | 4.00 | 268 | 269 |
| | | R51 | 0.746 | 500 | 0.250 | 4.00 | 304 | 308 |
| | | Mean | 0.704 | 578 | 0.250 | 4.00 | 283 | 286 |
| | Oral | R25 | 3.6 | 338 | 0.50 | 7 | 1350 | 1780 |
| | | R26 | 3.6 | 366.0 | 0.50 | 7 | 1430 | 1960 |
| | | R27 | 4.0 | 209.0 | 0.50 | 7 | 997 | 1450 |
| | | Mean | 3.7 | 304 | 0.50 | 7 | 1259 | 1730 |

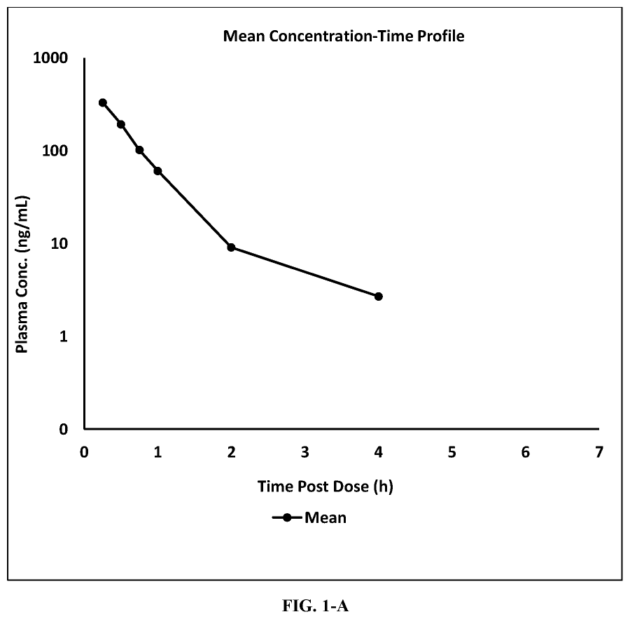

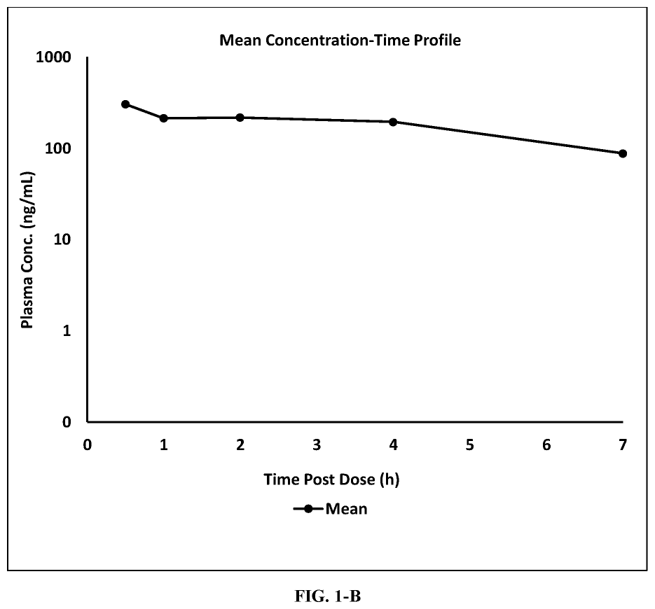

FIG. 1-A shows mean concentration-time profiles of 2-Br-LSD following IV dosing of 2-Br-LSD (1 mg/kg) to male Sprague Dawley (SD) rats.

FIG. 1-B shows mean concentration-time profiles of 2-Br-LSD following oral dosing of 2-Br-LSD (4 mg/Kg) to male Sprague Dawley (SD) rats.

### Example A-1-2: 2-Br—N-Boc Prodrug

| Species: | Rat |
|---|---|
| Dose Route: | PO |
| Dose Level (mg/kg) | 4 |

Chemical name: tert-butyl (6aR,9R)-5-bromo-9-(diethylcarbamoyl)-7-methyl-6a,7,8,9-tetrahydroindolo[4,3-fg]quinoline-4(6H)-carboxylate (Example 13)
Structural class: carbamate
Mechanistic class: presumed esterase

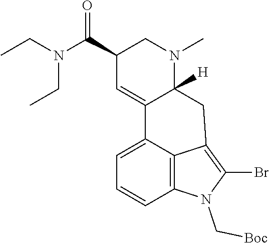 [SMILES: CCN(CC)C(=O)[C@@H]1C=C2c3cccc4c3c(c(Br)n4CC(=O)OC(C)(C)C)C[C@H]2N(C)C1]

TABLE 4
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R28 | 6.4 | 2.0 | 95.6 | 24 | 696 | 745 |
| | | R29 | 6.1 | 0.5 | 86.3 | 24 | 634 | 672 |
| | | R30 | 5.7 | 1.0 | 75.7 | 24 | 839 | 886 |
| | | Mean | 6.1 | 1 | 86 | 24 | 723 | 768 |

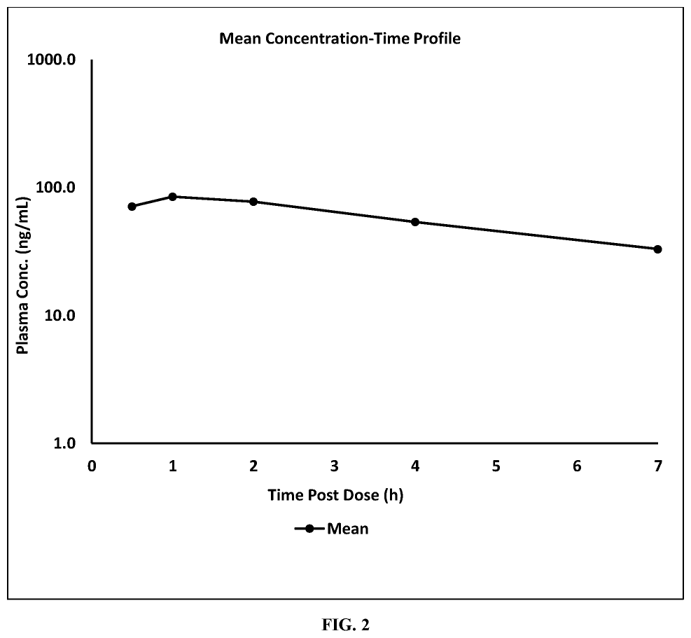

FIG. 2 shows mean concentration-time profiles of 2-Br-LSD following oral dosing of 2-Br—N-Boc prodrug (4 mg/kg) to male SD rats.

### Example A-1-3: 2-Br-LSD Pivalamide Prodrug

| Species: | Rat |
|---|---|
| Dose Route: | PO |
| Dose Level (mg/kg) | 4 |

Chemical name: ((6aR,9R)-5-bromo-N,N-diethyl-7-methyl-4-pivaloyl-4,6,6a,7,8,9-hexahydroindolo[4,3-fg]quinoline-9-carboxamide (Example 3)
Structural class: amide
Mechanistic class: presumed amidase

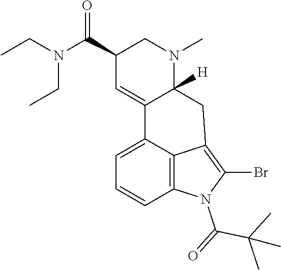 [SMILES: CCN(CC)C(=O)[C@@H]1C=C2c3cccc4c3c(c(Br)n4C(=O)C(C)(C)C)C[C@H]2N(C)C1]

TABLE 5
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R31 | NC | 0.5 | 113 | 7 | 416 | NC |
| | | R32 | 5.68 | 0.5 | 74.0 | 7 | 380 | 671 |
| | | R33 | NC | 0.5 | 113 | 7 | 452 | NR |
| | | Mean | 5.68 | 0.5 | 100 | 7 | 416 | 671 |

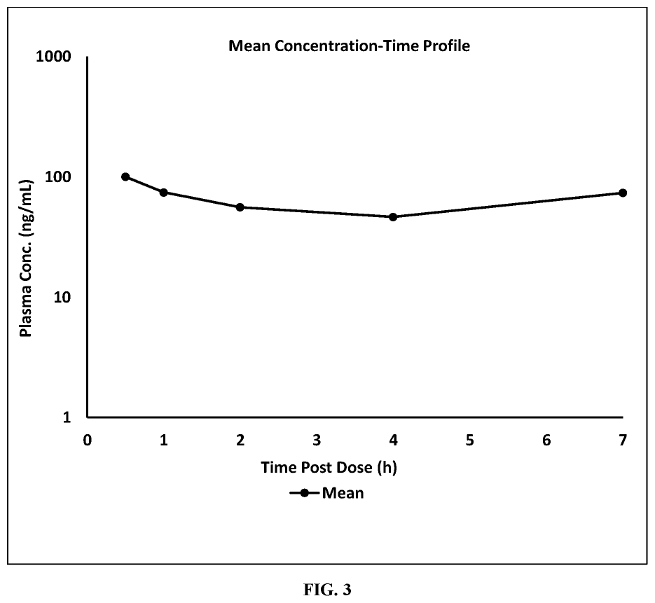

FIG. 3 shows mean concentration-time profiles of 2-Br-LSD following oral dosing of 2-Br-LSD pivalamide prodrug (4 mg/kg) to male SD rats.

Example A-1-4: 2-Br-LSD oxymethyl pivalate prodrug

| Species: | Rat |
|---|---|
| Dose Route: | PO |
| Dose Level (mg/kg) | 4 |

Chemical name: (pivaloyloxy)methyl (6aR,9R)-5-bromo-9-(diethylcarbamoyl)-7-methyl-6a,7,8,9-tetrahydroindolo[4,3-fg]quinoline-4(6H)-carboxylate (Example 2)
Structural class: acyloxymethyl carbamate
Mechanistic class: presumed esterase+chemical breakdown

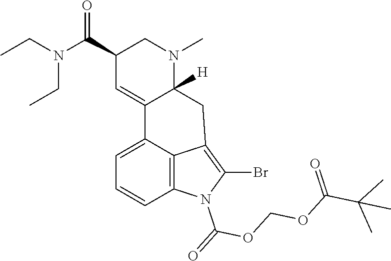 [SMILES: CCN(CC)C(=O)[C@@H]1C=C2c3cccc4c3c(c(Br)n4C(=O)OCOC(=O)C(C)(C)C)C[C@H]2N(C)C1]

TABLE 6
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R34 | NR | 4.0 | 105 | 7 | 512 | NC |
| | | R35 | NR | 2.0 | 152 | 7 | 750 | NC |
| | | R36 | 2.2 | 0.5 | 168 | 7 | 849 | 955 |
| | | Mean | 2.2 | 2.2 | 142 | 7 | 704 | 955 |

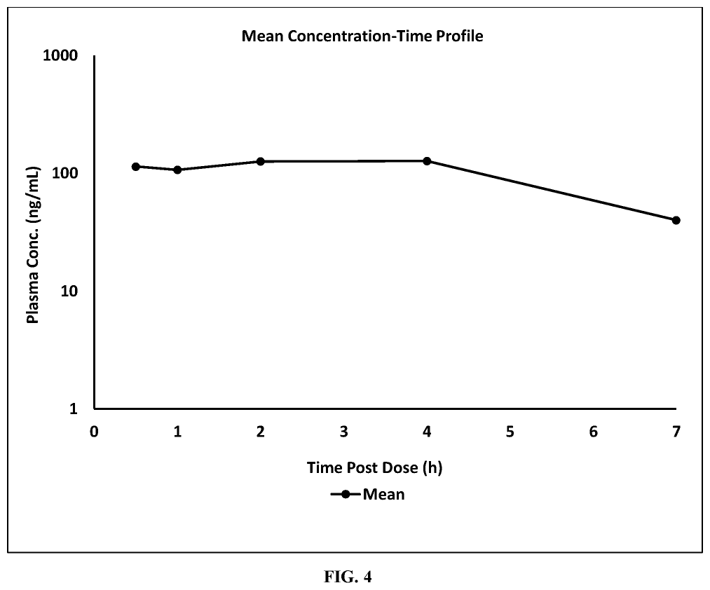

FIG. 4 shows mean concentration-time profiles of 2-Br-LSD following oral dosing of 2-Br-LSD oxymethyl pivalate prodrug (4 mg/kg) to male SD rats.

### Example A-1-5: 2-Br-LSD Acetamide Prodrug

| Species: | Rat |
|---|---|
| Dose Route: | PO |
| Dose Level (mg/kg) | 4 |

Chemical name: (6aR,9R)-4-acetyl-5-bromo-N,N-diethyl-7-methyl-4,6,6a,7,8,9-hexahydroindolo[4,3-fg]quinoline-9-carboxamide (Example 17)
Structural class: amide
Mechanistic class: presumed amidase

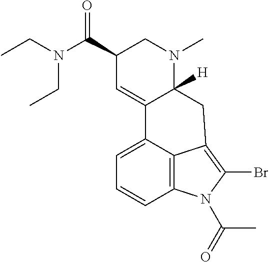 [SMILES: CCN(CC)C(=O)[C@@H]1C=C2c3cccc4c3c(c(Br)n4C(C)=O)C[C@H]2N(C)C1]

TABLE 7
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R37 | NC | 2.0 | 138 | 7 | 703 | NC |
| | | R38 | 10.1 | 0.50 | 160 | 7 | 951 | 2560 |
| | | R39 | 5.77 | 0.50 | 268 | 7 | 1100 | 2070 |
| | | Mean | 7.94 | 1.0 | 189 | 7 | 918 | 2315 |

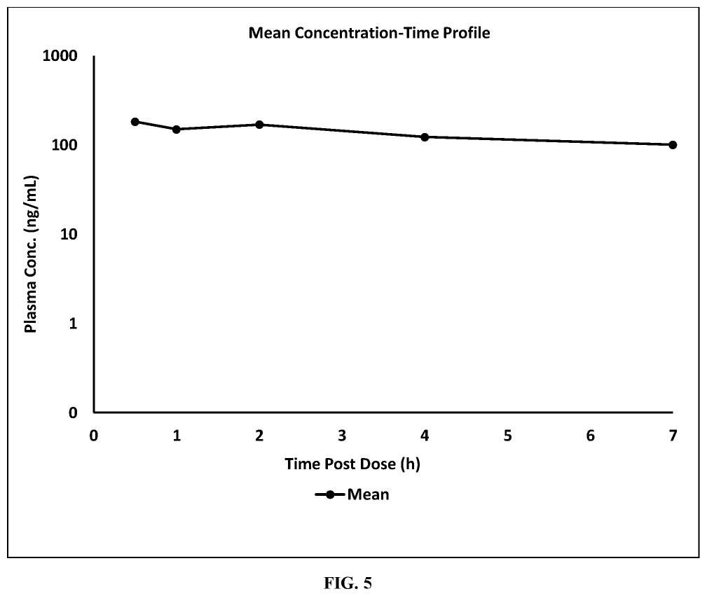

FIG. 5 shows mean concentration-time profiles of 2-Br-LSD following oral dosing of 2-Br-LSD acetamide prodrug (4 mg/kg) to male SD rats.

### Example A-1-6: 2-Br-LSD ethyl carbamate Prodrug

| Species: | Rat |
|---|---|
| Dose Route: | PO |
| Dose Level (mg/kg) | 4 |

Chemical name: ethyl (6aR,9R)-5-bromo-9-(diethylcarbamoyl)-7-methyl-6a,7,8,9-tetrahydroindolo[4,3-fg]quinoline-4(6H)-carboxylate (Example 18)
Structural class: carbamate
Mechanistic class: presumed esterase

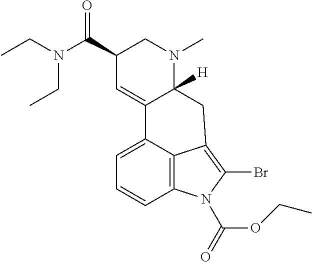 [SMILES: CCOC(=O)n1c(Br)c2c3c(cccc31)C1=C[C@@H](C(=O)N(CC)CC)CN(C)[C@@H]1C2]

TABLE 8
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R40 | NC | 2 | 180 | 7 | 972 | NC |
| | | R41 | NC | 2 | 164 | 7 | 973 | NC |
| | | R42 | NC | 7 | 132 | 7 | 796 | NC |
| | | Mean | NC | 3.67 | 159 | 7 | 914 | NC |

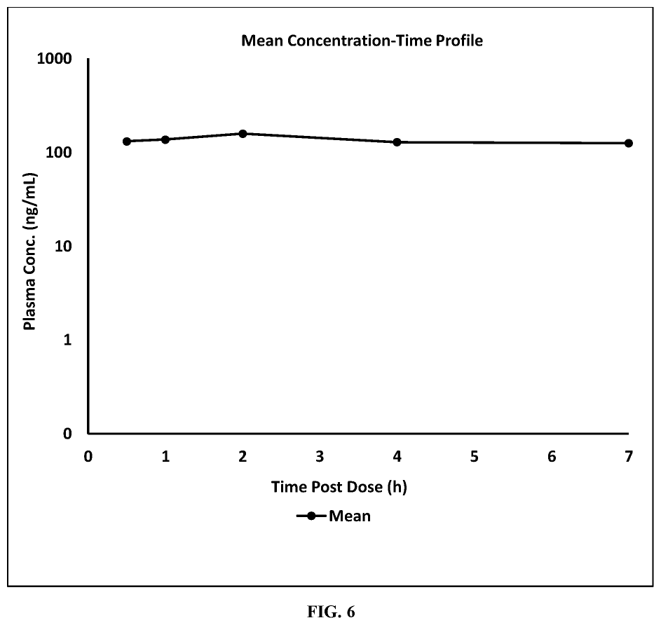

FIG. 6 shows mean concentration-time profiles of 2-Br-LSD following oral dosing of 2-Br-LSD ethyl carbamate prodrug (4 mg/kg) to male SD rats.

### Example A-1-7: 2-Br-LSD Methyl Pivalate Prodrug

| Species: | Rat |
|---|---|
| Dose Route: | PO |
| Dose Level (mg/kg) | 4 |

Chemical name: ((6aR,9R)-5-bromo-9-(diethylcarbamoyl)-7-methyl-6a,7,8,9-tetrahydroindolo[4,3-fg]quinolin-4(6H)-yl) methyl pivalate (Example 6)
Structural class: pivaloyloxymethyl (POM)
Mechanistic class: presumed esterase+chemical breakdown

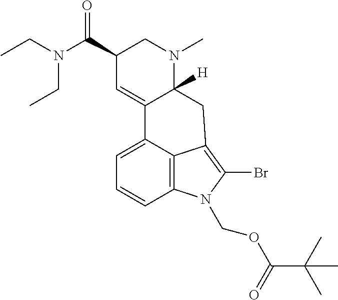 [SMILES: CCN(CC)C(=O)[C@@H]1C=C2c3cccc4c3c(c(Br)n4COC(=O)C(C)(C)C)C[C@H]2N(C)C1]

TABLE 9
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R43 | NC | 4.0 | 28 | 7.00 | 154 | NC |
| | | R44 | NC | 2.0 | 41 | 7.00 | 199 | NC |
| | | R45 | 15.3 | 0.500 | 28 | 7.00 | 149 | 552 |
| | | Mean | 15.3 | 2.2 | 32 | 7.00 | 167 | 552 |

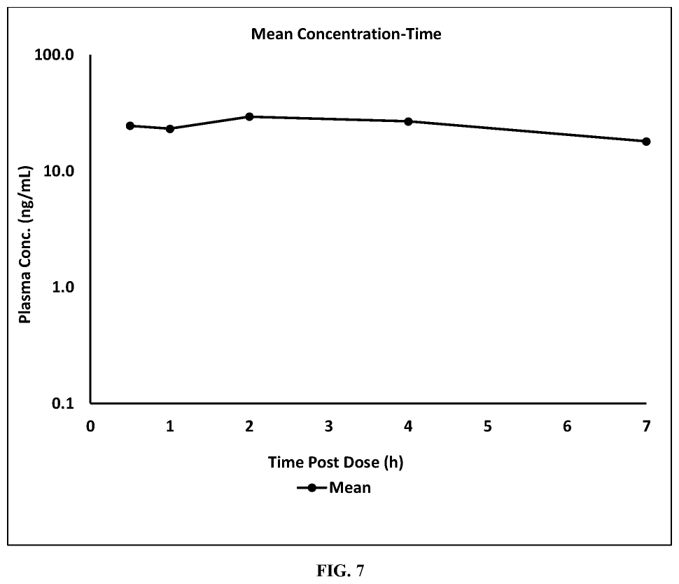

FIG. 7 shows mean concentration-time profiles of 2-Br-LSD following oral dosing of 2-Br-LSD methyl pivalate prodrug (4 mg/kg) to male SD rats.

### Example A-1-8: 2-Br-LSD Isopropyl Carbamate Prodrug

| Species: | Rat |
|---|---|
| Dose Route: | PO |
| Dose Level (mg/kg) | 4 |

Chemical name: isopropyl (6aR,9R)-5-bromo-9-(diethylcarbamoyl)-7-methyl-6a,7,8,9-tetrahydroindolo[4,3-fg]quinoline-4(6H)-carboxylate (Example 4)
Structural class: carbamate
Mechanistic class: presumed esterase

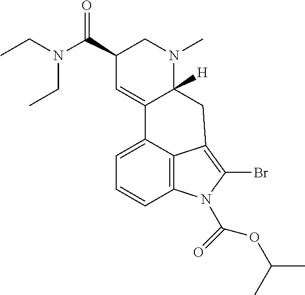 [SMILES: CCN(CC)C(=O)[C@@H]1C=C2c3cccc4c3c(c(Br)n4C(=O)OC(C)C)C[C@H]2N(C)C1]

TABLE 10
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R1 | 3.83 | 1.0 | 63.9 | 24 | 749 | 758 |
| | | R2 | NC | 7.0 | 49.2 | 24 | 686 | NC |
| | | R3 | NC | 7.0 | 37.1 | 24 | 491 | NC |
| | | Mean | 3.83 | 5.00 | 50.1 | 24 | 642 | 758 |

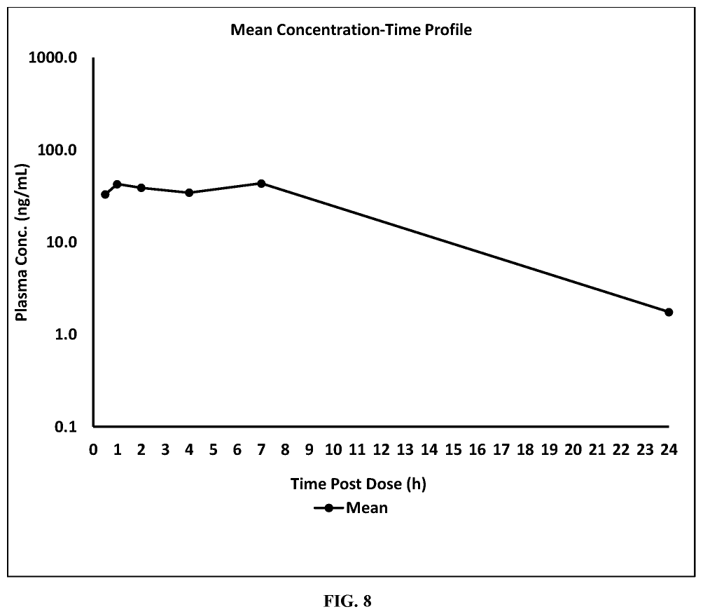

FIG. 8 shows mean concentration-time profiles of 2-Br-LSD following oral dosing of 2-Br-LSD isopropyl carbamate prodrug (4 mg/kg) to male SD rats.

### Example A-1-9: 2-Br-LSD-N-Boc-Ala Prodrug

| Species: | Rat |
|---|---|
| Dose Route: | PO |
| Dose Level (mg/kg) | 4 |

Chemical name: tert-butyl ((S)-1-((6aR,9R)-5-bromo-9-(diethylcarbamoyl)-7-methyl-6a,7,8,9-tetrahydroindolo[4,3-fg]quinolin-4(6H)-yl)-1-oxopropan-2-yl)carbamate (Example 27)
Structural class: amide
Mechanistic class: presumed amidase

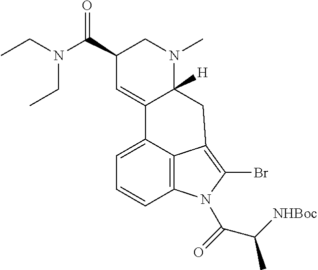 [SMILES: CCN(CC)C(=O)[C@@H]1C=C2c3cccc4c3c(c(Br)n4C(=O)[C@H](C)NC(=O)OC(C)(C)C)C[C@H]2N(C)C1]

TABLE 11
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R4 | 9.22 | 0.50 | 190 | 7 | 966 | 2310 |
| | | R5 | NC | 1.00 | 200 | 7 | 960 | NC |
| | | R6 | NC | 7.00 | 146 | 7 | 815 | NC |
| | | Mean | 9.22 | 2.83 | 179 | 7 | 914 | 2310 |

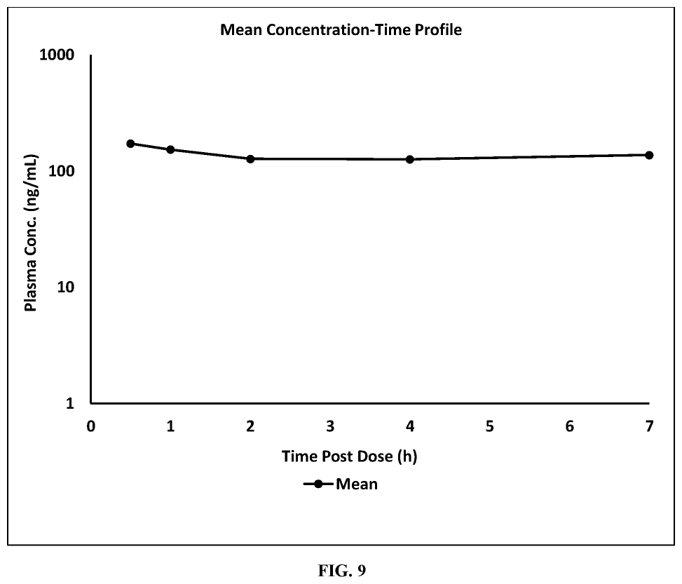

FIG. 9 shows mean concentration-time profiles of 2-Br-LSD following oral dosing of 2-Br-LSD-N-Boc-Ala prodrug (4 mg/kg) to male SD rats.

### Example A-1-10: 2-Br-LSD N-Isobutyramide Prodrug

| Species: | Rat |
|---|---|
| Dose Route: | PO |
| Dose Level (mg/kg) | 4 |

Chemical name: (6aR,9R)-5-bromo-N,N-diethyl-4-isobutyryl-7-methyl-4,6,6a,7,8,9-hexahydroindolo[4,3-fg]quinoline-9-carboxamide (Example 15)
Structural class: amide
Mechanistic class: presumed amidase

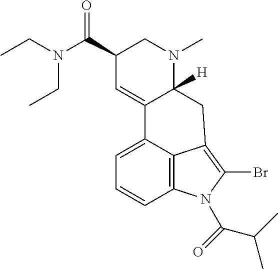 [SMILES: CCN(CC)C(=O)[C@@H]1C=C2c3cccc4c3c(c(Br)n4C(=O)C(C)C)C[C@H]2N(C)C1]

TABLE 12
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R7 | 3.22 | 0.500 | 278 | 7 | 1140 | 1420 |
| | | R8 | 2.78 | 0.500 | 258 | 24 | 1630 | 1640 |
| | | R9 | 2.51 | 0.500 | 200 | 24 | 2070 | 2070 |
| | | Mean | 2.84 | 0.500 | 245 | 18 | 1613 | 1710 |

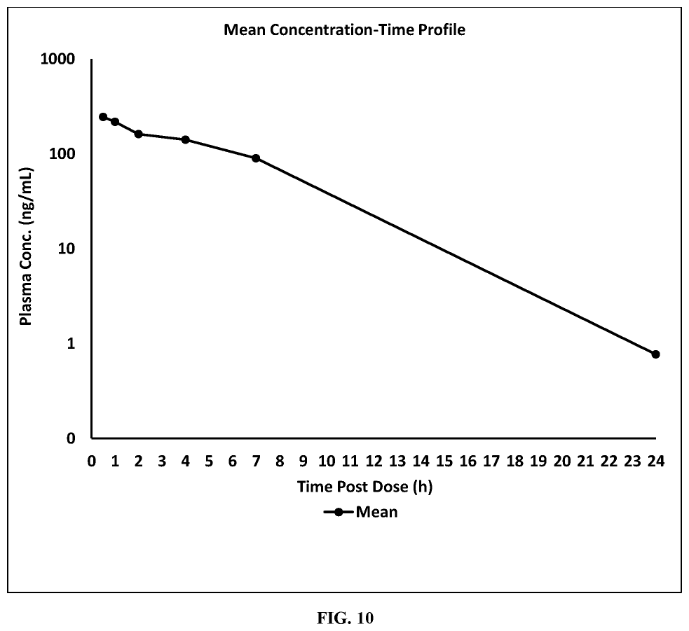

FIG. 10 shows mean concentration-time profiles of 2-Br-LSD following oral dosing of 2-Br-LSD N-isobutyramide prodrug (4 mg/kg) to male SD rats.

### Example A-1-11: 2-Br-LSD Tetrahydropyran Carboxylic Acid Prodrug

| Species: | Rat |
|---|---|
| Dose Route: | PO |
| Dose Level (mg/kg) | 4 |

Chemical name: (6aR,9R)-5-bromo-N,N-diethyl-7-methyl-4-(tetrahydro-2H-pyran-4-carbonyl)-4,6,6a,7,8,9-hexahydroindolo[4,3-fg]quinoline-9-carboxamide (Example 16)
Structural class: amide
Mechanistic class: presumed amidase

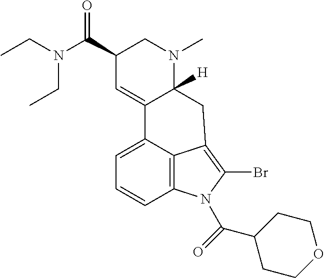 [SMILES: CCN(CC)C(=O)[C@@H]1C=C2c3cccc4c3c(c(Br)n4C(=O)C3CCOCC3)C[C@H]2N(C)C1]

TABLE 13
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R10 | NC | 0.500 | 348 | 7.00 | 1130 | NC |
| | | R11 | NC | 0.500 | 236 | 7.00 | 1090 | NC |
| | | R12 | NC | 0.500 | 328 | 7.00 | 1300 | NC |
| | | Mean | NC | 0.500 | 304 | 7.00 | 1173 | NC |

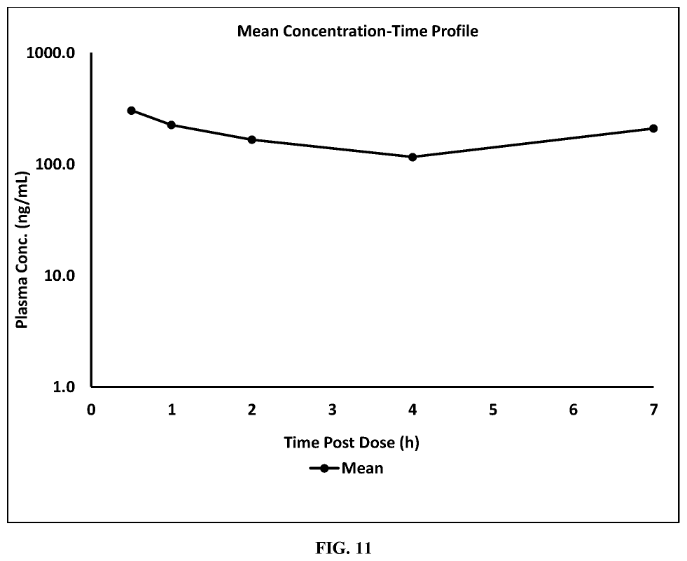

FIG. 11 shows mean concentration-time profiles of 2-Br-LSD following oral dosing of 2-Br-LSD tetrahydropyran carboxylic acid prodrug (4 mg/kg) to male SD rats.

### Example A-1-12: 2-Br-LSD Boc-Val Formate Prodrug

| Species: | Rat |
|---|---|
| Dose Route: | PO |
| Dose Level (mg/kg) | 4 |

Chemical name: tert-butyl ((S′)-1-((6aR,9R)-5-bromo-9-(diethylcarbamoyl)-7-methyl-6a,7,8,9-tetrahydroindolo[4,3-fg]quinolin-4(6H)-yl)-3-methyl-1-oxobutan-2-yl)carbamate (Example 29)
Structural class: amide
Mechanistic class: presumed amidase

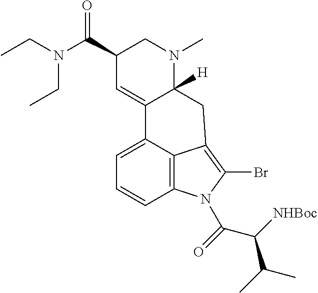 [SMILES: CCN(CC)C(=O)[C@@H]1C=C2c3cccc4c3c(c(Br)n4C(=O)[C@@H](NC(=O)OC(C)(C)C)C(C)C)C[C@H]2N(C)C1]

TABLE 14
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R13 | 5.46 | 0.500 | 145 | 24.0 | 1030 | 1070 |
| | | R14 | 11.9 | 0.500 | 190 | 24.0 | 1000 | 1290 |
| | | R15 | 5.06 | 0.500 | 183 | 24.0 | 1740 | 1780 |
| | | Mean | 7.47 | 0.500 | 173 | 24.0 | 1257 | 1380 |

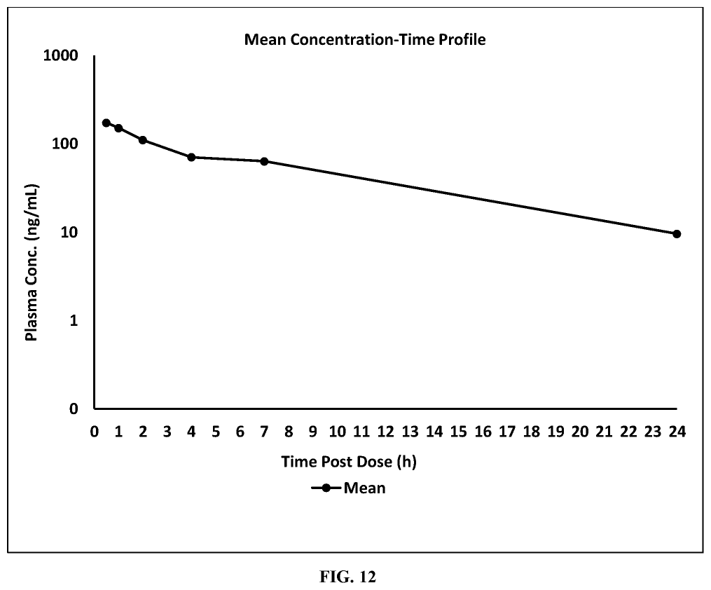

FIG. 12 shows mean concentration-time profiles of 2-Br-LSD following oral dosing of 2-Br-LSD Boc-Val formate prodrug (4 mg/kg) to male SD rats.

### Example A-1-13: 2-Br-LSD hydroxymethyl prodrug

| Species: | Rat |
|---|---|
| Dose Route: | PO |
| Dose Level (mg/kg) | 4 |

Chemical name: (6aR,9R)-5-bromo-N,N-diethyl-4-(hydroxymethyl)-7-methyl-4,6,6a,7,8,9-hexahydroindolo[4,3-fg]quinoline-9-carboxamide (Example 5)
Structural class: hydroxymethyl
Mechanistic class: presumed chemical breakdown

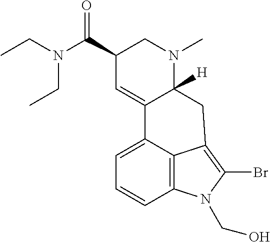 [SMILES: CCN(CC)C(=O)[C@@H]1C=C2c3cccc4c3c(c(Br)n4CO)C[C@H]2N(C)C1]

TABLE 15
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R16 | 2.94 | 0.500 | 331 | 24.0 | 2510 | 2510 |
| | | R17 | 4.57 | 0.500 | 296 | 7.00 | 1330 | 2050 |
| | | R18 | 4.56 | 0.500 | 292 | 7.00 | 854 | 1330 |
| | | Mean | 4.02 | 0.500 | 306 | 12.7 | 1565 | 1963 |

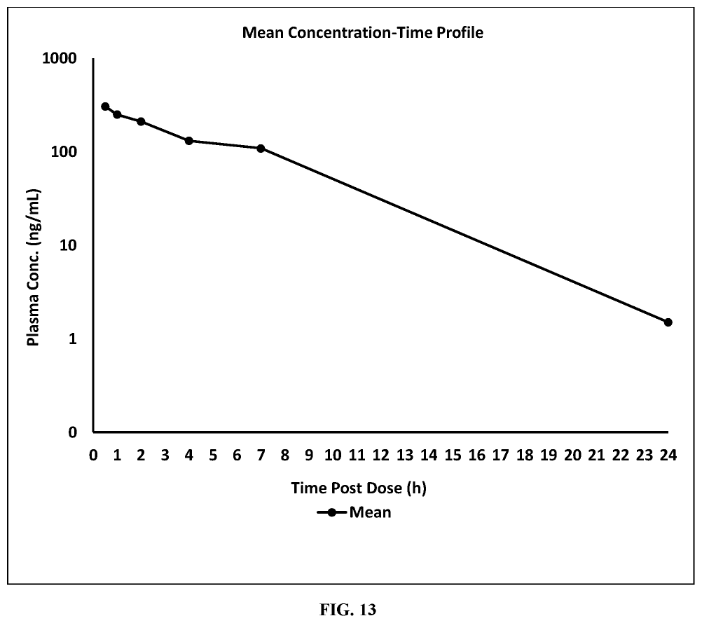

FIG. 13 shows mean concentration-time profiles of 2-Br-LSD following oral dosing of 2-Br-LSD hydroxymethyl prodrug (4 mg/kg) to male SD rats.

### Example A-1-14: 2-Br-LSD methyl ethyl carbonate prodrug

| Species: | Rat |
|---|---|
| Dose Route: | PO |
| Dose Level (mg/kg) | 4 |

Chemical name: ((6aR,9R)-5-bromo-9-(diethylcarbamoyl)-7-methyl-6a,7,8,9-tetrahydroindolo[4,3-fg]quinolin-4(6H)-yl)methyl ethyl carbonate (Example 7)
Structural class: oxymethyl carbonate
Mechanistic class: presumed esterase+chemical breakdown

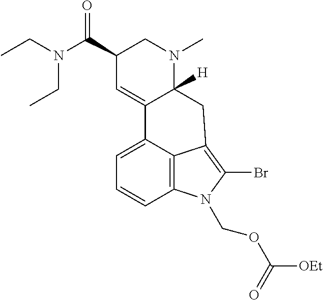 [SMILES: CCOC(=O)OCn1c(Br)c2c3c(cccc31)C1=C[C@@H](C(=O)N(CC)CC)CN(C)[C@@H]1C2]

TABLE 16
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R19 | 78.7 | 0.500 | 66.0 | 7.00 | 118 | 1730 |
| | | R20 | 2.43 | 0.500 | 146 | 7.00 | 168 | 182 |
| | | R21 | 4.35 | 0.500 | 97.6 | 7.00 | 228 | 335 |
| | | Mean | 28.5 | 0.500 | 103 | 7.00 | 171 | 749 |

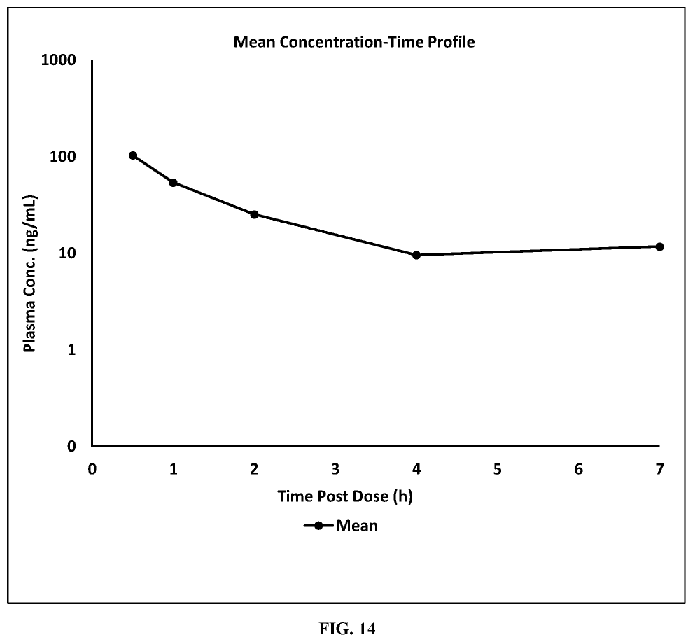

FIG. 14 shows mean concentration-time profiles of 2-Br-LSD following oral dosing of 2-Br-LSD methyl ethyl carbonate prodrug (4 mg/kg) to male SD rats.

### Example A-1-15: 2-Br-LSD methylene acetamide prodrug

| Species: | Rat |
|---|---|
| Dose Route: | PO |
| Dose Level (mg/kg) | 4 |

Chemical name: (6aR,9R)-4-(acetamidomethyl)-5-bromo-N,N-diethyl-7-methyl-4,6,6a,7,8,9-hexahydroindolo[4,3-fg]quinoline-9-carboxamide (Example 19)
Structural class: Mannich base
Mechanistic class: presumed amidase+chemical breakdown

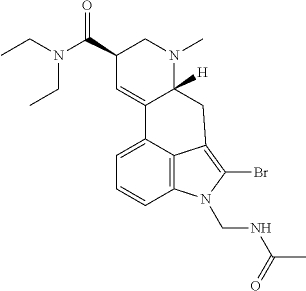 [SMILES: CCN(CC)C(=O)[C@@H]1C=C2c3cccc4c3c(c(Br)n4CNC(C)=O)C[C@H]2N(C)C1]

TABLE 17
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R22 | 2.60 | 0.500 | 10.2 | 7.00 | 29.7 | 34.9 |
| | | R23 | 5.34 | 0.500 | 21.5 | 7.00 | 50.9 | 87.9 |
| | | R24 | 2.25 | 0.500 | 17.4 | 7.00 | 57.5 | 64.2 |
| | | Mean | 3.40 | 0.500 | 16.4 | 7.00 | 46.0 | 62.3 |

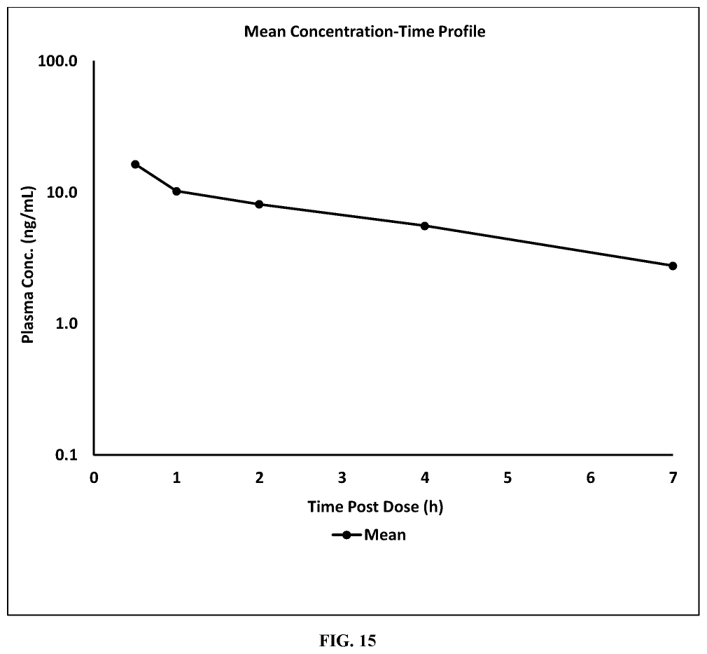

FIG. 15 shows mean concentration-time profiles of 2-Br-LSD following oral dosing of 2-Br-LSD methylene acetamide prodrug (4 mg/kg) to male SD rats.

### Example A-1-16: 2-Br-LSD trimethyl lock prodrug

| Species: | Rat |
|---|---|
| Dose Route: | PO |
| Dose Level (mg/kg) | 4 |

Chemical name: 2-(4-((6aR,9R)-5-bromo-9-(diethylcarbamoyl)-7-methyl-6a,7,8,9-tetrahydroindolo[4,3-fg]quinolin-4(6H)-yl)-2-methyl-4-oxobutan-2-yl)-3,5-dimethylphenyl acetate (Example 9)
Structural class: amide (trimethyllock)
Mechanistic class: presumed esterase+cyclization

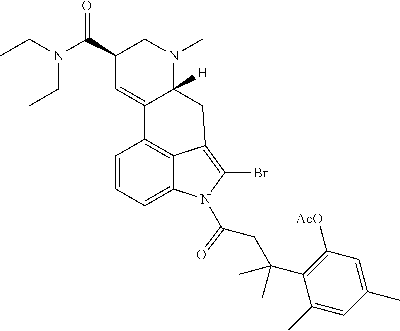 [SMILES: CCN(CC)C(=O)[C@@H]1C=C2c3cccc4c3c(c(Br)n4C(=O)CC(C)(C)c3c(C)cc(C)cc3OC(C)=O)C[C@H]2N(C)C1]

TABLE 18
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R13 | 2.77 | 0.500 | 232 | 24.0 | 1830 | 1830 |
| | | R14 | 6.69 | 0.500 | 226 | 24.0 | 2060 | 2250 |
| | | R15 | 3.79 | 0.500 | 217 | 24.0 | 2030 | 2040 |
| | | Mean | 4.42 | 0.500 | 225 | 24.0 | 1973 | 2040 |

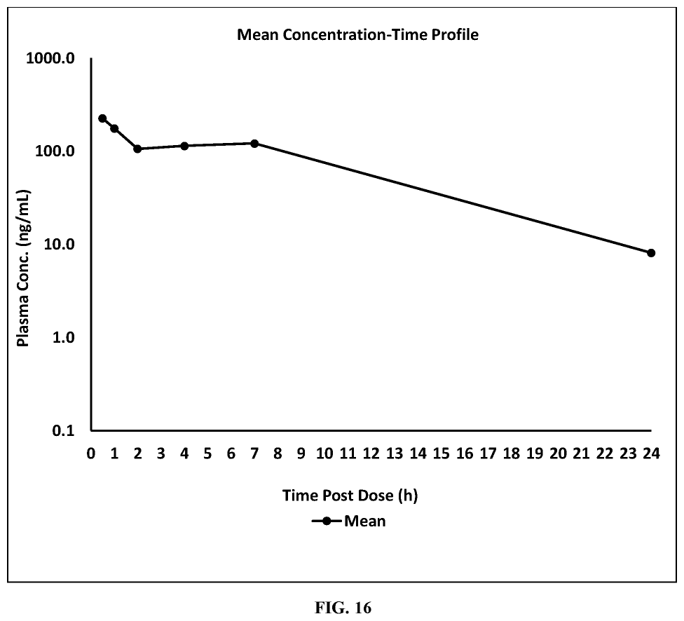

FIG. 16 shows mean concentration-time profiles of 2-Br-LSD following oral dosing of 2-Br-LSD trimethyl lock prodrug (4 mg/kg) to male SD rats.

### Example A-1-17: 2-Br-LSD-Propylamide Prodrug

| Species: | Rat |
|---|---|
| Dose Route: | PO |
| Dose Level (mg/kg) | 4 |

Chemical name: (6aR,9R)-5-bromo-N,N-diethyl-7-methyl-4-propionyl-4,6,6a,7,8,9-hexahydroindolo[4,3-fg]quinoline-9-carboxamide (Example 11)
Structural class: amide
Mechanistic class: presumed amidase

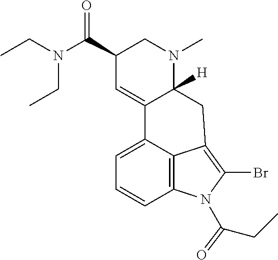 [SMILES: CCC(=O)n1c(Br)c2c3c(cccc31)C1=C[C@@H](C(=O)N(CC)CC)CN(C)[C@@H]1C2]

TABLE 19
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R16 | 4.46 | 0.500 | 186 | 7.00 | 771 | 1170 |
| | | R17 | NR | 0.500 | 165 | 7.00 | 709 | NR |
| | | R18 | 4.15 | 0.500 | 133 | 7.00 | 464 | 659 |
| | | Mean | 4.31 | 0.500 | 161 | 7.00 | 648 | 915 |

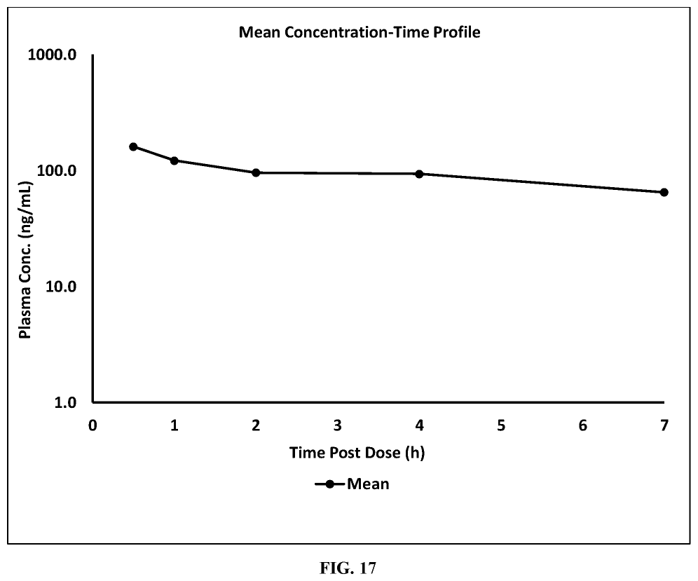

FIG. 17 shows mean concentration-time profiles of 2-Br-LSD following oral dosing of 2-Br-LSD-propylamide prodrug (4 mg/kg) to male SD rats.

### Example A-1-18: 2-Br-LSD n-Butyl Amide Prodrug

| Species: | Rat |
|---|---|
| Dose Route: | PO |
| Dose Level (mg/kg) | 4 |

Chemical name: (6aR,9R)-5-bromo-4-butyryl-N,N-diethyl-7-methyl-4,6,6a,7,8,9-hexahydroindolo[4,3-fg]quinoline-9-carboxamide (Example 1)
Structural class: amide
Mechanistic class: presumed amidase

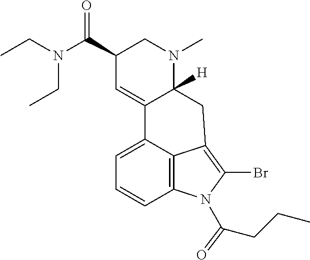 [SMILES: CCCC(=O)n1c(Br)c2c3c(cccc31)C1=C[C@@H](C(=O)N(CC)CC)CN(C)[C@@H]1C2]

TABLE 20
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R19 | 4.15 | 0.500 | 133 | 7.00 | 464 | 659 |
| | | R20 | NR | 2.00 | 93.6 | 7.00 | 526 | NR |
| | | R21 | 2.66 | 0.500 | 177 | 24.0 | 1140 | 1150 |
| | | Mean | 3.41 | 1.00 | 135 | 12.7 | 710 | 905 |

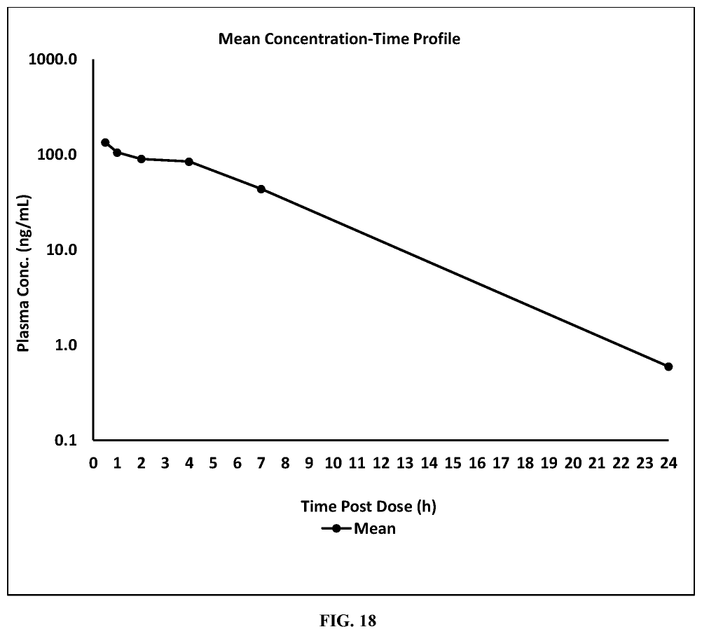

FIG. 18 shows mean concentration-time profiles of 2-Br-LSD following oral dosing of 2-Br-LSD n-butyl amide prodrug (4 mg/kg) to male SD rats.

### Example A-1-19: 2-Br-LSD oxymethyl tetrahydropyran prodrug

| Species: | Rat |
|---|---|
| Dose Route: | PO |
| Dose Level (mg/kg) | 4 |

Chemical name: (tetrahydro-2H-pyran-4-carbonyl)oxy)methyl (6aR,9R)-5-bromo-9-(diethylcarbamoyl)-7-methyl-6a,7,8,9-tetrahydroindolo[4,3-fg]quinoline-4(6H)-carboxylate (Example 22)
Structural class: acyloxymethyl
Mechanistic class: presumed esterase+chemical breakdown

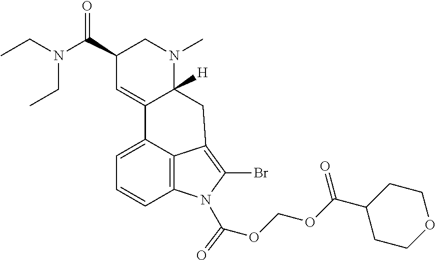 [SMILES: CCN(CC)C(=O)[C@@H]1C=C2c3cccc4c3c(c(Br)n4C(=O)OCOC(=O)C3CCOCC3)C[C@H]2N(C)C1]

TABLE 21
2-Br-LSD Pharmacokinetic Parameters

| | Dose | Animal | T½ | Tmax | Cmax | Tlast | AUClast | AUCINF_obs |
|---|---|---|---|---|---|---|---|---|
| Analyte | Route | ID | (hr) | (hr) | (ng/mL) | (hr) | (ng/ml*hr) | (ng/ml*hr) |
| 2-Br-LSD | Oral | R22 | 6.20 | 0.500 | 139 | 7.00 | 600 | 1070 |
| | | R23 | 3.12 | 0.500 | 164 | 7.00 | 679 | 876 |
| | | R24 | 6.31 | 0.500 | 120 | 7.00 | 602 | 1050 |
| | | Mean | 5.21 | 0.500 | 141 | 7.00 | 627 | 999 |

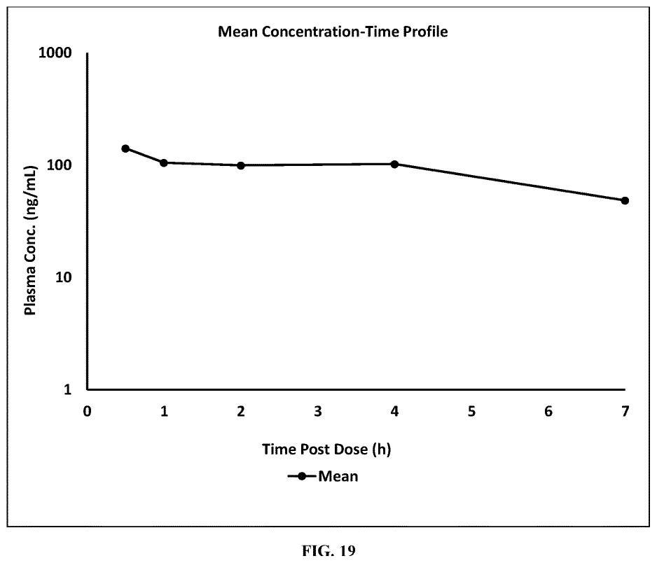

FIG. 19 shows mean concentration-time profiles of 2-Br-LSD following oral dosing of 2-Br-LSD oxymethyl tetrahydropyran prodrug (4 mg/kg) to male SD rats.

### Example B: Evaluation of Metabolic Stability in Human Liver Microsomes

Microsomal Assay: Human liver microsomes (20 mg/mL) are obtained. (3-nicotinamide adenine dinucleotide phosphate, reduced form (NADPH), magnesium chloride (MgCl₂), and dimethyl sulfoxide (DMSO) are purchased.

Determination of Metabolic Stability: 7.5 mM stock solutions of test compounds of the above structural formula (e.g., of an embodiment or aspect of embodiment thereof described herein), or pharmaceutically acceptable salt thereof, are prepared in DMSO.

The 7.5 mM stock solutions are diluted to 12.5-50 μM in acetonitrile (ACN). The 20 mg/mL human liver microsomes are diluted to 0.625 mg/mL in 0.1 μM potassium phosphate buffer, pH 7.4, containing 3 mM MgCl₂. The diluted microsomes are added to wells of a 96-well deep-well polypropylene plate in triplicate. A 10 pL aliquot of the 12.5-50 μM test compound is added to the microsomes and the mixture is pre-warmed for 10 minutes. Reactions are initiated by addition of pre-warmed NADPH solution. The final reaction volume is 0.5 mL and contains 4.0 mg/mL human liver microsomes, 0.25 μM test compound, and 2 mM NADPH in 0.1 M potassium phosphate buffer, pH 7.4, and 3 mM MgCl₂. The reaction mixtures are incubated at 37° C., and 50 pL aliquots are removed at 0, 5, 10, 20, and 30 minutes and added to shallow-well 96-well plates which contain 50 μL of ice-cold ACN (acetonitrile) with internal standard to stop the reactions. The plates are stored at 4° C. for 20 minutes after which 100 μL of water is added to the wells of the plate before centrifugation to pellet precipitated proteins. Supernatants are transferred to another 96-well plate and analyzed for amounts of parent remaining by LC-MS/MS using an Applied Bio-systems API 4000 mass spectrometer. The same procedure is followed for the non-enriched counterpart of the compound and the positive control, 7-ethoxycoumarin (1 M). Testing is done in triplicate.

Data analysis: The in vitro T_{1/2}s for test compounds are calculated from the slopes of the linear regression of % parent remaining (In) vs incubation time relationship.

$$in vitro {T}_{1 / 2} = 0 \text{.693} / k$$

k=−[slope of linear regression of % parent remaining(In) vs incubation time]

The apparent intrinsic clearance is calculated using the following equation:

$$C {L}_{i n t} ( mL / min / kg ) = ( 0.693 / in vitro T ) ( Incubation Volume / mg of microsomes ) ( 45 mg microsomes / gram of liver ) ( 20 gm of liver / kg b . w .$$

Data analysis is performed using Microsoft Excel Software.

In these experiments, values equal to or more than a 15% increase in half-life are considered to be a significant difference if the apparent intrinsic clearance ratio (prodrug of 2-Br-LSD or of an isotopically enriched analog of 2-Br-LSD/2-Br-LSD) is >1.15 or <0.85, then there is considered to be significant differentiation.

### Example C: Biological Assays and Methods

Head-Twitch Response (HTR). The head-twitch response assay is performed as is known to those of skill in the art using both male and female C57BL/6J mice (2 per treatment). The mice are obtained and are approximately 8 weeks old at the time of the experiments. Compounds are administered via intraperitoneal injection (5 mL/kg) using 0.9% saline as the vehicle. As a positive control, LSD (lysergic acid diethylamide) 0.3 mg/kg is utilized. Behavior is videotaped, later scored by two blinded observers, and the results are averaged (Pearson correlation coefficient=0.93).

Serotonin and Opioid Receptor Functional Assays. Functional assay screens at 5-HT and opioid receptors are performed in parallel using the same compound dilutions and 384-well format high-throughput assay platforms. Assays assess activity at all human isoforms of the receptors, except where noted for the mouse 5-HT_{2A} receptor. Receptor constructs in pcDNA vectors are generated from the Presto-Tango GPCR library with minor modifications. All compounds are serially diluted in drug buffer (HBSS, 20 mM HEPES, pH 7.4 supplemented with 0.1% bovine serum albumin and 0.01% ascorbic acid) and dispensed into 384-well assay plates using a FLIPR^{TETRA} (Molecular Devices). Every plate included a positive control such as 5-HT (for all 5-HT receptors), DADLE (DOR), salvinorin A (KOR), and DAMGO (MOR). For measurements of 5-HT_{2A}, 5-HT2B, and 5-HT2C Gq-mediated calcium flux function, HEK Flp-In 293 T-Rex stable cell lines (Invitrogen) are loaded with Fluo₄ dye for one hour, stimulated with compounds and read for baseline (0-10 seconds) and peak fold-over-basal fluorescence (5 minutes) at 25° C. on the FLIPR^{TETRA}. For measurement of 5-HT6 and 5-HT7a functional assays, Gs-mediated cAMP accumulation is detected using the split-luciferase GloSensor assay in HEKT cells measuring luminescence on a Microbeta Trilux (Perkin Elmer) with a 15 min drug incubation at 25° C. For 5-HT1A, 5-HT1B, 5-HT1F, MOR, KOR, and DOR functional assays, Gi/o-mediated cAMP inhibition is measured using the split-luciferase GloSensor assay in HEKT cells, conducted similarly as above, but in combination with either 0.3 μM isoproterenol (5-HT1A, 5-HT1B, 5-HT1F) or 1 μM forskolin (MOR, KOR, and DOR) to stimulate endogenous cAMP accumulation. For measurement of 5-HT1D, 5-HT1E, 5-HT4, and 5-HT5A functional assays, P-arrestin2 recruitment is measured by the Tango assay utilizing HTLA cells expressing TEV fused-P-arrestin2, as described previously with minor modifications. Data for all assays are plotted and non-linear regression is performed using “log(agonist) vs. response” in Graphpad Prism to yield Emax and EC₅₀ parameter estimates.

5HT_{2A} Sensor Assays. HEK293T (ATCC) 5HT_{2A} sensor stable line (sLight1.3s) is generated via lentiviral transduction of HIV-EF1α-sLight1.3 and propagated from a single colony. Lentivirus is produced using 2^{nd} generation lentiviral plasmids pHIV-EF1α-sLight1.3, pHCMV-G, and pCMV-deltaR8.2.

For the screening of the compounds, sLightl1.3s cells are plated in 96-well plates at a density of 40000 24-hours prior to imaging. On the day of imaging, compounds solubilized in DMSO are diluted from the 100 mM stock solution to working concentrations of 1 mM, 100 mM and 1 μM with a DMSO concentration of 1%. Immediately prior to imaging, cells growing in DMEM (Gibco) are washed 2× with HBSS (Gibco) and in agonist mode 180 μL of HBSS or in antagonist mode 160 μL of HBSS is added to each well after the final wash. For agonist mode, images are taken before and after the addition of the 20 μL compound working solution into the wells containing 180 μL HBSS. This produces final compound concentrations of 100 mM, 10 mM and 100 nM with a DMSO concentration of 0.1%. For antagonist mode, images are taken before and after addition of 20 μL of 900 nM 5-HT and again after 20 μL of the compound working solutions to produce final concentrations of 100 nM for 5HT and 100 mM, 10 mM and 100 nM for the compounds with a DMSO concentration of 0.1%. Each compound is tested in triplicate (3 wells) for each concentration (100 mM, 10 mM and 100 nM). Additionally, within each plate, 100 nM 5HT and 0.1% DMSO controls are also imaged.

Imaging is performed using the Leica DMi8 inverted microscope with a 40× objective using the FITC preset with an excitation of 460 nm and emission of 512-542 nm. For each well, the cellular membrane where the 5HT_{2A} sensor is targeted is autofocused using the adaptive focus controls and 5 images from different regions within the well are taken with each image processed from a 2×2 binning.

For data processing, the membranes from each image are segmented and analyzed using a custom algorithm written in MATFAB producing a single raw fluorescence intensity value. For each well the 5 raw fluorescence intensity values generated from the 5 images are averaged and the change in fluorescence intensity (dFF) is calculated as:

dFF=(Fₛₐₜ−Fₐₚₒ)/Fₐₚₒ

For both agonist and antagonist modes, the fluorescence intensity values before compound addition in FIBSS only are used as the Fₐₚₒ values while the fluorescence intensity values after compound addition are used as the Fₛₐₜ values.

For agonist mode, data are as percent activation relative to 5HT, where 0 is the average of the DMSO wells and 100 is the average of the 100 μM 5HT wells. For antagonist mode, the inactivation score is calculated as:

$$Inactivation score = ( dFFF ( Compound + 5 HT ) - dFF ( 5 HT ) ) / dFF ( 5 HT )$$

Plasticity Effects: Treatment of rat embryonic cortical neurons with compounds disclosed herein or a metabolite thereof is evaluated for increased dendritic arbor complexity at 6 days in vitro (DIV6) as measured by Sholl analysis. The effect of the present compounds on dendritic growth can be determined to be 5-HT_{2A}-dependent, if pretreatment with ketanserin—a 5-HT_{2A} antagonist—inhibits their effects.

In addition to promoting dendritic growth, the present compounds also are evaluated for increased dendritic spine density to a comparable extent as ibogaine in mature cortical cultures (DIV20). The effects of the compounds on cortical dendritic spine dynamics in vivo using transcranial 2-photon imaging is assessed. First, spines are imaged on specific dendritic loci defined by their relation to blood vessel and dendritic architectures. Next, the animals are systemically administered vehicle, a compound of the present invention, or the hallucinogenic 5-HT_{2A} agonist 2,5-dimethoxy-4-iodoamphetamine (DOI). After 24 h, the same dendritic segments are re-imaged, and the number of spines gained or lost is quantified. Examples of the presently disclosed compounds increase spine formation in mouse primary sensory cortex, suggesting that the present compounds support neuronal plasticity.

As increased cortical structural plasticity in the anterior parts of the brain mediates the sustained (>24 h) antidepressant-like effects of ketamine and play a role in the therapeutic effects of 5-HT_{2A} agonists, the impact of the present compounds on forced swim test (FST) behavior is evaluated. First, a pretest is used to induce a depressive phenotype. Compounds are administered 24 h after the pre-test, and the FST is performed 24 h and 7 d post compound administration. Effective compounds of the invention, like ketamine, significantly reduce immobility 24 h after administration.

Dendritogenesis Assays. Compounds disclosed herein are evaluated for their ability to increase dendritic arbor complexity in cultures of cortical neurons using a phenotypic assay. Following treatment, neurons are fixed and visualized using an antibody against MAP2—a cytoskeletal protein localized to the somatodendritic compartment of neurons. Sholl analysis is then performed, and the maximum number of crossings (Nₘₐₓ) is used as a quantitative metric of dendritic arbor complexity. For statistical comparisons between specific compounds, the raw Nₘₐₓ values are compared. Percent efficacies are determined by setting the Nₘₐₓ values for the vehicle (DMSO) and positive (ketamine) controls equal to 0% and 100%, respectively.

Animals. For the dendritogenesis experiments, timed pregnant Sprague Dawley rats are obtained. For the head-twitch response assay, male and female C57BL/6J mice are obtained.

Dendritogenesis—Sholl Analysis. Dendritogenesis experiments are performed following a previously published methods with slight modifications. Neurons are plated in 96-well format (200 μL of media per well) at a density of approximately 15,000 cells/well in Neurobasal (Life Technologies) containing 1% penicillin-streptomycin, 10% heat-inactivated fetal bovine serum, and 0.5 mM glutamine. After 24 h, the medium is replaced with Neurobasal containing 1× B27 supplement (Life Technologies), 1% penicillin-streptomycin, 0.5 mM glutamine, and 12.5 μM glutamate. After 3 days in vitro (DIV3), the cells are treated with compounds. All compounds tested in the dendritogenesis assays are treated at 10 μM. Stock solutions of the compounds in DMSO are first diluted 100-fold in Neurobasal before an additional 10-fold dilution into each well (total dilution=1:1000; 0.1% DMSO concentration). Treatments are randomized. After 1 h, the media is removed and replaced with new Neurobasal media containing 1× B27 supplement, 1% penicillin-streptomycin, 0.5 mM glutamine, and 12.5 mM glutamate. The cells are allowed to grow for an additional 71 h. At that time, neurons are fixed by removing 80% of the media and replacing it with a volume of 4% aqueous paraformaldehyde (Alfa Aesar) equal to 50% of the working volume of the well. Then, the cells are incubated at room temperature for 20 min before the fixative is aspirated and each well washed twice with DPBS. Cells are permeabilized using 0.2% Triton X-100 (ThermoFisher) in DPBS for 20 minutes at room temperature without shaking. Plates are blocked with antibody diluting buffer (ADB) containing 2% bovine serum albumin (BSA) in DPBS for 1 h at room temperature. Then, plates are incubated overnight at 4° C. with gentle shaking in ADB containing a chicken anti-MAP2 antibody (1:10,000; EnCor, CPCA-MAP2). The next day, plates are washed three times with DPBS and once with 2% ADB in DPBS. Plates are incubated for 1 h at room temperature in ADB containing an anti-chicken IgG secondary antibody conjugated to Alexa Fluor 488 (Life Technologies, 1:500) and washed five times with DPBS. After the final wash, 100 μL of DPBS is added per well and imaged on an ImageXpress Micro XL High-Content Screening System (Molecular Devices, Sunnyvale, CA) with a 20× objective. Images are analyzed using ImageJ Fiji (version 1.51 W). First, images corresponding to each treatment are sorted into individual folders that are then blinded for data analysis. Plate controls (both positive and negative) are used to ensure that the assay is working properly as well as to visually determine appropriate numerical values for brightness/contrast and thresholding to be applied universally to the remainder of the randomized images. Next, the brightness/contrast settings are applied, and approximately 1-2 individual pyramidal-like neurons per image (i.e., no bipolar neurons) are selected using the rectangular selection tool and saved as separate files. Neurons are selected that do not overlap extensively with other cells or extend far beyond the field of view.

In Vivo Spine Dynamics. Male and female Thyl-GFP-M line mice (n=5 per condition) are purchased and maintained. In vivo transcranial two-photon imaging and data analysis are performed as previously described. Briefly, mice are anesthetized with an intraperitoneal (i.p.) injection of a mixture of ketamine (87 mg/kg) and xylazine (8.7 mg/kg). A small region of the exposed skull is manually thinned down to 20-30 pm for optical access. Spines on apical dendrites in mouse primary sensory cortices are imaged using a Bruker Ultima IV two-photon microscope equipped with an Olympus water-immersion objective (40×, NA=0.8) and a Ti:Sapphire laser (Spectra-Physics Mai-Tai, excitation wavelength 920 nm). Images are taken at a zoom of 4.0 (pixel size 0.143×0.143 pm) and Z-step size of 0.7 pm. The mice receive an i.p. injection (injection volume=5 mL/kg) of DOI (10 mg/kg) or test compound (50 mg/kg) immediately after they recover from anesthesia given prior to the first imaging session. The animals are re-imaged 24 h after drug administration. Dendritic spine dynamics are analyzed using ImageJ. Spine formation and elimination are quantified as percentages of spine number on day 0.

Forced Swim Test (FST). Male C57/BL6J mice (9-10 weeks old at time of experiment) are obtained. After 1 week in the vivarium each mouse is handled for approximately 1 minute by the experimenter for 3 consecutive days leading up to the first FST. All experiments are carried out by the same experimenter who performs handling. During the FST, mice undergo a 6 min swim session in a clear Plexiglas cylinder 40 cm tall, 20 cm in diameter, and filled with 30 cm of 24±1° C. water. Fresh water is used for every mouse. After handling and habituation to the experimenter, drug-naive mice first undergo a pretest swim to more reliably induce a depressive phenotype in the subsequent FST sessions. Immobility scores for all mice are determined after the pre-test and mice are randomly assigned to treatment groups to generate groups with similar average immobility scores to be used for the following two FST sessions. The next day, the animals receive intraperitoneal injections of experimental compounds (20 mg/kg), a positive control (ketamine, 3 mg/kg), or vehicle (saline). The animals are subjected to the FST 30 mins after injection and then returned to their home cages. All FSTs are performed between the hours of 8 am and 1 pm. Experiments are video-recorded and manually scored offline. Immobility time defined as passive floating or remaining motionless with no activity other than that needed to keep the mouse's head above water is scored for the last 4 min of the 6 min trial.

Statistical analysis. Treatments are randomized, and data are analyzed by experimenters blinded to treatment conditions. Statistical analyses are performed using GraphPad Prism (version 8.1.2). The specific tests are F-statistics and degrees of freedom. All comparisons are planned prior to performing each experiment. For dendritogenesis experiments a one way ANOVA with Dunnett's post hoc test is deemed most appropriate. Ketamine is included as a positive control to ensure that the assay is working properly.

Alcohol Use Disorder Model: To assess the anti-addictive potential of the present compounds, an alcohol drinking paradigm that models heavy alcohol use and binge drinking behavior in humans is employed. Using a 2-bottle choice setup (20% ethanol (v/v), EtOH vs. water, H₂O), mice are subjected to repeated cycles of binge drinking and withdrawal over the course of 7 weeks.

This schedule results in heavy EtOH consumption, binge drinking-like behavior, and generates blood alcohol content equivalent to that of human subjects suffering from alcohol use disorder (AUD). Next, compounds of the invention are administered via intraperitoneal injection 3 h prior to a drinking session, and EtOH and H₂O consumption is monitored. Effective compounds of the invention robustly reduce binge drinking during the first 4 h, decreasing EtOH consumption. With exemplary compounds, consumption of ethanol is lower for at least two days following administration with no effect on water intake. Efficacy in this assay suggests the present compounds are useful for the treatment of AUD.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A compound of Formula (I): 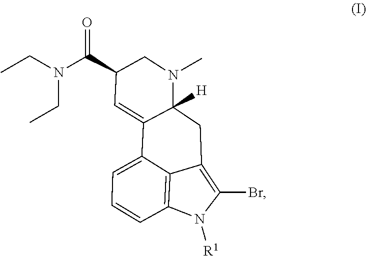
or a pharmaceutically acceptable salt thereof, wherein:
R¹ is —C(O)OR³, —C(O)R⁴, —CH(R⁵)OR⁶, —C(O)OCH(R⁵)OC(O)R⁴, —C(O)OCH(R⁵)OC(O)OR⁴, —C(O)OCH(R⁵)OC(O)NHR⁴, —CH(R⁵)NHC(O)R⁶, —CH(R⁵)C(O)R⁶, —S(O)₂R⁷, —S(O)₂OR⁷, —P(O)OR⁸[N(R⁹)R¹⁰], —C(O)N(R⁹)R¹⁰, —P(O)OR¹¹(OR¹²), —CH(R⁴)OP(O)OR⁸[N(R⁹)R¹⁰], —CH(R⁴)OP(O)OR¹¹(OR¹²), —C(O)OCH(R⁵)OP(O)OR²⁰(OR²¹);
each of R³, R⁴, R⁵, R⁶, R⁷, and R⁸ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A},
or R³ is 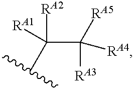
wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁵)R¹⁹, —N(R¹³)C(O)OR¹⁴, —N(R¹³)C(O)R¹⁴, —C(O)R¹⁴, —OC(O)R¹⁵, or —OC(O)OR¹⁶,
or R⁴ is 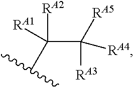
wherein each of R^{A1} and R^{A2} is independently hydrogen, alkyl, cycloalkyl, aryl or heteroaryl, or R^{A1} and R^{A2} together with the atom to which they are attached form a cycloalkyl ring; each of R^{A3} and R^{A4} is independently hydrogen, alkyl, cycloalkyl, aryl or heteroaryl, or R^{A3} and R^{A4} together with the atom to which they are attached form a cycloalkyl ring; and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.
or R⁴ is 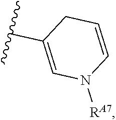
wherein R^{A7} is hydrogen or alkyl;
or R⁴ is —CH(R^{A1})NH₂, wherein R^{A1} is an amino acid side chain;
each of R⁹ and R¹⁰ is independently hydrogen, alkyl, heteroalkyl, haloalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}, or R⁹ and R¹⁰ together with the atom to which they are attached form a heterocycloalkyl ring or a heteroaryl ring that is unsubstituted or substituted with one or more R^{A};
or R⁹ is 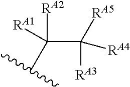
wherein each of R^{A1}, R^{A2}, R^{A3}, and R^{A4} is independently hydrogen or alkyl, and R^{A5} is heteroalkyl, heterocycloalkyl, heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶;
each of R¹¹ and R¹² is independently hydrogen, alkyl, heteroalkyl, cycloalkyl, heterocycloalkyl, aryl, heteroaryl, —CH(R⁵)C(O)R¹⁴, —CH(R⁵)C(O)OR¹, —CH(R⁵)OC(O)R¹⁵, or —CH(R⁵)OC(O)OR¹⁶, wherein alkyl, heteroalkyl, cycloalkyl, heterocycloalkyl, aryl, or heteroaryl is unsubstituted or substituted with one or more R^{A}, or R¹¹ and R¹² together with the atoms to which they are attached form a heterocycloalkyl ring that is unsubstituted or substituted with one or more R^{A};
each R^{A} is independently alkyl, heteroalkyl, cycloalkyl, heterocycloalkyl, aryl, heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or —S(O)₂R²², wherein alkyl, heteroalkyl, cycloalkyl, heterocycloalkyl, aryl, or heteroaryl is unsubstituted or substituted with alkyl, aryl, halogen, oxo, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B};
or R¹⁴ or R¹⁶ is 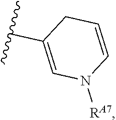
wherein R^{A7} is hydrogen or alkyl;
or R¹⁵ is —CH(R^{A1})NH₂, wherein R^{A1} is an amino acid side chain;
each of R¹⁸ and R¹⁹ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B}; or R¹⁸ and R¹⁹ together with the atom to which they are attached form a heterocycloalkyl ring or heteroaryl ring, each of which is unsubstituted or substituted with one or more R^{B};
each of R²⁰, R²¹ and R²² is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{B} or R²⁰ and R²¹ together with the atoms to which they are attached form a heterocycloalkyl ring that is unsubstituted or substituted with one or more R^{B};
each R^{B} is independently halogen, amino, cyano, hydroxyl, alkyl, heteroalkyl, cycloalkyl, heterocycloalkyl, aryl, heteroaryl, arylalkyl, acyl or heteroarylalkyl, wherein cycloalkyl, heterocycloalkyl, acyl, aryl, or heteroaryl is unsubstituted or substituted with one or more halogen, amino, cyano, hydroxyl, alkyl, acetyl, or benzoyl; and
each R^{C} is independently hydrogen or alkyl

2. The compound of claim 1, wherein the compound is a compound of Formula (Ia): 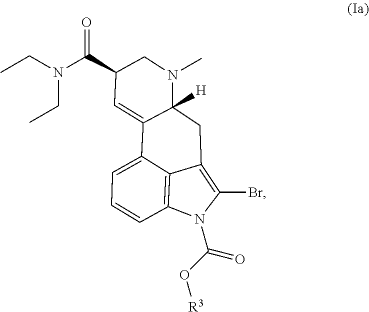
or a pharmaceutically acceptable salt thereof, wherein:
R³ is alkyl, alkenyl, haloalkyl, heteroalkyl, cycloalkyl, heterocycloalkyl, aryl, or heteroaryl, each of which is substituted or unsubstituted.

3. The compound of claim 1 or claim 2, wherein the compound is a compound of Formula (Ia1): 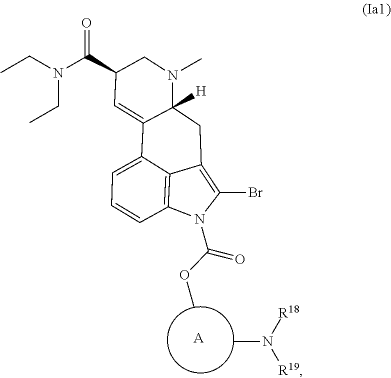
or a pharmaceutically acceptable salt thereof, wherein: 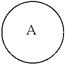
is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

4. The compound of claim 3, wherein the compound is a compound of Formula (Ia2): 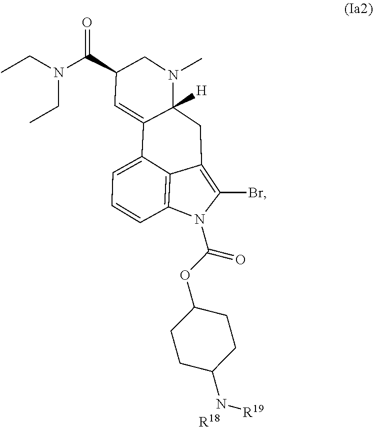
or a pharmaceutically acceptable salt thereof, wherein:
each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

5. The compound of claim 1, wherein the compound is a compound of Formula (Ia3), or a pharmaceutically acceptable salt thereof:

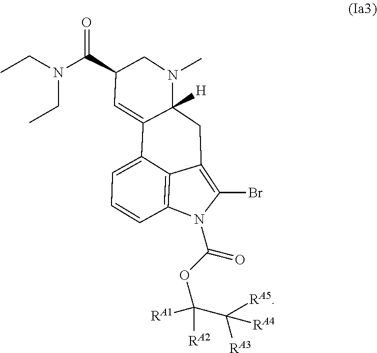

6. The compound of claim 2, wherein the compound is: 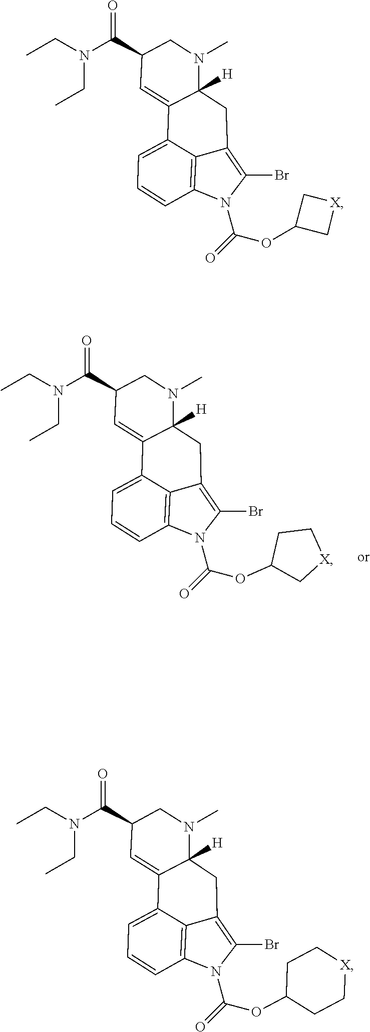
or a pharmaceutically acceptable salt thereof, wherein:
each X is independently —O—, —S—, —S(O)—, —S(O)₂—, —NH—, or —NR^{A}.

7. The compound of claim 2, or a pharmaceutically acceptable salt thereof, wherein R³ is alkyl or cycloalkyl, each of which is optionally substituted with one or two R^{A}.

8. The compound of claim 2, wherein the compound is: 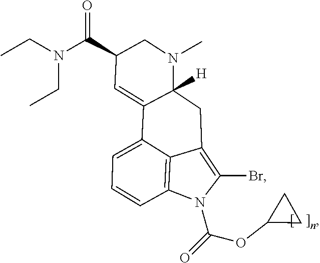
or a pharmaceutically acceptable salt thereof, wherein:
n is an integer from 1 to 6.

9. The compound of claim 2, wherein the compound is: 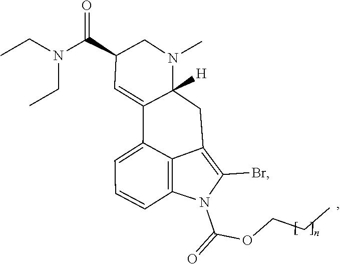
or a pharmaceutically acceptable salt thereof, wherein:
n is an integer from 1 to 20.

10. The compound of claim 1, wherein the compound is a compound of Formula (Ib): 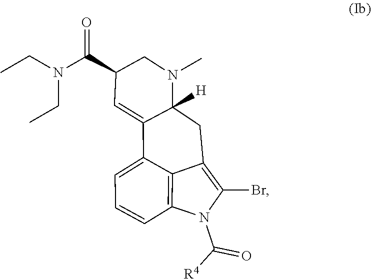
or a pharmaceutically acceptable salt thereof, wherein:
R⁴ is alkyl, alkenyl, haloalkyl, heteroalkyl, cycloalkyl, heterocycloalkyl, aryl, or heteroaryl, each of which is substituted or unsubstituted.

11. The compound of claim 10, or a pharmaceutically acceptable salt thereof, wherein

12. R⁴ is —CH(R^{A1})NH₂, wherein R^{A1} is an amino acid side chain. The compound of claim 11, or a pharmaceutically acceptable salt thereof, wherein the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

13. The compound of claim 10, wherein the compound is a compound of Formula (Ib1):

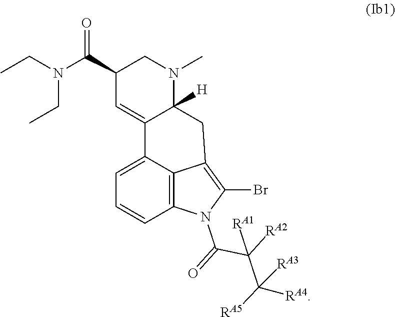

14. The compound of claim 10, wherein the compound is: 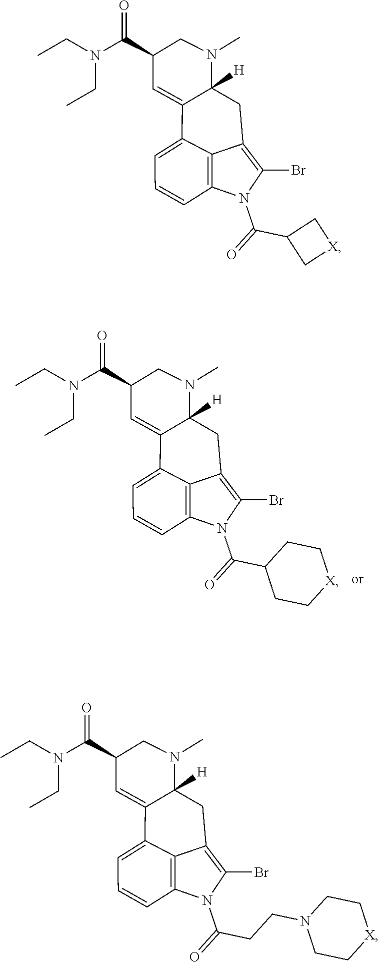
or a pharmaceutically acceptable salt thereof, wherein:
X is independently —O—, —S—, —S(O)—, —S(O)₂—, —NH—, or —NR^{A}.

15. The compound of claim 10, wherein the compound is: 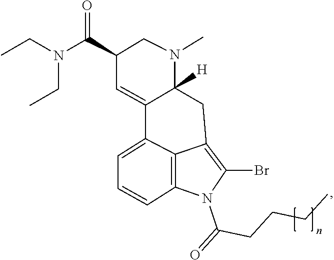
or a pharmaceutically acceptable salt thereof, wherein:
n is an integer from 1 to 20.

16. The compound of claim 1, wherein the compound is a compound of Formula (Ic): 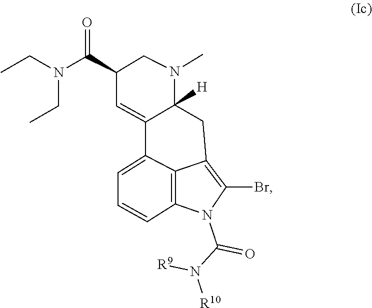
or a pharmaceutically acceptable salt thereof, wherein:
each of R⁹ and R¹⁰ is independently hydrogen, alkyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A} or R⁹ and R¹⁰ together with the atom to which they are attached form a heterocycloalkyl ring or a heteroaryl ring that is unsubstituted or substituted with one or more R^{A}.

17. The compound claim 16, or a pharmaceutically acceptable salt thereof, wherein:
R⁹ is haloalkyl.

18. The compound of claim 16, wherein the compound is a compound of Formula (Ic1): 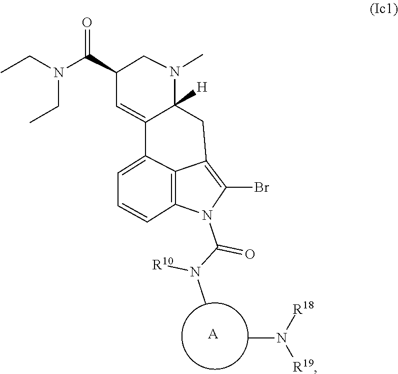
or a pharmaceutically acceptable salt thereof wherein: 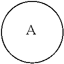
is cycloalkyl or heterocycloalkyl, and each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

19. The compound of claim 18, wherein the compound is a compound of Formula (Ic2): 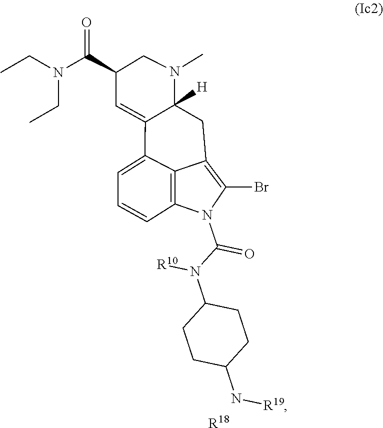
or a pharmaceutically acceptable salt thereof, wherein:
each of R¹⁸ and R¹⁹ is independently hydrogen, alkyl, cycloalkyl, or heteroalkyl; or R¹⁸ and R¹⁹ together with the atom to which they are attached form a heterocycloalkyl ring.

20. The compound of claim 1, wherein the compound is a compound of Formula (Ic3), or a pharmaceutically acceptable salt thereof:

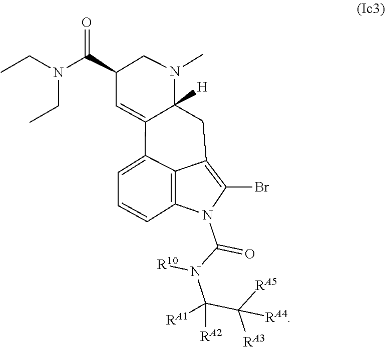

21. The compound of claim 16, wherein the compound is: 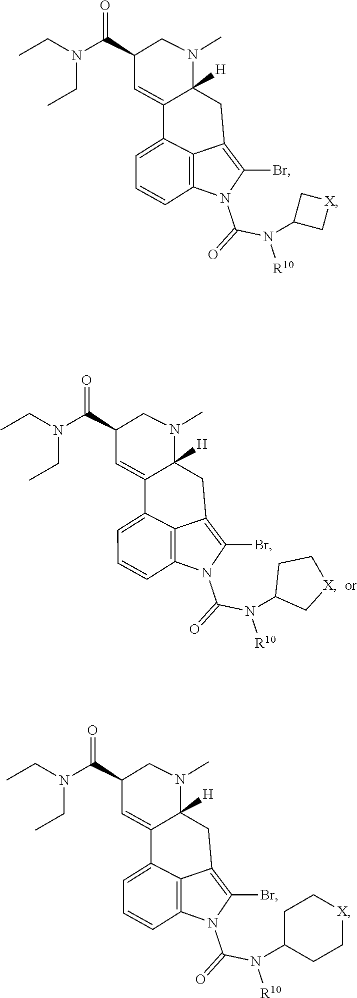
or a pharmaceutically acceptable salt thereof, wherein:
each X is independently —O—, —S—, —S(O)—, —S(O)₂—, —NH—, or —NR^{A}.

22. The compound of claim 16, wherein the compound is: 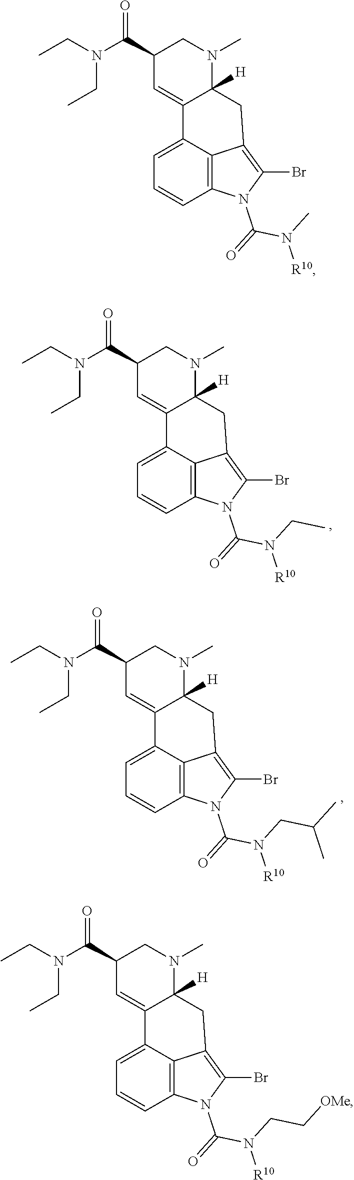 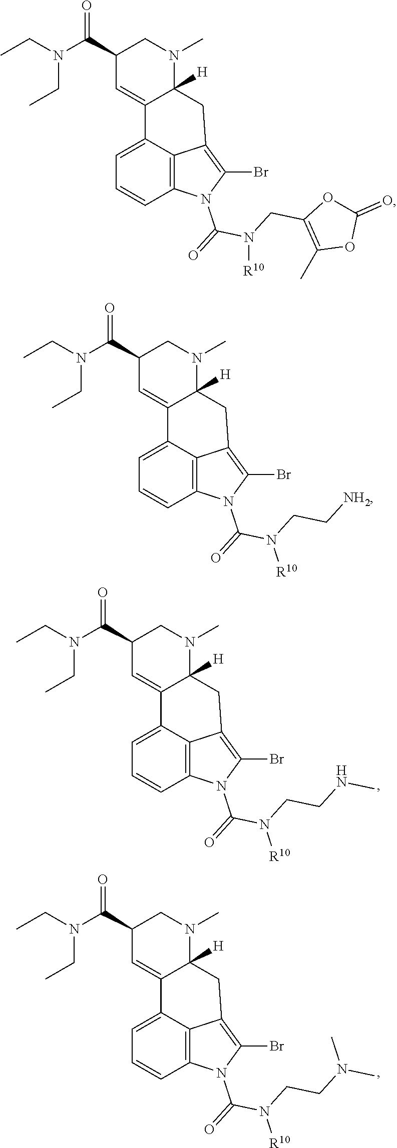 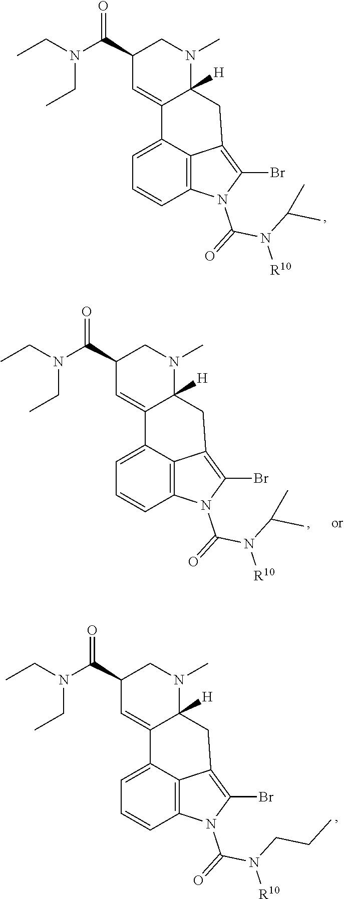
or a pharmaceutically acceptable salt thereof.

23. The compound of claim 16, wherein the compound is: 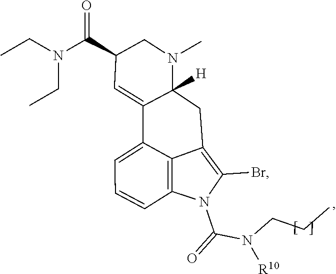
or a pharmaceutically acceptable salt thereof, wherein:
n is an integer from 1 to 20.

24. The compound of claim 1, wherein the compound is a compound of Formula (Id), or a pharmaceutically acceptable salt thereof:

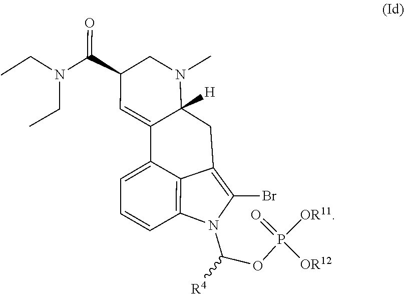

25. The compound of claim 24, wherein the compound is a compound of Formula (Id1), or a pharmaceutically acceptable salt thereof:

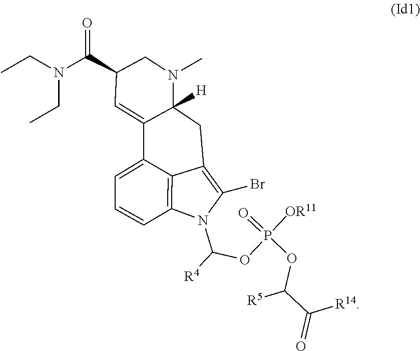

26. The compound of claim 24, wherein the compound is a compound of Formula (Id2), or a pharmaceutically acceptable salt thereof:

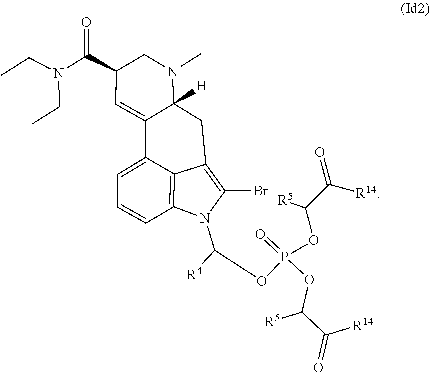

27. The compound of claim 24 or claim 26, or a pharmaceutically acceptable salt thereof, wherein R¹⁴ is

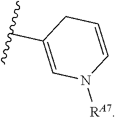

28. The compound of claim 24, wherein the compound is a compound of Formula (Id3), or a pharmaceutically acceptable salt thereof:

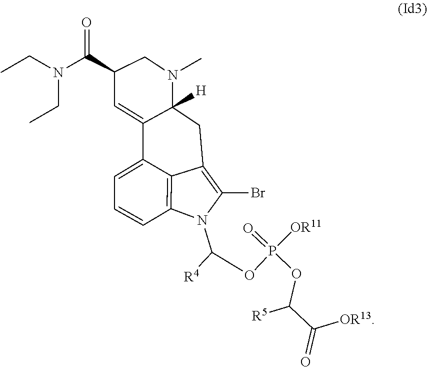

29. The compound of claim 24, wherein the compound is a compound of Formula (Id4), or a pharmaceutically acceptable salt thereof:

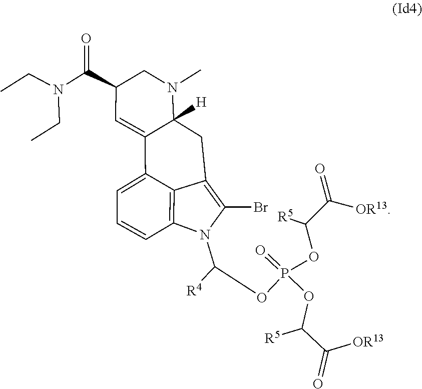

30. The compound of claim 1, wherein the compound is a compound of Formula (Ie), or a pharmaceutically acceptable salt thereof:

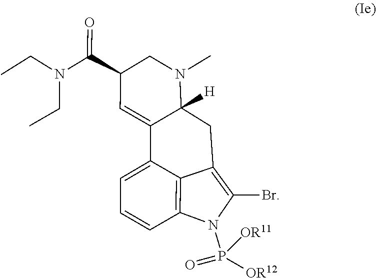

31. The compound of claim 30, wherein the compound is a compound of Formula (Ie1), or a pharmaceutically acceptable salt thereof:

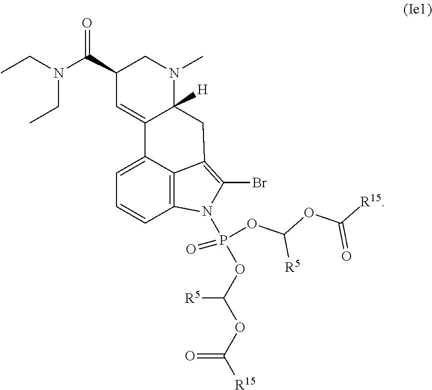

32. The compound of claim 30, wherein the compound is a compound of Formula (Ie2), or a pharmaceutically acceptable salt thereof:

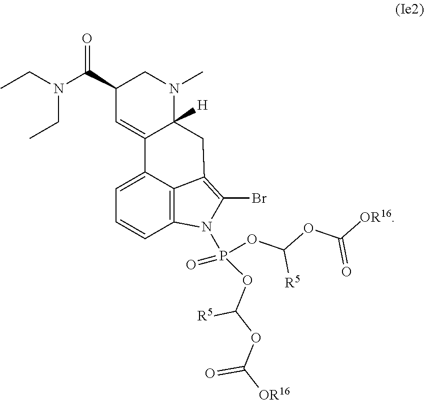

33. The compound of claim 30, or a pharmaceutically acceptable salt thereof, wherein:
R¹¹ and R¹² together with the atoms to which they are attached form a heterocycloalkyl ring that is unsubstituted or substituted with one or more R^{A}.

34. The compound of claim 33, or a pharmaceutically acceptable salt thereof, wherein:
the heterocycloalkyl ring is substituted with R^{A}, wherein R^{A} is aryl.

35. The compound of claim 35, or a pharmaceutically acceptable salt thereof, wherein:
R^{A} is substituted with halogen.

36. The compound of claim 32, or a pharmaceutically acceptable salt thereof, wherein: R¹⁶ is

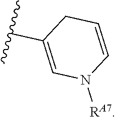

37. The compound of claim 1, wherein the compound is a compound of Formula (If) 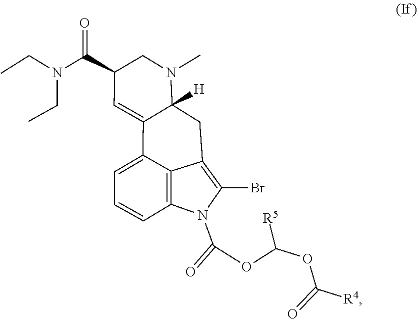
or a pharmaceutically acceptable salt thereof, wherein:

38. R⁴ is alkyl, alkenyl, haloalkyl, cycloalkyl, heterocycloalkyl, aryl, heteroaryl, or heteroalkyl, each of which is substituted or unsubstituted; and R⁵ is alkyl, or cycloalkyl, each of which is substituted or unsubstituted, or hydrogen. The compound of claim 37, or a pharmaceutically acceptable salt thereof, wherein:
R⁴ is

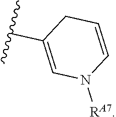

39. The compound of claim 37, wherein the compound is: 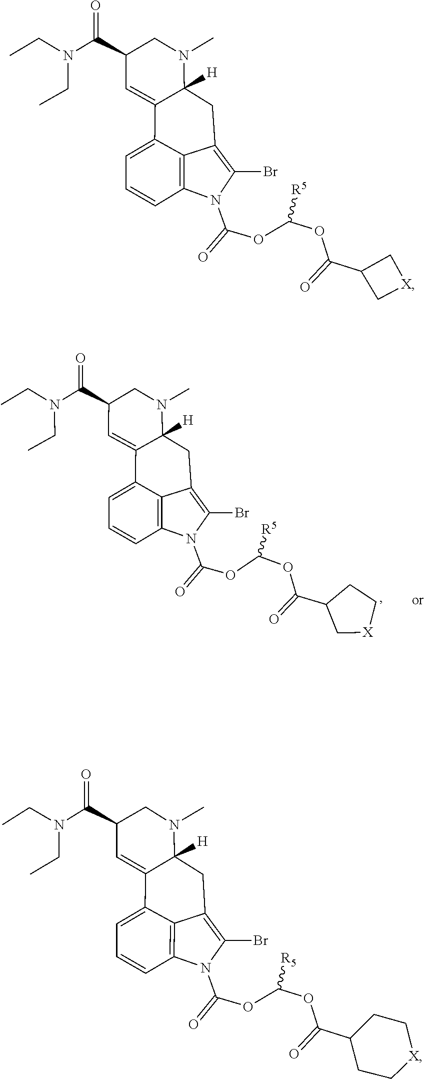
or a pharmaceutically acceptable salt thereof, wherein:
each X is independently —O—, —S—, —S(O)—, —S(O)₂—, —NH—, or —NR^{A}.

40. The compound of claim 37, or a pharmaceutically acceptable salt thereof, wherein:
R⁴ is —CH(R^{A1})NH₂, wherein R^{A1} is an amino acid side chain.

41. The compound of claim 40, or a pharmaceutically acceptable salt thereof, wherein the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

42. The compound of claim 1, wherein the compound is a compound of Formula (Ig): 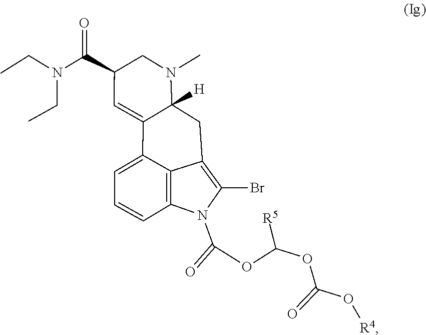
or a pharmaceutically acceptable salt thereof, wherein:
R⁴ is selected from alkyl, alkenyl, heteroalkyl, cycloalkyl, haloalkyl, heterocycloalkyl, aryl, and heteroaryl, each of which is substituted or unsubstituted; and
R⁵ is alkyl that is substituted or unsubstituted, or hydrogen.

43. The compound of claim 42, or a pharmaceutically acceptable salt thereof, wherein:
R⁴ is selected from 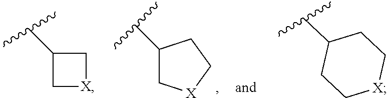
and each X is independently —O—, —S—, —S(O)—, —S(O)₂—, —NH—, or —NR^{A}.

44. The compound of claim 42, or a pharmaceutically acceptable salt thereof, wherein:
R⁴ is selected from

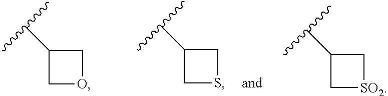 [SMILES: *C1COC1 ; *C1CSC1 ; *C1CS(=O)(=O)C1]

45. The compound of claim 42, or a pharmaceutically acceptable salt thereof, wherein:
R⁴ is 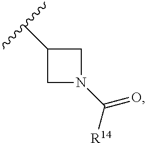
and R¹⁴ is alkyl, cycloalkyl, or aryl, each of which is substituted or unsubstituted with one or more R^{B}.

46. The compound of claim 45, or a pharmaceutically acceptable salt thereof, wherein:
R¹⁴ is selected from methyl, ethyl, n-propyl, isopropyl, phenyl and CH₂CH₂OMe.

47. The compound of claim 42, or a pharmaceutically acceptable salt thereof, wherein:
R⁴ is

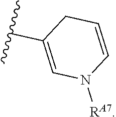

48. The compound of claim 1, wherein the compound is a compound of Formula (Ih): 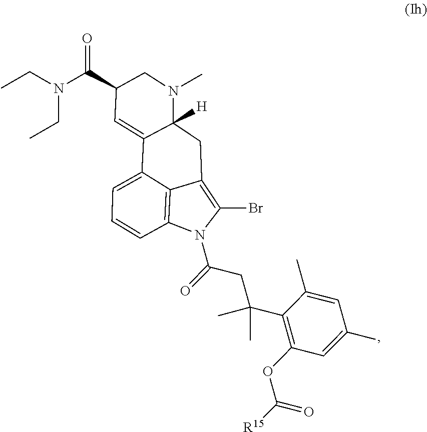
or a pharmaceutically acceptable salt thereof, wherein R¹⁵ is selected from alkyl, alkenyl, haloalkyl, heteroalkyl, cycloalkyl, heterocycloalkyl, aryl, and heteroaryl.

49. The compound of claim 48, or a pharmaceutically acceptable salt thereof, wherein:
R¹⁵ is —CH(R^{A1})NH₂, wherein R^{A1} is an amino acid side chain.

50. The compound of claim 49, or a pharmaceutically acceptable salt thereof, wherein the amino acid side chain is —CH₃, —CH₂CH₂CH₂—NH—C(═NH)NH₂, —CH₂C(O)NH₂, —CH₂CO₂H, —CH₂SH, —CH₂CH₂C(O)NH₂, —CH₂CH₂CO₂H, —H, —CH₂-(2-pyrrole), —CH(CH₃)CH₂CH₃, —CH₂—CH(CH₃)₂, —CH₂CH₂CH₂CH₂—NH₂, —CH₂CH₂SCH₃, —CH₂phenyl, —CH₂OH, —CH(OH)CH₃, —CH₂-(3-indole), —CH₂(4-hydroxyphenyl), or —CH₂(CH₃)₂.

51. The compound of claim 1, wherein the compound is a compound of Formula (Ii): 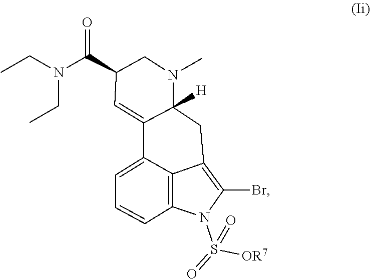
or a pharmaceutically acceptable salt thereof,
wherein R⁷ is independently hydrogen, alkyl, alkenyl, haloalkyl, heteroalkyl, cycloalkyl, heterocycloalkyl, aryl, or heteroaryl, wherein alkyl, heteroalkyl, cycloalkyl, heterocycloalkyl, aryl, or heteroaryl is unsubstituted or substituted with one or more R^{A}.

52. The compound of claim 51, wherein the compound is a compound of Formula (Ii1): 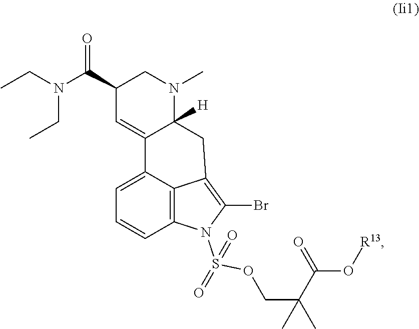
or a pharmaceutically acceptable salt thereof, wherein:
R¹³ is independently hydrogen, alkyl, heteroalkyl, cycloalkyl, heterocycloalkyl, aryl, or heteroaryl, wherein alkyl, heteroalkyl, cycloalkyl, heterocycloalkyl, aryl, and heteroaryl is unsubstituted or substituted with one or more R^{B}.

53. The compound of claim 52, or a pharmaceutically acceptable salt thereof, wherein:
R¹³ is selected from 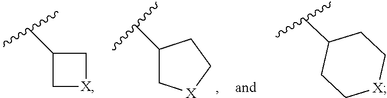
and
each X is independently —O—, —S—, —S(O)—, —S(O)₂—, —NH—, or —NR^{B}.

54. The compound of claim 51, wherein the compound is a compound of Formula (Ii2): 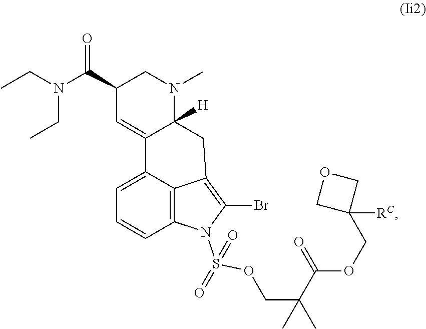
or a pharmaceutically acceptable salt thereof, wherein:
R^{C} is selected from hydrogen and alkyl.

55. The compound of claim 1, wherein the compound is a compound of Formula (Ij): 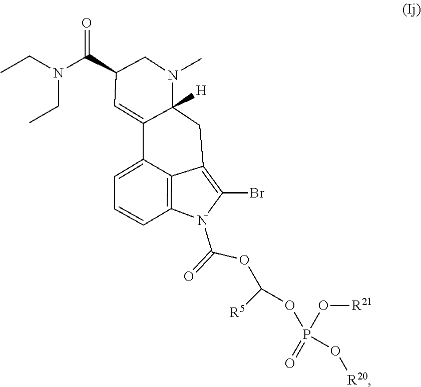
or a pharmaceutically acceptable salt thereof, wherein:
each R^{B} is independently halogen, amino, cyano, hydroxyl, alkyl, heteroalkyl, cycloalkyl, heterocycloalkyl, acyl, aryl, heteroaryl, arylalkyl, or heteroarylalkyl.

56. The compound of claim 1, or a pharmaceutically acceptable salt thereof, wherein:
each of R³, R⁴, R⁵, R⁶, R⁷, and R⁸ is independently hydrogen, C₁-C₁₀ alkyl, C₂-C₁₀ alkenyl, C₁-C₆ haloalkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A};
or R³ is 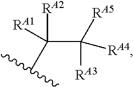
wherein each of R^{A1}, R^{A2}, R^{A3}, and R^{A4} is independently hydrogen or C₁-C₁₀ alkyl; and R^{A5} is C₃-C₆ heteroalkyl, 3- to 6-membered heterocycloalkyl, monocyclic heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶;
or R⁴ is 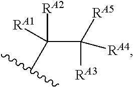
wherein each of R^{A1} and R^{A2} is independently hydrogen, C₁-C₁₀ alkyl, C₃-C₆ cycloalkyl, phenyl, or monocyclic heteroaryl, or R^{A1} and R^{A2} together with the atom to which they are attached form a C₃-C₆ cycloalkyl ring; each of R^{A3} and R^{A4} is independently hydrogen, C₁-C₁₀ alkyl, C₃-C₆ cycloalkyl, phenyl, or monocyclic heteroaryl, or R^{A3} and R^{A4} together with the atom to which they are attached form a C₃-C₆ cycloalkyl ring; and R^{A5} is C₃-C₆ heteroalkyl, 3- to 6-membered heterocycloalkyl, monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶;
or R⁴ is 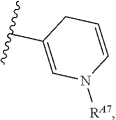
wherein R^{A7} is hydrogen or C₁-C₁₀ alkyl;
or R⁴ is —CH(R^{A1})NH₂, wherein R^{A1} is an amino acid side chain;
each of R⁹ and R¹⁰ is independently hydrogen, C₁-C₁₀ alkyl, C₃-C₆ heteroalkyl, C₁-C₆ haloalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{A}, or R⁹ and R¹⁰ together with the atom to which they are attached form a 3- to 6-membered heterocycloalkyl ring or a heteroaryl ring that is unsubstituted or substituted with one to five R^{A};
or R⁹ is 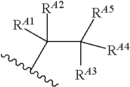
wherein each of R^{A1}, R^{A2}, R^{A3}, and R^{A4} is independently hydrogen or C₁-C₁₀ alkyl, and R^{A5} is C₃-C₆ heteroalkyl, 3- to 6-membered heterocycloalkyl, monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶;
each of R¹¹ and R¹² is independently hydrogen, C₁-C₁₀ alkyl, C₃-C₆ heteroalkyl, C₃-C₈ cycloalkyl, 3- to 6-membered heterocycloalkyl, phenyl, monocyclic heteroaryl, —CH(R⁵)C(O)R¹⁴, —CH(R⁵)C(O)OR¹³, —CH(R⁵)OC(O)R¹⁶, or —CH(R⁵)OC(O)OR¹⁶, wherein alkyl, heteroalkyl, cycloalkyl, heterocycloalkyl, phenyl, or heteroaryl is unsubstituted or substituted with one to five R^{A}, or R¹¹ and R¹² together with the atoms to which they are attached form a 3- to 6-membered heterocycloalkyl ring that is unsubstituted or substituted with one to five R^{A};
each R^{A} is independently C₁-C₁₀ alkyl, C₃-C₆ heteroalkyl, C₃-C₈ cycloalkyl, 3- to 6-membered heterocycloalkyl, phenyl, monocyclic heteroaryl, an amino acid side chain, —R⁵, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or S(O)₂R²², wherein alkyl, heteroalkyl, cycloalkyl, heterocycloalkyl, phenyl, or heteroaryl is unsubstituted or substituted with C₁-C₆ alkyl, phenyl, halogen, oxo, —OR¹³, —NR(R¹⁸)R¹⁹, —C(O)R¹⁴, —OC(O)R¹⁵, —OC(O)OR¹⁶, or —OC(O)N(R¹⁸)R¹⁹;
each of R¹³, R¹⁴, R¹⁵, R¹⁶, or R¹⁷ is independently hydrogen, C₁-C₁₀ alkyl, C₃-C₆ heteroalkyl, C₃-C₈ cycloalkyl, 3- to 6-membered heterocycloalkyl, phenyl, or monocyclic heteroaryl, wherein alkyl, heteroalkyl, cycloalkyl, heterocycloalkyl, phenyl, and heteroaryl is unsubstituted or substituted with one to five R^{B};
or R¹⁴ or R¹⁶ is 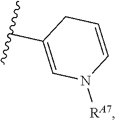
wherein R^{A7} is hydrogen or C₁-C₁₀ alkyl;
or R¹⁵ is —CH(R^{A1})NH₂, wherein R^{A1} is an amino acid side chain;
each of R¹⁸ and R¹⁹ is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}; or R¹⁸ and R¹⁹ together with the atom to which they are attached form a 3- to 6-membered heterocycloalkyl ring or heteroaryl ring, each of which is unsubstituted or substituted with one to five R^{B};
each of R²⁰ and R²¹ is independently hydrogen, C₁-C₁₀ alkyl, C₃-C₆ heteroalkyl, C₃-C₅ cycloalkyl, 3- to 6-membered heterocycloalkyl, phenyl, or monocyclic heteroaryl, wherein alkyl, heteroalkyl, cycloalkyl, heterocycloalkyl, phenyl, or heteroaryl is unsubstituted or substituted with one to five R^{B}, or R²⁰ and R²¹ together with the atoms to which they are attached form a 3- to 6-membered heterocycloalkyl ring that is unsubstituted or substituted with one to five R^{B};
each R^{B} is independently halogen, amino, cyano, hydroxyl, C₁-C₁₀ alkyl, C₃-C₆ heteroalkyl, C₃-C₈ cycloalkyl, 3- to 6-membered heterocycloalkyl, phenyl, monocyclic heteroaryl, benzyl, —C(O)CH₃, —C(O)Ph, or (monocyclic heteroaryl)-C₁-C₄ alkyl wherein cycloalkyl, heterocycloalkyl, phenyl, —C(O)CH₃, or heteroaryl is unsubstituted or substituted with one to five halogen, amino, cyano, hydroxyl, C₁-C₆ alkyl, C₁-C₆ acetyl, or benzoyl; and
each R^{C} is independently hydrogen or C₁-C₁₀ alkyl.

57. The compound of claim 1, or a pharmaceutically acceptable salt thereof, wherein:
each of R³, R⁴, R⁵, R⁶, R⁷, and R⁸ is independently C₁-C₆ alkyl, C₂-C₆ alkenyl, C₁-C₆ haloalkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A};
or R³ is 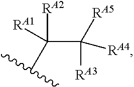
wherein each of R^{A1}, R^{A2}, R^{A3}, and R^{A4} is independently hydrogen or C₁-C₆ alkyl; and R^{A5} is C₃-C₆ heteroalkyl, 3- to 6-membered heterocycloalkyl, 5-membered monocyclic heteroaryl, 6-membered monocyclic heteroaryl, or —C(O)OR¹³, —N(R¹³)C(O)OR¹⁴, —N(R¹³)C(O)R¹⁴, —C(O)R¹⁴, —OC(O)R¹⁵, or —OC(O)OR¹⁶;
or R⁴ is 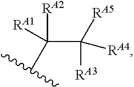
wherein each of R^{A1} and R^{A2} is independently hydrogen, C₁-C₆ alkyl, C₃-C₆ cycloalkyl, phenyl, 5-membered monocyclic heteroaryl, or 6-membered monocyclic heteroaryl, or R^{A1} and R^{A2} together with the atom to which they are attached form a C₃-C₆ cycloalkyl ring; each of R^{A3} and R^{A4} is independently hydrogen, C₁-C₆ alkyl, C₃-C₆ cycloalkyl, phenyl, 5-membered monocyclic heteroaryl, or 6-membered monocyclic heteroaryl, or R^{A3} and R^{A4} together with the atom to which they are attached form a C₃-C₆ cycloalkyl ring; and R^{A5} is C₃-C₆ heteroalkyl, 3- to 6-membered heterocycloalkyl, 5-membered monocyclic heteroaryl, 6-membered monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶.
or R⁴ is 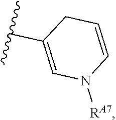
wherein R^{A7} is hydrogen or C₁-C₆ alkyl;
or R⁴ is —CH(R^{A1})NH₂, wherein R^{A1} is an amino acid side chain;
each of R⁹ and R¹⁰ is independently hydrogen, C₁-C₆ alkyl, C₃-C₆ heteroalkyl, C₁-C₆ haloalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{A}, or R⁹ and R¹⁰ together with the atom to which they are attached form a 3- to 6-membered heterocycloalkyl ring or a heteroaryl ring that is unsubstituted or substituted with one to three R^{A};
or R⁹ is 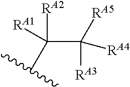
wherein each of R^{A1}, R^{A2}, R^{A3}, and R^{A4} is independently hydrogen or C₁-C₆ alkyl, and R^{A5} is C₃-C₆ heteroalkyl, 3- to 6-membered heterocycloalkyl, 5-membered, 6-membered monocyclic heteroaryl, —C(O)OR¹³, —NR(R¹⁸)R¹⁹, —N(R¹³)C(O)OR¹⁴, —N(R¹³)C(O)R¹⁴, —C(O)R¹⁴, —OC(O)R¹⁵, or —OC(O)OR¹⁶;
each of R¹¹ and R¹² is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, —CH(R⁵)C(O)R¹⁴, —CH(R⁵)C(O)OR¹, —CH(R⁵)OC(O)R¹⁵, or —CH(R⁵)OC(O)OR¹⁶, wherein alkyl, heteroalkyl, cycloalkyl, heterocycloalkyl, phenyl, or heteroaryl is unsubstituted or substituted with one to three R^{A}, or R¹ and R¹² together with the atoms to which they are attached form a 3- to 6-membered heterocycloalkyl ring that is unsubstituted or substituted with one to three R^{A};
each R^{A} is independently C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, an amino acid side chain, —OR¹³, —N(R¹⁸)R¹⁹, —C(O)OR¹³, —N(R¹³)C(O)OR¹⁴, —N(R¹³)C(O)R¹⁴, —C(O)R¹⁴, —OC(O)R¹⁵, —OC(O)OR¹⁶, —OP(O)OR¹⁷[N(R¹⁸)R¹⁹], —C(O)N(R¹⁸)R¹⁹, —OC(O)N(R¹⁸)R¹⁹, —OP(O)OR²⁰(OR²¹), or S(O)₂R²², wherein alkyl, heteroalkyl, cycloalkyl, heterocycloalkyl, phenyl, or heteroaryl is unsubstituted or substituted with C₁-C₆ alkyl, phenyl, halogen, oxo, —OR¹³, —NR(R¹⁸)R¹⁹, —C(O)R¹⁴, —OC(O)R¹⁵, —OC(O)OR¹⁶, or —OC(O)N(R¹⁵)R¹⁹;
each of R¹³, R¹⁴, R¹⁵, R¹⁶, or R¹⁷ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, and heteroaryl is unsubstituted or substituted with one to three R^{B};
or R¹⁴ or R¹⁶ is 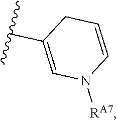
wherein R^{A7} is hydrogen or C₁-C₆ alkyl;
or R¹⁵ is —CH(R^{A1})NH₂, wherein R^{A1} is an amino acid side chain;
each of R¹⁸ and R¹⁹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}; or R¹⁸ and R¹⁹ together with the atom to which they are attached form a 3- to 6-membered heterocycloalkyl ring or heteroaryl ring, each of which is unsubstituted or substituted with one to three R^{B};
each of R²⁰ and R²¹ is independently hydrogen, C₁-C₆ alkyl, C₃-C₆ heteroalkyl, C₃-C₆ cycloalkyl, 3- to 6-membered heterocycloalkyl, phenyl, 5-membered monocyclic heteroaryl, or 6-membered monocyclic heteroaryl, wherein alkyl, heteroalkyl, cycloalkyl, heterocycloalkyl, phenyl, or heteroaryl is unsubstituted or substituted with one to three R^{B}, or R²⁰ and R²¹ together with the atoms to which they are attached form a 3- to 6-membered heterocycloalkyl ring that is unsubstituted or substituted with one to three R^{B};
each R^{B} is independently halogen, amino, cyano, hydroxyl, C₁-C₆ alkyl, C₃-C₆ heteroalkyl, C₃-C₆ cycloalkyl, 3- to 6-membered heterocycloalkyl, phenyl, 5-membered monocyclic heteroaryl, 6-membered monocyclic heteroaryl, benzyl, —C(O)CH₃, —C(O)Ph, or (5- or 6-membered monocyclic heteroaryl) —CH₂—, wherein cycloalkyl, heterocycloalkyl, phenyl, —C(O)CH₃, or heteroaryl is unsubstituted or substituted with one to three halogen, amino, cyano, hydroxyl, C₁-C₆ alkyl, C₁-C₆ acetyl, or benzoyl; and
each R^{C} is independently hydrogen or C₁-C₆ alkyl.

58. The compound of claim 1, wherein the compound is: 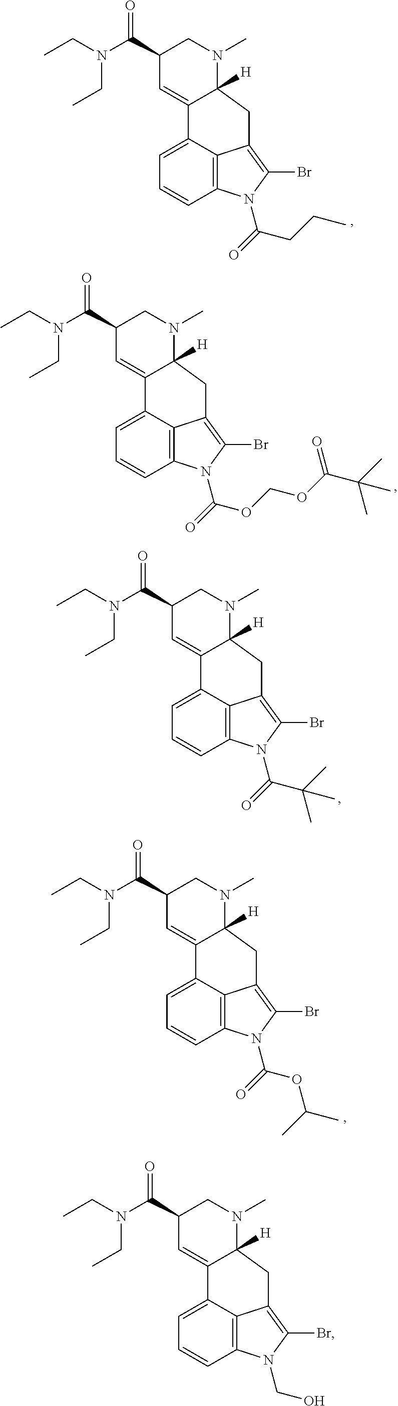 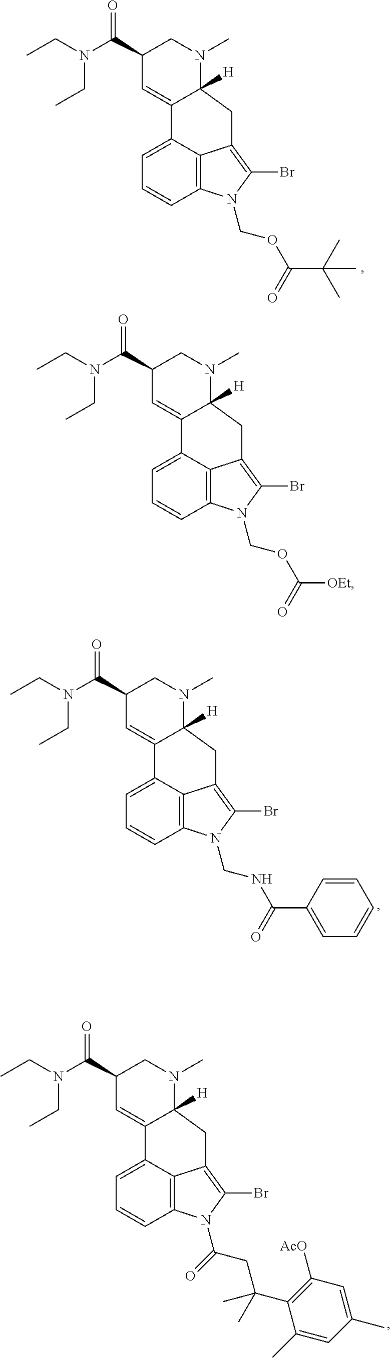 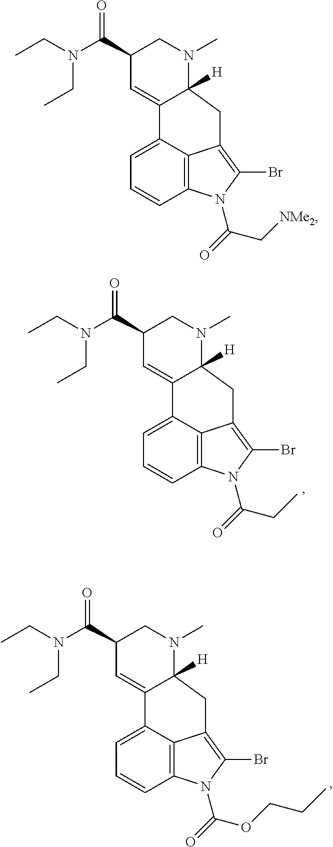 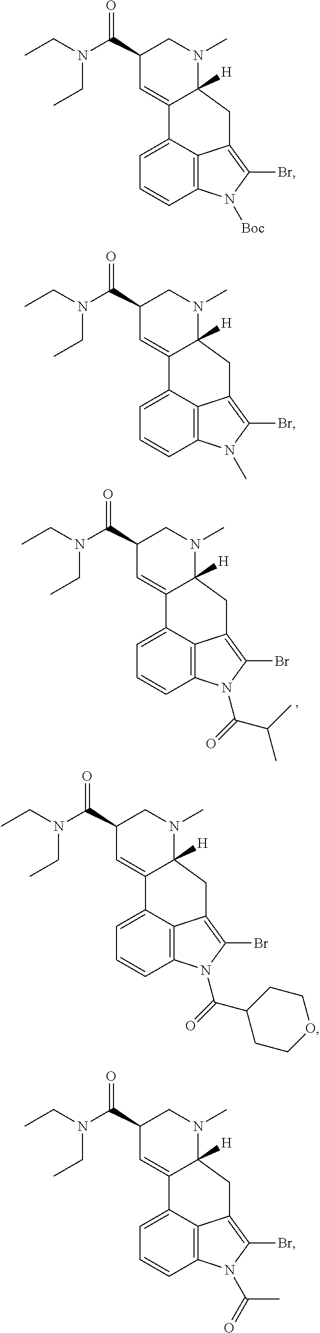 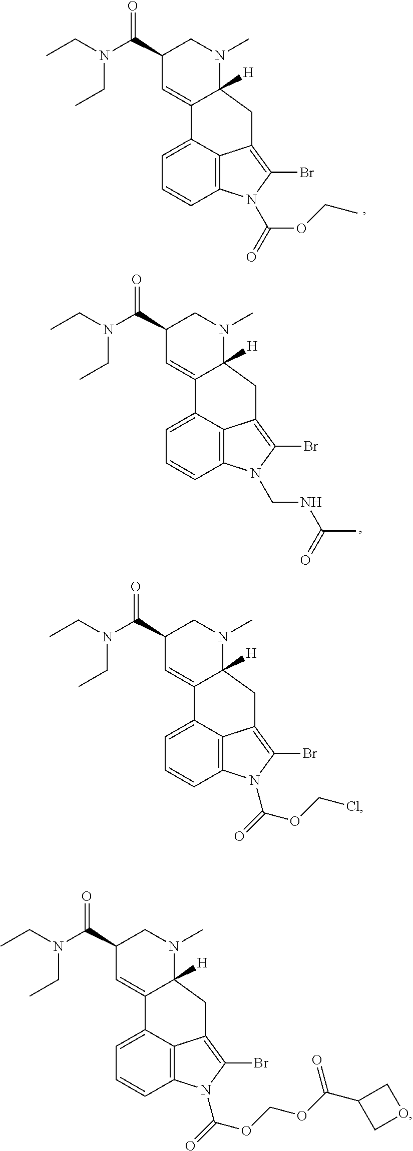 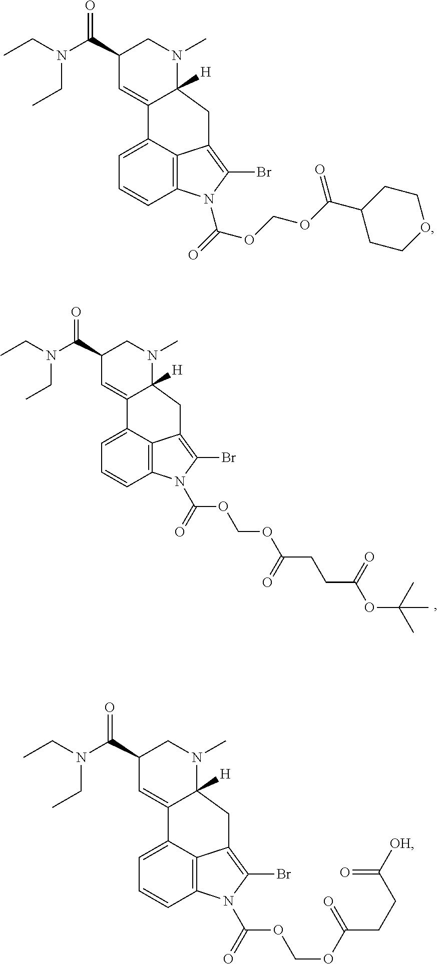 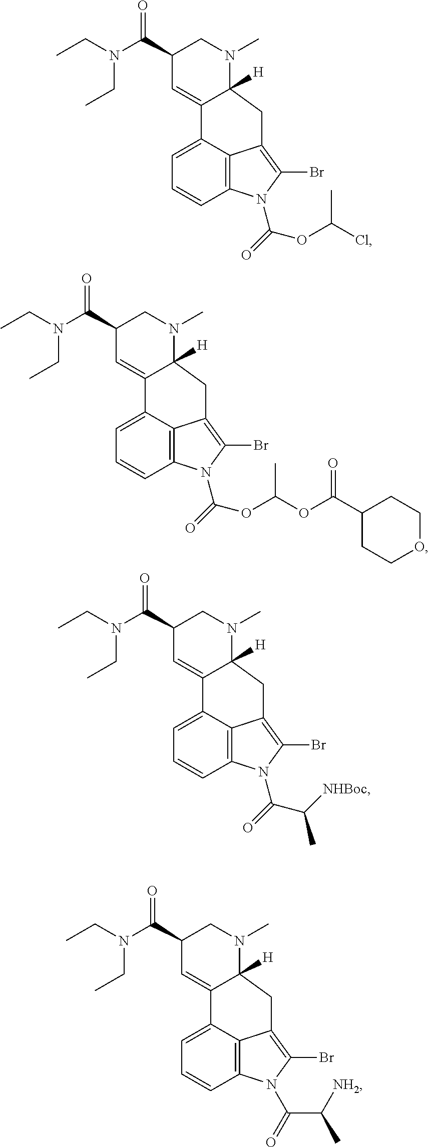 [SMILES: CCN(CC)C(=O)[C@@H]1C=C2c3cccc4c3c(c(Br)n4C(=O)OC(C)Cl)C[C@H]2N(C)C1 ; CCN(CC)C(=O)[C@@H]1C=C2c3cccc4c3c(c(Br)n4C(=O)OC(C)OC(=O)C3CCOCC3)C[C@H]2N(C)C1 ; CCN(CC)C(=O)[C@@H]1C=C2c3cccc4c3c(c(Br)n4C(=O)[C@H](C)NC(=O)OC(C)(C)C)C[C@H]2N(C)C1 ; CCN(CC)C(=O)[C@@H]1C=C2c3cccc4c3c(c(Br)n4C(=O)[C@H](C)N)C[C@H]2N(C)C1] 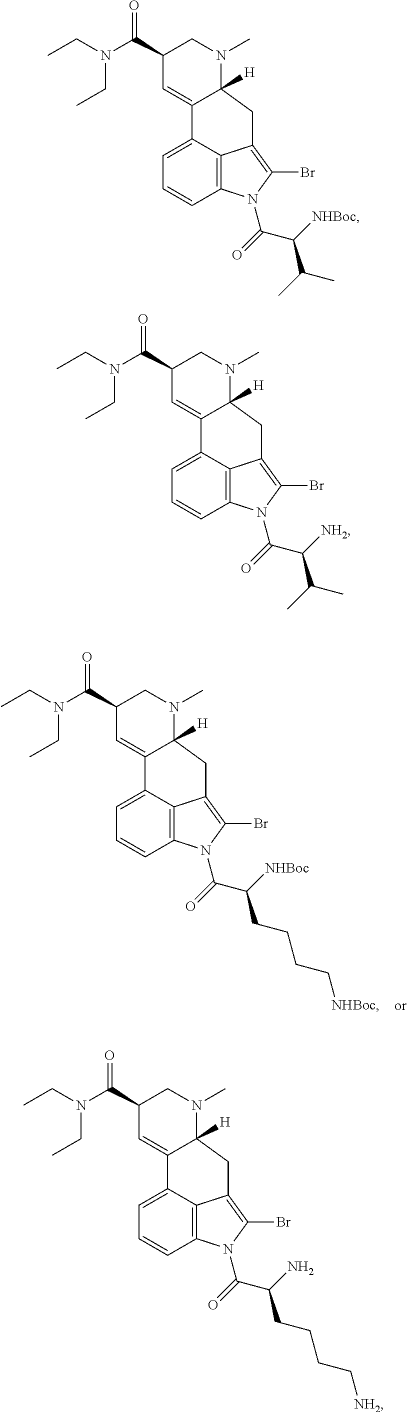
or a pharmaceutically acceptable salt thereof.

59. The compound of any one of claims 1-58, wherein the compound is enriched in deuterium.

60. The compound of claim 1, wherein the compound is: 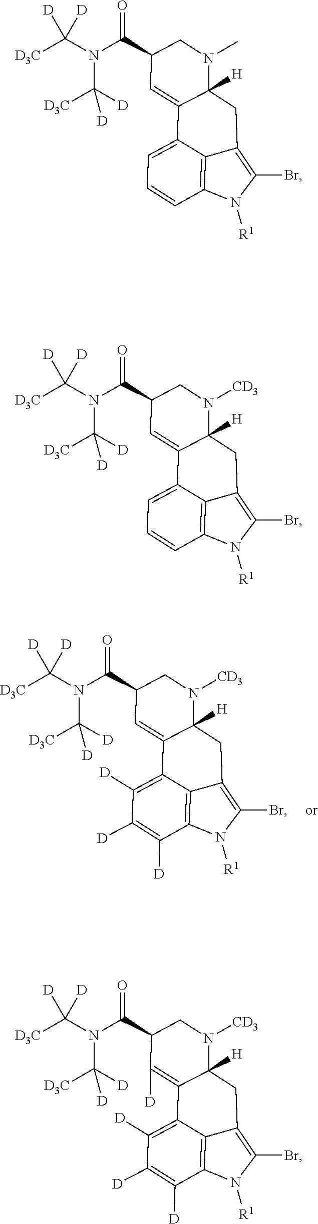
or a pharmaceutically acceptable salt thereof.

61. The compound of any one of claims 1-60, wherein the compound is in the form of a pharmaceutically acceptable salt.

62. A solvate of the compound of any one of claims 1-60, or a pharmaceutically acceptable salt thereof.

63. A pharmaceutical composition comprising a compound of any one of claims 1-60, or a pharmaceutically acceptable salt thereof.

64. A method for increasing neuronal plasticity, comprising contacting a neuron with an effective amount of a compound according to any one of claims 1-60, or a pharmaceutically acceptable salt thereof, or a pharmaceutical composition according to claim 63.

65. The method of claim 64, wherein contacting comprises administering the compound to a subject.

66. A method for treating a neurological disorder, a psychiatric disorder, or both, comprising contacting a subject having the neurological disorder, psychiatric disorder or both with an effective amount of a compound according to any one of claims 1-60, or a pharmaceutically acceptable salt thereof, or a pharmaceutical composition according to claim 63.

67. The method of claim 66, wherein the neurological disorder is a neurodegenerative disorder.

68. The method of claim 66, wherein the neurological disorder, psychiatric disorder, or both, comprises depression, addiction, anxiety, or a post-traumatic stress disorder.

69. The method of claim 66, wherein the neurological disorder, psychiatric disorder, or both, comprises treatment resistant depression, suicidal ideation, major depressive disorder, bipolar disorder, schizophrenia, or substance use disorder.

70. The method of claim 66, wherein the neurological disorder, psychiatric disorder, or both, comprises stroke, traumatic brain injury, or a combination thereof.

71. The method of claim 66, further comprising administering to the subject an effective amount of an empathogenic agent.

72. The method of claim 71, wherein the empathogenic agent is MDMA.

73. The method of claim 66, further comprising administering a 5-HT_{2A} antagonist to the subject.

74. The method of claim 73, wherein the 5-HT_{2A} antagonist is selected from MDL-11,939, eplivanserin (SR-46,349), ketanserin, ritanserin, altanserin, acepromazine, mianserin, mirtazapine, quetiapine, SB204741, SB206553, SB242084, LY272015, SB243213, blonanserin, SB200646, RS102221, nefazodone, volinanserin (MDL-100,907), olanzapine, risperidone, pimavanserin, nelotanserin and lorcaserin.
